(19) United States
(12) Patent Application Publication
(10) Pub. No.: US 2008/0168572 A1
(43) Pub. Date: Jul. 10, 2008

(54) Nanoarchaeum genome, Nanoarchaeum polypeptides and nucleic acids encoding them and methods for making and using them

(75) Inventors: Karl O. Stetter, Regensubrg (DE); Elizabeth Waters, San Diego, CA (US); Keith Kretz, San Marcos, CA (US); Mircea Podar, San Diego, CA (US); Toby Richardson, San Diego, CA (US); Michiel Noordewier, San Diego, CA (US)

Correspondence Address: VERENIUM C/O MOFO S.D., 12531 HIGH BLUFF DRIVE, SUITE 100, SAN DIEGO, CA, 92130-2040, US

(73) Assignee: Diversa Corporation, San Diego, CA (US)

(21) Appl. No.: 10/428,340

(22) Filed: May 1, 2003

Related U.S. Application Data
(60) Provisional application No. 60/377,447, filed on May 1, 2002.

(51) Int. Cl. A01K 67/00; C07H 21/04; C07K 16/00; C12N 15/00; C12N 1/34; C12Q 1/68; C40B 50/06; G01N 33/48; G06G 7/48; C40B 40/10; G01N 33/53; C12N 5/06; C12N 5/00; A01H 5/00

(52) U.S. Cl. 800/8; 536/23.2; 536/24.3; 536/24.33; 530/350; 435/320.1; 435/325; 800/298; 536/24.5; 435/375; 435/183; 530/387.3; 506/18; 435/7.1; 435/6; 703/11; 702/19; 536/25.41; 506/26; 800/288; 435/252.1

(57) ABSTRACT

The invention provides a genome of the hyperthermophile Nanoarchaeum equitans, polypeptides, including enzymes, structural protein and binding proteins, derived from this genome, polynucleotides encoding these polypeptides, methods of making and using these polynucleotides and polypeptides. The invention also provides isolated hyperthermophile Nanoarchaeum equitans.

### RELATED APPLICATIONS

This application claims the benefit of priority under 35 U.S.C. § 119(e) of U.S. Provisional Application No. 60/377,447, filed May 1, 2002. The aforementioned application is explicitly incorporated herein by reference in its entirety and for all purposes.

### REFERENCE TO SEQUENCE LISTING SUBMITTED ON A COMPACT DISC

This application includes a compact disc (submitted in duplicate) containing a sequence listing. The entire content of the sequence listing is herein incorporated by reference. The sequence listing is identified on the compact disc as follows.

| File Name | Date of Creation | Size (bytes) |
|---|---|---|
| Sequence Listing.txt | May 1, 2003 | 2,592,438 |

### FIELD OF THE INVENTION

This invention relates generally to microbiology. In alternative aspects, the invention provides a genome of the hyperthermophile Nanoarchaeum equitans, polypeptides, including enzymes, structural proteins and binding proteins derived from this genome, polynucleotides encoding these polypeptides, methods of making and using the genome and these polynucleotides and polypeptides. The invention also provides isolated hyperthermophile Nanoarchaeum equitans.

### BACKGROUND

Several theoretical and experimental studies have endeavored to derive the minimal set of genes that are necessary and sufficient to sustain a functioning cell under ideal conditions, that is, in the presence of unlimited amounts of all essential nutrients and in the absence of any adverse factors, including competition. A comparison of the first two completed bacterial genomes, those of the parasites Haemophilus influenzae and Mycoplasma genitalium, produced a version of the minimal gene set consisting of approximately 250 genes. Very similar estimates were obtained by analyzing viable gene knockouts in Bacillus subtilis, M. genitalium, and Mycoplasma pneumoniae. With the accumulation and comparison of multiple complete genome sequences, it became clear that only about 80 genes of the 250 in the original minimal gene set are represented by orthologs in all life forms. For approximately 15% of the genes from the minimal gene set, viable knockouts were obtained in M. genitalium. Unexpectedly, these included even some of the universal genes. Thus, some of the genes that were included in the first version of the minimal gene set, based on a limited genome comparison, could be, in fact, dispensable. The majority of these genes, however, are likely to encode essential functions but, in the course of evolution, are subject to non-orthologous gene displacement, that is, recruitment of unrelated or distantly related proteins for the same function. Further theoretical and experimental studies within the framework of the minimal-gene-set concept and the ultimate construction of a minimal genome are expected to advance our understanding of the basic principles of cell functioning by systematically detecting non-orthologous gene displacement and deciphering the roles of essential but functionally uncharacterized genes. Global knockout mutagenesis of the mycoplasmal genes, aimed at delineating a minimal gene set, has resulted in estimates that are very similar to those produced by original comparative genomic analysis but has also shown that even some of the universal or highly conserved genes can be dispensable. These results could indicate that even absolute evolutionary conservation does not automatically entail indispensability of a gene under any conditions, but their definitive interpretation requires further experiments.

### SUMMARY OF THE INVENTION

The invention provides an isolated and/or a recombinant genome of a hyperthermophile Nanoarchaeum equitans (SEQ ID NO:1). The invention also provides an isolated hyperthermophile Nanoarchaeum equitans deposited as ATCC accession no. ______.

The invention provides isolated or recombinant nucleic acids comprising a nucleic acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention, e.g., SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, over a region of at least about 10, 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, or more residues, encodes at least one polypeptide having an enzyme, structural or binding activity, and the sequence identities are determined by analysis with a sequence comparison algorithm or by a visual inspection.

In alternative aspects, the isolated or recombinant nucleic acid encodes a polypeptide comprising a sequence as set forth in SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, and all polypeptides disclosed in the SEQ ID listing, which include all odd numbered SEQ ID NO:s from SEQ ID NO:3 through SEQ ID NO:1073. In one aspect these polypeptides have an enzyme, structural or binding activity.

In one aspect, the sequence comparison algorithm is a BLAST algorithm, such as a BLAST version 2.2.2 algorithm. In one aspect, the filtering setting is set to blastall -p blastp -d “nr pataa”-F F and all other options are set to default.

In alternative aspects, the enzyme, structural or binding activity comprises a recombinase activity, a helicase activity, a DNA replication activity, a DNA recombination activity, an isomerase, a transisomerase activity or topoisomerase activity, a methyl transferase activity, an aminotransferase activity, a uracil-5-methyl transferase activity, a cysteinyl tRNA synthetase activity, a hydrolase, an esterase activity, a phosphoesterase activity, an acetylmuramyl pentapeptide phosphotransferase activity, a glycosyltransferase activity, an acetyltransferase activity, an acetylglucosamine phosphate transferase activity, a centromere binding activity, a telomerase activity or a transcriptional regulatory activity, a heat shock protein activity, a protease activity, a proteinase activity, a peptidase activity, a carboxypeptidase activity, an endonuclease activity, an exonuclease activity, a RecB family exonuclease activity, a polymerase activity, a carbamoyl phosphate synthetase activity, a methyl-thioadenine synthetase activity, an oxidoreductase activity, an Fe—S oxidoreductase activity, a flavodoxin reductase activity, a permease activity, a thymidylate activity, a dehydrogenase activity, a pyrophosphorylase activity, a coenzyme metabolism activity, a dinucleotide-utilizing enzyme activity, a molybdopterin or thiamine biosynthesis activity, a beta-lactamase activity, a ligand binding activity, an ion transport activity, an ion metabolism activity, a tellurite resistance protein activity, an inorganic ion transport activity, a nucleotide transport activity, a nucleotide metabolism activity, an actin or myosin activity, a lipase activity or a lipid acyl hydrolase (LAH) activity, a cell envelop biogenesis activity, an outer membrane synthesis activity, a ribosomal structure synthesis activity, a translational processing activity, a transcriptional initiation activity, a TATA-binding activity, a signal transduction activity, an energy metabolism activity, an ATPase activity, an information storage and/or processing activity, and/or any of the polypeptides activities as set forth in Table 3, below.

In one aspect, the isolated or recombinant nucleic acid encodes a polypeptide having an enzyme, structural or binding activity which is thermostable. The polypeptide can retain an enzyme, structural or binding activity under conditions comprising a temperature range of between about 37° C. to about 95° C.; between about 55° C. to about 85° C., between about 70° C. to about 95° C., or, between about 90° C. to about 95° C., about 96° C., about 97° C., about 98° C., or more. In another aspect, the isolated or recombinant nucleic acid encodes a polypeptide having an enzyme, structural or binding activity which is thermotolerant. The polypeptide can retain an enzyme, structural or binding activity after exposure to a temperature in the range from greater than 37° C. to about 95° C., about 96° C., about 97° C., about 98° C., or more or anywhere in the range from greater than 55° C. to about 85° C. In one aspect, the polypeptide retains an enzyme, structural or binding activity after exposure to a temperature in the range from greater than 90° C. to about 95° C., about 96° C., about 97° C., about 98° C., or more, at about pH 7, pH 6.5, pH 6.0, pH 5.5, pH 5, or pH 4.5.

The polypeptide can retain an enzyme, structural or binding activity under conditions comprising about pH 7, pH 6.5, pH 6.0, pH 5.5, pH 5, or pH 4.5. The polypeptide can retain an enzyme, structural or binding activity under these conditions comprising a temperature range of between about 40° C. to about 70° C., or, between about 90° C. to about 95° C., or more.

In one aspect, the isolated or recombinant nucleic acid comprises a sequence that hybridizes under stringent conditions to a sequence of the invention, wherein the nucleic acid encodes a polypeptide having an enzyme, structural or binding activity. The nucleic acid can at least about 10, 20, 30, 40, 50, 60, 70, 80, 90, 100, 150, 200, 250, 300, 350, 400, 450, 500, 550, 600, 650, 700, 750, 800, 850 or residues in length or the full length of the gene or transcript, with or without a signal sequence, or a propro sequence (e.g., as with a protease), as described herein. The stringent conditions can be highly stringent, moderately stringent or of low stringency, as described herein. The stringent conditions can include a wash step, e.g., a wash step comprising a wash in 0.2×SSC at a temperature of about 65° C. for about 15 minutes.

The invention provides a nucleic acid probe for identifying a nucleic acid encoding a polypeptide, e.g., with an enzyme, structural or binding activity, wherein the probe comprises at least 10, 20, 30, 40, 50, 60, 70, 80, 90, 100, 150, 200, 250, 300, 350, 400, 450, 500, 550, 600, 650, 700, 750, 800, 850, or more, consecutive bases of a sequence of the invention and the probe identifies the nucleic acid by binding or hybridization. The probe can comprise an oligonucleotide comprising at least about 10 to 50, about 20 to 60, about 30 to 70, about 40 to 80, or about 60 to 100 consecutive bases of a sequence of the invention.

The invention provides a nucleic acid probe for identifying a nucleic acid encoding a polypeptide with an enzyme, structural or binding activity, wherein the probe comprises a nucleic acid of the invention, e.g., a nucleic acid having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all additional nucleic acids disclosed in the SEQ ID listing, which include all even numbered SEQ ID NO:s from SEQ ID NO:2 through SEQ ID NO:1073, or a subsequence thereof, over a region of at least about 10, 20, 30, 40, 50, 60, 70, 80, 90, 100, 150, 200, 250, 300, 350, 400, 450, 500, 550, 600, 650, 700, 750, 800, 850 or more consecutive residues, wherein the sequence identities are determined by analysis with a sequence comparison algorithm or by visual inspection.

The invention provides an amplification primer sequence pair for amplifying a nucleic acid encoding a polypeptide having, e.g., an enzyme, structural or binding activity, wherein the primer pair is capable of amplifying a nucleic acid comprising a sequence of the invention, or fragments or subsequences thereof. One or each member of the amplification primer sequence pair can comprise an oligonucleotide comprising at least about 10 to 50 consecutive bases of the sequence, or about 12, 13, 14, 15, 16, 17, 18, 19, 20, 21, 22, 23, 24, or 25 consecutive bases of the sequence.

The invention provides amplification primer pairs, wherein the primer pair comprises a first member having a sequence as set forth by about the first (the 5′) 12, 13, 14, 15, 16, 17, 18, 19, 20, 21, 22, 23, 24, or 25 residues of a nucleic acid of the invention, and a second member having a sequence as set forth by about the first (the 5′) 12, 13, 14, 15, 16, 17, 18, 19, 20, 21, 22, 23, 24, or 25 residues of the complementary strand of the first member.

The invention provides polypeptides generated by amplification, e.g., polymerase chain reaction (PCR), using an amplification primer pair of the invention. The invention provides methods of making a polypeptide by amplification, e.g., polymerase chain reaction (PCR), using an amplification primer pair of the invention. In one aspect, the amplification primer pair amplifies a nucleic acid from a library, e.g., a gene library, such as an environmental library.

The invention provides methods of amplifying a nucleic acid encoding a polypeptide comprising amplification of a template nucleic acid with an amplification primer sequence pair capable of amplifying a nucleic acid sequence of the invention, or fragments or subsequences thereof. The amplification primer pair can be an amplification primer pair of the invention.

The invention provides expression cassettes comprising a nucleic acid of the invention or a subsequence thereof. In one aspect, the expression cassette can comprise the nucleic acid that is operably linked to a promoter. The promoter can be a viral, bacterial, mammalian or plant promoter. In one aspect, the plant promoter can be a potato, rice, corn, wheat, tobacco or barley promoter. The promoter can be a constitutive promoter. The constitutive promoter can comprise CaMV35S. In another aspect, the promoter can be an inducible promoter. In one aspect, the promoter can be a tissue-specific promoter or an environmentally regulated or a developmentally regulated promoter. Thus, the promoter can be, e.g., a seed-specific, a leaf-specific, a root-specific, a stem-specific or an abscission-induced promoter. In one aspect, the expression cassette can further comprise a plant or plant virus expression vector.

The invention provides cloning vehicles comprising an expression cassette (e.g., a vector) of the invention or a nucleic acid of the invention. The cloning vehicle can be a viral vector, a plasmid, a phage, a phagemid, a cosmid, a fosmid, a bacteriophage or an artificial chromosome. The viral vector can comprise an adenovirus vector, a retroviral vector or an adeno-associated viral vector. The cloning vehicle can comprise a bacterial artificial chromosome (BAC), a plasmid, a bacteriophage P1-derived vector (PAC), a yeast artificial chromosome (YAC), or a mammalian artificial chromosome (MAC).

The invention provides transformed cell comprising a nucleic acid of the invention or an expression cassette (e.g., a vector) of the invention, or a cloning vehicle of the invention. In one aspect, the transformed cell can be a bacterial cell, a mammalian cell, a fungal cell, a yeast cell, an insect cell or a plant cell. In one aspect, the plant cell can be a potato, wheat, rice, corn, tobacco or barley cell.

The invention provides transgenic non-human animals comprising a nucleic acid of the invention or an expression cassette (e.g., a vector) of the invention. In one aspect, the animal is a mouse.

The invention provides transgenic plants comprising a nucleic acid of the invention or an expression cassette (e.g., a vector) of the invention. The transgenic plant can be a corn plant, a potato plant, a tomato plant, a wheat plant, an oilseed plant, a rapeseed plant, a soybean plant, a rice plant, a barley plant or a tobacco plant. The invention provides transgenic seeds comprising a nucleic acid of the invention or an expression cassette (e.g., a vector) of the invention. The transgenic seed can be a corn seed, a wheat kernel, an oilseed, a rapeseed (a canola plant), a soybean seed, a palm kernel, a sunflower seed, a sesame seed, a peanut or a tobacco plant seed.

The invention provides an antisense oligonucleotide comprising a nucleic acid sequence complementary to or capable of hybridizing under stringent conditions to a nucleic acid of the invention. The invention provides methods of inhibiting the translation of a polypeptide message in a cell comprising administering to the cell or expressing in the cell an antisense oligonucleotide comprising a nucleic acid sequence complementary to or capable of hybridizing under stringent conditions to a nucleic acid of the invention.

The invention provides an antisense oligonucleotide comprising a nucleic acid sequence complementary to or capable of hybridizing under stringent conditions to a nucleic acid of the invention. The invention provides methods of inhibiting the translation of a polypeptide message in a cell comprising administering to the cell or expressing in the cell an antisense oligonucleotide comprising a nucleic acid sequence complementary to or capable of hybridizing under stringent conditions to a nucleic acid of the invention. The antisense oligonucleotide can be between about 10 to 50, about 20 to 60, about 30 to 70, about 40 to 80, about 60 to 100, about 70 to 110, or about 80 to 120 bases in length.

The invention provides methods of inhibiting the translation of message in a cell comprising administering to the cell or expressing in the cell an antisense oligonucleotide comprising a nucleic acid sequence complementary to or capable of hybridizing under stringent conditions to a nucleic acid of the invention. The invention provides double-stranded inhibitory RNA (RNAi) molecules comprising a subsequence of a sequence of the invention. In one aspect, the RNAi is about 15, 16, 17, 18, 19, 20, 21, 22, 23, 24, 25 or more duplex nucleotides in length. The invention provides methods of inhibiting the expression of a message in a cell comprising administering to the cell or expressing in the cell a double-stranded inhibitory RNA (iRNA), wherein the RNA comprises a subsequence of a sequence of the invention.

The invention provides isolated or recombinant polypeptides encoded by a nucleic acid of the invention. In alternative aspects, the polypeptide can have a sequence as set forth in SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, and all polypeptides disclosed in the SEQ ID listing, which include all odd numbered SEQ ID NO:s from SEQ ID NO:3 through SEQ ID NO:1073. The polypeptide can have an enzyme, structural or binding activity.

In alternative aspects, the enzyme, structural or binding activity comprises a recombinase activity, a helicase activity, a DNA replication activity, a DNA recombination activity, an isomerase, a transisomerase activity or topoisomerase activity, a methyl transferase activity, an aminotransferase activity, a uracil-5-methyl transferase activity, a cysteinyl tRNA synthetase activity, a hydrolase, an esterase activity, a phosphoesterase activity, an acetylmuramyl pentapeptide phosphotransferase activity, a glycosyltransferase activity, an acetyltransferase activity, an acetylglucosamine phosphate transferase activity, a centromere binding activity, a telomerase activity or a transcriptional regulatory activity, a heat shock protein activity, a protease activity, a proteinase activity, a peptidase activity, a carboxypeptidase activity, an endonuclease activity, an exonuclease activity, a RecB family exonuclease activity, a polymerase activity, a carbamoyl phosphate synthetase activity, a methyl-thioadenine synthetase activity, an oxidoreductase activity, an Fe—S oxidoreductase activity, a flavodoxin reductase activity, a permease activity, a thymidylate activity, a dehydrogenase activity, a pyrophosphorylase activity, a coenzyme metabolism activity, a dinucleotide-utilizing enzyme activity, a molybdopterin or thiamine biosynthesis activity, a beta-lactamase activity, a ligand binding activity, an ion transport activity, an ion metabolism activity, a tellurite resistance protein activity, an inorganic ion transport activity, a nucleotide transport activity, a nucleotide metabolism activity, an actin or myosin activity, a lipase activity or a lipid acyl hydrolase (LAH) activity, a cell envelop biogenesis activity, an outer membrane synthesis activity, a ribosomal structure synthesis activity, a translational processing activity, a transcriptional initiation activity, a TATA-binding activity, a signal transduction activity, an energy metabolism activity, an ATPase activity, an information storage and/or processing activity, and/or any of the polypeptides activities as set forth in Table 3, below.

The invention provides isolated or recombinant polypeptides comprising a polypeptide of the invention lacking a signal sequence and/or a prepro sequence.

Another aspect of the invention provides an isolated or recombinant polypeptide or peptide including at least 10, 15, 20, 25, 30, 35, 40, 45, 50, 55, 60, 65, 70, 75, 80, 85, 90, 95 or 100 or more consecutive bases of a polypeptide or peptide sequence of the invention, sequences substantially identical thereto, and the sequences complementary thereto. The peptide can be, e.g., an immunogenic fragment, a motif (e.g., a binding site) or an active site.

In one aspect, the isolated or recombinant polypeptide of the invention (with or without a signal sequence or a prepro sequence) has an enzyme, structural or binding activity.

In one aspect, the enzyme, structural or binding activity is thermostable. The polypeptide can retain an enzyme, structural or binding activity under conditions comprising a temperature range of between about 37° C. to about 95° C., between about 55° C. to about 85° C., between about 70° C. to about 95° C., or between about 90° C. to about 95° C. In another aspect, the enzyme, structural or binding activity can be thermotolerant. The polypeptide can retain an enzyme, structural or binding activity after exposure to a temperature in the range from greater than 37° C. to about 95° C., or in the range from greater than 55° C. to about 85° C. In one aspect, the polypeptide can retain an enzyme, structural or binding activity after exposure to a temperature in the range from greater than 90° C. to about 95° C. at pH 4.5.

In one aspect, the polypeptide can retain an enzyme, structural or binding activity under conditions comprising about pH 6.5, pH 6, pH 5.5, pH 5, pH 4.5 or pH 4. In another aspect, the polypeptide can retain an enzyme, structural or binding activity under conditions comprising about pH 7, pH 7.5 pH 8.0, pH 8.5, pH 9, pH 9.5, pH 10, pH 10.5 or pH 11.

In one aspect, the isolated or recombinant polypeptide can comprise the polypeptide of the invention that lacks a signal sequence and/or a prepro domain. In one aspect, the isolated or recombinant polypeptide can comprise the polypeptide of the invention comprising a heterologous signal sequence or a heterologous preprosequence, such as a heterologous enzyme or non-enzyme signal sequence.

The invention provides isolated or recombinant peptides comprising an amino acid sequence having at least 95%, 96%, 97%, 98%, 99%, or more sequence identity to a signal sequence and/or a prepro sequence of the invention, wherein the sequence identities are determined by analysis with a sequence comparison algorithm or by visual inspection. In one aspect, the peptides act as signal sequences on its endogenous polypeptide, on another enzyme, or a heterologous protein. In one aspect, the invention provides chimeric proteins comprising a first domain comprising a signal sequence of the invention and at least a second domain. The protein can be a fusion protein. The second domain can comprise an enzyme.

The invention provides chimeric polypeptides comprising at least a first domain comprising signal peptide (SP) and/or a prepro sequence of the invention or a catalytic domain (CD), or active site, of an enzyme of the invention and at least a second domain comprising a heterologous polypeptide or peptide, wherein the heterologous polypeptide or peptide is not naturally associated with the signal peptide (SP), prepro sequence or catalytic domain (CD). In one aspect, the heterologous polypeptide or peptide is not an enzyme. The heterologous polypeptide or peptide can be amino terminal to, carboxy terminal to or on both ends of the signal peptide (SP), prepro sequence or catalytic domain.

The invention provides isolated or recombinant nucleic acids encoding a chimeric polypeptide, wherein the chimeric polypeptide comprises at least a first domain comprising signal peptide (SP), prepro sequence or a catalytic domain (CD), or active site, of a polypeptide of the invention, and at least a second domain comprising a heterologous polypeptide or peptide, wherein the heterologous polypeptide or peptide is not naturally associated with the signal peptide (SP), prepro sequence or catalytic domain (CD).

In one aspect, the enzyme, structural or binding activity comprises a specific activity at about 37° C. in the range from about 1 to about 1000 units, or about 10 to 100 units per milligram of protein. In another aspect, the enzyme, structural or binding activity comprises a specific activity from about 500 to about 750 units per milligram of protein. Alternatively, the enzyme, structural or binding activity comprises a specific activity at 37° C. in the range from about 1 to 1000, or about 500 to about 1200 units per milligram of protein. In one aspect, the enzyme, structural or binding activity comprises a specific activity at 37° C. in the range from about 750 to about 1000 units per milligram of protein. In another aspect, the thermotolerance comprises retention of at least half of the specific activity of the enzyme, structural or binding at 37° C. after being heated to the elevated temperature. Alternatively, the thermotolerance can comprise retention of specific activity at 37° C. in the range from about 500 to about 1200 units per milligram of protein after being heated to the elevated temperature.

The invention provides the isolated or recombinant polypeptide of the invention, wherein the polypeptide comprises at least one glycosylation site. In one aspect, glycosylation can be an N-linked glycosylation. In one aspect, the polypeptide can be glycosylated after being expressed in a P. pastoris or a S. pombe.

The invention provides protein preparations comprising a polypeptide of the invention, wherein the protein preparation comprises a liquid, a solid or a gel.

The invention provides heterodimers comprising a polypeptide of the invention and a second protein or domain. The second member of the heterodimer can be a different enzyme, a different enzyme or another protein. In one aspect, the second domain can be a polypeptide and the heterodimer can be a fusion protein. In one aspect, the second domain can be an epitope or a tag. In one aspect, the invention provides homodimers comprising a polypeptide of the invention.

The invention provides immobilized polypeptides having an enzyme, structural or binding activity, wherein the polypeptide comprises a polypeptide of the invention, a polypeptide encoded by a nucleic acid of the invention, or a polypeptide comprising a polypeptide of the invention and a second domain. In one aspect, the polypeptide can be immobilized on a cell, a metal, a resin, a polymer, a ceramic, a glass, a microelectrode, a graphitic particle, a bead, a gel, a plate, an array or a capillary tube.

The invention provides arrays comprising an immobilized polypeptide of the invention or a polypeptide encoded by a nucleic acid of the invention. The invention provides arrays comprising an immobilized nucleic acid of the invention. The invention provides an array comprising an immobilized antibody of the invention.

The invention provides isolated or recombinant antibodies that specifically bind to a polypeptide of the invention or to a polypeptide encoded by a nucleic acid of the invention. The antibody can be a monoclonal or a polyclonal antibody. The invention provides hybridomas comprising an antibody of the invention.

The invention provides methods of isolating or identifying a polypeptide with an enzyme, structural or binding activity comprising the steps of: (a) providing an antibody of the invention; (b) providing a sample comprising polypeptides; and, (c) contacting the sample of step (b) with the antibody of step (a) under conditions wherein the antibody can specifically bind to the polypeptide, thereby isolating or identifying polypeptide. The invention provides methods of making an antibody comprising administering to a non-human animal a nucleic acid of the invention, or a polypeptide of the invention, in an amount sufficient to generate a humoral immune response, thereby making an antibody.

The invention provides methods of producing a recombinant polypeptide comprising the steps of: (a) providing a nucleic acid of the invention operably linked to a promoter; and, (b) expressing the nucleic acid of step (a) under conditions that allow expression of the polypeptide, thereby producing a recombinant polypeptide. The method can further comprise transforming a host cell with the nucleic acid of step (a) followed by expressing the nucleic acid of step (a), thereby producing a recombinant polypeptide in a transformed cell. The method can further comprise inserting into a host non-human animal the nucleic acid of step (a) followed by expressing the nucleic acid of step (a), thereby producing a recombinant polypeptide in the host non-human animal.

The invention provides methods for identifying a polypeptide having an enzyme activity comprising the following steps: (a) providing a polypeptide of the invention or a polypeptide encoded by a nucleic acid of the invention, or a fragment or variant thereof, (b) providing an enzyme substrate; and, (c) contacting the polypeptide or a fragment or variant thereof of step (a) with the substrate of step (b) and detecting an increase in the amount of substrate or a decrease in the amount of reaction product, wherein a decrease in the amount of the substrate or an increase in the amount of the reaction product detects a polypeptide having an enzyme activity.

The invention provides methods for identifying an enzyme substrate comprising the following steps: (a) providing a polypeptide of the invention or a polypeptide encoded by a nucleic acid of the invention; (b) providing a test substrate; and, (c) contacting the polypeptide of step (a) with the test substrate of step (b) and detecting an increase in the amount of substrate or a decrease in the amount of reaction product, wherein a decrease in the amount of the substrate or an increase in the amount of the reaction product identifies the test substrate as an enzyme substrate.

The invention provides methods of determining whether a compound specifically binds to a polypeptide comprising the following steps: (a) expressing a nucleic acid or a vector comprising the nucleic acid under conditions permissive for translation of the nucleic acid to a polypeptide, wherein the nucleic acid and vector comprise a nucleic acid or vector of the invention; or, providing a polypeptide of the invention (b) contacting the polypeptide with the test compound; and, (c) determining whether the test compound specifically binds to the polypeptide, thereby determining that the compound specifically binds to the polypeptide.

The invention provides methods for identifying a modulator of a polypeptide activity comprising the following steps: (a) providing a polypeptide of the invention or a polypeptide encoded by a nucleic acid of the invention; (b) providing a test compound; (c) contacting the polypeptide of step (a) with the test compound of step (b); and, measuring an activity of the polypeptide, wherein a change in the polypeptide activity measured in the presence of the test compound compared to the activity in the absence of the test compound provides a determination that the test compound modulates the polypeptide activity.

In one aspect, the enzyme activity is measured by providing an enzyme substrate and detecting an increase in the amount of the substrate or a decrease in the amount of a reaction product. The decrease in the amount of the substrate or the increase in the amount of the reaction product with the test compound as compared to the amount of substrate or reaction product without the test compound identifies the test compound as an activator of enzyme activity. The increase in the amount of the substrate or the decrease in the amount of the reaction product with the test compound as compared to the amount of substrate or reaction product without the test compound identifies the test compound as an inhibitor of enzyme activity.

The invention provides computer systems comprising a processor and a data storage device wherein said data storage device has stored thereon a polypeptide sequence of the invention or a nucleic acid sequence of the invention.

In one aspect, the computer system can further comprise a sequence comparison algorithm and a data storage device having at least one reference sequence stored thereon. The sequence comparison algorithm can comprise a computer program that indicates polymorphisms. The computer system can further comprising an identifier that identifies one or more features in said sequence.

The invention provides computer readable mediums having stored thereon a sequence comprising a polypeptide sequence of the invention or a nucleic acid sequence of the invention.

The invention provides methods for identifying a feature in a sequence comprising the steps of: (a) reading the sequence using a computer program which identifies one or more features in a sequence, wherein the sequence comprises a polypeptide sequence of the invention or a nucleic acid sequence of the invention; and, (b) identifying one or more features in the sequence with the computer program.

The invention provides methods for comparing a first sequence to a second sequence comprising the steps of: (a) reading the first sequence and the second sequence through use of a computer program which compares sequences, wherein the first sequence comprises a polypeptide sequence of the invention or a nucleic acid sequence of the invention; and, (b) determining differences between the first sequence and the second sequence with the computer program. In one aspect, the step of determining differences between the first sequence and the second sequence further comprises the step of identifying polymorphisms. In one aspect, the method further comprises an identifier (and use of the identifier) that identifies one or more features in a sequence. In one aspect, the method comprises reading the first sequence using a computer program and identifying one or more features in the sequence.

The invention provides methods for isolating or recovering a nucleic acid encoding a polypeptide with an activity, e.g., an enzyme, structural or transcriptional control activity, from an environmental sample comprising the steps of: (a) providing an amplification primer sequence pair for amplifying a nucleic acid encoding a polypeptide with an activity, wherein the primer pair is capable of amplifying a nucleic acid of the invention; (b) isolating a nucleic acid from the environmental sample or treating the environmental sample such that nucleic acid in the sample is accessible for hybridization to the amplification primer pair; and, (c) combining the nucleic acid of step (b) with the amplification primer pair of step (a) and amplifying nucleic acid from the environmental sample, thereby isolating or recovering a nucleic acid encoding a polypeptide with an activity from an environmental sample. In one aspect, each member of the amplification primer sequence pair comprises an oligonucleotide comprising at least about 10 to 50 consecutive bases of a nucleic acid sequence of the invention. In one aspect, the amplification primer sequence pair is an amplification pair of the invention.

The invention provides methods for isolating or recovering a nucleic acid encoding a polypeptide with an activity, e.g., an enzyme, structural or transcriptional control activity, from an environmental sample comprising the steps of: (a) providing a polynucleotide probe comprising a nucleic acid sequence of the invention, or a subsequence thereof; (b) isolating a nucleic acid from the environmental sample or treating the environmental sample such that nucleic acid in the sample is accessible for hybridization to a polynucleotide probe of step (a); (c) combining the isolated nucleic acid or the treated environmental sample of step (b) with the polynucleotide probe of step (a); and, (d) isolating a nucleic acid that specifically hybridizes with the polynucleotide probe of step (a), thereby isolating or recovering a nucleic acid encoding a polypeptide with an activity from the environmental sample. In alternative aspects, the environmental sample comprises a water sample, a liquid sample, a soil sample, an air sample or a biological sample. In alternative aspects, the biological sample is derived from a bacterial cell, a protozoan cell, an insect cell, a yeast cell, a plant cell, a fungal cell or a mammalian cell.

The invention provides methods of generating a variant of a nucleic acid encoding a polypeptide comprising the steps of: (a) providing a template nucleic acid comprising a nucleic acid of the invention; (b) modifying, deleting or adding one or more nucleotides in the template sequence, or a combination thereof, to generate a variant of the template nucleic acid. In one aspect, the method further comprises expressing the variant nucleic acid to generate a variant polypeptide. In alternative aspects, the modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR) and/or a combination thereof. In alternative aspects, the modifications, additions or deletions are introduced by a method selected from the group consisting of recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and/or a combination thereof.

In one aspect, the method is iteratively repeated until a polypeptide (e.g., an enzyme) having an altered or different activity or an altered or different stability from that of a polypeptide encoded by the template nucleic acid is produced. In one aspect, the altered or different activity is a polypeptide (e.g., an enzyme) activity under an acidic condition, wherein the polypeptide encoded by the template nucleic acid is not active under the acidic condition. In one aspect, the altered or different activity is a polypeptide (e.g., an enzyme) activity under a high temperature, wherein the polypeptide encoded by the template nucleic acid is not active under the high temperature. In one aspect, the method is iteratively repeated until a polypeptide coding sequence having an altered codon usage from that of the template nucleic acid is produced. The method can be iteratively repeated until a gene having higher or lower level of message expression or stability from that of the template nucleic acid is produced.

The invention provides methods for modifying codons in a nucleic acid encoding an enzyme to increase its expression in a host cell, the method comprising (a) providing a nucleic acid of the invention encoding an enzyme; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying codons in a nucleic acid encoding an enzyme, the method comprising (a) providing a nucleic acid of the invention encoding an enzyme; and, (b) identifying a codon in the nucleic acid of step (a) and replacing it with a different codon encoding the same amino acid as the replaced codon, thereby modifying codons in a nucleic acid encoding an enzyme.

The invention provides methods for modifying codons in a nucleic acid encoding an enzyme to increase its expression in a host cell, the method comprising (a) providing a nucleic acid of the invention encoding an enzyme; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying a codon in a nucleic acid encoding an enzyme to decrease its expression in a host cell, the method comprising (a) providing a nucleic acid of the invention encoding an enzyme; and, (b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell. In alternative aspects, the host cell is a bacterial cell, a fungal cell, an insect cell, a yeast cell, a plant cell or a mammalian cell.

The invention provides methods for producing a library of nucleic acids encoding a plurality of modified enzyme active sites or substrate binding sites, wherein the modified active sites or substrate binding sites are derived from a first nucleic acid comprising a sequence encoding a first active site or a first substrate binding site the method comprising: (a) providing a first nucleic acid encoding a first active site or first substrate binding site, wherein the first nucleic acid sequence comprises a nucleic acid of the invention; (b) providing a set of mutagenic oligonucleotides that encode naturally-occurring amino acid variants at a plurality of targeted codons in the first nucleic acid; and, (c) using the set of mutagenic oligonucleotides to generate a set of active site-encoding or substrate binding site-encoding variant nucleic acids encoding a range of amino acid variations at each amino acid codon that was mutagenized, thereby producing a library of nucleic acids encoding a plurality of modified enzyme active sites or substrate binding sites. In alternative aspects, the method comprises mutagenizing the first nucleic acid of step (a) by a method comprising an optimized directed evolution system, gene site-saturation mutagenesis (GSSM), and synthetic ligation reassembly (SLR). The method can further comprise mutagenizing the first nucleic acid of step (a) or variants by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR) and a combination thereof. The method can further comprise mutagenizing the first nucleic acid of step (a) or variants by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

The invention provides methods for making a small molecule comprising the steps of: (a) providing a plurality of biosynthetic enzymes capable of synthesizing or modifying a small molecule, wherein one of the enzymes comprises an enzyme encoded by a nucleic acid of the invention; (b) providing a substrate for at least one of the enzymes of step (a); and, (c) reacting the substrate of step (b) with the enzymes under conditions that facilitate a plurality of biocatalytic reactions to generate a small molecule by a series of biocatalytic reactions.

The invention provides methods for modifying a small molecule comprising the steps: (a) providing a enzyme encoded by a nucleic acid of the invention; (b) providing a small molecule; and, (c) reacting the enzyme of step (a) with the small molecule of step (b) under conditions that facilitate an enzymatic reaction catalyzed by the enzyme, thereby modifying a small molecule by an enzymatic reaction. In one aspect, the method comprises providing a plurality of small molecule substrates for the enzyme of step (a), thereby generating a library of modified small molecules produced by at least one enzymatic reaction catalyzed by the enzyme. In one aspect, the method further comprises a plurality of additional enzymes under conditions that facilitate a plurality of biocatalytic reactions by the enzymes to form a library of modified small molecules produced by the plurality of enzymatic reactions. In one aspect, the method further comprises the step of testing the library to determine if a particular modified small molecule that exhibits a desired activity is present within the library. The step of testing the library can further comprises the steps of systematically eliminating all but one of the biocatalytic reactions used to produce a portion of the plurality of the modified small molecules within the library by testing the portion of the modified small molecule for the presence or absence of the particular modified small molecule with a desired activity, and identifying at least one specific biocatalytic reaction that produces the particular modified small molecule of desired activity.

The invention provides methods for determining a functional fragment of an enzyme comprising the steps of: (a) providing an enzyme comprising an amino acid sequence of the invention; and, (b) deleting a plurality of amino acid residues from the sequence of step (a) and testing the remaining subsequence for an enzyme activity, thereby determining a functional fragment of an enzyme. In one aspect, the enzyme activity is measured by providing an enzyme substrate and detecting an increase in the amount of the substrate or a decrease in the amount of a reaction product. In one aspect, a decrease in the amount of an enzyme substrate or an increase in the amount of the reaction product with the test compound as compared to the amount of substrate or reaction product without the test compound identifies the test compound as an activator of enzyme activity.

The invention provides methods for whole cell engineering of new or modified phenotypes by using real-time metabolic flux analysis, the method comprising the following steps: (a) making a modified cell by modifying the genetic composition of a cell, wherein the genetic composition is modified by addition to the cell of a nucleic acid comprising a sequence of the invention; (b) culturing the modified cell to generate a plurality of modified cells; (c) measuring at least one metabolic parameter of the cell by monitoring the cell culture of step (b) in real time; and, (d) analyzing the data of step (c) to determine if the measured parameter differs from a comparable measurement in an unmodified cell under similar conditions, thereby identifying an engineered phenotype in the cell using real-time metabolic flux analysis. In one aspect, the genetic composition of the cell is modified by a method comprising deletion of a sequence or modification of a sequence in the cell, or, knocking out the expression of a gene. The method can further comprise selecting a cell comprising a newly engineered phenotype. The method can further comprise culturing the selected cell, thereby generating a new cell strain comprising a newly engineered phenotype.

The invention provides isolated or recombinant signal sequences consisting of a sequence as set forth in residues 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30 or 1 to 31, 1 to 32, 1 to 33, 1 to 34, 1 to 35, 1 to 36, 1 to 37, 1 to 38, 1 to 39, 1 to 40, 1 to 41, 1 to 42, 1 to 43, 1 to 44, 1 to 45, 1 to 46, 1 to 47, 1 to 48, 1 to 49, 1 to 50, 1 to 51, 1 to 52, 1 to 53, 1 to 54, 1 to 55, of SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, and all polypeptides disclosed in the SEQ ID listing, which include all odd numbered SEQ ID NO:s from SEQ ID NO:3 through SEQ ID NO:1073. The invention provides chimeric polypeptides comprising at least a first domain comprising signal peptide (SP) of the invention, and at least a second domain comprising a heterologous polypeptide or peptide, wherein the heterologous polypeptide or peptide is not naturally associated with the signal peptide (SP). In one aspect, the heterologous polypeptide or peptide is not an enzyme. In one aspect, the heterologous polypeptide or peptide is amino terminal to, carboxy terminal to or on both ends of the signal peptide (SP) or a catalytic domain (CD). The invention provides isolated or recombinant nucleic acids encoding a chimeric polypeptide, wherein the chimeric polypeptide comprises at least a first domain comprising signal peptide of the invention and at least a second domain comprising a heterologous polypeptide or peptide, wherein the heterologous polypeptide or peptide is not naturally associated with the signal peptide (SP).

The invention provides methods of increasing thermotolerance or thermostability of an enzyme polypeptide, the method comprising glycosylating an enzyme, wherein the polypeptide comprises at least thirty contiguous amino acids of a polypeptide of the invention, or a polypeptide encoded by a nucleic acid of the invention, thereby increasing the thermotolerance or thermostability of the enzyme. The invention provides methods of overexpressing a recombinant enzyme in a cell comprising expressing a vector comprising a nucleic acid sequence of the invention, wherein overexpression is effected by use of a high activity promoter, a dicistronic vector or by gene amplification of the vector.

The invention provides methods of making a transgenic plant comprising the following steps: (a) introducing a heterologous nucleic acid sequence into the cell, wherein the heterologous nucleic sequence comprises a sequence of the invention, thereby producing a transformed plant cell; (b) producing a transgenic plant from the transformed cell. In one aspect, step (a) further comprises introducing the heterologous nucleic acid sequence by electroporation or microinjection of plant cell protoplasts. In one aspect, step (a) step (a) comprises introducing the heterologous nucleic acid sequence directly to plant tissue by DNA particle bombardment or by using an Agrobacterium tumefaciens host.

The invention provides methods of expressing a heterologous nucleic acid sequence in a plant cell comprising the following steps: (a) transforming the plant cell with a heterologous nucleic acid sequence operably linked to a promoter, wherein the heterologous nucleic sequence comprises a sequence of the invention; (b) growing the plant under conditions wherein the heterologous nucleic acids sequence is expressed in the plant cell.

The invention provides methods for a building a genome having a desired biological requirement, a desired biological property or a desired metabolic pathway comprising the following steps: (a) providing a minimal autonomous genome, wherein the genome comprises a sequence as set forth in SEQ ID NO:1; and (b) adding back to the minimal autonomous genome of step (a) one or more desired genes, thereby building a genome having a desired biological requirement, a desired biological property or a desired metabolic pathway. In one aspect the desired biological property comprises performing a metabolic function, synthesizing a structure or composition or regulating a cell cycle.

The invention provides methods for a building a minimal autonomous genome comprising the following steps: (a) providing a genome comprising a sequence as set forth in SEQ ID NO:1; and (b) performing global knockout mutagenesis on the genome, or adding genes to the genome, and determining whether a cell comprising the genome can survive or replicate autonomously, thereby building an autonomous minimal genome.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The following drawings are illustrative of embodiments of the invention and are not meant to limit the scope of the invention as encompassed by the claims.

FIG. 1 is a block diagram of a computer system, as described in detail, below.

FIG. 2 is a flow diagram illustrating one aspect of a process 200 for comparing a new nucleotide or protein sequence with a database of sequences in order to determine the homology levels between the new sequence and the sequences in the database, as described in detail, below.

FIG. 3 is a flow diagram illustrating one embodiment of a process in a computer for determining whether two sequences are homologous, as described in detail, below.

FIG. 4 is a flow diagram illustrating one aspect of an identifier process 300 for detecting the presence of a feature in a sequence.

FIG. 5, summarizes data from the alanylation of unfractionated M. jannaschii tRNA by alanyl-tRNA synthetase, as described in detail, below.

FIG. 6 schematically illustrates the phylogenetic position of Nanoarchaeum equitans within the Archaea, as described in detail, below.

Like reference symbols in the various drawings indicate like elements.

### DETAILED DESCRIPTION OF THE INVENTION

The invention provides an isolated and a recombinant genome of a hyperthermophile Nanoarchaeum equitans (SEQ ID NO:1). The invention also provides an isolated hyperthermophile Nanoarchaeum equitans deposited as ATCC accession no. ______. N. equitans is an obligate parasite growing on the crenarchaeon Ignicoccus. A ribosomal protein based phylogenetic analysis places its branching point closest to the root of the archaeal tree.

The invention also provides isolated and recombinant nucleic acids encoding polypeptides, e.g., SEQ ID NO:2, SEQ ID NO:4, SEQ ID NO:6, SEQ ID NO:8, SEQ ID NO: 10, etc., and all additional nucleic acids disclosed in the SEQ ID listing, which include all even numbered SEQ ID NO:s from SEQ ID NO:2 through SEQ ID NO:1073. The invention also provides isolated and recombinant polypeptides, SEQ ID NO:3, SEQ ID NO:5, SEQ ID NO:7, SEQ ID NO:9, SEQ ID NO:11, etc., and all polypeptides disclosed in the SEQ ID listing, which include all odd numbered SEQ ID NO:s from SEQ ID NO:3 through SEQ ID NO:1073.

The N. equitans genome (SEQ ID NO:1) is 490,885 base pairs and encodes the machinery for information processing and repair, but lacks that for lipid, cofactor, amino acid or nucleotide biosynthesis. Unusual features include a quite a few split genes, little evidence of lateral gene transfer, and few operons. The small genome size may be a consequence of genome reduction, and a lack of pseudogenes suggests an early adaptation to the parasitic lifestyle.

To shed light on the phylogenetic relationship among the Archaea, we concatenated and aligned the amino acid sequences of 35 ribosomal proteins. N. equitans was placed with high support at the most basal position within the Archaea in the maximum likelihood, maximum parsimony, and Bayseian trees suggesting that the Nanoarchaeota are the basal kingdom within the Archaea. In contrast to the bacterial small genome parasites, no free-living close relative to N. equitans could be detected so far. Therefore one cannot deduce the genome size of its possible precursor. The assumption of N. equitans being a non-reduced primitive genome would provide exciting possibilities for research about ancient features of parasitism, early organismal development, and also a starting point for the generation of a minimal autonomous genome.

The assumption of the genome of the invention (SEQ ID NO:1), an N. equitans genome, being a non-reduced primitive genome would provide exciting possibilities for research about ancient features of parasitism, early organismal development, and also a starting point for the generation of a minimal autonomous genome. Based on the fact that N. equitans possesses all the machinery for information processing and repair, it would be a perfect model to rebuild a universal genome based on different biological requirements. In contrast to minimize genomes, such as E. coli or Mycoplasma genitalium, in one aspect the invention provides methods for adding back to the genome of the invention (SEQ ID NO:1) all genes necessary to perform one or more desired metabolisms, synthesis of structures, performing cell cycles and other cellular and metabolic functions and the like.

The invention also provides methods using the genome of the invention (SEQ ID NO:1) for experimental studies within the framework of the minimal-gene-set concept and the ultimate construction of a minimal genome, as described, e.g., by Eugene V. Koonin, Annu. Rev. Genomics Hum. Genet. (2000) 01:99-116. These studies will advance our understanding of the basic principles of cell functioning by systematically detecting non-orthologous gene displacement and deciphering the roles of essential but functionally uncharacterized genes. The invention also provides methods using the genome of the invention (SEQ ID NO:1) in global knockout mutagenesis to analyze essential genes.

The invention's discovery of the archaeal kingdom Nanoarchaeota, and its sole species, Nanoarchaeum equitans, raised new questions about the evolution of the Archaea. Although ribosomal RNA sequences placed this species in a basal branch of the Archaea, analyses did not allow reliable placement of the organism's phylogenetic position in the 16S rRNA based tree of life. In addition, it was unclear whether N. equitans is a primitive or a derived member of the Archaea. SEQ ID NO:1, the genome of N. equitans, was deposited as GenBank accession no. ______. In one aspect, the genome of the invention has a single, circular chromosome of 490,885 basepairs (bp) and has an average G+C content of 31.6% (4). All 61 sense codons are used, but in line with the low G+C content the third codon position is mainly A or T. 539 open reading frames (ORFs) were identified that cover 92% of the genome; average ORF length is 835 bp. Functional roles were assigned to 66.8% of the annotated genes. Of the ORFs of unknown function only 18.3% have homologs in other organisms, while the remaining ones are unique to N. equitans (see Table 3, below).

Genes encoding single copies of 5S, 16S and 23S rRNA, and 35 tRNAs were identified. These stable RNAs exhibit much higher G+C content (65-80%) than the rest of the genome. This is likely due to the need to form stable secondary structures at the high growth temperature of N. equitans. The rRNA genes were not found in an operon. In fact, a low occurrence of operons is a notable feature of the N. equitans genome (SEQ ID NO:1). There is little evidence of significant lateral gene transfer in the genome; less than 5% of the N. equitans ORFs have close bacterial homologs and only about 14% have close crenarchaeal homologs. Finally, as no extrachromosomal elements could be detected (either by biochemical methods or during sequencing), the N. equitans genome of the invention (SEQ ID NO:1) represents the smallest sequenced genome of a cellular organism.

Given its small genome size it is not surprising that N. equitans lacks the metabolic capacity to synthesize many cell components, see Table 1.

TABLE 1
Comparison of N. equitans and other organisms with small genomes.

| | Nanoarchaeum | | | | |
|---|---|---|---|---|---|
| | equitans | M. genitalium^{a} | Buchnera sp. APS⁷ | R. prowazokii⁴ | T. paliidum⁹ |
| Genome size | 490,885 | 580,070 | 540,681 | 1,111,523 | 1,138,008 |
| (base pairs) | | | | | |
| G-C content | 31.6% | 31.6% | 26.3% | 29.1% | 52.8% |
| Glycolysis | − | + | + | − | + |
| Pentose Phoshate | − | Limited | + | − | + |
| Pathway | | | | | |
| TCA cycle | − | − | − | + | − |
| ATP production by | Limited | − | + | + | − |
| respiration | | | | | |
| Amino acid synthesis | − | − | + | − | − |
| Nucleotide synthesis | − | − | + | − | − |
| Lipid synthesis | − | Limited | − | + | + |
| DNA recombination/ | + | Limited | Limited | Limited | Limited |
| repair | | | | | |

(+) indicates all components of the pathway are present
(−) indicates that none of the components are present
^{a}Limited indicates that some but not all of the components are present.

Based on our current knowledge of metabolism, no ORFs were found that represent enzymes for the de novo synthesis of amino acids, nucleotides, cofactors or lipids, and known genes are absent for glycolysis, the pentose phosphate pathway and the citric acid cycle. This absence of metabolic capacity must necessitate the transport of many cellular components and metabolites from the host Ignicoccus, but it is currently unclear how this is accomplished. It remains enigmatic how N. equitans gains energy. At least a basic A1A0-type ATPase consisting of only 5 subunits as well as some components of an electron transport chain could be identified. Only five transport proteins have been identified in this genome: a Na+/Ca2+ exchange protein, a Mg2+- and Co2+-transporter, a C-4 dicarboxylate transporter, one ABC type transporter system involved in lipoprotein release, and an uncharacterized iron-regulated ABC transporter. N. equitans may acquire its lipids directly from its host Ignicoccus; a striking feature of this organism is the vast formation of vesicles at its cytoplasmic membrane, which may be part of a supply mechanism of cell components to N. equitans. (see, e.g., Huber (2000) Int. J. Syst. Evol. Microbiol. 50:2093). As similar features are found in parasites, the genome of N. equitans (SEQ ID NO:1) points to a parasitic life style. As a matter of fact, the presence of several N. equitans cells prevented multiplication of the Ignicoccus host.

A common feature of bacterial obligate parasites is their small genome size (see, e.g., Fraser (1995) Science 270:397; Shigenobu (2000) Nature 407:81; Andersson (1998) Nature 396:133; Fraser (1998) Science 281:375) as a consequence of genome reduction during adaptation to parasitic life style. This may also be true for N. equitans. The absence of many generally conserved operons (e.g., rRNAs, ATPase and RNA polymerase subunits) as well as the lack of duplicated genes is evidence for multiple genome rearrangements which usually accompany the reduction process (see, e.g., Andersson (1998) Trends Microbiol. 6:263).

In contrast to many obligate parasites, N. equitans has a repertoire of repair enzymes for base and nucleotide excision including endonucleases III, IV, and V (NEQ126a, NEQ398, NEQ077a, NEQ368, NEQ346a), rad2 (NEQ088) and rad25 (NEQ369). In addition, the presence of homologs of radA (NEQ426), Rad50 (NEQ256) and a Holliday-junction resolvase (NEQ424) may indicate that N. equitans can undergo homologous recombination. The presence of these enzymes sets N. equitans apart from other organisms with small genomes in significant ways. First, N. equitans may be able to repair damage to DNA that is likely to occur in its high temperature habitat. In addition, it appears to have the potential to acquire new genes via lateral gene transfer. In addition, despite its small genome N. equitans might have the capacity to adapt to changing environments. It is noteworthy that many organisms with small genomes have lost recombination/repair enzymes even though these losses incur significant negative effects. Organisms that lack these enzymes are at best evolutionarily stable (see, e.g., Tamas (2002) Science 296:2376) and at worst subject to the negative effects of increased occurrence and fixation of deleterious mutations (see, e.g., Moran (1996) Proc. Natl. Acad. Sci U.S.A. 93:2873).

N. equitans possesses a large and reasonably complete set of components for information processing (replication, transcription and translation) and completion of the cell cycle. For transcription, a4DNA-dependent RNA polymerase consisting of 14 subunits, and the archaeal genre proteins involved in transcription initiation, elongation and termination could be identified. The gene sets for DNA replication and cell cycle are similar to those found in Euryarchaeota and contain several components usually absent from Crenarchaeota (e.g., DNA polymerase II, two copies of ftsZ, and histones). The translational machinery of N. equitans is similar to other Archaea. However, three tRNA genes (for glutamate, histidine and tryptophan) were not found in N. equitans. These tRNA species may have an unusual sequence/structure (resembling e.g., mitochondrial tRNAs or M kandleri TRNA Glu) causing them to be missed by tRNA scan-SE (see, e.g., Lowe (1997) Nucleic Acids Res. 25:955). tRNA import from the host, the possibility of a tRNA species with dual function (based on different nucleotide modification) or of an anticodon change by RNA editing may also be plausible. Four tRNA species (for serine, tyrosine, isoleucine, and methionine) contain single introns in the expected position. Also present are two homologs of EndA (NEQ261, NEQ205), an intron excision enzyme that is found in all other Archaea. In some Euryarchaeota an in-frame gene duplication of EndA has occurred followed by a specialization of each domain (see, e.g., Lykke-Andersen (1997) EMBO J. 16:6290). While NEQ205 is most similar to Methanococcus jannaschii EndA (which does not have the gene duplication), NEQ261 shows more similarity to the N-terminal domain of the duplicated endA of Methanosarcina acetivorans. Also identified were some genes linked to snoRNA-dependent RNA modifications, including pseudouridine synthase (truB family, CBF5; NEQ454) and fibrillarin (NEQ125). This is in agreement with the recent reports that some archaea, possess snoRNAs (Omer (2000) Science 288:517). Thus, snoRNA metabolism may be an ancient characteristic of archaea that was present in a predecessor of all known archaeal phyla.

An unusual characteristic of the N. equitans genome (SEQ ID NO:1) is the high number of split genes, whose gene product is encoded by two physically separated ORFs, see Table 2.

TABLE 2
Split genes in N. equitans.

| | ORF encoding | ORF encoding | Distance between |
|---|---|---|---|
| Gene | N-terminal part | C-terminal part | ORFs |
| Reverse gyrase^{a} | NEQ434 | NEQ318 | 95,625 bp |
| Topoisomerase I | NEQ045 | NEQ324 | 241,346 bp |
| Glu-tRNA^{Gla} amidotransferase (gatE) | NEQ245 | NEQ396 | 126,409 bp |
| Alanyl-tRNA synthetase | NEQ547 | NEQ211 | 185,194 bp |
| DNA polymerase B^{b} | NEQ068 | NEQ528 | 83,301 bp |
| RNA polymerase subunit B^{c} | NEQ173 | NEQ156 | 13,388 bp |
| Large helicase related protein | NEQ003 | NEQ409 | 134,395 bp |
| archaeosine tRNA-guanine transglycosylase | NEQ124 | NEQ305 | 164,284 bp |
| Conserved hypothetical protein (RNA-binding | NEQ438 | NEQ506 | 62,414 bp |
| protein homologous to eukaryotic snRNP) | | | |

^{a}also split in Methanopyrus kandleri (different site)
^{b}also split in Methanothermobacter thermoautotrophicus
^{c}also split in methanogens, Archaeoglobales, and extreme halophiles

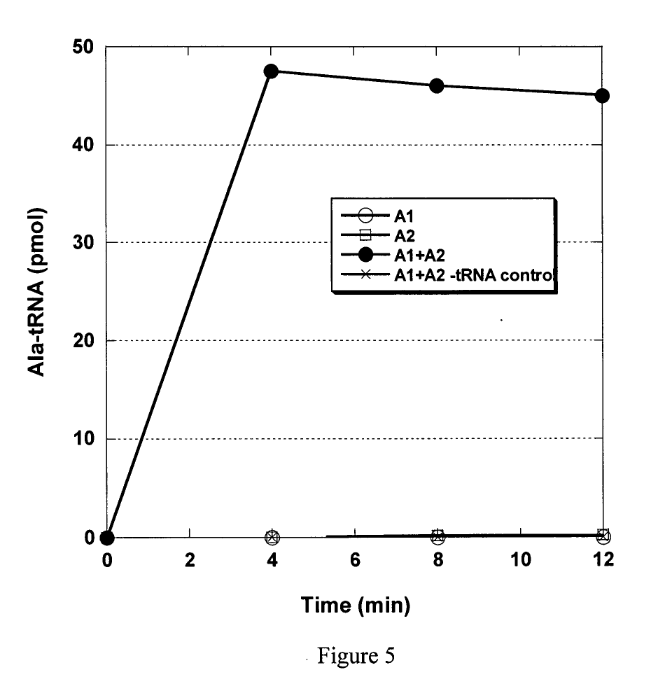

In most cases the site of the split lies between functional domains of the encoded proteins; thus it seems likely that the two separated ORFs encode parts of the corresponding enzyme, which assemble to a functional protein after independent expression. The split gene separated by the largest segment of DNA (see Table 2) encodes alanyl-tRNA synthetase. This gene provided the opportunity to test the idea that the individual protein parts are catalytically inactive, but that they reconstitute activity when combined (see, e.g., Burbaum (1991) Biochemistry 30:319). Only a combination of both parts of the split protein yielded a fully active enzyme as checked by the standard aminoacylation assay (FIG. 5, see also, discussion, below). Thus, in this case trans-splicing is not a prerequisite for enzyme activity (see, e.g., Ahel (2002) J. Biol. Chem. 277:34743).

The N. equitans reverse gyrase is split into two distinct genes encoding a helicase (NEQ434) and a topoisomerase (NEQ318) domain. Reverse gyrase appears to be the fusion product of a helicase and a5topoisomerase domain, and catalyzes positive supercoil formation in DNA (see, e.g., Krah (1996) Proc. Natl. Acad. Sci. U.S.A. 93:106; Forterre (2002) Trends Genet. 18:236). Since this enzyme is only present in hyperthermophiles, it was concluded that hyperthermophily appeared secondarily in the evolution of life (see, e.g., Forterre (2000) Trends Genet. 16:152). In light of the presence of independent helicase and topoisomerase domains in the deep rooted N. equitans, the evolution of hyperthermophily may have been a very early event in agreement with the view of a hot primeval earth (see, e.g., Stetter, in Commentarii Pontifica Academica Scientiarium, Vatican City (1997) vol. IV).

Another split gene encodes DNA-directed polymerase I. The N-terminal (NEQ068) and C-terminal (NEQ528) ORFs are separated by 83,301 bp and are located on opposite DNA strands. The C-terminal part (294 bp) of NEQ068 together with the N-terminal region (93 bp) of NEQ528 encode a mini-intein (129 aa). It is predicted that the two parts of the DNA polymerase are expressed separately and then covalently linked after the reassembled intein has been excised by a trans-splicing mechanism (see, e.g., Wu (1998) Proc. Natl. Acad. Sci. U.S.A. 95:9226).

It is unlikely that the split genes are solely the by-product of a small or reduced genome as the other known small genomes do not contain the number and type of split genes found in N. equitans. These split domain proteins may be early remnants of protein evolution that depended on domain fusion for the generation of modern/complex proteins (see, e.g., Doolittle (1995) Annu. Rev. Biochem. 64:287; Doolittle (1978) Nature 272:581; Gilbert (1997) Proc. Natl. Acad. Sci. U.S.A. 94:7698).

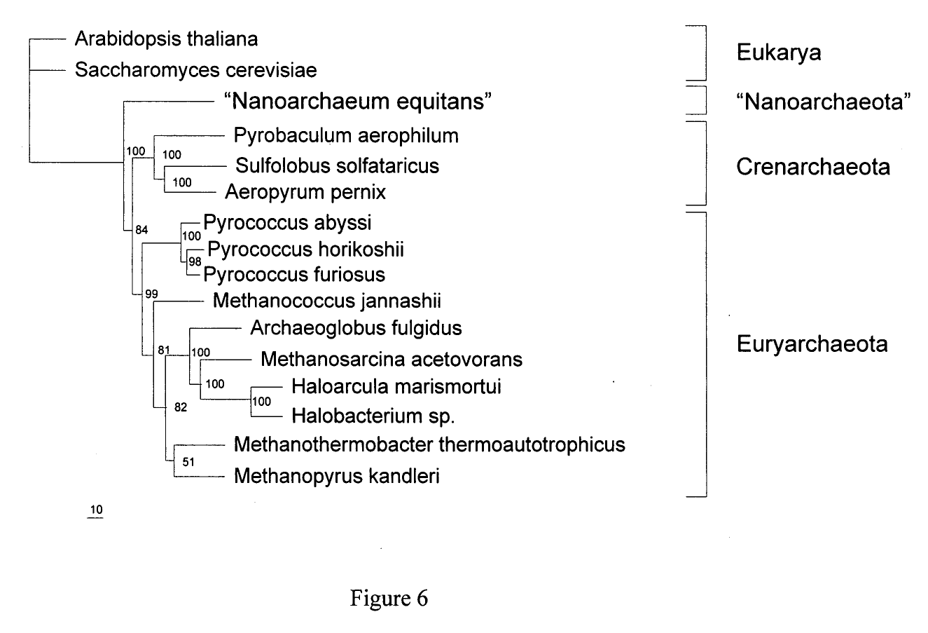

To shed light on the phylogenetic relationship among the Archaea the amino acid sequences of 35 ribosomal proteins were concatenated and aligned. A concatenated alignment of 35 ribosomal proteins was analyzed by maximum likelihood, Bayseian and parsimony analysis. N. equitans was placed with high support at the most basal position within the Archaea in the maximum likelihood, maximum parsimony, and Bayseian trees (see FIG. 6) suggesting that the Nanoarchaeota are the basal kingdom within the Archaea. FIG. 6 schematically illustrates the phylogenetic position of Nanoarchaeum equitans within the Archaea. The tree was determinedly the maximum likelihood method, based on 35 concatenated ribosomal protein sequences. Numbers indicate percentage of bootstrap resamplings. Scale bar corresponds to estimated substitutions per 100 positions.

Recent evidence shows that N. equitans is not the only extant member of the Nanoarchaeota. Two sequences from Caldera Uzon (Kamchatka) and Yellowstone National Park (USA) exhibited 83% sequence similarity to N. equitans and therefore represent a distinct group within the Nanoarchaeota (Hohn (2002) Syst. Appl. Micro. 25:551). Light microscopy and fluorescence in situ hybridization reveal that these novel Nanoarchaeota are tiny cocci like N. equitans attached to other archaeal species. Similar to Ignicoccus, these hosts may gain energy by S/H-autotrophy, a metabolism considered to be primitive (Fischer (1983) Nature 301:511). In contrast to the bacterial small genome parasites, no free-living close relative to N. equitans could be detected so far. Therefore one cannot deduce the genome size of its possible precursor. The assumption of N. equitans being a non-reduced primitive genome would provide exciting possibilities for research about ancient features of parasitism, early organismal development, and also a starting point for the generation of a minimal autonomous genome.

TABLE 3

| SEQ | | | | | | | |
|---|---|---|---|---|---|---|---|
| ID | | Blast Hit/Manual | Genbank Definition | Blastp | Pfam & TIGR | EC | |
| NO: | Name | Annotation | of top Blastp hit | Evalue | Domains | Number | COG Hit |
| 1 | N/A | N/A - SEQ ID NO: 1 | N/A - SEQ ID NO: 1 is the DNA | | N/A - SEQ ID NO: 1 is the DNA | | |
| | | is the DNA sequence | sequence of the whole genome | | sequence of the whole genome | | |
| | | of the whole genome | | | | | |
| 2, 3 | NEQ001 | uncharacterized | conserved | 5.00E−27 | DUF57 Protein of unknown | | Poorly |
| | | conserved protein | hypothetical | | function DUF57 2.00e−06 | | characterized; |
| | | | [Methanococcus | | | | Function |
| | | | jannaschii]. | | | | unknown; Uncharacterized |
| | | | | | | | ACR 3e−28 |
| 4, 5 | NEQ003 | large helicase- | large helicase-related | 0 | DEAD DEAD/DEAH | 3.6.1.— | Poorly |
| | | related protein | protein [Pyrococcus | | box helicase 4.80e−47 | | characterized; |
| | | [Pyrococcus abyssi]. | abyssi]. | | :helicase₋ C | | General |
| | | SPLIT See SEQ ID | | | Helicase conserved | | function |
| | | NOS: 798, 799 | | | C-terminal domain | | prediction |
| | | | | | 1.70e−18 :recq recq | | only; Lhr-like |
| | | | | | ATP-dependent | | helicases 0 |
| | | | | | DNA helicase RecQ | | |
| | | | | | 4.50e−06 | | |
| 6, 7 | NEQ002 | fkbp-type peptidyl- | fkbp-type peptidyl- | 5.00E−13 | 0 | 5.2.1.8 | Cellular |
| | | prolyl cis- | prolyl cis-transisomerase | | | | processes; Posttranslational |
| | | transisomerase | [Methanothermobacter | | | | modification, |
| | | | thermautotrophicus]. | | | | protein |
| | | | | | | | turnover, |
| | | | | | | | chaperones; FKBP- |
| | | | | | | | type |
| | | | | | | | peptidyl-prolyl |
| | | | | | | | cis-transisomerases 2 |
| | | | | | | | 4e−14 |
| 8, 9 | NEQ004 | Uncharacterized | conserved | 3.00E−20 | DUF101 Protein of unknown | | Poorly |
| | | conserved | hypothetical protein | | function DUF101 2.90e−27 | | characterized; |
| | | protein[Methanopyrus | [Methanococcus | | | | Function |
| | | kandleri AV19] | jannaschii]. | | | | unknown; Uncharacterized |
| | | | | | | | ACR 3e−21 |
| 10, | NEQ005 | hypothetical | 288aa long | 6.00E−29 | DUF425 Protein of unknown | | Poorly |
| 11 | | carbamoylphosphate | hypothetical protein | | function (DUF425) 1.60e−13 | | characterized; |
| | | synthetase | [Sulfolobus tokodaii]. | | | | Function |
| | | [Aeropyrum pernix]. | | | | | unknown; Uncharacterized |
| | | | | | | | ACR 2e−25 |
| 12, | NEQ007 | methylated-DNA- | methylated-DNA- | 3.00E−12 | Methyltransf₋ 1 6-O- | 2.1.1. | Information |
| 13 | | [protein]-cysteineS- | [protein]-cysteine S- | | methylguanine DNA | 63 | storage and |
| | | methyltransferase | methyltransferase | | methyltransferase, | | processing; DNA |
| | | (ogt) | (ogt) [Pyrobaculum | | DNA binding domain | | replication, |
| | | | aerophilum]. | | 1.60e−07 :ogt ogt | | recombination |
| | | | | | methylated-DNA- | | and |
| | | | | | protein-cysteine | | repair; Methylated |
| | | | | | methyltransferase | | DNA- |
| | | | | | 9.60e−10 | | protein |
| | | | | | | | cysteine |
| | | | | | | | methyltransferase |
| | | | | | | | 8e−13 |
| 14, | NEQ006 | hypothetical protein | hypothetical protein | 3.00E−04 | 0 | | |
| 15 | | | [Clostridium | | | | |
| | | | perfringens]. | | | | |
| 16, | NEQ008 | hypothetical 2- | 418aa long conserved | 2.00E−82 | UPF0004 | 1.8.—.— | Information |
| 17 | | methylthioadeninesynthase | hypothetical protein | | Uncharacterized | | storage and |
| | | | [Sulfolobus tokodaii]. | | protein family | | processing; Translation, |
| | | | | | UPF0004 1.60e−22 | | ribosomal |
| | | | | | :TIGR00089 | | structure and |
| | | | | | TIGR00089 | | biogenesis; 2- |
| | | | | | conserved | | methylthioadenine |
| | | | | | hypothetical protein | | synthetase |
| | | | | | 3.30e−123 | | 6e−80 |
| | | | | | :TIGR01125 | | |
| | | | | | TIGR01125 | | |
| | | | | | conserved | | |
| | | | | | hypothetical protein | | |
| | | | | | TIGR01125 6.70e−19 | | |
| | | | | | :TIGR01212 | | |
| | | | | | TIGR01212 | | |
| | | | | | conserved | | |
| | | | | | hypothetical protein | | |
| | | | | | TIGR01212 3.60e−3 | | |
| | | | | | :Radical₋ SAM | | |
| | | | | | Radical SAM | | |
| | | | | | superfamily 5.10e−27 | | |
| 18, | NEQ009 | hypothetical protein | conserved | 8.00E−13 | 0 | | Poorly |
| 19 | | | hypothetical protein | | | | characterized; |
| | | | [Pyrobaculum | | | | General |
| | | | aerophilum]. | | | | function |
| | | | | | | | prediction |
| | | | | | | | only; Predicted |
| | | | | | | | acetyltransferase |
| | | | | | | | 1e−07 |
| 20, | NEQ010 | hypothetical protein | hypothetical protein | 0.01 | 0 | | |
| 21 | | | predicted by | | | | |
| | | | GeneMark [Bacillus | | | | |
| | | | anthracis A2012]. | | | | |
| 22, | NEQ011 | desulfoferrodoxin | SUPEROXIDE | 7.00E−27 | Desulfoferrodox | 1.15.—.— | Metabolism; Energy |
| 23 | | [Pyrococcus abyssi] | REDUCTASE (SOR). | | Desulfoferrodoxin | | production |
| | | | | | 6.20e−24 | | and |
| | | | | | :neela₋ ferrous | | conversion; Desulfoferrodoxin |
| | | | | | neela₋ ferrous | | 4e−28 |
| | | | | | desulfoferrodoxin | | |
| | | | | | ferrous iron-binding | | |
| | | | | | domain 2.00e−18 | | |
| 24, | NEQ012 | glycosyltransferase | glycosyltransferase | 2.00E−19 | Glycos₋ transf₋ 1 Glycosyl | | Cellular |
| 25 | | [Pyrococcusfuriosus | [Methanosarcina | | transferases group 1 2.60e−06 | | processes; Cell |
| | | DSM 3638] | acetivorans str. C2A]. | | | | envelope |
| | | | | | | | biogenesis, |
| | | | | | | | outer |
| | | | | | | | membrane; Predicted |
| | | | | | | | glycosyltransferases |
| | | | | | | | 1e−13 |
| 26, | NEQ013 | hypothetical | 377aa long | 6.00E−17 | Radical₋ SAM Radical SAM | | Poorly |
| 27 | | coenzyme PQQ | hypothetical | | superfamily 3.20e−21 | | characterized; |
| | | synthesisprotein | coenzyme PQQ | | | | General |
| | | [Sulfolobus tokodaii] | synthesis protein | | | | function |
| | | | [Sulfolobus tokodaii]. | | | | prediction |
| | | | | | | | only; Predicted |
| | | | | | | | Fe—S |
| | | | | | | | oxidoreductases |
| | | | | | | | 8e−17 |
| 28, | NEQ014 | C4-dicarboxylate | hypothetical protein | 3.00E−60 | tdt tdt C4-dicarboxylate | | Cellular |
| 29 | | transporter, | [Magnetococcus sp. | | transporter/malic acid | | processes; Inorganic |
| | | putative[Pyrococcus | MC-1]. | | transport protein 7.80e−33 | | ion |
| | | abyssi] | | | :C4dic₋ mal₋ tran C4- | | transport and |
| | | | | | dicarboxylate | | metabolism; Tellurite |
| | | | | | transporter/malic acid | | resistance |
| | | | | | transport protein 6.60e−32 | | protein and |
| | | | | | | | related |
| | | | | | | | permeases |
| | | | | | | | 3e−29 |
| 30, | NEQ015 | hypothetical | 257aa long conserved | 2.00E−22 | Thy1 Thymidylate synthase | | Metabolism; Nucleotide |
| 31 | | thymidylate | hypothetical protein | | complementing protein | | transport and |
| | | synthasecomplementing | [Sulfolobus tokodaii]. | | 1.20e−09 | | metabolism; Predicted |
| | | protein | | | | | alternative |
| | | | | | | | thymidylate |
| | | | | | | | synthase 1e−16 |
| 32, | NEQ016 | hypothetical protein | hypothetical protein | 1.00E−12 | DUF196 Uncharacterized | | Poorly |
| 33 | | | [Methanosarcina | | ACR, COG1343 7.60e−17 | | characterized; |
| | | | barkeri]. | | | | Function |
| | | | | | | | unknown; Uncharacterized |
| | | | | | | | ACR 4e−13 |
| 34, | NEQ017 | conserved | hypothetical protein | 3.00E−60 | DUF48 Protein of unknown | | Poorly |
| 35 | | hypothetical | [Pyrococcus | | function DUF48 8.20e−96 | | characterized; |
| | | protein[Pyrococcus | horikoshii]. | | :TIGR00287 TIGR00287 | | Function |
| | | horikoshii] | | | conserved hypothetical | | unknown; Uncharacterized |
| | | | | | protein TIGR00287 3.80e−79 | | ACR 2e−61 |
| 36, | NEQ018 | hypothetical protein | maturase [Euglena | 9.00E−05 | 0 | | |
| 37 | | | gracilis]. | | | | |
| 38, | NEQ019 | conserved | hypothetical protein | 1.00E−15 | | | Poorly |
| 39 | | hypothetical | [Pyrococcus | | | | characterized; |
| | | protein[Pyrococcus | horikoshii]. | | | | Function |
| | | horikoshii] | | | | | unknown; Uncharacterized |
| | | | | | | | ACR 7e−17 |
| 40, | NEQ020 | hypothetical protein | hypothetical protein | 6.00E−03 | 0 | | |
| 41 | | | [Methanosarcina | | | | |
| | | | mazei Goe1]. | | | | |
| 42, | NEQ022 | ATP-dependent RNA | hypothetical protein | 2.00E−53 | DEAD DEAD/DEAH | 2.7.7.— | Poorly |
| 43 | | helicase[Fusobacterium | [Pyrococcus | | box helicase 9.50e−06 | | characterized; |
| | | nucleatum] | horikoshii]. | | :helicase₋ C | | General |
| | | | | | Helicase conserved | | function |
| | | | | | C-terminal domain | | prediction |
| | | | | | 5.80e−4 | | only; Predicted |
| | | | | | | | helicases |
| | | | | | | | 1e−54 |
| 44, | NEQ021 | RecB family | hypothetical protein | 1.00E−30 | DUF83 Domain of unknown | | Information |
| 45 | | exonuclease | [Pyrococcus | | function DUF83 2.50e−25 | | storage and |
| | | | horikoshii]. | | :TIGR00372 TIGR00372 | | processing; DNA |
| | | | | | conserved hypothetical | | replication, |
| | | | | | protein TIGR00372 3.80e−40 | | recombination |
| | | | | | | | and |
| | | | | | | | repair; RecB |
| | | | | | | | family |
| | | | | | | | exonuclease |
| | | | | | | | 1e−31 |
| 46, | NEQ023 | protease | protease | 5.00E−17 | 9.70E−16 | 3.4.—.— | Poorly |
| 47 | | [Pyrobaculum | [Pyrobaculum | | | | characterized; |
| | | aerophilum] | aerophilum]. | | | | General |
| | | | | | | | function |
| | | | | | | | prediction |
| | | | | | | | only; Predicted |
| | | | | | | | Zn- |
| | | | | | | | dependent |
| | | | | | | | peptidases |
| | | | | | | | 2e−13 |
| 48, | NEQ024 | hypothetical sulfide | flavoprotein | 2.00E−88 | pyr₋ redox Pyridine | 1.6.99.3 | Poorly |
| 49 | | dehydrogenase[flavo | reductase, conjectural | | nucleotide- | | characterized; |
| | | cytochrome c] | [Pyrobaculum | | disulphide | | General |
| | | flavoprotein | aerophilum]. | | oxidoreductase | | function |
| | | | | | 2.20e−05 | | prediction |
| | | | | | | | only; Uncharacterized |
| | | | | | | | NAD(FAD)- |
| | | | | | | | dependent |
| | | | | | | | dehydrogenases |
| | | | | | | | 3e−25 |
| 50, | NEQ025 | glucose-1- | glucose-1-phosphate | 4.00E−45 | NTP₋ transferase | 2.7.7. | Cellular |
| 51 | | phosphatethymidylyltransferase | thymidylyltransferase | | Nucleotidyl | 24 | processes; Cell |
| | | (graD-1) | (graD-1) | | transferase 1.10e−23 | | envelope |
| | | [A. | [Archaeoglobus | | :galU galU UTP- | | biogenesis, |
| | | | fulgidus]. | | glucose-1- | | outer |
| | | | | | phosphate | | membrane; Nucleoside- |
| | | | | | uridylyltransferase | | diphosphate- |
| | | | | | 1.90e−07 :rmlA rmlA | | sugar |
| | | | | | glucose-1- | | pyrophosphorylases |
| | | | | | phosphate | | involved in |
| | | | | | thymidylyltransferase | | lipopolysaccharide |
| | | | | | 2.60e−3 | | biosynthesis/translation |
| | | | | | :rmlA₋ long | | initiation |
| | | | | | rmlA₋ long glucose- | | factor eIF2B |
| | | | | | 1-phosphate | | subunits 3e−46 |
| | | | | | thymidyltransferase | | |
| | | | | | 4.10e−3 | | |
| 52, | NEQ026 | hypothetical protein | hypothetical protein | 0.31 | 0 | | |
| 53 | | | [Clostridium | | | | |
| | | | thermocellum ATCC | | | | |
| | | | 27405]. | | | | |
| 54, | NEQ028 | hypothetical protein | hypothetical protein | 2.00E−03 | 0 | | |
| 55 | | | [Plasmodium yoelii | | | | |
| | | | yoelii]. | | | | |
| 56, | NEQ027 | hypothetical protein | putative bir1 protein | 0.5 | 0 | | |
| 57 | | | [Plasmodium yoelii | | | | |
| | | | yoelii]. | | | | |
| 58, | NEQ029 | hypothetical protein | protein disulfide | 0.03 | 0 | | |
| 59 | | | isomerase 4 [Giardia | | | | |
| | | | intestinalis]. | | | | |
| 60, | NEQ030 | holocytochrome-c | holocytochrome-c | 3.00E−13 | DsbD Cytochrome C | | Cellular |
| 61 | | synthase[Methanosarcina | synthase | | biogenesis protein | | processes; Posttranslational |
| | | acetivorans str. | [Methanosarcina | | transmembrane region | | modification, |
| | | C2A] | acetivorans str. C2A]. | | 4.10e−4 | | protein |
| | | | | | | | turnover, |
| | | | | | | | chaperones; Cytochrome c |
| | | | | | | | biogenesis |
| | | | | | | | protein 2e−13 |
| 62, | NEQ031 | Predicted RNA- | 181aa long conserved | 3.00E−18 | | | Poorly |
| 63 | | binding | hypothetical protein | | | | characterized; |
| | | proteincontaining KH | [Sulfolobus tokodaii]. | | | | General |
| | | domain) [M. kandleri] | | | | | function |
| | | | | | | | prediction |
| | | | | | | | only; Predicted |
| | | | | | | | RNA- |
| | | | | | | | binding |
| | | | | | | | protein |
| | | | | | | | (contains KH |
| | | | | | | | domains) 3e−16 |
| 64, | NEQ032 | hypothetical protein | repeat organellar | 4.00E−03 | 0 | | |
| 65 | | | protein-related | | | | |
| | | | [Plasmodium yoelii | | | | |
| | | | yoelii]. | | | | |
| 66, | NEQ033 | hypothetical protein | hypothetical protein | 9.00E−07 | 0 | | |
| 67 | | | [Cytophaga | | | | |
| | | | hutchinsonii]. | | | | |
| 68, | NEQ034 | hypothetical protein | similar to Plasmodium | 1.00E−05 | 0 | | |
| 69 | | | falciparum (isolate | | | | |
| | | | 3D7). Hypothetical | | | | |
| | | | protein [Dictyostelium | | | | |
| | | | discoideum]. | | | | |
| 70, | NEQ035 | hypothetical protein | hypothetical protein | 3.00E−03 | 0 | | |
| 71 | | | [Lactobacillus | | | | |
| | | | gasseri]. | | | | |
| 72, | NEQ036 | hypothetical protein | Hypothetical protein | 0.02 | 0 | | |
| 73 | | | [Clostridium | | | | |
| | | | acetobutylicum]. | | | | |
| 74, | NEQ038 | transcription initiation | Ribosomal protein | 5.00E−20 | Ribosomal₋ L37ae | | Information |
| 75 | | factor IID, TATA-box | L37AE/L43A | | Ribosomal L37ae protein | | storage and |
| | | binding protein (TBP) | [Methanopyrus | | family 2.20e−26 :L37a L37a | | processing; Translation, |
| | | | kandleri AV19]. | | ribosomal protein L37a | | ribosomal |
| | | | | | 9.10e−27 | | structure and |
| | | | | | | | biogenesis; Ribosomal |
| | | | | | | | protein |
| | | | | | | | L37AE/L43A |
| | | | | | | | 7e−20 |
| 76, | NEQ037 | Small nuclear | putative U6 snRNA- | 9.00E−05 | Sm Sm protein 1.30e−11 | | |
| 77 | | ribonucleoprotein(sn | ASSOCIATED SM- | | | | |
| | | RNP) homolog | LIKE PROTEIN | | | | |
| | | | [Encephalitozoon | | | | |
| | | | cuniculi]. | | | | |
| 78, | NEQ039 | hypothetical protein | transcription initiation | 1.00E−24 | TBP Transcription factor | | Information |
| 79 | | | factor IID, TATA-box | | TFIID (or TATA-binding | | storage and |
| | | | binding protein | | protein, TBP) 4.60e−21 :TBP | | processing; Transcription; |
| | | | [Methanococcus | | Transcription factor TFIID (or | | Transcription |
| | | | jannaschii]. | | TATA-binding protein, TBP) | | initiation |
| | | | | | 1.10e−06 | | factor TFIID |
| | | | | | | | (TATA- |
| | | | | | | | binding |
| | | | | | | | protein) 1e−25 |
| 80, | NEQ041 | hypothetical protein | predicted protein | 0.36 | 0 | | |
| 81 | | | [Methanosarcina | | | | |
| | | | acetivorans str. C2A]. | | | | |
| 82, | NEQ040 | hypothetical protein | hypothetical protein | 6.00E−05 | PAP2 PAP2 superfamily 6.00e−05 | | |
| 83 | | | T13C5.6- | | | | |
| | | | Caenorhabditis | | | | |
| | | | elegans. | | | | |
| 84, | NEQ042 | thermostable | thermostable | 1.00E−135 | Peptidase₋ M32 | 3.4.17. | Metabolism; Amino |
| 85 | | carboxypeptidase | carboxypeptidase | | Carboxypeptidase | 19 | acid |
| | | [P. aerophilum] | [Pyrobaculum | | Taq (M32) | | transport and |
| | | | aerophilum]. | | metallopeptidase | | metabolism; Zn- |
| | | | | | 2.80e−174 | | dependent |
| | | | | | | | carboxypeptidases |
| | | | | | | | 1e−129 |
| 86, | NEQ043 | hypothetical protein | ribosomal protein S26 | 0.09 | 0 | | |
| 87 | | | [Spodoptera | | | | |
| | | | frugiperda]. | | | | |
| 88, | NEQ044 | O-sialoglycoprotein | O-sialoglycoprotein | 4.00E−23 | RIO1 | 3.4.24. | Cellular |
| 89 | | endopeptidase[Methanosarcina | endopeptidase | | RIO1/ZK632.3/MJ04 | 57 | processes; Signal |
| | | acetivorans str. C2A] | [Methanosarcina | | 44 family 1.60e−4 | | transduction |
| | | | acetivorans str. C2A]. | | | | mechanisms; |
| | | | | | | | Mn2+- |
| | | | | | | | dependent |
| | | | | | | | serine/threonine |
| | | | | | | | protein |
| | | | | | | | kinase 1e−22 |
| 90, | NEQ045 | DNA topoisomerase I | DNA topoisomerase I | 6.00E−75 | Topoisom₋ bac DNA | 5.99. | Information |
| 91 | | [Pyrococcushorikoshii] | [Pyrococcus | | topoisomerase | 1.2 | storage and |
| | | SPLIT see SEQ ID | horikoshii]. | | 2.20e−23 :Toprim | | processing; DNA |
| | | NOS: 632, 633 | | | Toprim domain | | replication, |
| | | | | | 4.00e−11 :topA₋ bact | | recombination |
| | | | | | topA₋ bact DNA | | and |
| | | | | | topoisomerase I | | repair; Topoisomerase |
| | | | | | 2.90e−09 :topB topB | | IA |
| | | | | | DNA topoisomerase | | 4e−76 |
| | | | | | III 6.00e−05 | | |
| | | | | | :topA₋ arch | | |
| | | | | | topA₋ arch DNA | | |
| | | | | | topoisomerase I | | |
| | | | | | 1.10e−31 | | |
| 92, | NEQ047 | hypothetical HESA | HESA protein | 2.00E−10 | ThiF ThiF family 3.00e−4 | | Metabolism; Coenzyme |
| 93 | | protein[Aeropyrum | [Aeropyrum pernix]. | | | | metabolism; Dinucleotide- |
| | | pernix] | | | | | utilizing |
| | | | | | | | enzymes |
| | | | | | | | involved in |
| | | | | | | | molybdopterin |
| | | | | | | | and thiamine |
| | | | | | | | biosynthesis |
| | | | | | | | family 2 1e−11 |
| 94, | NEQ046 | hypothetical protein | Unknown | 0.03 | 0 | | |
| 95 | | | [Streptococcus | | | | |
| | | | agalactiae NEM316] | | | | |
| 96, | NEQ048 | hypothetical protein | hypothetical protein | 5.00E−04 | 0 | | |
| 97 | | | [Plasmodium | | | | |
| | | | falciparum 3D7]. | | | | |
| 98, | NEQ050 | hypothetical protein | hypothetical protein | 4.00E−06 | NTP₋ transf₋ 2 Nucleotidyltransferase domain | | |
| 99 | | | [Plasmodium | | | | |
| | | | falciparum 3D7]. | | | | |
| 100, | NEQ049 | Predicted Fe—S | conserved | 3.00E−14 | TIGR01212 TIGR01212 | | Poorly |
| 101 | | oxidoreductase[Thermoplasma | hypothetical protein | | conserved hypothetical | | characterized; |
| | | volcanium] | [Archaeoglobus | | 3.20e−4 protein TIGR01212 6.00e−3 | | General |
| | | | fulgidus]. | | :Radical₋ SAM Radical SAM | | function |
| | | | | | superfamily 4.10e−16 | | prediction |
| | | | | | | | only; Predicted |
| | | | | | | | Fe—S |
| | | | | | | | oxidoreductases |
| | | | | | | | 2e−15 |
| 102, | NEQ051 | ferredoxin-NADP | hypothetical protein | 3.00E−17 | FAD₋ binding₋ 6 | 1.18. | Metabolism; Energy |
| 103 | | reductase[Neisseria | [Cytophaga | | Oxidoreductase | 1.2 | production |
| | | meningitidis MC58] | hutchinsonii]. | | FAD-binding domain | | and |
| | | | | | 1.20e−13 | | conversion; Flavodoxin |
| | | | | | :NAD₋ binding₋ 1 | | reductases |
| | | | | | Oxidoreductase | | (ferredoxin- |
| | | | | | NAD-binding | | NADPH |
| | | | | | domain 1.50e−06 | | reductases) |
| | | | | | | | family 1 5e−14 |
| 104, | NEQ052 | peptide chain | peptide chain release | 1.00E−77 | eRF1₋ 1 eRF1 domain 1 | | Information |
| 105 | | release factor | factor aRF, subunit 1 | | 2.40e−31 :eRF1₋ 2 eRF1 | | storage and |
| | | aRF, subunit 1 | [Pyrococcus abyssi]. | | domain 2 1.40e−45 :eRF1₋ 3 | | processing; Translation, |
| | | [Pyrococcus abyssi] | | | eRF1 domain 3 9.70e−08 | | ribosomal |
| | | | | | :eRF eRF peptide chain | | structure and |
| | | | | | release factor eRF/aRF, | | biogenesis; Peptide |
| | | | | | subunit 1 2.10e−67 :pelA | | chain |
| | | | | | pelA cell division protein | | release factor |
| | | | | | pelota 3.00e−3 | | eRF1 7e−79 |
| 106, | NEQ053 | RNA | RNA | 2.00E−86 | ygcA ygcA RNA | 2.1.1.— | Information |
| 107 | | methyltransferase | methyltransferase | | methyltransferase, | | storage and |
| | | [Pyrococcushorikoshii] | [Pyrococcus | | TrmA family 4.30e−55 | | processing; Translation, |
| | | | horikoshii]. | | | | ribosomal |
| | | | | | | | structure and |
| | | | | | | | biogenesis; SAM- |
| | | | | | | | dependent |
| | | | | | | | methyltransferases |
| | | | | | | | related |
| | | | | | | | to tRNA |
| | | | | | | | (uracil-5-)- |
| | | | | | | | methyltransferase |
| | | | | | | | 1e−87 |
| 108, | NEQ054 | Conserved | hypothetical protein | 9.00E−07 | | | Poorly |
| 109 | | hypothetical | [Synechocystis sp. | | | | characterized; |
| | | protein[Pyrococcus | PCC 6803]. | | | | Function |
| | | abyssi] | | | | | unknown; Predicted |
| | | | | | | | membrane |
| | | | | | | | protein 1e−07 |
| 110, | NEQ055 | cysteinyl-tRNA | cysteinyl-tRNA | 1.00E−117 | tRNA-synt₋ 1e tRNA | 6.1.1. | Information |
| 111 | | synthetase[Pyrococcus | synthetase | | synthetases class I | 16 | storage and |
| | | furiosus DSM | [Pyrococcus furiosus | | (C) 3.70e−139 :metG | | processing; Translation, |
| | | 3638] | DSM 3638]. | | metG methionyl- | | ribosomal |
| | | | | | tRNA synthetase | | structure and |
| | | | | | 2.90e−07 :cysS cysS | | biogenesis; Cysteinyl- |
| | | | | | cysteinyl-tRNA | | tRNA |
| | | | | | synthetase 1.70e−140 | | synthetase |
| | | | | | | | 1e−115 |
| 112, | NEQ057 | Cell division control | 398aa long | 4.00E−61 | AAA ATPase family | | Information |
| 113 | | 6/orc1 | hypothetical cell | | associated with various | | storage and |
| | | proteinhomolog | division control protein | | cellular activities (AAA) | | processing; DNA |
| | | (cdc6-1) [S solfataricus] | 6 [Sulfolobus | | 1.10e−4 | | replication, |
| | | | tokodaii]. | | | | recombination |
| | | | | | | | and |
| | | | | | | | repair; Cdc6- |
| | | | | | | | related |
| | | | | | | | protein, AAA |
| | | | | | | | superfamily |
| | | | | | | | ATPase 2e−61 |
| 114, | NEQ056 | hypothetical protein | hypothetical protein | 4.00E−03 | 0 | | |
| 115 | | | [Plasmodium | | | | |
| | | | falciparum 3D7]. | | | | |
| 116, | NEQ058 | SSU ribosomal | SSU ribosomal | 1.00E−49 | Ribosomal₋ S12 Ribosomal | | Information |
| 117 | | protein | protein S12P | | protein S12 2.40e−57 | | storage and |
| | | S12P[Pyrococcus | [Pyrococcus abyssi]. | | :rpsL₋ bact rpsL₋ bact | | processing; Translation, |
| | | abyssi] | | | ribosomal protein S12 1.30e−4 | | ribosomal |
| | | | | | :S23₋ S12₋ E₋ A | | structure and |
| | | | | | S23₋ S12₋ E₋ A ribosomal | | biogenesis; Ribosomal |
| | | | | | protein S23 (S12) 3.60e−75 | | protein S12 |
| | | | | | | | 1e−50 |
| 118, | NEQ059 | Predicted ATPase | TWITCHING | 1.00E−163 | KH KH domain 7.40e−05 | | Poorly |
| 119 | | (PilT family) | MOBILITY (PILT) | | :PIN PIN domain 5.60e−08 | | characterized; |
| | | | RELATED PROTEIN | | | | General |
| | | | (PILT) [Pyrococcus | | | | function |
| | | | abyssi]. | | | | prediction |
| | | | | | | | only; ATPases |
| | | | | | | | of the PilT |
| | | | | | | | family 1e−164 |
| 120, | NEQ060 | hypothetical protein | RNA helicase | 0.11 | 0 | | |
| 121 | | | [Plasmodium yoelii | | | | |
| | | | yoelii]. | | | | |
| 122, | NEQ061 | UDP-N- | UDP-N- | 4.00E−21 | Glycos₋ transf₋ 4 | 2.7.8. | Cellular |
| 123 | | acetylglucosamine- | acetylglucosamine- | | Glycosyl transferase | 15 | processes; Cell |
| | | dolichyl-phosphateN- | dolichyl-phosphate N- | | 3.90e−10 | | envelope |
| | | acetylglucosaminephosphotransferase | acetylglucosaminephosphotransferase | | | | biogenesis, |
| | | | (gnptA) [Sulfolobus | | | | outer |
| | | | solfataricus]. | | | | membrane; UDP- |
| | | | | | | | N- |
| | | | | | | | acetylmuramyl |
| | | | | | | | pentapeptide |
| | | | | | | | phosphotransferase/ |
| | | | | | | | UDP- |
| | | | | | | | N- |
| | | | | | | | acetylglucosamine- |
| | | | | | | | 1- |
| | | | | | | | phosphate |
| | | | | | | | transferase |
| | | | | | | | 9e−21 |
| 124, | NEQ062 | Hypothetical protein; | virulence factor | 7.00E−05 | 0 | | |
| 125 | | permease? | homolog MviB | | | | |
| | | | [Aquifex aeolicus]. | | | | |
| 126, | NEQ063 | ribonuclease HII | ribonuclease HII | 8.00E−34 | RNase₋ HII | 3.1.26.4 | Information |
| 127 | | (rnhB)[Pyrobaculum | (rnhB) [Pyrobaculum | | Ribonuclease HII | | storage and |
| | | aerophilum] | aerophilum]. | | 2.40e−34 :rnhC rnhC | | processing; DNA |
| | | | | | ribonuclease HIII | | replication, |
| | | | | | 3.40e−3 :TIGR00729 | | recombination |
| | | | | | TIGR00729 | | and |
| | | | | | ribonuclease HII | | repair; Ribonuclease |
| | | | | | 3.90e−45 | | HII 5e−34 |
| 128, | NEQ064 | arylsulfatase, | conserved | 1.00E−45 | lactamase₋ B Metallo-beta- | | Poorly |
| 129 | | putative | hypothetical protein | | lactamase superfamily | | characterized; |
| | | [Pyrococcusfuriosus | [Methanococcus | | 2.20e−06 | | General |
| | | DSM 3638] | jannaschii]. | | | | function |
| | | | | | | | prediction |
| | | | | | | | only; Metal- |
| | | | | | | | dependent |
| | | | | | | | hydrolases of |
| | | | | | | | the beta- |
| | | | | | | | lactamase |
| | | | | | | | superfamily III |
| | | | | | | | 8e−47 |
| 130, | NEQ065 | LSU ribosomal | 82aa long | 4.00E−12 | Ribosomal₋ L23 Ribosomal | | Information |
| 131 | | protein L23 | hypothetical 50S | | protein L23 1.40e−4 | | storage and |
| | | | ribosomal protein L23 | | | | processing; Translation, |
| | | | [Sulfolobus tokodaii]. | | | | ribosomal |
| | | | | | | | structure and |
| | | | | | | | biogenesis; Ribosomal |
| | | | | | | | protein L23 |
| | | | | | | | 8e−12 |
| 132, | NEQ066 | hypothetical protein | hypothetical protein | 3.00E−03 | 0 | | |
| 133 | | | [Plasmodium | | | | |
| | | | falciparum 3D7]. | | | | |
| 134, | NEQ067 | Predicted hydrolase | conserved protein | 7.00E−31 | | | Poorly |
| 135 | | of the metallo-beta- | [Methanosarcina | | | | characterized; |
| | | lactamase | mazei Goe1]. | | | | General |
| | | superfamily [M. | | | | | function |
| | | | | | | | prediction |
| | | | | | | | only; Predicted |
| | | | | | | | hydrolase of |
| | | | | | | | the metallo- |
| | | | | | | | beta- |
| | | | | | | | lactamase |
| | | | | | | | superfamily |
| | | | | | | | 3e−31 |
| 136, | NEQ068 | DNA-directed DNA | DNA-directed DNA | 6.00E−85 | DNA₋ pol₋ B DNA | 2.7.7.7 | Information |
| 137 | | polymerase I(family | polymerase | | polymerase family B | | storage and |
| | | B) [P furiosus] SPLIT | [Pyrococcus furiosus | | 3.70e−07 | | processing; DNA |
| | | see SEQ ID | DSM 3638]. | | :DNA₋ pol₋ B₋ exo | | replication, |
| | | NOS: 1028, 1029 | | | DNA polymerase | | recombination |
| | | | | | family B, | | and |
| | | | | | exonuclease domain | | repair; DNA |
| | | | | | 4.70e−40 | | polymerase |
| | | | | | | | elongation |
| | | | | | | | subunit |
| | | | | | | | (family B) 5e−85 |
| 138, | NEQ069 | SSU Ribosomal | Ribosomal protein | 1.00E−46 | Ribosomal₋ S11 Ribosomal | | Information |
| 139 | | protein | S11 [Methanopyrus | | protein S11 8.90e−50 | | storage and |
| | | S11[Methanopyrus | kandleri AV19]. | | | | processing; Translation, |
| | | kandleri AV19] | | | | | ribosomal |
| | | | | | | | structure and |
| | | | | | | | biogenesis; Ribosomal |
| | | | | | | | protein S11 |
| | | | | | | | 2e−46 |
| 140, | NEQ070 | hypothetical protein | expressed protein | 0.06 | 0 | | |
| 141 | | | [Arabidopsis thaliana]. | | | | |
| 142, | NEQ072 | hypothetical protein | P-type ATPase, | 0.39 | 0 | | |
| 143 | | | putative [Plasmodium | | | | |
| | | | falciparum 3D7]. | | | | |
| 144, | NEQ071 | conserved | conserved | 1.00E−11 | 0 | | Cellular |
| 145 | | hypothetical | hypothetical protein | | | | processes; Signal |
| | | protein[Archaeoglobus | [Archaeoglobus | | | | transduction |
| | | fulgidus] | fulgidus]. | | | | mechanisms; |
| | | | | | | | Predicted |
| | | | | | | | Ser/Thr |
| | | | | | | | protein kinase |
| | | | | | | | 1e−12 |
| 146, | NEQ073 | hypothetical protein | putative tyrosine- | 0.46 | 0 | | |
| 147 | | | protein kinase | | | | |
| | | | [Salmonella enterica | | | | |
| | | | subsp. enterica | | | | |
| | | | serovar Typhi]. | | | | |
| 148, | NEQ074 | ATPase subunit of | probable ATP- | 2.00E−17 | ABC₋ tran ABC transporter | | Poorly |
| 149 | | an iron- | dependent transport | | 5.10e−09 :3a0501s02 | | characterized; |
| | | regulatedABC-type | protein [imported]- | | 3a0501s02 Type II | | General |
| | | transporter [M. kandleri] | Pyrococcus furiosus. | | (General) Secretory | | function |
| | | | | | Pathway (IISP) Family | | prediction |
| | | | | | protein 7.80e−3 :3a0106s01 | | only; Iron- |
| | | | | | 3a0106s01 sulfate transport | | regulated |
| | | | | | system permease protein | | ABC |
| | | | | | 8.60e−4 :cbiO cbiO cobalt | | transporter |
| | | | | | transport protein ATP- | | ATPase |
| | | | | | binding subunit 1.30e−4 | | subunit SufC |
| | | | | | :ccmA ccmA heme exporter | | 8e−18 |
| | | | | | protein CcmA 4.90e−05 | | |
| 150, | NEQ076 | Predicted metal- | cleavage and | 1.00E−144 | lactamase₋ B Metallo-beta- | | Poorly |
| 151 | | dependent | polyadenylation | | lactamase superfamily | | characterized; |
| | | RNase, consists of a | specifity factor protein | | 2.40e−14 | | General |
| | | metallo-beta- | [Pyrococcus furiosus | | | | function |
| | | lactamase | DSM 3638]. | | | | prediction |
| | | | | | | | only; Predicted |
| | | | | | | | metal- |
| | | | | | | | dependent |
| | | | | | | | RNase, |
| | | | | | | | consists of a |
| | | | | | | | metallo-beta- |
| | | | | | | | lactamase |
| | | | | | | | domain and |
| | | | | | | | an RNA- |
| | | | | | | | binding KH |
| | | | | | | | domain 1e−144 |
| 152, | NEQ075 | LSU ribosomal | LSU ribosomal protein | 1.00E−68 | Ribosomal₋ L18p Ribosomal | | Information |
| 153 | | protein | L18P [Methanococcus | | L18p/L5e family 1.80e−35 | | storage and |
| | | L18P[Methanococcus | jannaschii]. | | | | processing; Translation, |
| | | jannaschii] | | | | | ribosomal |
| | | | | | | | structure and |
| | | | | | | | biogenesis; Ribosomal |
| | | | | | | | protein L18 |
| | | | | | | | 9e−70 |
| 154, | NEQ077 | glutamate | Glutamate | 1.00E−116 | GLFV₋ dehydrog | 1.4.1.3 | Metabolism; Amino |
| 155 | | dehydrogenase[Thermoplasma | dehydrogenase | | Glutamate/Leucine/ | | acid |
| | | volcanium] | [Thermoplasma | | Phenylalanine/Valine | | transport and |
| | | | volcanium]. | | dehydrogenase | | metabolism; Glutamate |
| | | | | | 1.40e−74 | | dehydrogenase/ |
| | | | | | :GLFV₋ dehydrog₋ N | | leucine |
| | | | | | Glu/Leu/Phe/Val | | dehydrogenase |
| | | | | | dehydrogenase, | | 1e−117 |
| | | | | | dimerisation domain | | |
| | | | | | 2.90e−61 | | |
| 156, | NEQ077a | Endonuclease IV | putative | 2.00E−32 | AP₋ endonuc₋ 2 AP | 3.1.21.2 | Information |
| 157 | | related protein | endonuclease IV | | endonuclease family | | storage and |
| | | [Pyrococcus abyssi] | [Pyrococcus furiosus | | 2 1.10e−4 | | processing; DNA |
| | | | DSM 3638]. | | | | replication, |
| | | | | | | | recombination |
| | | | | | | | and |
| | | | | | | | repair; Endonuclease |
| | | | | | | | IV 2e−33 |
| 158, | NEQ078 | conserved | hypothetical protein | 1.00E−161 | UPF0027 Uncharacterized | | Poorly |
| 159 | | hypothetical | [Aeropyrum pernix]. | | protein family UPF0027 | | characterized; |
| | | protein[Aeropyrum | | | 9.40e−267 | | Function |
| | | pernix] | | | | | unknown; Uncharacterized |
| | | | | | | | ACR 1e−162 |
| 160, | NEQ079 | hypothetical protein | conserved | 0.14 | 0 | | |
| 161 | | | hypothetical protein | | | | |
| | | | [Clostridium | | | | |
| | | | perfringens]. | | | | |
| 162, | NEQ080 | hypothetical protein | hypothetical protein | 0.15 | 0 | | |
| 163 | | | [Rhodopseudomonas | | | | |
| | | | palustris]. | | | | |
| 164, | NEQ081 | hypothetical protein | hypothetical protein | 1.2 | 0 | | |
| 165 | | | [Plasmodium yoelii | | | | |
| | | | yoelii]. | | | | |
| 166, | NEQ082 | translation | translation elongation | 1.00E−161 | GTP_EFTU | 3.6.1. | Information |
| 167 | | elongation factor eF- | factor eF-1, subunit | | Elongation factor Tu | 48 | storage and |
| | | 1, subunit alpha (tuf) | alpha (tuf) | | GTP binding domain | | processing; Translation, |
| | | [P furiosus] | [Pyrococcus furiosus | | 2.70e−92 | | ribosomal |
| | | | DSM 3638]. | | :GTP_EFTU_D2 | | structure and |
| | | | | | Elongation factor Tu | | biogenesis; GTPases- |
| | | | | | domain 2 8.50e−27 | | translation |
| | | | | | :GTP_EFTU_D3 | | elongation |
| | | | | | Elongation factor Tu | | factors 1e−162 |
| | | | | | C-terminal domain | | |
| | | | | | 9.90e−38 | | |
| | | | | | :small_GTP | | |
| | | | | | small_GTP small | | |
| | | | | | GTP-binding protein | | |
| | | | | | domain 7.40e−07 | | |
| | | | | | :selB selB | | |
| | | | | | selenocysteine- | | |
| | | | | | specific translation | | |
| | | | | | elongation factor | | |
| | | | | | 9.80e−07 :EF- | | |
| | | | | | 1_alpha EF-1_alpha | | |
| | | | | | translation | | |
| | | | | | elongation factor | | |
| | | | | | EF-1, subunit alpha | | |
| | | | | | 8.50e−242 :EF-Tu | | |
| | | | | | EF-Tu translation | | |
| | | | | | elongation factor Tu | | |
| | | | | | 1.10e−61 :FOLD979 | | |
| | | | | | No CATH | | |
| | | | | | Annotation 5.70e−08 | | |
| 168, | NEQ083 | SSU ribosomal | SSU ribosomal | 1.00E−21 | Ribosomal_S10 Ribosomal | | Information |
| 169 | | protein | protein S10AB | | protein S10p/S20e 5.40e−30 | | storage and |
| | | S10AB(rps10AB) | (rps10AB) [Sulfolobus | | :S10_Arc_S20_Euk | | processing; Translation, |
| | | [Sulfolobus | solfataricus]. | | S10_Arc_S20_Euk | | ribosomal |
| | | solfataricus] | | | ribosomal protein S10 7.10e−29 | | structure and |
| | | | | | :rpsJ_bact rpsJ_bact | | biogenesis; Ribosomal |
| | | | | | ribosomal protein S10 8.50e−09 | | protein S10 |
| | | | | | | | 2e−21 |
| 170, | NEQ084 | coenzyme F420 - | coenzyme F420-- | 4.00E−19 | CBS CBS domain 3.60e−3 | | Poorly |
| 171 | | quinoneoxidoreductase | quinone | | :CBS CBS domain 2.20e−4 | | characterized; |
| | | (EC1.6.5.-—) 41K | oxidoreductase (EC | | | | General |
| | | chain | 1.6.5.—) 41K chain | | | | function |
| | | | [validated] - | | | | prediction |
| | | | Archaeoglobus | | | | only; CBS |
| | | | fulgidus. | | | | domains 3e−20 |
| 172, | NEQ085 | hypothetical protein | GTA = Global | 0.38 | 0 | | |
| 173 | | | Transactivator = AcMN | | | | |
| | | | PV orf42 [Bombyxmori | | | | |
| | | | nuclear | | | | |
| | | | polyhedrosis virus]. | | | | |
| 174, | NEQ086 | hypothetical protein | hypothetical protein | 3.00E−11 | 0 | | |
| 175 | | | [Plasmodium | | | | |
| | | | falciparum 3D7]. | | | | |
| 176, | NEQ088 | DNA repair protein | DNA repair protein | 1.00E−102 | XPG_I XPG I-region 5.80e−42 | | Information |
| 177 | | RAD2[Pyrococcus | RAD2 [Pyrococcus | | :XPG_N XPG N-terminal | | storage and |
| | | abyssi] | abyssi]. | | domain 3.40e−27 | | processing; DNA |
| | | | | | | | replication, |
| | | | | | | | recombination |
| | | | | | | | and repair; 5′- |
| | | | | | | | 3′ |
| | | | | | | | exonuclease |
| | | | | | | | (including N- |
| | | | | | | | terminal |
| | | | | | | | domain of |
| | | | | | | | Poll) 1e−103 |
| 178, | NEQ087 | lysyl-tRNA | lysyl-tRNA | 5.00E−68 | tRNA-synt_1f tRNA | 6.1.1.6 | Information |
| 179 | | synthetase; LysS | synthetase; LysS | | synthetases class I | | storage and |
| | | (class- | [Halobacterium sp. | | (K) 3.60e−86 | | processing; Translation, |
| | | 1)[Halobacterium sp. | NRC-1]. | | :lysS_arch | | ribosomal |
| | | NRC-1] | | | lysS_arch lysyl- | | structure and |
| | | | | | tRNA synthetase | | biogenesis; Lysyl- |
| | | | | | 1.50e−75 | | tRNA |
| | | | | | | | synthetase |
| | | | | | | | class I 3e−69 |
| 180, | NEQ089 | hypothetical protein | similar to midasin, a | 0.18 | 0 | | |
| 181 | | | large protein with an | | | | |
| | | | N-terminal domain, a | | | | |
| | | | central AAA domain | | | | |
| | | | (with similarity to | | | | |
| | | | dynein) composed of | | | | |
| | | | 6 tandem AAA | | | | |
| | | | protomers, and a C- | | | | |
| | | | terminal M-domain | | | | |
| | | | containing MIDAS | | | | |
| | | | (Metal Ion Dependent | | | | |
| | | | Adhesion Site) | | | | |
| | | | sequence motifs; | | | | |
| | | | Mdn1p [Sacc | | | | |
| 182, | NEQ090 | polysaccharide | polysaccharide | 1.00E−15 | Polysacc_synt | | Poorly |
| 183 | | biosynthesis | biosynthesis protein, | | Polysaccharide biosynthesis | | characterized; |
| | | protein, putative | putative | | protein 4.30e−3 | | General |
| | | [Archaeoglobus | [Archaeoglobus | | | | function |
| | | fulgidus] | fulgidus]. | | | | prediction |
| | | | | | | | only; Membrane |
| | | | | | | | protein |
| | | | | | | | involved in |
| | | | | | | | the export of |
| | | | | | | | O-antigen |
| | | | | | | | and teichoic |
| | | | | | | | acid 9e−17 |
| 184, | NEQ091 | LSU ribosomal | LSU ribosomal protein | 3.00E−46 | Ribosomal_L10 | 4.2.99. | Information |
| 185 | | protein | L10E [Methanococcus | | Ribosomal protein | 18 | storage and |
| | | L10E[Methanococcus | jannaschii]. | | L10 1.30e−19 | | processing; Translation, |
| | | jannaschii] | | | | | ribosomal |
| | | | | | | | structure and |
| | | | | | | | biogenesis; Ribosomal |
| | | | | | | | protein L10 |
| | | | | | | | 2e−47 |
| 186, | NEQ092 | LSU ribosomal | LSU ribosomal protein | 3.00E−35 | Ribosomal_L14 Ribosomal | | Information |
| 187 | | protein L14P | L14P (rplN) | | protein L14p/L23e 2.60e−49 | | storage and |
| | | (rplN)[Methanococcus | [Methanococcus | | :rplN_bact rplN_bact | | processing; Translation, |
| | | jannaschii] | jannaschii]. | | ribosomal protein L14 9.50e−26 | | ribosomal |
| | | | | | | | structure and |
| | | | | | | | biogenesis; Ribosomal |
| | | | | | | | protein L14 |
| | | | | | | | 3e−36 |
| 188, | NEQ094 | hypothetical protein | conserved protein | 0.01 | 0 | | |
| 189 | | | [Methanosarcina | | | | |
| | | | mazei Goe1]. | | | | |
| 190, | NEQ093 | LSU ribosomal | LSU ribosomal protein | 6.00E−49 | Ribosomal_L5 Ribosomal | | Information |
| 191 | | protein | L5P [Pyrococcus | | protein L5 9.20e−19 | | storage and |
| | | L5P[Pyrococcus | abyssi]. | | :Ribosomal_L5_C ribosomal | | processing; Translation, |
| | | abyssi] | | | L5P family C-terminus | | ribosomal |
| | | | | | 1.80e−39 | | structure and |
| | | | | | | | biogenesis; Ribosomal |
| | | | | | | | protein L5 5e−50 |
| 192, | NEQ095 | hypothetical protein | Cucumber Basic | 0.33 | DUF124 Protein of unknown | | |
| | | | | | function DUF124 3.70e−3 | | |
| 193 | | | Protein, A Blue | | | | |
| | | | Copper Protein. | | | | |
| 194, | NEQ096 | Predicted P-loop | Predicted P-loop | 7.00E−60 | Acetyltransf | | Poorly |
| 195 | | ATPase fused to | ATPase fused to an | | Acetyltransferase (GNAT) | | characterized; |
| | | anacetyltransferase | acetyltransferase | | family 2.70e−3 | | General |
| | | [M. kandler] SPLIT | [Methanopyrus | | | | function |
| | | see SEQ ID NO: 964, | kandleri AV19]. | | | | prediction |
| | | 965 | | | | | only; Predicted |
| | | | | | | | P-loop |
| | | | | | | | ATPase fused |
| | | | | | | | to an |
| | | | | | | | acetyltransferase |
| | | | | | | | 1e−53 |
| 196, | NEQ097 | nuclease, putative | nuclease, putative | 2.00E−07 | SNase Staphylococcal | | Information |
| 197 | | [Haemophilusinfluenzae | [Haemophilus | | nuclease homologue 3.70e−07 | | storage and |
| | | Rd] | influenzae Rd]. | | | | processing; DNA |
| | | | | | | | replication, |
| | | | | | | | recombination |
| | | | | | | | and |
| | | | | | | | repair; Micrococcal |
| | | | | | | | nuclease |
| | | | | | | | (thermonuclease) |
| | | | | | | | homologs 1e−08 |
| 198, | NEQ098 | Predicted | hypothetical protein | 5.00E−18 | DUF118 Helix-turn-helix | | Information |
| 199 | | transcription | [Pyrococcus furiosus | | family DUF118 9.60e−16 | | storage and |
| | | regulator[Halobacterium | DSM 3638]. | | | | processing; Transcription; |
| | | sp. NRC-1] | | | | | Predicted |
| | | | | | | | transcriptional |
| | | | | | | | regulators 3e−18 |
| 200, | NEQ099 | hypothetical protein | wsv079 [shrimp white | 0.5 | 0 | | |
| 201 | | | spot syndrome virus]. | | | | |
| 202, | NEQ100 | hypothetical protein | hemolectin | 0.55 | 0 | | |
| 203 | | | [Drosophila | | | | |
| | | | melanogaster]. | | | | |
| 204, | NEQ101 | LSU Ribosomal | Ribosomal protein | 1.00E−41 | Ribosomal_L11 Ribosomal | | Information |
| 205 | | protein | L11 [Methanopyrus | | protein L11, RNA binding | | storage and |
| | | L11[Methanopyrus | kandleri AV19]. | | domain 2.10e−17 | | processing; Translation, |
| | | kandleri AV19] | | | :Ribosomal_L11_N | | ribosomal |
| | | | | | Ribosomal protein L11, N- | | structure and |
| | | | | | terminal domain 1.90e−17 | | biogenesis; Ribosomal |
| | | | | | | | protein L11 |
| | | | | | | | 3e−41 |
| 206, | NEQ103 | Archaeal/vacuolar- | V-TYPE ATP | 1.00E−180 | ATP-synt_ab ATP | 3.6.3. | Metabolism; Energy |
| 207 | | type H+- | SYNTHASE ALPHA | | synthase alpha/beta | 14 | production |
| | | ATPasesubunit A | CHAIN (V-TYPE | | family, nucleotide- | | and |
| | | [Methanopyrus | ATPASE SUBUNIT | | binding domain | | conversion; Archaeal/ |
| | | kandleri] | A). | | 4.30e−84 :ATP- | | vacuolar- |
| | | | | | synt_ab_C ATP | | type H+- |
| | | | | | synthase alpha/beta | | ATPase |
| | | | | | chain, C terminal | | subunit A 1e−176 |
| | | | | | domain 2.90e−18 | | |
| | | | | | :ATP-synt_ab_N | | |
| | | | | | ATP synthase | | |
| | | | | | alpha/beta family, | | |
| | | | | | beta-barrel domain | | |
| | | | | | 6.00e−17 :rho rho | | |
| | | | | | transcription | | |
| | | | | | termination factor | | |
| | | | | | Rho 5.50e−3 | | |
| | | | | | :fliI_yscN fliI_yscN | | |
| | | | | | ATPase FliI/YscN | | |
| | | | | | family 6.00e−09 | | |
| | | | | | :atpD atpD ATP | | |
| | | | | | synthase F1, beta | | |
| | | | | | subunit 2.00e−11 :V- | | |
| | | | | | ATPase_V1_B V- | | |
| | | | | | ATPase_V1_B V- | | |
| | | | | | type ATPase, | | |
| | | | | | subunit B 3.00e−09 | | |
| | | | | | :ATP_syn_B_arch | | |
| | | | | | ATP_syn_B_arch | | |
| | | | | | ATP synthase | | |
| | | | | | archaeal, B subunit | | |
| | | | | | 5.50e−10 :V- | | |
| | | | | | ATPase_V1_A V- | | |
| | | | | | ATPase_V1_A V- | | |
| | | | | | type ATPase, | | |
| | | | | | subunit A 2.10e−222 | | |
| | | | | | :ATP_syn_A_arch | | |
| | | | | | ATP_syn_A_arch | | |
| | | | | | ATP synthase | | |
| | | | | | archaeal, A subunit | | |
| | | | | | 0.0 | | |
| 208, | NEQ102 | histidyl-tRNA | histidyl-tRNA | 8.00E−88 | HGTP_anticodon | 6.1.1. | Information |
| 209 | | synthetase (class | synthetase | | Anticodon binding | 21 | storage and |
| | | 2)[Aeropyrum pernix] | [Aeropyrum pernix]. | | domain 2.40e−10 | | processing; Translation, |
| | | | | | :tRNA-synt_2b tRNA | | ribosomal |
| | | | | | synthetase class II | | structure and |
| | | | | | core domain (G, H, | | biogenesis; Histidyl- |
| | | | | | P, S and T) 5.80e−32 | | tRNA |
| | | | | | :hisS hisS | | synthetase |
| | | | | | histidyl-tRNA | | 5e−89 |
| | | | | | synthetase 4.40e−77 | | |
| | | | | | :hisS_second | | |
| | | | | | hisS_second | | |
| | | | | | histidyl-tRNA | | |
| | | | | | synthetase 2, | | |
| | | | | | putative 4.10e−33 | | |
| 210, | NEQ104 | hypothetical protein | conserved | 4.00E−05 | 0 | | |
| 211 | | | hypothetical protein | | | | |
| | | | [Methanococcus | | | | |
| | | | jannaschii]. | | | | |
| 212, | NEQ105 | SSU Ribosomal | Ribosomal protein | 6.00E−26 | Ribosomal_S6e Ribosomal | | Information |
| 213 | | protein S6E | S6E (S10) | | protein S6e 1.20e−23 | | storage and |
| | | (S10)[Methanopyrus | [Methanopyrus | | | | processing; Translation, |
| | | kandleri AV19]. | kandleri AV19]. | | | | ribosomal |
| | | | | | | | structure and |
| | | | | | | | biogenesis; Ribosomal |
| | | | | | | | protein S6E |
| | | | | | | | (S10) 9e−24 |
| 214, | NEQ107 | N2,N2- | hypothetical protein | 8.00E−04 | 0 | | |
| 215 | | dimethylguanosine | [Plasmodium | | | | |
| | | tRNAmethyltransferase | falciparum 3D7]. | | | | |
| | | [Aquifex aeolicus] | | | | | |
| 216, | NEQ109 | conserved | cell division protein | 2.00E−10 | eRF1_1 eRF1 domain 1 | | Poorly |
| 217 | | hypothetical | pelota (pelA), | | 8.40e−4: pelA pelA cell | | characterized; |
| | | protein [Archaeoglobus | conjectural | | division protein pelota 6.80e−3 | | General |
| | | fulgidus] | [Pyrobaculum | | | | function |
| | | | aerophilum]. | | | | prediction |
| | | | | | | | only; Predicted |
| | | | | | | | RNA- |
| | | | | | | | binding |
| | | | | | | | proteins 2e−10 |
| 218, | NEQ108 | cell division protein | N2,N2- | 4.00E−56 | TRM N2,N2- | 2.1.1. | Information |
| 219 | | pelota | dimethylguanosine | | dimethylguanosine | 32 | storage and |
| | | (pelA), conjectural | tRNA | | tRNA | | processing; Translation, |
| | | [Pyrobaculum | methyltransferase | | methyltransferase | | ribosomal |
| | | aerophilum] | [Aquifex aeolicus]. | | 1.10e−80 :TRM1 | | structure and |
| | | | | | TRM1 N2,N2- | | biogenesis; N2, |
| | | | | | dimethylguanosine | | N2- |
| | | | | | tRNA | | dimethylguanosine |
| | | | | | methyltransferase | | tRNA |
| | | | | | 1.30e−65 | | methyltransferase |
| | | | | | | | 3e−57 |
| 220, | NEQ110 | Predicted exosome | conserved | 1.00E−09 | | | Poorly |
| 221 | | subunit, | hypothetical protein | | | | characterized; |
| | | predictedexoribonuclease | [Archaeoglobus | | | | General |
| | | related to | fulgidus]. | | | | function |
| | | RNase PH | | | | | prediction |
| | | | | | | | only; Archaeal |
| | | | | | | | serine |
| | | | | | | | proteases 7e−10 |
| 222, | NEQ111 | GTP-binding protein, | conserved | 2.00E−26 | RNase_PH 3′ | 2.7.7. | Information |
| 223 | | gtp1/obg | hypothetical protein | | exoribonuclease | 56 | storage and |
| | | family[Pyrococcus | [Archaeoglobus | | family, domain 1 | | processing; Translation, |
| | | furiosus DSM 3638] | fulgidus]. | | 1.20e−12 | | ribosomal |
| | | | | | :RNase_PH_C 3′ | | structure and |
| | | | | | exoribonuclease | | biogenesis; RNase |
| | | | | | family, domain 2 | | PH- |
| | | | | | 6.30e−4 | | related |
| | | | | | | | exoribonuclease |
| | | | | | | | 1e−27 |
| 224, | NEQ112 | hypothetical protein | GTP-binding protein, | 8.00E−50 | GTP1_OBG GTP1/OBG | | Poorly |
| 225 | | [Pyrococcusfuriosus | gtp1/obg family | | family 1.70e−3 :TGS TGS | | characterized; |
| | | DSM 3638] | [Pyrococcus furiosus | | domain 1.20e−22 | | General |
| | | | DSM 3638]. | | | | function |
| | | | | | | | prediction |
| | | | | | | | only; Predicted |
| | | | | | | | GTPase 2e−49 |
| 226, | NEQ113 | hypothetical | hypothetical protein | 4.00E−06 | DUF54 Protein of unknown | | |
| | | | | | function DUF54 1.80e−3 | | |
| 227 | | nucleotidyltransferase | [Pyrococcus furiosus | | | | |
| | | [Pyrococcus | DSM 3638]. | | | | |
| | | furiosus DSM 3638] | | | | | |
| 228, | NEQ114 | Tryptophanyl-tRNA | 399aa long | 2.00E−39 | HD HD domain 3.00e−08 | | Poorly |
| 229 | | synthetase | hypothetical | | | | characterized; |
| | | (trpS) (class 1b) | interferon-gamma | | | | General |
| | | [Sulfolobus | inducible protein | | | | function |
| | | solfataricus] | [Sulfolobus tokodaii]. | | | | prediction |
| | | | | | | | only; HD |
| | | | | | | | superfamily |
| | | | | | | | phosphohydrolases |
| | | | | | | | 1e−40 |
| 230, | NEQ115 | hypothetical protein | Tryptophanyl-tRNA | 1.00E−111 | tRNA-synt_1b tRNA | 6.1.1.2 | Information |
| 231 | | | synthetase (trpS) | | synthetases class I | | storage and |
| | | | [Sulfolobus | | (W and Y) 7.10e−40 | | processing; Translation, |
| | | | solfataricus]. | | :trpS trpS | | ribosomal |
| | | | | | tryptophanyl-tRNA | | structure and |
| | | | | | synthetase 5.40e−55 | | biogenesis; Tryptophanyl- |
| | | | | | | | tRNA |
| | | | | | | | synthetase |
| | | | | | | | 1e−102 |
| 232, | NEQ116 | Predicted nucleotide | olfactory receptor 1-5 | 0.88 | 0 | | |
| 233 | | kinase related | [Takifugu rubripes]. | | | | |
| | | toCMP and AMP | | | | | |
| | | kinase | | | | | |
| | | [Methanopyrus | | | | | |
| 234, | NEQ118 | cell division inhibitor | phosphoesterase | 3.00E−04 | DHH DHH family 6.50e−07 | | |
| 235 | | minD | [Methanosarcina | | | | |
| | | homolog [Pyrococcus | acetivorans str. C2A]. | | | | |
| | | furiosus DSM 3638] | | | | | |
| 236, | NEQ117 | phosphoesterase | hypothetical protein | 3.00E−25 | SKI Shikimate kinase 2.70e−3 | | Metabolism; Nucleotide |
| 237 | | [Methanosarcinaacetivorans | [Pyrococcus | | | | transport and |
| | | str. C2A] | horikoshii]. | | | | metabolism; Predicted |
| | | | | | | | nucleotide |
| | | | | | | | kinase |
| | | | | | | | (related to |
| | | | | | | | CMP and |
| | | | | | | | AMP kinases) |
| | | | | | | | 2e−26 |
| 238, | NEQ119 | hypothetical protein | cell division inhibitor | 6.00E−36 | ArsA_ATPase Anion- | | Cellular |
| 239 | | | minD homolog | | transporting ATPase 4.50e−3 | | processes; Cell |
| | | | [Pyrococcus furiosus | | :fer4_NifH 4Fe-4S iron sulfur | | division and |
| | | | DSM 3638]. | | cluster binding proteins, | | chromosome |
| | | | | | NifH/frxC family 6.10e−3 | | partitioning; ATPases |
| | | | | | :ParA ParA family ATPase | | involved in |
| | | | | | 2.40e−17 :eps_fam eps_fam | | chromosome |
| | | | | | capsular exopolysaccharide | | partitioning |
| | | | | | family 5.90e−08 | | 3e−34 |
| 240, | NEQ120 | hypothetical protein | similar to Plasmodium | 0.83 | 0 | | |
| 241 | | | falciparum. | | | | |
| | | | Asparagine-rich | | | | |
| | | | antigen [Dictyostelium | | | | |
| | | | discoideum]. | | | | |
| 242, | NEQ121 | hypothetical protein | hypothetical protein | 4.00E−06 | 0 | | |
| 243 | | | [Plasmodium | | | | |
| | | | falciparum 3D7]. | | | | |
| 244, | NEQ123 | hypothetical protein | claustrin-chicken. | 2.00E−09 | 0 | | Information |
| 245 | | | | | | | storage and |
| | | | | | | | processing; Translation, |
| | | | | | | | ribosomal |
| | | | | | | | structure and |
| | | | | | | | biogenesis; Translation |
| | | | | | | | initiation |
| | | | | | | | factor 2 |
| | | | | | | | (GTPase) 1e−07 |
| 246, | NEQ122 | Metal-dependent | METAL DEPENDENT | 1.00E−40 | lactamase_B Metallo-beta- | | Poorly |
| 247 | | hydrolase of thebeta- | HYDROLASE | | lactamase superfamily | | characterized; |
| | | lactamase | [Pyrococcus abyssi]. | | 3.30e−07 | | General |
| | | superfamily | | | | | function |
| | | | | | | | prediction |
| | | | | | | | only; Metal- |
| | | | | | | | dependent |
| | | | | | | | hydrolases of |
| | | | | | | | the beta- |
| | | | | | | | lactamase |
| | | | | | | | superfamily I |
| | | | | | | | 1e−41 |
| 248, | NEQ124 | Queuine/archaeosine | queuine trna- | 4.00E−87 | TGT Queuine tRNA- | 2.4.2. | Information |
| 249 | | tRNA- | ribosyltransferase | | ribosyltransferase | 29 | storage and |
| | | ribosyltransferase [Methanopyrus | [Pyrococcus furiosus | | 1.20e−37 | | processing; Translation, |
| | | | DSM 3638]. | | :Q_tRNA_tgt | | ribosomal |
| | | | | | Q_tRNA_tgt | | structure and |
| | | | | | queuine tRNA- | | biogenesis; Queuine/ |
| | | | | | ribosyltransferase | | archaeosine |
| | | | | | 1.80e−10 | | tRNA- |
| | | | | | :arcsn_tRNA_tgt | | ribosyltransferase |
| | | | | | arcsn_tRNA_tgt | | 4e−86 |
| | | | | | archaeosine tRNA- | | |
| | | | | | ribosyltransferase | | |
| | | | | | 2.00e−15 | | |
| | | | | | :tgt_general | | |
| | | | | | tgt_general tRNA- | | |
| | | | | | guanine | | |
| | | | | | transglycosylases, | | |
| | | | | | various specificities | | |
| | | | | | 2.00e−60 | | |
| 250, | NEQ125 | fibrillarin-like pre- | fibrillarin-like pre- | 1.00E−65 | Fibrillarin Fibrillarin 9.60e−96 | | Information |
| 251 | | rRNA | rRNA processing | | | | storage and |
| | | processingprotein | protein [Pyrococcus | | | | processing; Translation, |
| | | [Pyrococcus | furiosus DSM 3638]. | | | | ribosomal |
| | | furiosus] | | | | | structure and |
| | | | | | | | biogenesis; Fibrillarin- |
| | | | | | | | like |
| | | | | | | | rRNA |
| | | | | | | | methylase 8e−67 |
| 252, | NEQ126 | L-asparaginase | L-asparaginase | 2.00E−70 | Asparaginase | 3.5.1.1 | Metabolism; Amino |
| 253 | | [Pyrococcushorikoshii] = GatD | [Pyrococcus | | Asparaginase | | acid |
| | | | horikoshii]. | | 7.80e−14 :asnASE_I | | transport and |
| | | | | | asnASE_I L- | | metabolism; L- |
| | | | | | asparaginases, type | | asparaginase/ |
| | | | | | I 1.40e−81 | | archaeal Glu- |
| | | | | | :asnASE_II | | tRNAGln |
| | | | | | asnASE_II L- | | amidotransferase |
| | | | | | asparaginases, type | | subunit D |
| | | | | | II 2.20e−08 | | 1e−71 |
| 254, | NEQ126a | PUTATIVE | PUTATIVE | 5.00E−25 | DUF123 Domain of | 4.2.99. | Poorly |
| 255 | | ENDONUCLEASE III | ENDONUCLEASE. | | unknown function | 18 | characterized; |
| | | [Pyrococcus abyssi] | [Pyrococcus abyssi]. | | DUF123 3.70e−33 | | Function |
| | | SPLIT see SEQ ID | | | | | unknown; Uncharacterized |
| | | NOS: 778, 779 | | | | | ACR 3e−26 |
| 256, | NEQ127 | Predicted carbamoyl | nodulation protein | 0 | CmcH_NodU | 2.—.—.— | Cellular |
| 257 | | transferase, NodU | [Methanococcus | | Carbamoyltransferase | | processes; Posttranslational |
| | | family | jannaschii]. | | 2.10e−147 | | modification, |
| | | [Methanopyrus | | | | | protein |
| | | | | | | | turnover, |
| | | | | | | | chaperones; Predicted |
| | | | | | | | carbamoyl |
| | | | | | | | transferase, |
| | | | | | | | NodU family 0 |
| 258, | NEQ128 | conserved | conserved | 2.00E−13 | 0 | | Poorly |
| 259 | | hypothetical | hypothetical protein | | | | characterized; |
| | | protein[Thermotoga | [Thermotoga | | | | General |
| | | maritima] | maritima]. | | | | function |
| | | | | | | | prediction |
| | | | | | | | only; Uncharacterized |
| | | | | | | | proteins of |
| | | | | | | | the AP |
| | | | | | | | superfamily |
| | | | | | | | 2e−14 |
| 260, | NEQ130 | translation initiation | translation initiation | 1.00E−14 | eIF-1a Eukaryotic initiation | | Information |
| 261 | | factor aIF- | factor aIF-1A | | factor 1A 2.00e−14 :eIF-1A | | storage and |
| | | 1A [Methanococcus | [Methanococcus | | eIF-1A translation initiation | | processing; Translation, |
| | | jannaschii] | jannaschii]. | | factor eIF-1A 7.40e−18 | | ribosomal |
| | | | | | | | structure and |
| | | | | | | | biogenesis; Translation |
| | | | | | | | initiation |
| | | | | | | | factor IF-1 8e−16 |
| 262, | NEQ129 | Predicted membrane | hypothetical protein | 7.00E−18 | UPF0051 Uncharacterized | | Poorly |
| 263 | | components ofan | [Desulfovibrio | | protein family (UPF0051) | | characterized; |
| | | uncharacterized iron- | desulfuricans G20]. | | 1.60e−07 | | General |
| | | regulated | | | | | function |
| | | | | | | | prediction |
| | | | | | | | only; Predicted |
| | | | | | | | membrane |
| | | | | | | | components |
| | | | | | | | of an |
| | | | | | | | uncharacterized |
| | | | | | | | iron- |
| | | | | | | | regulated |
| | | | | | | | ABC-type |
| | | | | | | | transporter |
| | | | | | | | SufB 9e−16 |
| 264, | NEQ131 | Predicted RNA- | conserved | 5.00E−43 | Translin Translin family | | Information |
| 265 | | binding protein of | hypothetical protein | | 3.70e−09 | | storage and |
| | | thetranslin family [M kandleri] | [Pyrobaculum | | | | processing; DNA |
| | | | aerophilum]. | | | | replication, |
| | | | | | | | recombination |
| | | | | | | | and |
| | | | | | | | repair; Translin |
| | | | | | | | (RNA- |
| | | | | | | | binding |
| | | | | | | | protein, |
| | | | | | | | recombination |
| | | | | | | | hotspot |
| | | | | | | | binding in |
| | | | | | | | eukaryotes) |
| | | | | | | | 1e−16 |
| 266, | NEQ133 | cell division protein | cell division protein | 1.00E−102 | tubulin Tubulin/FtsZ | 3.4.24.— | Cellular |
| 267 | | FtsZ | FtsZ [Pyrococcushorikoshii]. | | family, GTPase | | processes; Cell |
| | | [Pyrococcushorikoshii] | | | domain 2.40e−77 | | division and |
| | | | | | :ftsZ ftsZ cell | | chromosome |
| | | | | | division protein FtsZ | | partitioning; Cell |
| | | | | | 1.20e−125 | | division |
| | | | | | :tubulin_C | | GTPase 1e−103 |
| | | | | | Tubulin/FtsZ family, | | |
| | | | | | C-terminal domain | | |
| | | | | | 2.90e−16 | | |
| 268, | NEQ132 | hypothetical protein | RNA polymerase | 2.6 | 0 | | |
| 269 | | | beta-prime subunit | | | | |
| | | | [Candidatus | | | | |
| | | | Carsonella ruddii]. | | | | |
| 270, | NEQ134 | hypothetical protein | hypothetical protein | 1 | 0 | | |
| 271 | | | [Plasmodium yoelii | | | | |
| | | | yoelii]. | | | | |
| 272, | NEQ135 | hypothetical protein | unknown protein- | 3.00E−03 | 0 | | |
| 273 | | | related [Plasmodium | | | | |
| | | | yoelii yoelii]. | | | | |
| 274, | NEQ136 | hypothetical protein | putative retroelement | 2.4 | 0 | | |
| 275 | | | pol polyprotein | | | | |
| | | | [Arabidopsis thaliana]. | | | | |
| 276, | NEQ139 | hypothetical protein | hypothetical protein | 0.01 | 0 | | |
| 277 | | | [Plasmodium | | | | |
| | | | falciparum 3D7]. | | | | |
| 278, | NEQ139 | hypothetical protein | ymf77 [Tetrahymena | 2.00E−06 | 0 | | |
| 279 | | | thermophila]. | | | | |
| 280, | NEQ138 | hypothetical protein | 521aa long | 0.05 | 0 | | |
| 281 | | | hypothetical protein | | | | |
| | | | [Sulfolobus tokodaii]. | | | | |
| 282, | NEQ140 | hypothetical protein | ORF MSV156 | 2.00E−03 | 0 | | |
| 283 | | | hypothetical protein | | | | |
| | | | [Melanoplus | | | | |
| | | | sanguinipes | | | | |
| | | | entomopoxvirus]. | | | | |
| 284, | NEQ141 | HSP60 family | HSP60 family | 0 | cpn60_TCP1 TCP- | 2.7.1. | Cellular |
| 285 | | chaperonin[Methanopyrus | chaperonin | | 1/cpn60 chaperonin | 68 | processes; Posttranslational |
| | | kandleri AV19] | [Methanopyrus | | family 4.30e−219 | | modification, |
| | | | kandleri AV19]. | | | | protein |
| | | | | | | | turnover, |
| | | | | | | | chaperones; Chaperonin |
| | | | | | | | GroEL |
| | | | | | | | (HSP60 |
| | | | | | | | family) 0 |
| 286, | NEQ144 | DNA topoisomerase | DNA topoisomerase | 1.00E−107 | HATPase_c | 5.99. | Information |
| 287 | | VI, subunit B (top6B) | VI, subunit B (top6B) | | Histidine kinase-, | 1.3 | storage and |
| | | [Archaeoglobus | [Archaeoglobus | | DNA gyrase B-, and | | processing; DNA |
| | | fulgidus] | fulgidus]. | | HSP90-like ATPase | | replication, |
| | | | | | 3.70e−11 :top6b | | recombination |
| | | | | | top6b DNA | | and |
| | | | | | topoisomerase VI, B | | repair; DNA |
| | | | | | subunit 7.80e−160 | | topoisomerase |
| | | | | | | | VI, subunit |
| | | | | | | | B 1e−108 |
| 288, | NEQ143 | Predicted | 165aa long conserved | 4.00E−18 | HTH_3 Helix-turn-helix | | Information |
| 289 | | transcription | hypothetical protein | | 4.00e−16 :TIGR00270 | | storage and |
| | | factor, homolog of | [Sulfolobus tokodaii]. | | TIGR00270 conserved | | processing; Transcription; |
| | | eukaryotic MBF1 [M. | | | hypothetical protein | | Predicted |
| | | | | | TIGR00270 1.10e−15 | | transcription |
| | | | | | | | factor, |
| | | | | | | | homolog of |
| | | | | | | | eukaryotic |
| | | | | | | | MBF1 5e−17 |
| 290, | NEQ146 | LSU Ribosomal | Ribosomal protein L4 | 2.00E−55 | Ribosomal_L4 Ribosomal | | Information |
| 291 | | protein | [Methanopyrus | | protein L4/L1 family 4.60e−69 | | storage and |
| | | L4 [Methanopyrus | kandleri AV19]. | | | | processing; Translation, |
| | | kandleri AV19] | | | | | ribosomal |
| | | | | | | | structure and |
| | | | | | | | biogenesis; Ribosomal |
| | | | | | | | protein L4 1e−52 |
| 292, | NEQ145 | hypothetical protein | hypothetical protein | 0.36 | 0 | | |
| 293 | | | [Clostridium | | | | |
| | | | thermocellum ATCC | | | | |
| | | | 27405]. | | | | |
| 294, | NEQ147 | conserved | conserved | 7.00E−35 | UPF0047 Uncharacterised | | Poorly |
| 295 | | hypothetical | hypothetical protein | | protein family UPF0047 | | characterized; |
| | | protein [Archaeoglobus | [Archaeoglobus | | 2.40e−48 :TIGR00149 | | Function |
| | | fulgidus] | fulgidus]. | | TIGR00149 conserved | | unknown; Uncharacterized |
| | | | | | hypothetical protein | | ACR 4e−36 |
| | | | | | TIGR00149 3.30e−34 | | |
| 296, | NEQ148 | Fe—S oxidoreductase | hypothetical protein | 1.00E−163 | B12-binding B12 | 1.97. | Metabolism; Energy |
| 297 | | family | [Pyrococcus | | binding domain | 1.4 | production |
| | | protein [Methanopyrus | horikoshii]. | | 8.90e−19 | | and |
| | | kandleri AV19] | | | :TIGR00089 | | conversion; Fe—S |
| | | | | | TIGR00089 | | oxidoreductases |
| | | | | | conserved | | family 2 |
| | | | | | hypothetical protein | | 1e−164 |
| | | | | | 2.10e−06 | | |
| | | | | | :Radical_SAM | | |
| | | | | | Radical SAM | | |
| | | | | | superfamily 2.00e−26 | | |
| 298, | NEQ150 | DNA-binding protein | hypothetical protein | 2.00E−07 | DUF122 Protein of unknown | | Poorly |
| 299 | | [Aeropyrumpernix] | [Pyrococcus | | function DUF122 1.20e−05 | | characterized; |
| | | | horikoshii]. | | | | General |
| | | | | | | | function |
| | | | | | | | prediction |
| | | | | | | | only; DNA- |
| | | | | | | | binding |
| | | | | | | | protein 1e−08 |
| 300, | NEQ149 | adenylate kinase | adenylate kinase | 2.00E−12 | | 2.7.4.3 | Metabolism; Nucleotide |
| 301 | | (ATP- | (ATP-AMP | | | | transport and |
| | | AMPtransphosphorylase) | transphosphorylase) | | | | metabolism; Archaeal |
| | | [Pyrococcus | [Pyrococcus furiosus | | | | adenylate |
| | | | DSM 3638]. | | | | kinase 2e−13 |
| 302, | NEQ151 | hypothetical protein | AotM [Aeromonas | 0.11 | 0 | | |
| 303 | | | hydrophila]. | | | | |
| 304, | NEQ152 | tRNA | 413aa long | 4.00E−32 | NTP_transf_2 | 2.7.7. | Information |
| 305 | | nucleotidyltransferase | hypothetical tRNA | | Nucleotidyltransferase | 25 | storage and |
| | | (tRNAadenylyltransferase) | nucleotidyltransferase | | domain 5.80e−06 | | processing; Translation, |
| | | (tRNA CCA- | [Sulfolobus tokodaii]. | | | | ribosomal |
| | | pyrophosphorylase) | | | | | structure and |
| | | | | | | | biogenesis; tRNA |
| | | | | | | | nucleotidyltransferase |
| | | | | | | | (CCA-adding |
| | | | | | | | enzyme) 4e−31 |
| 306, | NEQ153 | hypothetical protein | hypothetical protein | 0.06 | 0 | | |
| 307 | | | [Mycoplasma | | | | |
| | | | pneumoniae]. | | | | |
| 308, | NEQ154 | hypothetical protein | RKST1. | 0.07 | 0 | | |
| 309 | | | | | | | |
| 310, | NEQ155 | oligosaccharyl | hypothetical protein | 6.00E−16 | 0 | | Poorly |
| 311 | | transferase stt3 | [Pyrococcus | | | | characterized; |
| | | subunitrelated | horikoshii]. | | | | General |
| | | protein [P. furiosus] | | | | | function |
| | | | | | | | prediction |
| | | | | | | | only; Uncharacterized |
| | | | | | | | membrane |
| | | | | | | | protein, |
| | | | | | | | required for |
| | | | | | | | N-linked |
| | | | | | | | glycosylation |
| | | | | | | | 4e−17 |
| 312, | NEQ156 | DNA-directed RNA | DNA-directed RNA | 0 | RNA_pol_Rpb2_6 | 2.7.7.6 | Information |
| 313 | | polymerase, subunit | polymerase, subunit B | | RNA polymerase | | storage and |
| | | B′ (rpoB1) [M. jannaschii] | [Pyrococcus abyssi]. | | Rpb2, domain 6 | | processing; Transcription; |
| | | | | | 1.90e−158 | | DNA- |
| | | | | | :RNA_pol_Rpb2_7 | | directed |
| | | | | | RNA polymerase | | RNA |
| | | | | | Rpb2, domain 7 | | polymerase |
| | | | | | 3.50e−31 | | beta |
| | | | | | :RNA_pol_Rpb2_4 | | subunit/140 kD |
| | | | | | RNA polymerase | | subunit |
| | | | | | Rpb2, domain 4 | | (split gene in |
| | | | | | 1.40e−30 | | Mjan, Mthe, |
| | | | | | :RNA_pol_Rpb2_5 | | Aful) 0 |
| | | | | | RNA polymerase | | |
| | | | | | Rpb2, domain 5 | | |
| | | | | | 7.10e−17 | | |
| 314, | NEQ157 | GTP-binding protein, | hypothetical protein | 4.00E−44 | MMR_HSR1 GTPase of | | Poorly |
| 315 | | GTP1/OBG- | [Pyrococcus | | unknown function 5.40e−05 | | characterized; |
| | | family [Archaeoglobus | horikoshii]. | | :small_GTP small_GTP | | General |
| | | fulgidus] | | | small GTP-binding protein | | function |
| | | | | | domain 1.10e−08 | | prediction |
| | | | | | | | only; Predicted |
| | | | | | | | GTPase 3e−45 |
| 316, | NEQ158 | conserved | hypothetical protein | 2.00E−41 | DUF437 Protein of unknown | | Poorly |
| 317 | | hypothetical | [Pyrococcus furiosus | | function (DUF437) 4.90e−27 | | characterized; |
| | | protein [Pyrococcus | DSM 3638]. | | | | Function |
| | | horikoshii] | | | | | unknown; Uncharacterized |
| | | | | | | | ACR 1e−41 |
| 318, | NEQ159 | Predicted | 149aa long conserved | 1.00E−19 | PBP | | Poorly |
| 319 | | phospholipid- | hypothetical protein | | Phosphatidylethanolamine- | | characterized; |
| | | bindingprotein | [Sulfolobus tokodaii]. | | binding protein 7.60e−07 | | General |
| | | [Thermoplasma | | | :TIGR00481 TIGR00481 | | function |
| | | volcanium] | | | conserved hypothetical | | prediction |
| | | | | | protein TIGR00481 8.10e−13 | | only; Phospholipid- |
| | | | | | | | binding |
| | | | | | | | protein 4e−18 |
| 320, | NEQ160 | hypothetical protein | M. jannaschii | 0.01 | 0 | | |
| 321 | | | predicted coding | | | | |
| | | | region MJ0793 | | | | |
| | | | [Methanococcus | | | | |
| | | | jannaschii]. | | | | |
| 322, | NEQ162 | conserved putative | hypothetical protein | 6.00E−29 | UPF0118 Domain of | | Poorly |
| 323 | | membrane | [Aquifex aeolicus]. | | unknown function DUF20 | | characterized; |
| | | protein, possibly a | | | 4.90e−38 | | General |
| | | permease [P. abyssi] | | | | | function |
| | | | | | | | prediction |
| | | | | | | | only; Predicted |
| | | | | | | | permease |
| | | | | | | | 4e−30 |
| 324, | NEQ161 | hypothetical protein | hypothetical protein | 2.00E−06 | 0 | | |
| 325 | | | [Plasmodium yoelii | | | | |
| | | | yoelii]. | | | | |
| 326, | NEQ163 | hypothetical protein | EVM140 (Ectromelia | 1.2 | 0 | | |
| 327 | | | virus]. | | | | |
| 328, | NEQ164 | hypothetical | asoB protein | 8.00E−04 | 0 | | |
| 329 | | protein maybe | [Methanococcus | | | | |
| | | membran protein | jannaschii]. | | | | |
| 330, | NEQ165 | Predicted RNA | hypothetical protein | 8.00E−21 | GidB Glucose | 2.1.1.— | Information |
| 331 | | methylase[Methanopyrus | [Pyrococcus furiosus | | inhibited division | | storage and |
| | | kandleri AV19] | DSM 3638]. | | protein 5.30e−3 | | processing; Translation, |
| | | | | | :gidB gidB glucose- | | ribosomal |
| | | | | | inhibited division | | structure and |
| | | | | | protein B 5.50e−05 | | biogenesis; Predicted |
| | | | | | | | RNA |
| | | | | | | | methylase 6e−20 |
| 332, | NEQ166 | H(+)-transporting | H(+)-transporting ATP | 2.00E−15 | ATP-synt_D ATP | 3.6.3. | Metabolism; Energy |
| 333 | | ATP synthasesubunit | synthase subunit D | | synthase subunit D | 14 | production |
| | | D (V-type ATPase | [Pyrococcus | | 1.60e−09 | | and |
| | | subunit D) | horikoshii]. | | :V_ATPase_subD | | conversion; Archaeal/ |
| | | | | | V_ATPase_subD | | vacuolar- |
| | | | | | V-type ATPase, | | type H+- |
| | | | | | subunit D 1.90e−18 | | ATPase |
| | | | | | | | subunit D 2e−16 |
| 334, | NEQ168 | Preprotein | Preprotein | 9.00E−60 | secY eubacterial secY | | Cellular |
| 335 | | translocase subunit | translocase subunit | | protein 9.90e−09 | | processes; Cell |
| | | SecY[Methanopyrus | SecY [Methanopyrus | | :3a0501s007 3a0501s007 | | motility and |
| | | kandleri AV19] | kandleri AV19]. | | preprotein translocase, SecY | | secretion; Preprotein |
| | | | | | subunit 1.20e−15 | | translocase |
| | | | | | | | subunit SecY |
| | | | | | | | 8e−61 |
| 336, | NEQ170 | activator 1, | activator 1, replication | 1.00E−102 | AAA ATPase family | 2.7.7.7 | Information |
| 337 | | replication factor C, | factor C, 35 KD | | associated with | | storage and |
| | | 35 KD subunit | subunit | | various cellular | | processing; DNA |
| | | [Archaeoglobus | [Archaeoglobus | | activities (AAA) | | replication, |
| | | fulgidus] | fulgidus]. | | 1.30e−15 :ruvB ruvB | | recombination |
| | | | | | Holliday junction | | and |
| | | | | | DNA helicase RuvB | | repair; ATPase |
| | | | | | 5.50e−3 :holB holB | | involved in |
| | | | | | DNA polymerase III, | | DNA |
| | | | | | delta prime subunit | | replication 1e−103 |
| | | | | | 1.00e−3 | | |
| 338, | NEQ169 | type II secretion | type II secretion | 1.00E−104 | GSPII_E Type II/IV secretion | | Cellular |
| 339 | | system protein(gspE- | system protein (gspE- | | system protein 7.10e−06 | | processes; Cell |
| | | 1) [Archaeoglobus | 1) [Archaeoglobus | | | | motility and |
| | | fulgidus] | fulgidus]. | | | | secretion; Type |
| | | | | | | | IV secretory |
| | | | | | | | pathway, |
| | | | | | | | VirB11 |
| | | | | | | | components, |
| | | | | | | | and related |
| | | | | | | | ATPases |
| | | | | | | | involved in |
| | | | | | | | archaeal |
| | | | | | | | flagella |
| | | | | | | | biosynthesis |
| | | | | | | | 1e−106 |
| 340, | NEQ171 | hypothetical protein | similar to Plasmodium | 3.00E−05 | 0 | | |
| 341 | | | falciparum. | | | | |
| | | | Hypothetical protein | | | | |
| | | | [Dictyostelium | | | | |
| | | | discoideum]. | | | | |
| 342, | NEQ173 | DNA-directed RNA | DNA-DIRECTED RNA | 8.00E−96 | RNA_pol_Rpb2_2 | 2.7.7.6 | Information |
| 343 | | polymerase, subunit | POLYMERASE | | RNA polymerase | | storage and |
| | | B″ (rpoB2) [M. jannaschii] | SUBUNIT B. | | Rpb2, domain 2 | | processing; Transcription; |
| | | | | | 3.50e−07 | | DNA- |
| | | | | | :RNA_pol_Rpb2_1 | | directed |
| | | | | | RNA polymerase | | RNA |
| | | | | | beta subunit 5.50e−33 | | polymerase |
| | | | | | :RNA_pol_Rpb2_3 | | beta |
| | | | | | RNA polymerase | | subunit/140 kD |
| | | | | | Rpb2, domain 3 | | subunit |
| | | | | | 4.60e−21 | | (split gene in |
| | | | | | | | Mjan, Mthe, |
| | | | | | | | Aful) 1e−94 |
| 344, | NEQ172 | {putative | Eps7F [Streptococcus | 2.00E−12 | Glycos_transf_2 Glycosyl | | Cellular |
| 345 | | glycosyltransferase} | thermophilus]. | | transferase 2.50e−24 | | processes; Cell |
| | | | | | | | envelope |
| | | | | | | | biogenesis, |
| | | | | | | | outer |
| | | | | | | | membrane; Glycosyltransferases |
| | | | | | | | involved |
| | | | | | | | in cell wall |
| | | | | | | | biogenesis |
| | | | | | | | 3e−12 |
| 346, | NEQ175 | ABC-type transport | hypothetical protein | 1.00E−60 | DUF214 Predicted | | Poorly |
| 347 | | systems, involvedin | [Methanococcus | | permease 1.30e−33 | | characterized; |
| | | lipoprotein release, | jannaschii]. | | | | General |
| | | permease | | | | | function |
| | | | | | | | prediction |
| | | | | | | | only; ABC- |
| | | | | | | | type transport |
| | | | | | | | systems, |
| | | | | | | | involved in |
| | | | | | | | lipoprotein |
| | | | | | | | release, |
| | | | | | | | permease |
| | | | | | | | components |
| | | | | | | | 7e−62 |
| 348, | NEQ174 | RecA-superfamily | hypothetical protein | 1.00E−96 | | | Cellular |
| 349 | | ATPase implicatedin | [Pyrococcus | | | | processes; Signal |
| | | signal transduction | horikoshii]. | | | | transduction |
| | | [Mp. kandleri] | | | | | mechanisms; |
| | | | | | | | RecA- |
| | | | | | | | superfamily |
| | | | | | | | ATPases |
| | | | | | | | implicated in |
| | | | | | | | signal |
| | | | | | | | transduction |
| | | | | | | | 1e−97 |
| 350, | NEQ176 | SSU ribosomal | SSU ribosomal | 9.00E−16 | Ribosomal_S27 Ribosomal | | Information |
| 351 | | protein | protein S27AE; | | protein S27a 4.10e−21 | | storage and |
| | | S27AE; (rps27AE) | (rps27AE) | | | | processing; Translation, |
| | | [Pyrococcus | [Pyrococcus furiosus | | | | ribosomal |
| | | furiosus] | DSM 3638]. | | | | structure and |
| | | | | | | | biogenesis; Ribosomal |
| | | | | | | | protein |
| | | | | | | | S27AE 5e−16 |
| 352, | NEQ177 | threonyl-tRNA | threonyl-tRNA | 1.00E−151 | HGTP_anticodon | 6.1.1.3 | Information |
| 353 | | synthetase (class | synthetase | | Anticodon binding | | storage and |
| | | 2)[Pyrobaculum | [Pyrobaculum | | domain 1.20e−37 | | processing; Translation, |
| | | aerophilum] | aerophilum]. | | :tRNA-synt_2b tRNA | | ribosomal |
| | | | | | synthetase class II | | structure and |
| | | | | | core domain (G, H, | | biogenesis; Threonyl- |
| | | | | | P, S and T) 2.10e−06 | | tRNA |
| | | | | | :glyS_dimeric | | synthetase |
| | | | | | glyS_dimeric glycyl- | | 1e−108 |
| | | | | | tRNA synthetase | | |
| | | | | | 1.30e−3 :proS_fam_l | | |
| | | | | | proS_fam_l prolyl- | | |
| | | | | | tRNA synthetase | | |
| | | | | | 3.00e−4 :thrS thrS | | |
| | | | | | threonyl-tRNA | | |
| | | | | | synthetase 5.10e−50 | | |
| 354, | NEQ178 | LSU ribosomal | LSU ribosomal protein | 6.00E−20 | KOW KOW motif 2.80e−05 | | Information |
| 355 | | protein | L14E [Methanococcus | | | | storage and |
| | | L14E[Methanococcus | jannaschii]. | | | | processing; Translation, |
| | | jannaschii]. | | | | | ribosomal |
| | | | | | | | structure and |
| | | | | | | | biogenesis; Ribosomal |
| | | | | | | | protein L14E |
| | | | | | | | 3e−21 |
| 356, | NEQ179 | LSU ribosomal | LSU ribosomal protein | 2.00E−13 | Ribosomal_L7Ae Ribosomal | | Information |
| 357 | | protein | L30E [Methanococcus | | protein | | storage and |
| | | L30E[Methanococcus | jannaschii]. | | L7Ae/L30e/S12e/Gadd45 | | processing; Translation, |
| | | jannaschii] | | | family 1.80e−10 | | ribosomal |
| | | | | | | | structure and |
| | | | | | | | biogenesis; Ribosomal |
| | | | | | | | protein L30E |
| | | | | | | | 9e−15 |
| 358, | NEQ180 | transcription | NUSA PROTEIN | 6.00E−09 | 0 | | Information |
| 359 | | termination factor | HOMOLOG. | | | | storage and |
| | | nusA- | | | | | processing; Transcription; |
| | | Methanococcus | | | | | Transcription |
| | | vannielii | | | | | elongation |
| | | | | | | | factor 2e−08 |
| 360, | NEQ182 | DNA-directed RNA | DNA-DIRECTED RNA | 1.00E−10 | RNA_pool_L RNA | 2.7.7.6 | Information |
| 361 | | polymerasesubunit L | POLYMERASE | | polymerases L/13 | | storage and |
| | | [Sulfolobus | SUBUNIT L. | | to 16 kDa subunit | | processing; Transcription; |
| | | acidocaldarius] | | | 2.90e−12 | | DNA- |
| | | | | | | | directed |
| | | | | | | | RNA |
| | | | | | | | polymerase, |
| | | | | | | | subunit L 2e−08 |
| 362, | NEQ181 | LSU ribosomal | LSU ribosomal protein | 2.00E−58 | Ribosomal_L15e Ribosomal | | Information |
| 363 | | protein | L15E; (rpl15E) | | L15 1.60e−78 | | storage and |
| | | L15E; (rpl15E) | [Pyrococcus furiosus | | | | processing; Translation, |
| | | [Pyrococcus | DSM 3638]. | | | | ribosomal |
| | | furiosus] | | | | | structure and |
| | | | | | | | biogenesis; Ribosomal |
| | | | | | | | protein L15E |
| | | | | | | | 9e−59 |
| 364, | NEQ183 | LSU ribosomal | 50S ribosomal protein | 4.00E−16 | Ribosomal_L44 Ribosomal | | Information |
| 365 | | protein | L44 related protein | | protein L44 2.00e−11 | | storage and |
| | | L44E; (rpl44E) | [Thermoplasma | | | | processing; Translation, |
| | | [Pyrococcus | acidophilum]. | | | | ribosomal |
| | | furiosus] | | | | | structure and |
| | | | | | | | biogenesis; Ribosomal |
| | | | | | | | protein L44E |
| | | | | | | | 2e−17 |
| 366, | NEQ184 | Predicted exosome | conserved | 1.00E−23 | KH KH domain 2.00e−06 | | Information |
| 367 | | subunit, RNA- | hypothetical protein | | | | storage and |
| | | bindingprotein Rrp4 | [Archaeoglobus | | | | processing; Translation, |
| | | (contain S1 | fulgidus]. | | | | ribosomal |
| | | | | | | | structure and |
| | | | | | | | biogenesis; RNA- |
| | | | | | | | binding |
| | | | | | | | protein Rrp4 |
| | | | | | | | and related |
| | | | | | | | proteins |
| | | | | | | | (contain S1 |
| | | | | | | | domain and |
| | | | | | | | KH domain) |
| | | | | | | | 7e−25 |
| 368, | NEQ185 | Glu-tRNA | Aspartyl/glutamyl- | 1.00E−77 | DUF186 GatB/Yqey | 6.3.5.— | Information |
| 369 | | amidotransferase, | tRNA(Asn/Gln) | | domain 4.40e−4 | | storage and |
| | | subunitB (gatB-2) | amidotransferase | | :GatB_N PET112 | | processing; Translation, |
| | | [Archaeoglobus | subunit B (Asp/Glu- | | family, N terminal | | ribosomal |
| | | fulgidus] | ADT subunit B). | | region 5.40e−106 | | structure and |
| | | | | | :gatB gatB | | biogenesis; Asp- |
| | | | | | glutamyl-tRNA(Gln) | | tRNAAsn/Glu- |
| | | | | | amidotransferase, B | | tRNAGln |
| | | | | | subunit 4.40e−104 | | amidotransferase |
| | | | | | :gatB_rel gatB_rel | | B subunit |
| | | | | | aspartyl-tRNA(Asn) | | (PET112 |
| | | | | | amidotransferase, B | | homolog) 7e−79 |
| | | | | | subunit, putative | | |
| | | | | | 1.10e−23 :gatB_rel | | |
| | | | | | gatB_rel aspartyl- | | |
| | | | | | tRNA(Asn) | | |
| | | | | | amidotransferase, B | | |
| | | | | | subunit, putative | | |
| | | | | | 5.00e−4 :gatB_rel | | |
| | | | | | gatB_rel aspartyl- | | |
| | | | | | tRNA(Asn) | | |
| | | | | | amidotransferase, B | | |
| | | | | | subunit, putative | | |
| | | | | | 3.80e−3 | | |
| 370, | NEQ186 | ATP-dependent 26S | Proteasome-activating | 1.00E−111 | AAA ATPase family | 3.6.1.3 | Cellular |
| 371 | | proteasomeregulatory | nucleotidase | | associated with | | processes; Posttranslational |
| | | subunit [M. kandleri | (Proteasome | | various cellular | | modification, |
| | | | regulatory subunit). | | activities (AAA) | | protein |
| | | | | | 8.10e−89 :FtsH_fam | | turnover, |
| | | | | | FtsH_fam ATP- | | chaperones; ATP- |
| | | | | | dependent | | dependent |
| | | | | | metalloprotease | | 26S |
| | | | | | FtsH 4.70e−21 | | proteasome |
| | | | | | :26Sp45 26Sp45 | | regulatory |
| | | | | | 26S proteasome | | subunit 1e−107 |
| | | | | | subunit P45 family | | |
| | | | | | 7.10e−182 | | |
| 372, | NEQ188 | hypothetical protein | rubrerythrin (rr1) | 0.04 | 0 | | |
| 373 | | | [Archaeoglobus | | | | |
| | | | fulgidus]. | | | | |
| 374, | NEQ187 | SSU ribosomal | SSU ribosomal | 3.00E−37 | Ribosomal_S19e Ribosomal | | Information |
| 375 | | protein | protein S19E; | | protein S19e 9.00e−45 | | storage and |
| | | S19E; (rps19E) | (rps19E) [Pyrococcus | | | | processing; Translation, |
| | | [Pyrococcus | furiosus DSM 3638]. | | | | ribosomal |
| | | furiosus] | | | | | structure and |
| | | | | | | | biogenesis; Ribosomal |
| | | | | | | | protein S19E |
| | | | | | | | (S16A) 5e−38 |
| 376, | NEQ190 | branched-chain | branched-chain amino | 3.00E−68 | aminotran_4 | 2.6.1. | Metabolism; Amino |
| 377 | | amino | acid aminotransferase | | Aminotransferase | 42 | acid |
| | | acidaminotransferase | (ilvE) [Pyrobaculum | | class IV 5.60e−72 | | transport and |
| | | (ilvE) [Pb | aerophilum]. | | :D_amino_aminoT | | metabolism; Branched- |
| | | | | | D_amino_aminoT | | chain amino |
| | | | | | D-amino acid | | acid |
| | | | | | aminotransferase | | aminotransferase/ |
| | | | | | 6.10e−11 :ilvE_I | | 4-amino- |
| | | | | | ilvE_I branched- | | 4- |
| | | | | | chain amino acid | | deoxychorismate |
| | | | | | aminotransferase | | lyase 1e−62 |
| | | | | | 3.20e−107 :ilvE_II | | |
| | | | | | ilvE_II branched- | | |
| | | | | | chain amino acid | | |
| | | | | | aminotransferase | | |
| | | | | | 2.20e−21 | | |
| 378, | NEQ189 | hemolysin-related | probable hemolysin- | 8.00E−31 | CBS CBS domain 1.00e−05 | | Cellular |
| 379 | | protein[Thermotoga maritima] | related protein | | :DUF21 Domain of unknown | | processes; Cell |
| | | | [Clostridium | | function DUF21 3.90e−16 | | motility and |
| | | | perfringens]. | | :CorC_HlyC Transporter | | secretion; Hemolysins |
| | | | | | associated domain 3.90e−14 | | and |
| | | | | | | | related |
| | | | | | | | proteins |
| | | | | | | | containing |
| | | | | | | | CBS domains |
| | | | | | | | 7e−31 |
| 380, | NEQ191 | alkyl hydroperoxide | alkyl hydroperoxide | 3.00E−90 | AhpC-TSA | 1.6.4.— | Cellular |
| 381 | | reductase[Methanococcus | reductase | | AhpC/TSA family | | processes; Posttranslational |
| | | jannaschii] | [Methanococcus | | 2.40e−39 | | modification, |
| | | | jannaschii]. | | | | protein |
| | | | | | | | turnover, |
| | | | | | | | chaperones; Peroxiredoxin |
| | | | | | | | 2e−91 |
| 382, | NEQ193 | hypothetical protein | hypothetical protein | 0.12 | 0 | | |
| 383 | | | [Clostridium | | | | |
| | | | thermocellum ATCC | | | | |
| | | | 27405]. | | | | |
| 384, | NEQ192 | chorismate | chorismate | 2.00E−87 | ACT ACT domain | 4.2.1. | Metabolism; Amino |
| 385 | | mutase/prephenatedehydratase | mutase/prephenate | | 1.30e−10 | 51 | acid |
| | | (pheA) | dehydratase (pheA) | | :Chorismate_mut | | transport and |
| | | [A. fulgidus] | [Archaeoglobus | | Chorismate mutase | | metabolism; Prephenate |
| | | | fulgidus]. | | 1.50e−06 :PDH | | dehydratase |
| | | | | | Prephenate | | 9e−54 |
| | | | | | dehydrogenase | | |
| | | | | | 3.10e−05 :PDT | | |
| | | | | | Prephenate | | |
| | | | | | dehydratase 2.50e−51 | | |
| 386, | NEQ195 | putative | putative | 2.00E−16 | Glycos_transf_1 | 2.4.1.— | Cellular |
| 387 | | glycosyltransferase | glycosyltransferase | | Glycosyl | | processes; Cell |
| | | [Actinobacillus | [Actinobacillus | | transferases group 1 | | envelope |
| | | | actinomycetemcomitans]. | | 1.50e−21 | | biogenesis, |
| | | | | | | | outer |
| | | | | | | | membrane; Predicted |
| | | | | | | | glycosyltransferases |
| | | | | | | | 1e−15 |
| 388, | NEQ196 | hypothetical protein | Conserved | 0.53 | 0 | | |
| 389 | | | hypothetical protein | | | | |
| | | | [Sulfolobus | | | | |
| | | | solfataricus]. | | | | |
| 390, | NEQ194 | hypothetical protein | NADH | 0.29 | 0 | | |
| 391 | | | dehydrogenase | | | | |
| | | | subunit 5 | | | | |
| | | | [Caenorhabditis | | | | |
| | | | elegans]. | | | | |
| 392, | NEQ197 | hypothetical protein | SecY-independent | 0.02 | 0 | | |
| 393 | | | transporter protein | | | | |
| | | | [Chondrus crispus]. | | | | |
| 394, | NEQ198 | putative inner | conserved | 1.00E−107 | MS_channel | | Cellular |
| 395 | | membrane protein | hypothetical protein | | Mechanosensitive ion | | processes; Cell |
| | | | [Methanococcus | | channel 1.60e−86 | | envelope |
| | | | jannaschii]. | | | | biogenesis, |
| | | | | | | | outer |
| | | | | | | | membrane; Small- |
| | | | | | | | conductance |
| | | | | | | | mechanosensitive |
| | | | | | | | channel |
| | | | | | | | 1e−108 |
| 396, | NEQ199 | replication factor A | replication factor A | 1.00E−07 | tRNA_anti OB-fold nucleic | | Information |
| 397 | | related | related protein | | acid binding domain 7.40e−10 | | storage and |
| | | protein[Methanococcus | [Methanococcus | | | | processing; DNA |
| | | jannaschii]. | jannaschii]. | | | | replication, |
| | | | | | | | recombination |
| | | | | | | | and |
| | | | | | | | repair; Replication |
| | | | | | | | factor A |
| | | | | | | | large subunit |
| | | | | | | | and related |
| | | | | | | | ssDNA- |
| | | | | | | | binding |
| | | | | | | | proteins 1e−08 |
| 398, | NEQ200 | hypothetical protein | hypothetical protein | 0.18 | 0 | | |
| 399 | | | [Plasmodium | | | | |
| | | | falciparum 3D7]. | | | | |
| 400, | NEQ201 | LSU ribosomal | LSU ribosomal protein | 1.00E−30 | 60s_ribosomal 60s Acidic | | Information |
| 401 | | protein L12A(rpl12A) | L12A [Pyrococcus | | ribosomal protein 9.80e−19 | | storage and |
| | | [Pyrococcus abyssi] | abyssi]. | | | | processing; Translation, |
| | | | | | | | ribosomal |
| | | | | | | | structure and |
| | | | | | | | biogenesis; Ribosomal |
| | | | | | | | protein |
| | | | | | | | L12E/L44/L45/ |
| | | | | | | | RPP1/RPP2 |
| | | | | | | | 6e−32 |
| 402, | NEQ204 | LSU Ribosomal | Ribosomal protein | 4.00E−22 | Ribosomal_L22 Ribosomal | | Information |
| 403 | | protein | L22 [Methanopyrus | | protein L22p/L17e 1.20e−09 | | storage and |
| | | L22[Methanopyrus | kandleri AV19]. | | :L22_arch L22_arch | | processing; Translation, |
| | | kandleri AV19] | | | ribosomal protein L22 1.20e−16 | | ribosomal |
| | | | | | | | structure and |
| | | | | | | | biogenesis; Ribosomal |
| | | | | | | | protein L22 |
| | | | | | | | 7e−17 |
| 404, | NEQ203 | proteasome, subunit | proteasome, subunit | 1.00E−33 | proteasome | 3.4.25.1 | Cellular |
| 405 | | beta | beta (psmB) | | Proteasome A-type | | processes; Posttranslational |
| | | (psmB)(Multicatalytic | [Methanococcus | | and B-type 6.80e−44 | | modification, |
| | | endopeptidase | jannaschii]. | | | | protein |
| | | | | | | | turnover, |
| | | | | | | | chaperones; Proteasome |
| | | | | | | | protease |
| | | | | | | | subunit 9e−35 |
| 406, | NEQ205 | tRNA intron | Chain A, Crystal | 4.00E−21 | tRNA_int_endo | 3.1.27.9 | Information |
| 407 | | endonuclease | Structure Of The Trna | | tRNA intron | | storage and |
| | | (endA) [M. jannaschii] | Splicing | | endonuclease, | | processing; Translation, |
| | | | Endonuclease From | | catalytic C-terminal | | ribosomal |
| | | | Methanococcus | | domain 7.00e−19 | | structure and |
| | | | Jannaschii. | | :endA endA tRNA | | biogenesis; tRNA |
| | | | | | intron endonuclease | | splicing |
| | | | | | 1.20e−10 | | endonuclease |
| | | | | | | | 3e−22 |
| 408, | NEQ206 | conserved protein | conserved protein | 1.00E−20 | PUA PUA domain 2.60e−17 | | Information |
| 409 | | with predicted | with predicted RNA | | :unchar_dom_2 | | storage and |
| | | RNAbinding PUA | binding PUA domain | | unchar_dom_2 | | processing; Translation, |
| | | domain [Pb | [Pyrobaculum | | uncharacterized domain 2 | | ribosomal |
| | | aerophilum] | aerophilum]. | | 9.40e−17 | | structure and |
| | | | | | | | biogenesis; PUA |
| | | | | | | | domain |
| | | | | | | | (predicted |
| | | | | | | | RNA-binding |
| | | | | | | | domain) 1e−14 |
| 410, | NEQ208 | arginyl-tRNA | hypothetical protein | 2.00E−80 | tRNA-synt_1d tRNA | 6.1.1. | Information |
| 411 | | synthetase (class | [Lactobacillus | | synthetases class I | 19 | storage and |
| | | 1)[Streptococcus | gasseri]. | | (R) 8.70e−60 :argS | | processing; Translation, |
| | | pneumoniae TIGR4] | | | argS arginyl-tRNA | | ribosomal |
| | | | | | synthetase 4.70e−71 | | structure and |
| | | | | | | | biogenesis; Arginyl- |
| | | | | | | | tRNA |
| | | | | | | | synthetase |
| | | | | | | | 9e−71 |
| 412, | NEQ207 | LSU Ribosomal | Ribosomal protein | 5.00E−33 | Ribosomal_L13 Ribosomal | | Information |
| 413 | | protein | L13 [Methanopyrus | | protein L13 5.60e−28 | | storage and |
| | | L13[Methanopyrus | kandleri AV19]. | | :rpIM_bact rpIM_bact | | processing; Translation, |
| | | kandleri AV19] | | | ribosomal protein L13 3.60e−05 | | ribosomal |
| | | | | | :L13_A_E L13_A_E | | structure and |
| | | | | | ribosomal protein L13 6.20e−47 | | biogenesis; Ribosomal |
| | | | | | | | protein L13 |
| | | | | | | | 7e−29 |
| 414, | NEQ209 | hypothetical purine | virulent strain | 4.00E−11 | 0 | | Information |
| 415 | | NTPase [Phorikoshii] | associated lipoprotein | | | | storage and |
| | | | [Borrelia burgdorferi]. | | | | processing; DNA |
| | | | | | | | replication, |
| | | | | | | | recombination |
| | | | | | | | and |
| | | | | | | | repair; ATPase |
| | | | | | | | involved in |
| | | | | | | | DNA repair |
| | | | | | | | 4e−11 |
| 416, | NEQ210 | Prolyl-tRNA | Prolyl-tRNA | 6.00E−95 | HGTP_anticodon | 6.1.1. | Information |
| 417 | | synthetase (class | synthetase | | Anticodon binding | 15 | storage and |
| | | 2)[Methanopyrus | [Methanopyrus | | domain 7.40e−24 | | processing; Translation, |
| | | kandleri AV19] | kandleri AV19]. | | :tRNA-synt_2b tRNA | | ribosomal |
| | | | | | synthetase class II | | structure and |
| | | | | | core domain (G, H, | | biogenesis; Prolyl- |
| | | | | | P, S and T) 8.40e−33 | | tRNA |
| | | | | | :proS_fam_I | | synthetase |
| | | | | | proS_fam_I prolyl- | | 6e−62 |
| | | | | | tRNA synthetase | | |
| | | | | | 3.00e−145 | | |
| | | | | | :proS_fam_II | | |
| | | | | | proS_fam_II prolyl- | | |
| | | | | | tRNA synthetase | | |
| | | | | | 3.00e−05 | | |
| 418, | NEQ212 | Predicted nucleic | hypothetical protein | 1.00E−11 | PIN PIN domain 4.70e−3 | | Poorly |
| 419 | | acid-binding | [Pyrococcus furiosus | | | | characterized; |
| | | protein[Thermoplasma | DSM 3638]. | | | | General |
| | | volcanium] | | | | | function |
| | | | | | | | prediction |
| | | | | | | | only; Predicted |
| | | | | | | | nucleic |
| | | | | | | | acid-binding |
| | | | | | | | protein, |
| | | | | | | | consists of a |
| | | | | | | | PIN domain |
| | | | | | | | and a Zn- |
| | | | | | | | ribbon |
| | | | | | | | module 2e−12 |
| 420, | NEQ211 | alanyl-tRNA | alanyl-tRNA | 7.00E−76 | tRNA-synt_2c tRNA | 6.1.1.7 | Information |
| 421 | | synthetase | synthetase | | synthetases class II | | storage and |
| | | [Pyrococcus abyssi]. | [Pyrococcus abyssi]. | | (A) 5.90e−11 | | processing; Translation, |
| | | | | | | | ribosomal |
| | | | | | | | structure and |
| | | | | | | | biogenesis; Alanyl- |
| | | | | | | | tRNA |
| | | | | | | | synthetase |
| | | | | | | | 5e−77 |
| 422, | NEQ213 | histidine triad protein | histidine triad protein | 4.00E−18 | HIT HIT family 1.00e−11 | | Metabolism; Nucleotide |
| 423 | | (HIT familyprotein) | [Methanosarcina | | | | transport and |
| | | [Ms acetivorans] | acetivorans str. C2A]. | | | | metabolism; Diadenosine |
| | | | | | | | tetraphosphate |
| | | | | | | | (Ap4A) |
| | | | | | | | hydrolase and |
| | | | | | | | other HIT |
| | | | | | | | family |
| | | | | | | | hydrolases |
| | | | | | | | 9e−16 |
| 424, | NEQ214 | hypothetical | DNA GyrAse a- | 2.00E−03 | 0 | | |
| 425 | | proteinS-Layer | subunit, putative | | | | |
| | | domain? | [Plasmodium | | | | |
| | | | falciparum 3D7]. | | | | |
| 426, | NEQ215 | hypothetical protein | hypothetical protein | 0.08 | 0 | | |
| 427 | | | [Staphylococcus | | | | |
| | | | aureus subsp. aureus | | | | |
| | | | Mu50]. | | | | |
| 428, | NEQ216 | hypothetical protein | integral membrane | 1.00E−03 | 0 | | |
| 429 | | | protein [Plasmodium | | | | |
| | | | falciparum 3D7]. | | | | |
| 430, | NEQ217 | H+-transporting ATP | Archaeal/vacuolar- | 5.00E−11 | ATP-synt_C ATP | 3.6.3. | Metabolism; Energy |
| 431 | | synthase, subunit K | type H+-ATPase | | synthase subunit C | 14 | production |
| | | (atpK-1/atpK-2) | subunit K | | 8.70e−20 | | and |
| | | homolog - | [Methanopyrus | | :ATP_synt_c | | conversion; F0 |
| | | | kandleri AV19]. | | ATP_synt_c ATP | | F1-type ATP |
| | | | | | synthase, F0 | | synthase c |
| | | | | | subunit c 9.50e−3 | | subunit/Archaeal/ |
| | | | | | | | vacuolar- |
| | | | | | | | type H+- |
| | | | | | | | ATPase |
| | | | | | | | subunit K 4e−12 |
| 432, | NEQ218 | hypothetical protein | conserved | 0.04 | | | |
| 433 | | | hypothetical protein | | | | |
| | | | [Clostridium | | | | |
| | | | perfringens]. | | | | |
| 434, | NEQ219 | SSU ribosomal | Ribosomal protein | 1.00E−14 | Ribosomal_S27e Ribosomal | | Information |
| 435 | | protein S27E | S27E [Methanopyrus | | protein S27 7.10e−23 | | storage and |
| | | | kandleri AV19]. | | | | processing; Translation, |
| | | | | | | | ribosomal |
| | | | | | | | structure and |
| | | | | | | | biogenesis; Ribosomal |
| | | | | | | | protein S27E |
| | | | | | | | 6e−12 |
| 436, | NEQ220 | Translation | Translation elongation | 1.00E−07 | EF1BD EF-1 guanine | | Information |
| 437 | | elongation factor EF- | factor EF-1beta | | nucleotide exchange domain | | storage and |
| | | 1beta[Methanopyrus | [Methanopyrus | | 1.50e−09 :aEF-1_beta aEF- | | processing; Translation, |
| | | kandleri AV19] | kandleri AV19]. | | 1_beta translation | | ribosomal |
| | | | | | elongation factor aEF-1 beta | | structure and |
| | | | | | 6.10e−10 | | biogenesis; Translation |
| | | | | | | | elongation |
| | | | | | | | factor EF- |
| | | | | | | | 1beta 2e−08 |
| 438, | NEQ221 | hypothetical protein | hypothetical protein | 0.69 | 0 | | |
| 439 | | | T26A5.5- | | | | |
| | | | Caenorhabditis | | | | |
| | | | elegans. | | | | |
| 440, | NEQ222 | hypothetical protein | 132aa long | 0.03 | 0 | | |
| 441 | | | hypothetical protein | | | | |
| | | | [Sulfolobus tokodaii]. | | | | |
| 442, | NEQ223 | agmatinase (speB) | agmatinase (speB) | 2.00E−16 | arginase Arginase | 3.5.3. | Metabolism; Amino |
| 443 | | (agmatineureohydrolase) | [Archaeoglobus | | family 1.70e−07 | 11 | acid |
| | | [Archaeoglobus | fulgidus]. | | :hutG hutG | | transport and |
| | | | | | formiminoglutamase | | metabolism; Arginase/ |
| | | | | | 9.50e−05 | | agmatinase/ |
| | | | | | :agmatinase | | formimionoglutamate |
| | | | | | agmatinase | | hydrolase, |
| | | | | | agmatinase, | | arginase |
| | | | | | putative 4.90e−12 | | family 1e−17 |
| | | | | | 0 | | |
| 444, | NEQ224 | hypothetical protein | MYOSIN HEAVY | 0.03 | 0 | | |
| 445 | | | CHAIN | | | | |
| | | | [Encephalitozoon | | | | |
| | | | cuniculi]. | | | | |
| 446, | NEQ225 | hypothetical protein | hypothetical protein | 1.00E−03 | 0 | | |
| 447 | | | [Plasmodium | | | | |
| | | | falciparum 3D7]. | | | | |
| 448, | NEQ226 | putative diphthamide | hypothetical protein | 2.00E−31 | Diphthamide_syn Putative | | Information |
| 449 | | synthesis | [Ferroplasma | | diphthamide synthesis | | storage and |
| | | protein[Methanosarcina | acidarmanus]. | | protein 9.50e−13 :diphth2_R | | processing; Translation, |
| | | acetivorans] | | | diphth2_R diphthamide | | ribosomal |
| | | | | | biosynthesis protein 2- | | structure and |
| | | | | | related domain 9.80e−22 | | biogenesis; Diphthamide |
| | | | | | | | synthase |
| | | | | | | | subunit DPH2 |
| | | | | | | | 2e−31 |
| 450, | NEQ227 | SSU Ribosomal | SSU ribosomal | 1.00E−13 | Ribosomal_S14 Ribosomal | | Information |
| 451 | | protein S14 | protein S14AB | | protein S14p/S29e 4.30e−06 | | storage and |
| | | | (rps14AB) [Sulfolobus | | | | processing; Translation, |
| | | | solfataricus]. | | | | ribosomal |
| | | | | | | | structure and |
| | | | | | | | biogenesis; Ribosomal |
| | | | | | | | protein S14 |
| | | | | | | | 6e−14 |
| 452, | NEQ229 | transcriptional | Transcriptional | 2.00E−25 | ASNC_trans_reg AsnC | | Information |
| 453 | | regulatory | regulator Ptr2. | | family 1.30e−21 | | storage and |
| | | protein, AsnC family | | | | | processing; Transcription; |
| | | [M. jannaschii] | | | | | Transcriptional |
| | | | | | | | regulators 2e−26 |
| 454, | NEQ228 | met-10+ protein | met-10+ protein | 2.00E−32 | Met_10 Met-10+ | 2.1.1.— | Poorly |
| 455 | | [Pyrococcus | [Pyrococcus furiosus | | like-protein 4.20e−26 | | characterized; |
| | | furiosus DSM 3638] | DSM 3638]. | | | | General |
| | | | | | | | function |
| | | | | | | | prediction |
| | | | | | | | only; Predicted |
| | | | | | | | methyltransferase |
| | | | | | | | 8e−28 |
| 456, | NEQ230 | isoleucyl-tRNA | isoleucyl-tRNA | 1.00E−175 | tRNA-synt_1 tRNA | 6.1.1.5 | Information |
| 457 | | synthetase (class | synthetase | | synthetases class I | | storage and |
| | | 1a)[Pyrococcus | [Pyrococcus furiosus | | (I, L, M and V) | | processing; Translation, |
| | | furiosus DSM 3638] | DSM 3638]. | | 4.80e−188 :ileS ileS | | ribosomal |
| | | | | | isoleucyl-tRNA | | structure and |
| | | | | | synthetase 3.80e−199 | | biogenesis; Isoleucyl- |
| | | | | | :leuS_bact | | tRNA |
| | | | | | leuS_bact leucyl- | | synthetase |
| | | | | | tRNA synthetase | | 1e−175 |
| | | | | | 2.20e−05 :metG | | |
| | | | | | metG methionyl- | | |
| | | | | | tRNA synthetase | | |
| | | | | | 7.30e−07 :valS valS | | |
| | | | | | valyl-tRNA | | |
| | | | | | synthetase 9.80e−25 | | |
| 458, | NEQ231 | DNA-directed RNA | DNA-directed RNA | 9.00E−10 | RpoE2 Archaeal | 2.7.7.6 | Information |
| 459 | | polymerase, subunit | polymerase, subunit E | | DNA-directed RNA | | storage and |
| | | E″ (rpoE2) [S | (rpoE2) [Sulfolobus | | polymerase subunit | | processing; Transcription; |
| | | solfataricus] | solfataricus]. | | E″ (RpoE″ or | | DNA- |
| | | | | | RpoE2) 1.90e−17 | | directed |
| | | | | | | | RNA |
| | | | | | | | polymerase |
| | | | | | | | subunit E′ 2e−07 |
| 460, | NEQ233 | hypothetical protein | hypothetical protein | 0.22 | 0 | | |
| 461 | | | [Plasmodium yoelii | | | | |
| | | | yoelii]. | | | | |
| 462, | NEQ232 | hypothetical protein | hypothetical protein | 1.00E−04 | 0 | | |
| 463 | | | [Pyrococcus | | | | |
| | | | horikoshii]. | | | | |
| 464, | NEQ234 | methanol | methanol | 8.00E−73 | AAA ATPase family | | Poorly |
| 465 | | dehydrogenase | dehydrogenase | | associated with various | | characterized; |
| | | regulator; (moxR) | regulator; (moxR) | | cellular activities (AAA) | | General |
| | | [Pyrococcus | [Pyrococcus furiosus | | 6.90e−4 :Mg_chelatase | | function |
| | | furiosus] | DSM 3638]. | | Magnesium chelatase, | | prediction |
| | | | | | subunit ChII 4.20e−09 | | only; MoxR- |
| | | | | | | | like ATPases |
| | | | | | | | 2e−73 |
| 466, | NEQ235 | NMD protein | conserved protein | 1.00E−17 | 1.30E−18 | | Information |
| 467 | | affecting | [Methanothermobacter | | | | storage and |
| | | ribosomestability and | thermautotrophicus]. | | | | processing; Translation, |
| | | mRNA decay | | | | | ribosomal |
| | | | | | | | structure and |
| | | | | | | | biogenesis; NMD |
| | | | | | | | protein |
| | | | | | | | affecting |
| | | | | | | | ribosome |
| | | | | | | | stability and |
| | | | | | | | mRNA decay |
| | | | | | | | 1e−18 |
| 468, | NEQ236 | hypothetical protein | ELM2 domain, | 3.00E−09 | 0 | | |
| 469 | | | putative [Plasmodium | | | | |
| | | | yoelii yoelii]. | | | | |
| 470, | NEQ237 | hypothetical | conserved | 1.00E−04 | 0 | | |
| 471 | | proteinmaybe | hypothetical protein | | | | |
| | | membran protein | [Methanosarcina | | | | |
| | | | acetivorans str. C2A]. | | | | |
| 472, | NEQ238 | protoporphyrinogen | protoporphyrinogen | 2.00E−26 | hemK_rel_arch | | Information |
| 473 | | oxidase | oxidase (hemK) | | hemK_rel_arch methylase, | | storage and |
| | | (hemK)[Methanococcus | [Methanococcus | | putative 5.80e−41 | | processing; Translation, |
| | | jannaschii] | jannaschii]. | | | | ribosomal |
| | | | | | | | structure and |
| | | | | | | | biogenesis; Predicted |
| | | | | | | | rRNA |
| | | | | | | | or tRNA |
| | | | | | | | methylase 1e−27 |
| 474, | NEQ239 | leucyl-tRNA | leucyl-tRNA | 1.00E−168 | tRNA-synt_1 tRNA | 6.1.1.4 | Information |
| 475 | | synthetase (leuS) | synthetase (leuS) | | synthetases class I | | storage and |
| | | (class1a) | [Methanococcus | | (I, L, M and V) | | processing; Translation, |
| | | [Methanococcus | jannaschii]. | | 1.60e−12 :ileS ileS | | ribosomal |
| | | jannaschii] | | | isoleucyl-tRNA | | structure and |
| | | | | | synthetase 1.00e−06 | | biogenesis; Leucyl- |
| | | | | | :leuS_arch | | tRNA |
| | | | | | leuS_arch leucyl- | | synthetase |
| | | | | | tRNA synthetase | | 1e−169 |
| | | | | | 6.10e−170 | | |
| | | | | | :leuS_bact | | |
| | | | | | leuS_bact leucyl- | | |
| | | | | | tRNA synthetase | | |
| | | | | | 1.50e−3 :metG metG | | |
| | | | | | methionyl-tRNA | | |
| | | | | | synthetase 1.50e−06 | | |
| | | | | | :valS valS valyl- | | |
| | | | | | tRNA synthetase | | |
| | | | | | 4.10e−07 | | |
| 476, | NEQ240 | DNA polymerase II | DNA polymerase | 3.00E−67 | Metallophos | 2.7.7.7 | Information |
| 477 | | small subunit (PolII) | delta small subunit | | Calcineurin-like | | storage and |
| | | [Mc. jannaschii] | [Methanococcus | | phosphoesterase | | processing; DNA |
| | | | jannaschii]. | | 4.80e−13 :tRNA_anti | | replication, |
| | | | | | OB-fold nucleic acid | | recombination |
| | | | | | binding domain | | and |
| | | | | | 2.80e−10 | | repair; DNA |
| | | | | | | | polymerase II |
| | | | | | | | small subunit |
| | | | | | | | (predicted |
| | | | | | | | phosphatase) |
| | | | | | | | 2e−68 |
| 478, | NEQ241 | LSU ribosomal | 50S ribosomal protein | 2.00E−38 | Ribosomal_L6 Ribosomal | | Information |
| 479 | | protein | L6 [Pyrococcus | | protein L6 2.60e−17 | | storage and |
| | | L6[Pyrococcus | horikoshii]. | | :Ribosomal_L6 Ribosomal | | processing; Translation, |
| | | horikoshii] | | | protein L6 2.70e−06 | | ribosomal |
| | | | | | | | structure and |
| | | | | | | | biogenesis; Ribosomal |
| | | | | | | | protein L6 1e−39 |
| 480, | NEQ242 | SSU Ribosomal | Ribosomal protein S7 | 8.00E−52 | Ribosomal_S7 Ribosomal | | Information |
| 481 | | protein | [Methanopyrus | | protein S7p/S5e 9.00e−50 | | storage and |
| | | S7[Methanopyrus | kandleri AV19]. | | :S7_S5_E_A S7_S5_E_A | | processing; Translation, |
| | | kandleri AV19] | | | ribosomal protein S7 4.20e−95 | | ribosomal |
| | | | | | :rpsG_bact rpsG_bact | | structure and |
| | | | | | ribosomal protein S7 2.70e−09 | | biogenesis; Ribosomal |
| | | | | | | | protein S7 8e−51 |
| 482, | NEQ243 | hypothetical protein | hypothetical protein | 4.00E−04 | 0 | | |
| 483 | | | [Ferroplasma | | | | |
| | | | acidarmanus]. | | | | |
| 484, | NEQ245 | Glutamyl-tRNA-Gln | 628aa long conserved | 2.00E−80 | GatB_N PET112 | 6.3.5.— | Information |
| 485 | | amidotransferase | hypothetical protein | | family, N terminal | | storage and |
| | | (gatE) [Sulfolobus | [Sulfolobus tokodaii]. | | region 2.30e−90 | | processing; Translation, |
| | | solfataricus] SPLIT | | | :gatB gatB | | ribosomal |
| | | See SEQ ID | | | glutamyl-tRNA(Gln) | | structure and |
| | | NOS: 772, 773 | | | amidotransferase, B | | biogenesis; Archaeal |
| | | | | | subunit 1.90e−27 | | Glu- |
| | | | | | :gatB_rel gatB_rel | | tRNAGln |
| | | | | | aspartyl-tRNA(Asn) | | amidotransferase |
| | | | | | amidotransferase, B | | subunit E |
| | | | | | subunit, putative | | (contains |
| | | | | | 5.10e−109 | | GAD domain) |
| | | | | | | | 1e−79 |
| 486, | NEQ244 | conserved | conserved | 1.00E−29 | UPF0099 Domain of | | Poorly |
| 487 | | hypothetical | hypothetical protein | | unknown function UPF0099 | | characterized; |
| | | protein[Thermoplasma | [Thermoplasma | | 1.50e−37 :TIGR00283 | | Function |
| | | acidophilum] | acidophilum]. | | TIGR00283 conserved | | unknown; Uncharacterized |
| | | | | | hypothetical protein | | ACR 8e−31 |
| | | | | | TIGR00283 6.30e−41 | | |
| 488, | NEQ246 | hypothetical protein | hypothetical protein | 0.09 | 0 | | |
| 489 | | | [Plasmodium | | | | |
| | | | falciparum 3D7]. | | | | |
| 490, | NEQ247 | SSU ribosomal | 30S ribosomal protein | 4.00E−49 | S4 S4 domain 1.60e−18 | | Information |
| 491 | | protein | S4 [Pyrococcus | | :rpsD_bact rpsD_bact | | storage and |
| | | S4P[Pyrococcus | horikoshii]. | | ribosomal protein S4 7.20e−4 | | processing; Translation, |
| | | horikoshii] | | | :rpsD_arch rpsD_arch | | ribosomal |
| | | | | | ribosomal protein S4 3.00e−79 | | structure and |
| | | | | | | | biogenesis; Ribosomal |
| | | | | | | | protein S4 |
| | | | | | | | and related |
| | | | | | | | proteins 3e−50 |
| 492, | NEQ248 | Predicted exosome | ribonuclease ph (rph) | 1.00E−42 | RNase_PH 3′ | 2.7.7. | Information |
| 493 | | subunit, RNase | [Pyrococcus furiosus | | exoribonuclease | 56 | storage and |
| | | PH[Methanopyrus | DSM 3638]. | | family, domain 1 | | processing; Translation, |
| | | kandleri AV19] | | | 1.10e−27 | | ribosomal |
| | | | | | :RNase_PH_C 3′ | | structure and |
| | | | | | exoribonuclease | | biogenesis; RNase |
| | | | | | family, domain 2 | | PH 9e−44 |
| | | | | | 1.30e−3 | | |
| 494, | NEQ249 | hypothetical protein | | | | | |
| 495 | | | | | | | |
| 496, | NEQ250 | hypothetical protein | putative hemolysin III | 0.04 | | | |
| 497 | | | [Streptococcus | | | | |
| | | | mutans UA159]. | | | | |
| 498, | NEQ252 | valyl-tRNA | valyl-tRNA synthetase | 1.00E−166 | tRNA-synt_1 tRNA | 6.1.1.9 | Information |
| 499 | | synthetase (class | [Aeropyrum pernix]. | | synthetases class I | | storage and |
| | | 1a)[Aeropyrum | | | (I, L, M and V) | | processing; Translation, |
| | | pernix] | | | 3.20e−73 :ileS ileS | | ribosomal |
| | | | | | isoleucyl-tRNA | | structure and |
| | | | | | synthetase 5.00e−21 | | biogenesis; Valyl- |
| | | | | | :leuS_bact | | tRNA |
| | | | | | leuS_bact leucyl- | | synthetase |
| | | | | | tRNA synthetase | | 1e−167 |
| | | | | | 1.70e−07 :metG | | |
| | | | | | metG methionyl- | | |
| | | | | | tRNA synthetase | | |
| | | | | | 1.20e−09 :valS valS | | |
| | | | | | valyl-tRNA | | |
| | | | | | synthetase 1.70e−109 | | |
| 500, | NEQ254 | hypothetical protein | K10D2.3.p | 0.25 | 0 | | |
| 501 | | | [Caenorhabditis | | | | |
| | | | elegans]. | | | | |
| 502, | NEQ253 | hypothetical protein | thyrotropin-releasing | 0.02 | 0 | | |
| 503 | | | hormone receptor | | | | |
| | | | [Homo sapiens]. | | | | |
| 504, | NEQ256 | DNA double-strand | DNA double-strand | 6.00E−68 | SMC_N | 3.1.11.— | Information |
| 505 | | break repair | break repair rad50 | | RecF/RecN/SMC N | | storage and |
| | | rad50ATPase | ATPase. | | terminal domain | | processing; DNA |
| | | | | | 1.90e−07 | | replication, |
| | | | | | | | recombination |
| | | | | | | | and |
| | | | | | | | repair; ATPase |
| | | | | | | | involved in |
| | | | | | | | DNA repair |
| | | | | | | | 3e−66 |
| 506, | NEQ255 | hypothetical protein | unknown | 0.32 | 0 | | |
| 507 | | | [Fusobacterium | | | | |
| | | | nucleatum subsp. | | | | |
| | | | nucleatum ATCC | | | | |
| | | | 25586]. | | | | |
| 508, | NEQ257 | LSU ribosomal | LSU ribosomal protein | 1.00E−29 | KOW KOW motif 8.90e−08 | | Information |
| 509 | | protein | L24P [Pyrococcus | | :rplX_A_E rplX_A_E | | storage and |
| | | L24P[Pyrococcus | abyssi] | | ribosomal protein L24 2.00e−40 | | processing; Translation, |
| | | abyssi] | | | | | ribosomal |
| | | | | | | | structure and |
| | | | | | | | biogenesis; Ribosomal |
| | | | | | | | protein L24 |
| | | | | | | | 1e−30 |
| 510, | NEQ258 | hypothetical protein | conserved | 3.00E−05 | 8.50E−04 | | |
| 511 | | | hypothetical protein | | | | |
| | | | [Archaeoglobus | | | | |
| | | | fulgidus]. | | | | |
| 512, | NEQ259 | hypothetical protein | putative peptidoglycan | 2.00E−04 | 0 | | |
| 513 | | | bound protein | | | | |
| | | | (LPXTG motif) | | | | |
| | | | [Listeria innocua]. | | | | |
| 514, | NEQ261 | tRNA intron | hypothetical protein | 1.00E−05 | 0 | | |
| 515 | | endonuclease | [Methanosarcina | | | | |
| | | (endA) | barkeri]. | | | | |
| | | [M. acetovorans] | | | | | |
| 516, | NEQ260 | hypothetical protein | hypothetical protein 4- | 0.04 | 0 | | |
| 517 | | | Trypanosoma brucei | | | | |
| | | | mitochondrion. | | | | |
| 518, | NEQ262 | LSU Ribosomal | hypothetical protein | 8.00E−06 | Ribosomal_L29 Ribosomal | | |
| 519 | | protein L29 | [Clostridium | | L29 protein 1.80e−06 :L29 | | |
| | | | thermocellum ATCC | | L29 ribosomal protein L29 4.40e−15 | | |
| | | | 27405]. | | | | |
| 520, | NEQ264 | Translation initiation | Translation initiation | 4.00E−12 | SUI1 Translation initiation | | Information |
| 521 | | factor | factor [Pyrococcus | | factor SUI1 9.70e−10 | | storage and |
| | | (SUI1)[Pyrococcus | horikoshii]. | | :SUI1_rel SUI1_rel | | processing; Translation, |
| | | horikoshii] | | | translation initation factor | | ribosomal |
| | | | | | SUI1, putative 1.10e−10 | | structure and |
| | | | | | | | biogenesis; Translation |
| | | | | | | | initiation |
| | | | | | | | factor (SUI1) |
| | | | | | | | 2e−13 |
| 522, | NEQ263 | V-type H+- | H+-transporting ATP | 1.00E−100 | ATP-synt_ab ATP | 3.6.3. | Metabolism; Energy |
| 523 | | transporting ATP | synthase, subunit B | | synthase alpha/beta | 14 | production |
| | | synthasebeta chain | (atpB) | | family, nucleotide- | | and |
| | | (V-type ATPase | [Methanococcus | | binding domain | | conversion; Archaeal/ |
| | | subunit B) | jannaschii]. | | 2.10e−68 :ATP- | | vacuolar- |
| | | | | | synt_ab_C ATP | | type H+- |
| | | | | | synthase alpha/beta | | ATPase |
| | | | | | chain, C terminal | | subunit B 1e−101 |
| | | | | | domain 3.40e−08 | | |
| | | | | | :atpA atpA ATP | | |
| | | | | | synthase F1, alpha | | |
| | | | | | subunit 1.20e−3 | | |
| | | | | | :flil_yscN flil_yscN | | |
| | | | | | ATPase Flil/YscN | | |
| | | | | | family 2.00e−11 | | |
| | | | | | :atpD atpD ATP | | |
| | | | | | synthase F1, beta | | |
| | | | | | subunit 2.70e−11 :V- | | |
| | | | | | ATPase_V1_B V- | | |
| | | | | | ATPase_V1_B V- | | |
| | | | | | type ATPase, | | |
| | | | | | subunit B 2.70e−111 | | |
| | | | | | :ATP_syn_B_arch | | |
| | | | | | ATP_syn_B_arch | | |
| | | | | | ATP synthase | | |
| | | | | | archaeal, B subunit | | |
| | | | | | 4.70e−132 | | |
| | | | | | :ATP_syn_A_arch | | |
| | | | | | ATP_syn_A_arch | | |
| | | | | | ATP synthase | | |
| | | | | | archaeal, A subunit | | |
| | | | | | 6.30e−3 | | |
| 524, | NEQ265 | Hypothetical protein | predicted cytoskeletal | 1.00E−06 | 0 | | |
| 525 | | | protein [Mycoplasma | | | | |
| | | | penetrans] | | | | |
| 526, | NEQ266 | hypothetical protein | hypothetical protein | 0.01 | 0 | | |
| 527 | | | [Plasmodium | | | | |
| | | | falciparum 3D7]. | | | | |
| 528, | NEQ267 | SPLIT see SEQ ID | conserved | 8.00E−20 | | | Poorly |
| 529 | | NOS: 530, 531; | hypothetical protein | | | | characterized; |
| | | flagella | [Methanococcus | | | | Function |
| | | accessoryprotein | jannaschii]. | | | | unknown; Predicted |
| | | [Methanococcus | | | | | membrane |
| | | voltae] | | | | | protein 5e−21 |
| 530, | NEQ268 | SPLIT see SEQ ID | conserved | 2.00E−26 | DUF110 Integral membrane | | Poorly |
| 531 | | NOS: 528, 529; | hypothetical protein | | protein DUF110 8.30e−3 | | characterized; |
| | | FLAGELLAACCESSORY | [Archaeoglobus | | | | Function |
| | | PROTEIN J | fulgidus]. | | | | unknown; Predicted |
| | | | | | | | membrane |
| | | | | | | | protein 1e−27 |
| 532, | NEQ270 | translation initiation | Chain A, Structure Of | 1.00E−104 | GTP_EFTU | 3.6.1. | Information |
| 533 | | factor aIF-2, subunit | The Wild-Type Large | | Elongation factor Tu | 48 | storage and |
| | | gamma [Pyrococcus | Gamma Subunit Of | | GTP binding domain | | processing; Translation, |
| | | abyssi] | Initiation Factor Eif2 | | 1.60e−44 | | ribosomal |
| | | | From Pyrococcus | | :GTP_EFTU_D2 | | structure and |
| | | | Abyssi Complexed | | Elongation factor Tu | | biogenesis; GTPases- |
| | | | With Gdp-Mg2+. | | domain 2 2.00e−06 | | translation |
| | | | | | :small_GTP | | elongation |
| | | | | | small_GTP small | | factors 1e−106 |
| | | | | | GTP-binding protein | | |
| | | | | | domain 4.00e−06 | | |
| | | | | | :selB selB | | |
| | | | | | selenocysteine- | | |
| | | | | | specific translation | | |
| | | | | | elongation factor | | |
| | | | | | 8.20e−07 :EF- | | |
| | | | | | 1_alpha EF-1_alpha | | |
| | | | | | translation | | |
| | | | | | elongation factor | | |
| | | | | | EF-1, subunit alpha | | |
| | | | | | 3.70e−06 :EF-Tu EF- | | |
| | | | | | Tu translation | | |
| | | | | | elongation factor Tu | | |
| | | | | | 3.80e−09 :FOLD979 | | |
| | | | | | No CATH | | |
| | | | | | Annotation 1.60e−127 | | |
| 534, | NEQ271 | hypothetical protein | purine NTPase | 5.00E−06 | 0 | | Information |
| 535 | | | [Methanococcus | | | | storage and |
| | | | jannaschii]. | | | | processing; DNA |
| | | | | | | | replication, |
| | | | | | | | recombination |
| | | | | | | | and |
| | | | | | | | repair; ATPase |
| | | | | | | | involved in |
| | | | | | | | DNA repair |
| | | | | | | | 3e−07 |
| 536, | NEQ273 | hypothetical protein | NADH | 0.04 | 0 | | |
| 537 | | | dehydrogenase I, J | | | | |
| | | | subunit [Brucella suis | | | | |
| | | | 1330]. | | | | |
| 538, | NEQ275 | hypothetical protein | hypothetical protein | 3.00E−06 | 0 | | |
| 539 | | | [Plasmodium yoelii | | | | |
| | | | yoelii]. | | | | |
| 540, | NEQ274 | SSU ribosomal | SSU ribosomal | 2.00E−27 | Ribosomal_S8 Ribosomal | | Information |
| 541 | | protein S8P; | protein S8P; (rps8E) | | protein S8 7.00e−32 | | storage and |
| | | (rps8E)[Pyrococcus | [Pyrococcus furiosus | | | | processing; Translation, |
| | | furiosus DSM 3638] | DSM 3638]. | | | | ribosomal |
| | | | | | | | structure and |
| | | | | | | | biogenesis; Ribosomal |
| | | | | | | | protein S8 5e−28 |
| 542, | NEQ276 | transcription initiation | transcription initiation | 2.00E−69 | transcript_fac2 Transcription | | Information |
| 543 | | factor IIB(TFIIB) | factor IIB [Pyrococcus | | factor TFIIB repeat 1.60e−17 | | storage and |
| | | (TFB)[Pyrococcus | abyssi] | | :transcript_fac2 | | processing; Transcription; |
| | | abyssi] | | | Transcription factor TFIIB | | Transcription |
| | | | | | repeat 8.40e−14 | | initiation |
| | | | | | | | factor IIB 1e−70 |
| 544, | NEQ277 | hypothetical protein | hypothetical protein | 1.00E−03 | 0 | | |
| 545 | | [Archaeoglobus fulgidus] | [Archaeoglobus | | | | |
| | | | fulgidus]. | | | | |
| 546, | NEQ278 | hypothetical protein | putative outer | 0.35 | 0 | | |
| 547 | | | membrane protein; | | | | |
| | | | probably involved in | | | | |
| | | | nutrient binding | | | | |
| | | | [Bacteroides | | | | |
| | | | thetaiotaomicron VPI- | | | | |
| | | | 5482] | | | | |
| 548, | NEQ279 | hypothetical protein | hypothetical protein | 4.00E−04 | 0 | | |
| 549 | | | [Plasmodium | | | | |
| | | | falciparum 3D7]. | | | | |
| 550, | NEQ281 | hypothetical protein | hypothetical protein | 1.00E−10 | | | Poorly |
| 551 | | | [Pyrococcus | | | | characterized; |
| | | | horikoshii]. | | | | General |
| | | | | | | | function |
| | | | | | | | prediction |
| | | | | | | | only; Archaeal |
| | | | | | | | serine |
| | | | | | | | proteases 8e−12 |
| 552, | NEQ280 | hypothetical protein | hypothetical protein | 0.14 | 0 | | |
| 553 | | | [Plasmodium | | | | |
| | | | falciparum 3D7]. | | | | |
| 554, | NEQ282 | minichromosome | DNA replication | 1.00E−121 | MCM MCM2/3/5 family | | Information |
| 555 | | maintenance(MCM) | initiator | | 1.50e−172 :TIGR00368 | | storage and |
| | | protein [Sulfolobus | (Cdc21/Cdc54) | | TIGR00368 Mg chelatase- | | processing; DNA |
| | | | [Methanothermobacter | | related protein 9.00e−3 | | replication, |
| | | | thermautotrophicus]. | | | | recombination |
| | | | | | | | and |
| | | | | | | | repair; Predicted |
| | | | | | | | ATPase |
| | | | | | | | involved in |
| | | | | | | | replication |
| | | | | | | | control, |
| | | | | | | | Cdc46/Mcm |
| | | | | | | | family 1e−122 |
| 556, | NEQ283 | Predicted ATPase of | conserved | 6.00E−48 | PAPS_reduct | | Cellular |
| 557 | | the PP- | hypothetical protein | | Phosphoadenosine | | processes; Cell |
| | | loopsuperfamily | [Archaeoglobus | | phosphosulfate reductase | | division and |
| | | implicated in cell | fulgidus]. | | family 8.60e−4 :UPF0021 | | chromosome |
| | | cycle | | | Uncharacterized protein | | partitioning; Predicted |
| | | | | | family UPF0021 4.50e−15 | | ATPase of |
| | | | | | :TIGR00269 TIGR00269 | | the PP-loop |
| | | | | | conserved hypothetical | | superfamily |
| | | | | | protein TIGR00269 3.70e−4 | | implicated in |
| | | | | | | | cell cycle |
| | | | | | | | control 4e−49 |
| 558, | NEQ284 | hypothetical protein | methylase ermT | 0.02 | 0 | | |
| 559 | | | [Plasmid p121BS]. | | | | |
| 560, | NEQ285 | conserved | hypothetical protein | 1.00E−25 | 0 | | |
| 561 | | hypothetical | [Methanosarcina | | | | |
| | | protein[Sulfolobus | barkeri]. | | | | |
| | | tokodaii] | | | | | |
| 562, | NEQ288 | putative archeal | archaeal histone | 1.00E−09 | CBFD_NFYB_HMF Histone- | | Information |
| 563 | | histone | [Methanococcus | | like transcription factor | | storage and |
| | | | jannaschii]. | | (CBF/NF-Y) and archaeal | | processing; DNA |
| | | | | | histone 1.40e−12 | | replication, |
| | | | | | | | recombination |
| | | | | | | | and |
| | | | | | | | repair; Histones |
| | | | | | | | H3 and H4 |
| | | | | | | | 6e−11 |
| 564, | NEQ290 | hypothetical protein | putative transport | 0.11 | 0 | | |
| 565 | | | protein [Buchnera | | | | |
| | | | aphidicola (Baizongia | | | | |
| | | | pistaciae)] | | | | |
| 566, | NEQ289 | hypothetical protein | agCP6807 | 0.06 | 0 | | |
| 567 | | | [Anopheles gambiae | | | | |
| | | | str. PEST]. | | | | |
| 568, | NEQ291 | hypothetical protein | Uncharacterized | 3.00E−03 | 0 | | |
| 569 | | | protein conserved in | | | | |
| | | | bacteria | | | | |
| | | | [Wigglesworthia | | | | |
| | | | brevipalpis]. | | | | |
| 570, | NEQ292 | hypothetical protein | HEP27 PROTEIN | 6.9 | 0 | | |
| 571 | | | (PROTEIN D). | | | | |
| 572, | NEQ293 | conserved | hypothetical protein | 2.00E−40 | UPF0024 Uncharacterized | | Poorly |
| 573 | | hypothetical | [Aquifex aeolicus]. | | protein family UPF0024 | | characterized; |
| | | protein[Aquifex | | | 2.20e−46 :TIGR00094 | | Function |
| | | aeolicus] | | | TIGR00094 conserved | | unknown; Uncharacterized |
| | | Electrontransfer | | | hypothetical protein | | ACR 1e−41 |
| | | | | | TIGR00094 3.30e−19 | | |
| | | | | | :TIGR00094 TIGR00094 | | |
| | | | | | conserved hypothetical | | |
| | | | | | protein TIGR00094 5.40e−16 | | |
| 574, | NEQ294 | n-type ATP | hypothetical protein | 1.00E−35 | DUF71 Domain of unknown | | Poorly |
| 575 | | pyrophosphatasesuperfamily | [Pyrococcus | | function DUF71 5.10e−34 | | characterized; |
| | | [Pyrococcus | horikoshii]. | | :TIGR00289 TIGR00289 | | General |
| | | furiosus] | | | conserved hypothetical | | function |
| | | | | | protein TIGR00289 2.50e−21 | | prediction |
| | | | | | :MJ0570_dom MJ0570_dom | | only; Predicted |
| | | | | | MJ0570-related | | ATPases of |
| | | | | | uncharacterized domain | | PP-loop |
| | | | | | 3.80e−31 | | superfamily |
| | | | | | | | 1e−36 |
| 576, | NEQ295 | putative nucleolar | NUCLEOLAR | 2.00E−45 | Nol1_Nop2_Sun | 2.1.1.— | Information |
| 577 | | protein III (nol1- | PROTEIN | | NOL1/NOP2/sun | | storage and |
| | | nop2-sun family) | [Pyrococcus abyssi]. | | family 1.90e−38 | | processing; Translation, |
| | | | | | :nop2p nop2p | | ribosomal |
| | | | | | NOL1/NOP2/sun | | structure and |
| | | | | | family putative RNA | | biogenesis; tRNA |
| | | | | | methylase 5.90e−53 | | and rRNA |
| | | | | | :rsmB rsmB Sun | | cytosine-C5- |
| | | | | | protein 5.50e−06 | | methylases |
| | | | | | | | 1e−46 |
| 578, | NEQ297 | LSU Ribosomal | LSU ribosomal protein | 6.00E−17 | Ribosomal_L34e Ribosomal | | Information |
| 579 | | protein L34E | L34E [Methanococcus | | protein L34e 8.70e−15 | | storage and |
| | | | jannaschii]. | | | | processing; Translation, |
| | | | | | | | ribosomal |
| | | | | | | | structure and |
| | | | | | | | biogenesis; Ribosomal |
| | | | | | | | protein L34E |
| | | | | | | | 3e−18 |
| 580, | NEQ296 | hypothetical protein | MURF2 protein (AA 1- | 2.00E−04 | 0 | | |
| 581 | | | 348) [Crithidia | | | | |
| | | | fasciculata]. | | | | |
| 582, | NEQ298 | Acetyltransferase | hypothetical protein | 1.00E−10 | 0 | | |
| 583 | | [Fusobacteriumnucleatum | [Pyrococcus | | | | |
| | | subsp. | horikoshii]. | | | | |
| | | nucleatum] | | | | | |
| 584, | NEQ299 | Predicted ABC-class | transport protein | 1.00E−155 | ABC_tran ABC | 1.8.—.— | Poorly |
| 585 | | ATPase, RNaseL | [Pyrococcus | | transporter 1.80e−23 | | characterized; |
| | | inhibitor homolog [M. kandleri] | horikoshii]. | | :ABC_tran ABC | | General |
| | | | | | transporter 2.90e−18 | | function |
| | | | | | :fer4 4Fe-4S binding | | prediction |
| | | | | | domain 4.40e−09 | | only; RNase L |
| | | | | | :3a0501s02 | | inhibitor |
| | | | | | 3a0501s02 Type II | | homolog, |
| | | | | | (General) Secretory | | predicted |
| | | | | | Pathway (IISP) | | ATPase 1e−156 |
| | | | | | Family protein | | |
| | | | | | 5.30e−3 :3a0106s01 | | |
| | | | | | 3a0106s01 sulfate | | |
| | | | | | transport system | | |
| | | | | | permease protein | | |
| | | | | | 4.90e−05 :cbiO cbiO | | |
| | | | | | cobalt transport | | |
| | | | | | protein ATP-binding | | |
| | | | | | subunit 1.00e−05 | | |
| | | | | | :ntrCD ntrCD nitrate | | |
| | | | | | transport ATP- | | |
| | | | | | binding subunits C | | |
| | | | | | and D 7.90e−4 :drrA | | |
| | | | | | drrA daunorubicin | | |
| | | | | | resistance ABC | | |
| | | | | | transporter ATP- | | |
| | | | | | binding subunit | | |
| | | | | | 5.00e−05 :thiQ thiQ | | |
| | | | | | ABC transporter, | | |
| | | | | | ATP-binding protein, | | |
| | | | | | ThiQ subfamily | | |
| | | | | | 7.20e−3 :RLI | | |
| | | | | | Possible metal- | | |
| | | | | | binding domain in | | |
| | | | | | RNase L inhibitor, | | |
| | | | | | RLI 3.70e−14 | | |
| 586, | NEQ300 | hypothetical surface | surface layer protein | 3.00E−06 | 0 | | |
| 587 | | layer protein | [Methanothermococcus | | | | |
| | | [Mcthermolithotrophicus] | thermolithotrophicus]. | | | | |
| 588, | NEQ301 | hypothetical protein | EsV-1-147 | 0.04 | 0 | | |
| 589 | | | [Ectocarpus | | | | |
| | | | siliculosus virus]. | | | | |
| 590, | NEQ302 | glutamyl-tRNA | glutamyl-tRNA | 1.00E−122 | tRNA-synt_1c tRNA | 6.1.1. | Information |
| 591 | | synthetase (class | synthetase | | synthetases class I | 17 | storage and |
| | | 1)[Pyrococcus | [Pyrococcus abyssi]. | | (E and Q), catalytic | | processing; Translation, |
| | | abyssi] | | | domain 2.50e−80 | | ribosomal |
| | | | | | :glnS glnS | | structure and |
| | | | | | glutaminyl-tRNA | | biogenesis; Glutamyl- |
| | | | | | synthetase 3.00e−36 | | and |
| | | | | | :gltX_arch gltX_arch | | glutaminyl- |
| | | | | | glutamyl-tRNA | | tRNA |
| | | | | | synthetase 5.20e−147 | | synthetases |
| | | | | | :gltX_bact | | 1e−123 |
| | | | | | gltX_bact glutamyl- | | |
| | | | | | tRNA synthetase | | |
| | | | | | 1.40e−13 :tRNA- | | |
| | | | | | synt_1c_C tRNA | | |
| | | | | | synthetases class I | | |
| | | | | | (E and Q), anti- | | |
| | | | | | codon binding | | |
| | | | | | domain 2.00e−07 | | |
| 592, | NEQ303 | LSU Ribosomal | LSU ribosomal protein | 1.00E−19 | Ribosomal_L35Ae | | Information |
| 593 | | protein L35AE | L35AE. [Pyrococcus | | Ribosomal protein L35Ae | | storage and |
| | | | abyssi] | | 1.00e−20 | | processing; Translation, |
| | | | | | | | ribosomal |
| | | | | | | | structure and |
| | | | | | | | biogenesis; Ribosomal |
| | | | | | | | protein |
| | | | | | | | L35AE/L33A |
| | | | | | | | 7e−21 |
| 594, | NEQ304 | hypothetical | M. jannaschii | 0.01 | 0 | | |
| 595 | | | predicted coding | | | | |
| | | | region MJ0944 | | | | |
| | | | [Methanococcus | | | | |
| | | | jannaschii]. | | | | |
| 596, | NEQ305 | Archaeosine tRNA- | archaeosine tRNA- | 2.00E−08 | 0 | | Cellular |
| 597 | | ribosyltransferase[T | ribosyltransferase | | | | processes; Posttranslational |
| | | volcanium] SPLIT | [Methanosarcina | | | | modification, |
| | | See SEQ ID | mazei Goe1]. | | | | protein |
| | | NOS: 248, 249 | | | | | turnover, |
| | | | | | | | chaperones; Prefoldin, |
| | | | | | | | molecular |
| | | | | | | | chaperone |
| | | | | | | | implicated in |
| | | | | | | | de novo |
| | | | | | | | protein |
| | | | | | | | folding, alpha |
| | | | | | | | subunit 7e−08 |
| 598, | NEQ306 | 3-oxoacyl-[acyl- | 3-oxoacyl-[acyl- | 1.00E−30 | adh_short short | 1.1.1. | Metabolism; Secondary |
| 599 | | carrier- | carrier-protein] | | chain | 100 | metabolites |
| | | protein]reductase | reductase [Aquifex | | dehydrogenase | | biosynthesis, |
| | | [Aquifex aeolicus] | aeolicus]. | | 5.90e−45 | | transport and |
| | | | | | | | catabolism; Dehydrogenases |
| | | | | | | | with |
| | | | | | | | different |
| | | | | | | | specificities |
| | | | | | | | (related to |
| | | | | | | | short-chain |
| | | | | | | | alcohol |
| | | | | | | | dehydrogenases) |
| | | | | | | | 1e−31 |
| 600, | NEQ307 | nucleoside | Nucleoside | 8.00E−41 | NDK Nucleoside | 2.7.4.6 | Metabolism; Nucleotide |
| 601 | | diphosphate kinase | diphosphate kinase | | diphosphate kinase | | transport and |
| | | (ndk)[Pyrobaculum | (NDK) (NDP kinase) | | 1.10e−22 | | metabolism; Nucleoside |
| | | aerophilum] | (Nucleoside-2-P | | | | diphosphate |
| | | | kinase). | | | | kinase 1e−26 |
| 602, | NEQ309 | hypothetical protein | hypothetical protein | 0.45 | 0 | | |
| 603 | | | [Pyrococcus furiosus | | | | |
| | | | DSM 3638]. | | | | |
| 604, | NEQ308 | Seryl-tRNA | 453aa long | 1.00E−77 | Seryl_tRNA_N | 6.1.1. | Information |
| 605 | | synthetase (class | hypothetical seryl- | | Seryl-tRNA | 11 | storage and |
| | | 2)[Sulfolobus | tRNA synthetase | | synthetase N- | | processing; Translation, |
| | | tokodaii] | [Sulfolobus tokodaii]. | | terminal domain | | ribosomal |
| | | | | | 2.90e−10 :tRNA- | | structure and |
| | | | | | synt_2b tRNA | | biogenesis; Seryl- |
| | | | | | synthetase class II | | tRNA |
| | | | | | core domain (G, H, | | synthetase |
| | | | | | P, S and T) 5.60e−42 | | 1e−76 |
| | | | | | :serS serS seryl- | | |
| | | | | | tRNA synthetase | | |
| | | | | | 1.90e−81 | | |
| 606, | NEQ310 | hypothetical protein | hypothetical protein | 2.00E−14 | 0 | | Poorly |
| 607 | | [Pyrococcusabyssi] | [Pyrococcus abyssi]. | | | | characterized; |
| | | | | | | | Function |
| | | | | | | | unknown; Uncharacterized |
| | | | | | | | ACR 3e−15 |
| 608, | NEQ312 | hypothetical protein | hypothetical protein | 0.9 | 0 | | |
| 609 | | | [Plasmodium | | | | |
| | | | falciparum 3D7]. | | | | |
| 610, | NEQ311 | LSU ribosomal | LSU ribosomal protein | 1.00E−44 | Ribosomal_L30 Ribosomal | | Information |
| 611 | | protein L30P | L30P (rpmD) | | protein L30p/L7e 7.90e−09 | | storage and |
| | | (rpmD)[Methanococcus | [Methanococcus | | :L30P_arch L30P_arch | | processing; Translation, |
| | | jannaschii] | jannaschii]. | | ribosomal protein L30P | | ribosomal |
| | | | | | 7.90e−81 :L7 L7 60S | | structure and |
| | | | | | ribosomal protein L7 3.00e−3 | | biogenesis; Ribosomal |
| | | | | | | | protein |
| | | | | | | | L30/L7E 1e−45 |
| 612, | NEQ314 | hypothetical protein | conserved | 2.00E−06 | 5.30E−04 | | Poorly |
| 613 | | | hypothetical protein | | | | characterized; |
| | | | [Archaeoglobus | | | | Function |
| | | | fulgidus]. | | | | unknown; Uncharacterized |
| | | | | | | | ACR 1e−07 |
| 614, | NEQ313 | conserved | conserved protein | 6.00E−06 | 0 | | |
| 615 | | hypothetical | [Methanosarcina | | | | |
| | | protein[Methanosarcina | mazei Goe1]. | | | | |
| | | acetivorans str. | | | | | |
| | | C2A] | | | | | |
| 616, | NEQ316 | putative dUTPase | putative | 2.00E−38 | dUTPase dUTPase | 3.5.4. | Metabolism; Nucleotide |
| 617 | | | deoxynucleotide | | 1.40e−10 :dut dut | 13 | transport and |
| | | | triphosphate | | deoxyuridine 5′- | | metabolism; Deoxycytidine |
| | | | deaminase | | triphosphate | | deaminase |
| | | | [Streptomyces | | nucleotidohydrolase | | 8e−37 |
| | | | coelicolor A3(2)]. | | (dut) 1.30e−3 | | |
| 618, | NEQ315 | proteinase IV - | hypothetical protein | 3.00E−23 | Peptidase_U7 | 3.4.21.— | Cellular |
| 619 | | [Methanococcusjannaschii] | [Prochlorococcus | | Peptidase family U7 | | processes; Cell |
| | | Periplasmic | marinus subsp. | | 7.80e−25 | | motility and |
| | | serine | pastoris str. | | :SppA_dom | | secretion; Periplasmic |
| | | | CCMP1378]. | | SppA_dom signal | | serine |
| | | | | | peptide peptidase | | proteases |
| | | | | | SppA, 36K type | | (ClpP class) |
| | | | | | 2.50e−27 | | 3e−23 |
| 620, | NEQ317 | LSU ribosomal | 50S RIBOSOMAL | 7.00E−15 | | | Information |
| 621 | | protein | PROTEIN L15P. | | | | storage and |
| | | L15[Sulfolobus | | | | | processing; Translation, |
| | | acidocaldarius] | | | | | ribosomal |
| | | | | | | | structure and |
| | | | | | | | biogenesis; Ribosomal |
| | | | | | | | protein L15 |
| | | | | | | | 7e−15 |
| 622, | NEQ318 | reverse gyrase | reverse gyrase | 1.00E−175 | Topoisom_bac DNA | 5.99. | Information |
| 623 | | [Pyrococcus abyssi] | [Pyrococcus abyssi]. | | topoisomerase | 1.3 | storage and |
| | | SPLIT See SEQ ID | | | 5.60e−59 :Toprim | | processing; DNA |
| | | NOS: 848, 849 | | | Toprim domain | | replication, |
| | | | | | 1.00e−46 :topA_bact | | recombination |
| | | | | | topA_bact DNA | | and |
| | | | | | topoisomerase I | | repair; Reverse |
| | | | | | 4.30e−32 :rgy rgy | | gyrase 1e−176 |
| | | | | | reverse gyrase | | |
| | | | | | 2.20e−36 :topB topB | | |
| | | | | | DNA topoisomerase | | |
| | | | | | III 2.50e−06 | | |
| | | | | | :topA_arch | | |
| | | | | | topA_arch DNA | | |
| | | | | | topoisomerase I | | |
| | | | | | 6.20e−10 | | |
| 624, | NEQ319 | SSU hypothetical | 126aa long | 2.00E−46 | Ribosomal_L7Ae Ribosomal | | Information |
| 625 | | 30S ribosomalprotein | hypothetical 30S | | protein | | storage and |
| | | HS6 [Sulfolobus | ribosomal protein HS6 | | L7Ae/L30e/S12e/Gadd45 | | processing; Translation, |
| | | tokodaii] | [Sulfolobus tokodaii]. | | family 1.40e−45 | | ribosomal |
| | | | | | | | structure and |
| | | | | | | | biogenesis; Ribosomal |
| | | | | | | | protein HS6- |
| | | | | | | | type |
| | | | | | | | (S12/L30/L7a) |
| | | | | | | | 4e−46 |
| 626, | NEQ321 | C4-type Zn-finger- | hypothetical protein | 1.00E−17 | ZPR1 ZPR1 zinc-finger | | Poorly |
| 627 | | containing | [Pyrococcus furiosus | | domain 1.30e−11 :ZPR1_znf | | characterized; |
| | | protein[Methanopyrus | DSM 3638]. | | ZPR1_znf ZPR1 zinc finger | | General |
| | | kandleri AV19] | | | domain 5.50e−06 :zpr1_rel | | function |
| | | | | | zpr1_rel ZPR1-related zinc | | prediction |
| | | | | | finger protein 2.00e−06 | | only; C4-type |
| | | | | | | | Zn finger 3e−18 |
| 628, | NEQ320 | SSU Ribosomla | S ribosomal protein | 2.00E−13 | 0 | | Information |
| 629 | | protein S17E | S17E [Pyrococcus | | | | storage and |
| | | | horikoshii]. | | | | processing; Translation, |
| | | | | | | | ribosomal |
| | | | | | | | structure and |
| | | | | | | | biogenesis; Ribosomal |
| | | | | | | | protein S17E |
| | | | | | | | 1e−14 |
| 630, | NEQ322 | hypothetical protein | Putative Nonclathrin | 4.9 | 0 | | |
| 631 | | | coat protein gamma- | | | | |
| | | | like protein [Oryza | | | | |
| | | | sativa (japonica | | | | |
| | | | cultivar-group)]. | | | | |
| 632, | NEQ324 | DNA topoisomerase I | DNA topoisomerase I | 9.00E−34 | | 5.99. | Information |
| 633 | | [Pyrococcus horikoshii] | [Pyrococcus | | | 1.2 | storage and |
| | | SPLIT see SEQ ID | horikoshii]. | | | | processing; DNA |
| | | NOS: 90, 91 | | | | | replication, |
| | | | | | | | recombination |
| | | | | | | | and |
| | | | | | | | repair; Topoisomerase |
| | | | | | | | IA |
| | | | | | | | 8e−35 |
| 634, | NEQ323 | translation initiation | translation initiation | 1.00E−25 | eIF5_eIF2B Domain found in | | Information |
| 635 | | factor eIF-2 | factor eIF-2 beta | | IF2B/IF5 3.30e−27 :aIF- | | storage and |
| | | beta[Pyrococcus | [Pyrococcus | | 2beta aIF-2beta translation | | processing; Translation, |
| | | horikoshii] | horikoshii]. | | initiation factor aIF-2, beta | | ribosomal |
| | | | | | subunit, putative 7.90e−26 | | structure and |
| | | | | | | | biogenesis; Translation |
| | | | | | | | initiation |
| | | | | | | | factor eIF-2, |
| | | | | | | | beta |
| | | | | | | | subunit/eIF-5 |
| | | | | | | | N-terminal |
| | | | | | | | domain 1e−26 |
| 636, | NEQ325 | conserved | hypothetical protein | 2.00E−19 | DUF207 Uncharacterized | | Poorly |
| 637 | | hypothetical | [Pyrococcus furiosus | | ACR, COG1590 1.50e−27 | | characterized; |
| | | protein[Pyrococcus | DSM 3638]. | | | | Function |
| | | furiosus DSM 3638] | | | | | unknown; Uncharacterized |
| | | | | | | | ACR 3e−20 |
| 638, | NEQ326 | SSU ribosomal | 30S ribosomal protein | 8.00E−26 | Ribosomal_S17 Ribosomal | | Information |
| 639 | | protein | S17 [Pyrococcus | | protein S17 3.70e−23 | | storage and |
| | | S17[Pyrococcus | horikoshii]. | | | | processing; Translation, |
| | | horikoshii] | | | | | ribosomal |
| | | | | | | | structure and |
| | | | | | | | biogenesis; Ribosomal |
| | | | | | | | protein S17 |
| | | | | | | | 4e−27 |
| 640, | NEQ328 | transcriptional | transcriptional | 6.00E−12 | ASNC_trans_reg AsnC | | Information |
| 641 | | regulatory | regulatory protein, | | family 7.40e−3 | | storage and |
| | | protein, asnC family | asnC family | | | | processing; Transcription; |
| | | [Pyrococcus | [Pyrococcus furiosus | | | | Transcriptional |
| | | furiosus] | DSM 3638]. | | | | regulators 4e−13 |
| 642, | NEQ327 | hypothetical protein | cell division protein | 0.05 | 0 | | |
| 643 | | | [Chaetosphaeridium | | | | |
| | | | globosum]. | | | | |
| 644, | NEQ329 | dCTP deaminase | Deoxycytidine | 2.00E−10 | 0 | 3.5.4. | Metabolism; Nucleotide |
| 645 | | [Methanosarcinaacetivorans | triphosphate | | | 13 | transport and |
| | | str. C2A] | deaminase | | | | metabolism; Deoxycytidine |
| | | | [Methanosarcinamazei | | | | deaminase |
| | | | Goe1]. | | | | 2e−07 |
| 646, | NEQ330 | hypothetical protein | | | 0 | | |
| 647 | | | | | | | |
| 648, | NEQ331 | hypothetical protein | Similar to protein | 1.00E−03 | 0 | | |
| 649 | | | kinase C substrate | | | | |
| | | | 80K-H [Mus | | | | |
| | | | musculus]. | | | | |
| 650, | NEQ332 | hypothetical protein | | | 0 | | |
| 651 | | | | | | | |
| 652, | NEQ334 | Predicted RNA- | DNA-binding protein | 2.00E−42 | 1.00E−67 | | Information |
| 653 | | binding | [Pyrococcus furiosus | | | | storage and |
| | | protein[Methanopyrus | DSM 3638]. | | | | processing; Translation, |
| | | kandleri AV19] | | | | | ribosomal |
| | | | | | | | structure and |
| | | | | | | | biogenesis; Predicted |
| | | | | | | | RNA- |
| | | | | | | | binding |
| | | | | | | | protein 8e−42 |
| 654, | NEQ333 | pseudouridylate | tRNA pseudouridine | 4.00E−10 | PseudoU_synth_1 | 4.2.1. | Information |
| 655 | | synthase I | synthase A [Bacillus | | tRNA pseudouridine | 70 | storage and |
| | | (truA)[Methanococcus | cereus ATCC 14579] | | synthase 7.20e−07 | | processing; Translation, |
| | | jannaschii] | | | :hisT_truA hisT_truA | | ribosomal |
| | | | | | tRNA pseudouridine | | structure and |
| | | | | | synthase A 7.60e−22 | | biogenesis; Pseudouridylate |
| | | | | | | | synthase |
| | | | | | | | (tRNA psi55) |
| | | | | | | | 6e−11 |
| 656, | NEQ335 | hypothetical protein | hypothetical protein | 0.01 | 0 | | |
| 657 | | | [Arabidopsis thaliana]. | | | | |
| 658, | NEQ336 | hypothetical protein | Superfamily II | 0.02 | 0 | | |
| 659 | | | DNA/RNA helicase, | | | | |
| | | | SNF2 family | | | | |
| | | | [Clostridium | | | | |
| | | | acetobutylicum]. | | | | |
| 660, | NEQ338 | DNA-directed RNA | DNA-directed RNA | 6.00E−17 | RNA_pol_N RNA | 2.7.7.6 | Information |
| 661 | | polymerase, subunit | polymerase, subunit N | | polymerases N/8 kDa | | storage and |
| | | N [Aeropyrum pernix] | [Aeropyrum pernix]. | | subunit 3.60e−25 | | processing; Transcription; |
| | | | | | | | DNA- |
| | | | | | | | directed |
| | | | | | | | RNA |
| | | | | | | | polymerase, |
| | | | | | | | subunit N |
| | | | | | | | (RpoN/RPB10) |
| | | | | | | | 3e−18 |
| 662, | NEQ337 | L-isoaspartyl protein | L-isoaspartyl protein | 8.00E−12 | 0 | 2.1.1. | Information |
| 663 | | carboxylmethyltransferase | carboxyl | | | .77 | storage and |
| | | isolog (pimT) | methyltransferase | | | | processing; Translation, |
| | | | isolog (pimT) | | | | ribosomal |
| | | | [Methanococcus | | | | structure and |
| | | | jannaschii]. | | | | biogenesis; Predicted |
| | | | | | | | SAM- |
| | | | | | | | dependent |
| | | | | | | | methyltransferase |
| | | | | | | | involved |
| | | | | | | | in tRNA-Met |
| | | | | | | | maturation |
| | | | | | | | 6e−13 |
| 664, | NEQ340 | Predicted hydrolase | phosphoglycolate | 3.00E−03 | Hydrolase haloacid dehalogenase-like | | |
| 665 | | (HADsuperfamily) | phosphatase [Nostoc | | hydrolase 1.10e−07 | | |
| | | [Thermoplasma | sp. PCC 7120]. | | | | |
| 666, | NEQ339 | conserved | conserved | 1.00E−09 | Peptidase_M50 Peptidase | | Poorly |
| 667 | | hypothetical | hypothetical integral | | family M50 6.20e−05 | | characterized; |
| | | integralmembrane | membrane protein | | | | General |
| | | protein [M jannaschii] | [Methanococcus | | | | function |
| | | | jannaschii]. | | | | prediction |
| | | | | | | | only; Zn- |
| | | | | | | | dependent |
| | | | | | | | proteases 8e−11 |
| 668, | NEQ341 | primase DnaG-like | hypothetical protein | 3.00E−86 | Toprim Toprim | 2.7.7.— | Information |
| 669 | | [Pyrococcusabyssi] | [Pyrococcus furiosus | | domain 1.10e−06 | | storage and |
| | | | DSM 3638]. | | | | processing; DNA |
| | | | | | | | replication, |
| | | | | | | | recombination |
| | | | | | | | and |
| | | | | | | | repair; DNA |
| | | | | | | | primase |
| | | | | | | | (bacterial |
| | | | | | | | type) 1e−85 |
| 670, | NEQ342 | hypothetical | 409aa long | 1.00E−63 | Nop Putative snoRNA | | Information |
| 671 | | nucleolar protein | hypothetical nucleolar | | binding domain 9.50e−72 | | storage and |
| | | NOP56[Sulfolobus | protein [Sulfolobus | | | | processing; Translation, |
| | | tokodaii]; snoRNA | tokodaii]. | | | | ribosomal |
| | | | | | | | structure and |
| | | | | | | | biogenesis; Protein |
| | | | | | | | implicated in |
| | | | | | | | ribosomal |
| | | | | | | | biogenesis, |
| | | | | | | | Nop56p |
| | | | | | | | homolog 2e−63 |
| 672, | NEQ344 | small heat shock | small heat shock | 4.00E−13 | HSP20 Hsp20/alpha | | Cellular |
| 673 | | protein (class | protein (class I) | | crystallin family 1.10e−11 | | processes; Posttranslational |
| | | I)[Aquifex aeolicus] | [Aquifex aeolicus]. | | | | modification, |
| | | and other | | | | | protein |
| | | | | | | | turnover, |
| | | | | | | | chaperones; |
| | | | | | | | Molecular |
| | | | | | | | chaperone |
| | | | | | | | (small heat |
| | | | | | | | shock protein) |
| | | | | | | | 4e−14 |
| 674, | NEQ343 | intracellular protease | intracellular protease | 3.00E−07 | | | Poorly |
| 675 | | (Protease | [Pyrococcus abyssi]. | | | | characterized; |
| | | I)[Pyrococcus abyssi] | | | | | General |
| | | | | | | | function |
| | | | | | | | prediction |
| | | | | | | | only; Putative |
| | | | | | | | intracellular |
| | | | | | | | protease/amidase |
| | | | | | | | 2e−08 |
| 676, | NEQ345 | anaerobic | hypothetical protein | | 0 | 1.17. | Metabolism; Nucleotide |
| 677 | | ribonucleoside- | [Pyrococcus | | | 4.2 | transport and |
| | | triphosphatereductase | horikoshii]. | | | | metabolism; Oxygen- |
| | | [Pyrococcus | | | | | sensitive |
| | | abyssi] | | | | | ribonucleoside- |
| | | | | | | | triphosphate |
| | | | | | | | reductase 0 |
| 678, 679 | | putative | hypothetical protein | 8.00E−28 | Methyltransf_1 6-O- | 3.1.—.— | Information |
| | | endonuclease V | [Ferroplasma | | methylguanine DNA | | storage and |
| | | | acidarmanus]. | | methyltransferase, | | processing; DNA |
| | | | | | DNA binding domain | | replication, |
| | | | | | 2.30e−3 :Endonuc_V | | recombination |
| | | | | | Endonuclease V | | and |
| | | | | | 2.20e−29 | | repair; Deoxyinosine |
| | | | | | | | 3′endonuclease |
| | | | | | | | (endonuclease |
| | | | | | | | V) 3e−20 |
| 680, | NEQ346 | LSU Ribosomal | 50S ribosomal protein | 7.00E−09 | Ribosomal_LX Ribosomal | | Information |
| 681 | | protein L20A | LX [Pyrococcus | | LX protein 3.80e−10 | | storage and |
| | | | horikoshii]. | | | | processing; Translation, |
| | | | | | | | ribosomal |
| | | | | | | | structure and |
| | | | | | | | biogenesis; Ribosomal |
| | | | | | | | protein L20A |
| | | | | | | | (L18A) 4e−10 |
| 682, | NEQ347 | Predicted | conserved | 9.00E−44 | UPF0103 Protein of | | Poorly |
| 683 | | dioxygenase | hypothetical protein | | unknown function DUF52 | | characterized; |
| | | [Methanopyruskandleri | [Methanosarcina | | 4.10e−61 | | General |
| | | AV19] | acetivorans str. C2A]. | | | | function |
| | | | | | | | prediction |
| | | | | | | | only; Predicted |
| | | | | | | | dioxygenase |
| | | | | | | | 4e−39 |
| 684, | NEQ348 | putative archeal | archaeal histone A2 | 7.00E−12 | CBFD_NFYB_HMF Histone- | | Information |
| 685 | | histone | [Methanococcus | | like transcription factor | | storage and |
| | | | jannaschii]. | | (CBF/NF-Y) and archaeal | | processing; DNA |
| | | | | | histone 1.40e−21 | | replication, |
| | | | | | | | recombination |
| | | | | | | | and |
| | | | | | | | repair; Histones |
| | | | | | | | H3 and H4 |
| | | | | | | | 4e−13 |
| 686, | NEQ349 | ATP-dependent | ATP-dependent | 1.00E−177 | Sigma54_activat | 3.4.21. | Cellular |
| 687 | | protease La | protease Lon | | Sigma-54 interaction | 53 | processes; Posttranslational |
| | | (lon)[Archaeoglobus | [Thermococcus | | domain 5.40e−3 | | modification, |
| | | fulgidus] | kodakaraensis]. | | :lon_rel lon_rel | | protein |
| | | | | | ATP-dependent | | turnover, |
| | | | | | protease, putative | | chaperones; Predicted |
| | | | | | 8.40e−219 : 8.60e−11 | | ATP- |
| | | | | | | | dependent |
| | | | | | | | protease 1e−173 |
| 688, | NEQ350 | Predicted aromatic | Predicted aromatic | 8.00E−15 | DUF59 Domain of | 2.3.1. | Poorly |
| 689 | | ring | ring hydroxylating | | unknown function | 30 | characterized; |
| | | hydroxylatingenzyme | enzyme | | DUF59 2.50e−15 | | General |
| | | [Thermoplasma | [Thermoplasma | | | | function |
| | | volcanium] | volcanium]. | | | | prediction |
| | | | | | | | only; Predicted |
| | | | | | | | metal-sulfur |
| | | | | | | | cluster |
| | | | | | | | biosynthetic |
| | | | | | | | enzyme 5e−16 |
| 690, | NEQ351 | Predicted | hypothetical protein | 8.00E−03 | 0 | | |
| 691 | | transcriptional | [Pyrococcus furiosus | | | | |
| | | regulator | DSM 3638]. | | | | |
| 692, | NEQ352 | putative ABC | hypothetical protein | 1.00E−13 | DUF63 Membrane protein of | | Poorly |
| 693 | | transporter, transmembrane | [Pyrococcus furiosus | | unknown function DUF63 | | characterized; |
| | | component | DSM 3638]. | | 3.40e−4 | | Function |
| | | [P. | | | | | unknown; Predicted |
| | | | | | | | membrane |
| | | | | | | | proteins 2e−12 |
| 694, | NEQ353 | hypothetical protein | hypothetical protein | 5.00E−27 | 0 | | Poorly |
| 695 | | | [Archaeoglobus | | | | characterized; |
| | | | fulgidus]. | | | | General |
| | | | | | | | function |
| | | | | | | | prediction |
| | | | | | | | only; Predicted |
| | | | | | | | nucleic acid |
| | | | | | | | binding |
| | | | | | | | protein |
| | | | | | | | containing the |
| | | | | | | | AN1-type Zn- |
| | | | | | | | finger 3e−28 |
| 696, | NEQ354 | hypothetical protein | hypothetical protein | 1.00E−03 | 0 | | |
| 697 | | | [Plasmodium | | | | |
| | | | falciparum 3D7]. | | | | |
| 698, | NEQ356 | hypothetical | hypothetical protein | 0.01 | 0 | | |
| 699 | | proteinputative | [Plasmodium | | | | |
| | | vitamin B12 | falciparum 3D7]. | | | | |
| | | transporter? | | | | | |
| 700, | NEQ355 | predicted | conserved | 8.00E−11 | 0 | | Information |
| 701 | | transcriptional | hypothetical protein | | | | storage and |
| | | regulator | [Archaeoglobus | | | | processing; Transcription; |
| | | | fulgidus]. | | | | Predicted |
| | | | | | | | transcriptional |
| | | | | | | | regulator |
| | | | | | | | containing an |
| | | | | | | | HTH domain |
| | | | | | | | fused to a Zn- |
| | | | | | | | ribbon 4e−12 |
| 702, | NEQ358 | Predicted calcineurin | hypothetical protein | 6.00E−52 | Metallophos Calcineurin-like | | Poorly |
| 703 | | superfamilyphosphoesterase | [Pyrococcus abyssi]. | | phosphoesterase 4.80e−07 | | characterized; |
| | | [M. kandleri] | | | :TIGR00024 TIGR00024 | | General |
| | | | | | conserved hypothetical | | function |
| | | | | | protein TIGR00024 5.00e−38 | | prediction |
| | | | | | | | only; Predicted |
| | | | | | | | ICC-like |
| | | | | | | | phosphoesterases |
| | | | | | | | 4e−53 |
| 704, | NEQ357 | hypothetical protein | M. jannaschii | 0.05 | 0 | | |
| 705 | | | predicted coding | | | | |
| | | | region MJECL28 | | | | |
| | | | [Methanococcus | | | | |
| | | | jannaschii]. | | | | |
| 706, | NEQ359 | SSU Ribosomnal | SSU ribosomal | 1.00E−20 | Ribosomal_S28e Ribosomal | | Information |
| 707 | | protein S28E | protein S28E | | protein S28e 3.70e−32 | | storage and |
| | | | [Pyrococcus abyssi]. | | | | processing; Translation, |
| | | | | | | | ribosomal |
| | | | | | | | structure and |
| | | | | | | | biogenesis; Ribosomal |
| | | | | | | | protein |
| | | | | | | | S28E/S33 8e−22 |
| 708, | NEQ361 | LSU ribosomal | LSU ribosomal protein | 4.00E−66 | Ribosomal_L2 Ribosomal | | Information |
| 709 | | protein | L2P [Pyrococcus | | Proteins L2, RNA binding | | storage and |
| | | L2P[Pyrococcus | abyssi] | | domain 1.70e−10 :rplB_bact | | processing; Translation, |
| | | abyssi] | | | rplB_bact ribosomal protein | | ribosomal |
| | | | | | L2 3.00e−08 :FOLD1900 No | | structure and |
| | | | | | CATH Annotation 2.20e−12 | | biogenesis; Ribosomal |
| | | | | | :Ribosomal_L2_C | | protein L2 3e−67 |
| | | | | | Ribosomal Proteins L2, C- | | |
| | | | | | terminal domain 1.10e−46 | | |
| 710, | NEQ360 | Glu-tRNA | Glu-tRNA | 3.00E−80 | Amidase Amidase | 6.3.5.— | Information |
| 711 | | amidotransferase, | amidotransferase, | | 5.80e−87 :gatA gatA | | storage and |
| | | subunitA (gatA-2) | subunit A (gatA-2) | | glutamyl-tRNA(Gln) | | processing; Translation, |
| | | [Archaeoglobus | [Archaeoglobus | | amidotransferase, A | | ribosomal |
| | | fulgidus] | fulgidus]. | | subunit 6.20e−105 | | structure and |
| | | | | | | | biogenesis; Asp- |
| | | | | | | | tRNAAsn/Glu- |
| | | | | | | | tRNAGln |
| | | | | | | | amidotransferase |
| | | | | | | | A subunit |
| | | | | | | | and related |
| | | | | | | | amidases 2e−81 |
| 712, | NEQ362 | hypothetical protein | hypothetical protein | 4.00E−03 | NTP_transf_2 Nucleotidyltransferase | | |
| 713 | | | [Pyrococcus furiosus | | domain 2.60e−3 | | |
| | | | DSM 3638]. | | | | |
| 714, | NEQ363 | Archaea-specific | hypothetical protein | 5.00E−20 | DUF78 Protein of unknown | | Information |
| 715 | | DNA-bindingprotein | [Pyrococcus | | function DUF78 5.40e−34 | | storage and |
| | | [Methanopyrus | horikoshii]. | | :TIGR00285 TIGR00285 | | processing; Transcription; |
| | | kandleri] | | | conserved hypothetical | | Archaeal |
| | | | | | protein TIGR00285 3.90e−35 | | DNA- |
| | | | | | | | binding |
| | | | | | | | protein 3e−21 |
| 716, | NEQ364 | LSU Ribosomal | LSU ribosomal protein | 2.00E−12 | Ribosomal_L31e Ribosomal | | Information |
| 717 | | proten L31E | L31E [Methanococcus | | protein L31e 1.20e−11 | | storage and |
| | | | jannaschii]. | | | | processing; Translation, |
| | | | | | | | ribosomal |
| | | | | | | | structure and |
| | | | | | | | biogenesis; Ribosomal |
| | | | | | | | protein L31E |
| | | | | | | | 1e−13 |
| 718, | NEQ365 | hypothetical protein | hypothetical protein | 9.00E−15 | | 3.1.11.— | Information |
| 719 | | | [Plasmodium | | | | storage and |
| | | | falciparum 3D7]. | | | | processing; DNA |
| | | | | | | | replication, |
| | | | | | | | recombination |
| | | | | | | | and |
| | | | | | | | repair; ATPase |
| | | | | | | | involved in |
| | | | | | | | DNA repair |
| | | | | | | | 9e−14 |
| 720, | NEQ366 | GTP-binding protein | GTP-binding protein | 1.00E−39 | MMR_HSR1 GTPase of | | Poorly |
| 721 | | [Pyrococcushorikoshii] | [Pyrococcus | | unknown function 4.00e−25 | | characterized; |
| | | | horikoshii]. | | :MG442 MG442 GTP- | | General |
| | | | | | binding conserved | | function |
| | | | | | hypothetical protein 1.50e−13 | | prediction |
| | | | | | | | only; Predicted |
| | | | | | | | GTPases |
| | | | | | | | 7e−41 |
| 722, | NEQ367 | nucleotidyltransferase | 176aa long conserved | 9.00E−03 | CTP_transf_2 Cytidylyltransferase 6.60e−06 | | |
| 723 | | [Pyrococcus furiosus | hypothetical protein | | | | |
| | | DSM 3638] | [Sulfolobus tokodaii]. | | | | |
| 724, | NEQ368 | DNA-(apurinic or | M. jannaschii | 4.00E−48 | AP_endonuc_2 AP endonuclease | | |
| 725 | | apyrimidinic | predicted coding | | family 2 1.80e−3 | | |
| | | site)lyase | region MJ1455 | | | | |
| | | (endonuclease IV) | [Methanococcus | | | | |
| | | | jannaschii]. | | | | |
| 726, | NEQ369 | DNA repair protein | ATP-dependent | 3.00E−33 | DEAD DEAD/DEAH | 2.7.7.— | Information |
| 727 | | RAD25[Pyrococcus | helicase, putative | | box helicase 1.60e−11 | | storage and |
| | | abyssi] | [Sulfolobus | | :helicase_C | | processing; DNA |
| | | | solfataricus]. | | Helicase conserved | | replication, |
| | | | | | C-terminal domain | | recombination |
| | | | | | 8.30e−10 :SNF2_N | | and |
| | | | | | SNF2 family N- | | repair; DNA or |
| | | | | | terminal domain | | RNA |
| | | | | | 8.00e−3 | | helicases of |
| | | | | | | | superfamily II |
| | | | | | | | 5e−25 |
| 728, | NEQ370 | DNA-directed RNA | DNA-directed RNA | 5.00E−39 | S1 S1 RNA binding | 2.7.7.6 | Information |
| 729 | | polymerase, subunit | polymerase, subunit E | | domain 1.90e−10 | | storage and |
| | | E′ (rpoE1) [M. jannaschii] | (rpoE1) | | :rpoE rpoE DNA- | | processing; Transcription; |
| | | | [Methanococcus | | directed RNA | | DNA- |
| | | | jannaschii]. | | polymerase 2.80e−47 | | directed |
| | | | | | :RNA_pol_Rpb7_N | | RNA |
| | | | | | RNA polymerase | | polymerase |
| | | | | | Rpb7, N-terminal | | subunit E 4e−40 |
| | | | | | domain 4.60e−22 | | |
| 730, | NEQ372 | hypothetical DNA- | 220aa long | 5.00E−57 | UDG Uracil DNA | 2.7.7.7 | Information |
| 731 | | directed | hypothetical DNA- | | glycosylase | | storage and |
| | | DNApolymerase, | directed DNA | | superfamily 1.20e−37 | | processing; DNA |
| | | bacteriophage-type | polymerase, | | :SPO1polNrel | | replication, |
| | | | bacteriophage-type | | SPO1polNrel phage | | recombination |
| | | | [Sulfolobus tokodaii]. | | SPO1 DNA | | and |
| | | | | | polymerase-related | | repair; Uracil- |
| | | | | | protein 8.30e−94 | | DNA |
| | | | | | | | glycosylase |
| | | | | | | | 6e−54 |
| 732, | NEQ371 | conserved | Hypothetical protein | 7.00E−25 | | 3.1.3.3 | Poorly |
| 733 | | hypothetical | [Sulfolobus | | | | characterized; |
| | | protein[Sulfolobus | solfataricus]. | | | | Function |
| | | solfataricus] | | | | | unknown; Uncharacterized |
| | | | | | | | archaeal |
| | | | | | | | coiled-coil |
| | | | | | | | domain 4e−24 |
| 734, | NEQ373 | ferredoxin1 [3Fe-4S] - | FERREDOXIN. | 3.00E−07 | fer4 4Fe-4S binding domain | | Metabolism; Energy |
| 735 | | Thermococcuslitoralis | | | 3.00e−3 | | production |
| | | | | | | | and |
| | | | | | | | conversion; Ferredoxin |
| | | | | | | | 1 6e−07 |
| 736, | NEQ375 | SSU Ribosomal | Ribosomal protein | 2.00E−23 | Ribosomal_S3Ae Ribosomal | | Information |
| 737 | | protein | S3AE [Methanopyrus | | S3Ae family 5.80e−15 | | storage and |
| | | S3AE[Methanopyrus | kandleri AV19]. | | | | processing; Translation, |
| | | kandleri AV19] | | | | | ribosomal |
| | | | | | | | structure and |
| | | | | | | | biogenesis; Ribosomal |
| | | | | | | | protein S3AE |
| | | | | | | | 3e−23 |
| 738, | NEQ376 | hypothetical protein | Unknown (protein for | 0.29 | 0 | | |
| 739 | | | MGC: 27006) [Homo | | | | |
| | | | sapiens]. | | | | |
| 740, | NEQ377 | DNA-directed RNA | 112aa long | 8.00E−23 | RNA_POL_M_15 KD | 2.7.7.6 | Information |
| 741 | | polymerasesubunit | hypothetical DNA- | | RNA polymerases | | storage and |
| | | M [Sulfolobus | directed RNA | | M/15 Kd subunit | | processing; Transcription; |
| | | tokodaii] | polymerase subunit M | | 7.60e−08 :TFIIS | | DNA- |
| | | | [Sulfolobus tokodaii]. | | Transcription factor | | directed |
| | | | | | S-II (TFIIS) 2.50e−15 | | RNA |
| | | | | | | | polymerase |
| | | | | | | | subunit |
| | | | | | | | M/Transcription |
| | | | | | | | elongation |
| | | | | | | | factor TFIIS |
| | | | | | | | 5e−21 |
| 742, | NEQ378 | Predicted Zn- | hypothetical protein | 1.00E−54 | lactamase_B Metallo-beta- | | Poorly |
| 743 | | dependent hydrolase | [Pyrococcus abyssi]. | | lactamase superfamily | | characterized; |
| | | ofthe beta-lactamase | | | 9.80e−11 | | General |
| | | superfamily | | | | | function |
| | | | | | | | prediction |
| | | | | | | | only; Predicted |
| | | | | | | | Zn- |
| | | | | | | | dependent |
| | | | | | | | hydrolases of |
| | | | | | | | the beta- |
| | | | | | | | lactamase |
| | | | | | | | fold 7e−56 |
| 744, | NEQ379 | LSU ribosomal | 50S ribosomal protein | 2.00E−42 | Ribosomal_L19e Ribosomal | | Information |
| 745 | | protein | L19 [Pyrococcus | | protein L19e 6.10e−67 | | storage and |
| | | L19E[Pyrococcus | horikoshii]. | | | | processing; Translation, |
| | | horikoshii] | | | | | ribosomal |
| | | | | | | | structure and |
| | | | | | | | biogenesis; Ribosomal |
| | | | | | | | protein L19E |
| | | | | | | | 3e−43 |
| 746, | NEQ380 | hypothetical protein | hypothetical protein | 4.00E−03 | 0 | | |
| 747 | | | [Plasmodium | | | | |
| | | | falciparum 3D7]. | | | | |
| 748, | NEQ381 | pyruvate fromate- | PYRUVATE | 4.00E−71 | Radical_SAM | 1.97. | Cellular |
| 749 | | lyase | FORMATE-LYASE | | Radical SAM | 1.4 | processes; Posttranslational |
| | | activatingenzyme | ACTIVATING | | superfamily 4.30e−15 | | modification, |
| | | related protein [P. abyssi] | ENZYME RELATED | | | | protein |
| | | | PROTEIN | | | | turnover, |
| | | | [Pyrococcus abyssi]. | | | | chaperones; Pyruvate- |
| | | | | | | | formate |
| | | | | | | | lyase- |
| | | | | | | | activating |
| | | | | | | | enzyme 3e−72 |
| 750, | NEQ383 | DNA repair protein | hypothetical protein | 1.00E−13 | Metallophos Calcineurin-like | | Information |
| 751 | | RAD32[Pyrococcus | [Pyrococcus | | phosphoesterase 7.10e−15 | | storage and |
| | | abyssi] | horikoshii]. | | :sbcd sbcd exonuclease | | processing; DNA |
| | | | | | SbcD 5.60e−05 | | replication, |
| | | | | | | | recombination |
| | | | | | | | and |
| | | | | | | | repair; DNA |
| | | | | | | | repair |
| | | | | | | | exonuclease |
| | | | | | | | 8e−15 |
| 752, | NEQ382 | Predicted Fe—S | hypothetical protein | 1.00E−73 | Radical_SAM Radical SAM | | Metabolism; Energy |
| 753 | | oxidoreductase[Thermoplasma | [Pyrococcus furiosus | | superfamily 7.50e−15 | | production |
| | | volcanium] | DSM 3638]. | | | | and |
| | | | | | | | conversion; Fe—S |
| | | | | | | | oxidoreductases |
| | | | | | | | 2e−74 |
| 754, | NEQ384 | SpoU rRNA | spoU protein homolog | 1.00E−36 | SpoU_methylase | 2.1.1.— | Information |
| 755 | | methylase[Methanos | [imported] - | | SpoU rRNA | | storage and |
| | | arcina acetivorans | Pyrococcus sp. | | Methylase family | | processing; Translation, |
| | | str. C2A] | | | 1.60e−07 | | ribosomal |
| | | | | | :rRNA_methyl_1 | | structure and |
| | | | | | rRNA_methyl_1 | | biogenesis; rRNA |
| | | | | | RNA | | methylase |
| | | | | | methyltransferase, | | 5e−32 |
| | | | | | TrmH family, group | | |
| | | | | | 1 5.10e−40 | | |
| | | | | | :rRNA_methyl_2 | | |
| | | | | | rRNA_methyl_2 | | |
| | | | | | RNA | | |
| | | | | | methyltransferase, | | |
| | | | | | TrmH family, group | | |
| | | | | | 2 2.30e−3 | | |
| 756, | NEQ386 | hypothetical protein | conserved | 7.00E−06 | DUF153 MTH1175-like domain | | |
| 757 | | [Pyrococcusabyssi] | hypothetical protein | | (DUF153/COG1433) 2.70e−05 | | |
| | | | [Thermoanaerobacter | | | | |
| | | | tengcongensis]. | | | | |
| 758, | NEQ385 | conserved | hypothetical protein | 1.00E−05 | DUF232 Putative transcriptional | | |
| 759 | | hypothetical | [Aeropyrum pernix]. | | regulator 4.40e−11 | | |
| | | protein[Aeropyrum | | | | | |
| | | pernix] | | | | | |
| 760, | NEQ387 | ATP-dependent RNA | ATP-dependent RNA | 1.00E−127 | DEAD DEAD/DEAH | 2.7.7.— | Information |
| 761 | | helicase, EIF- | helicase, EIF-4A | | box helicase 8.30e−08 | | storage and |
| | | 4AFAMILY | FAMILY [Pyrococcus | | :ERCC4 ERCC4 | | processing; DNA |
| | | [Pyrococcus abyssi] | abyssi]. | | domain 8.70e−23 | | replication, |
| | | | | | :helicase_C | | recombination |
| | | | | | Helicase conserved | | and |
| | | | | | C-terminal domain | | repair; ERCC4- |
| | | | | | 9.30e−25 :SNF2_N | | like helicases |
| | | | | | SNF2 family N- | | 3e−97 |
| | | | | | terminal domain | | |
| | | | | | 1.40e−3 | | |
| 762, | NEQ388 | SSU ribosomal | ribosomal protein S5 | 2.00E−55 | Ribosomal_S5 Ribosomal | | Information |
| 763 | | protein | [Pyrobaculum | | protein S5, N-terminal | | storage and |
| | | S5[Pyrobaculum | aerophilum]. | | domain 1.40e−10 :rpsE_arch | | processing; Translation, |
| | | aerophilum] | | | rpsE_arch ribosomal protein | | ribosomal |
| | | | | | S5 4.10e−97 :rpsE_bact | | structure and |
| | | | | | rpsE_bact ribosomal protein | | biogenesis; Ribosomal |
| | | | | | S5 4.80e−12 | | protein S5 2e−56 |
| | | | | | :Ribosomal_S5_C | | |
| | | | | | Ribosomal protein S5, C- | | |
| | | | | | terminal domain 7.30e−21 | | |
| 764, | NEQ389 | tyrosyl-tRNA | tyrosyl-tRNA | 1.00E−100 | tRNA-synt_1b tRNA | 6.1.1.1 | Information |
| 765 | | synthetase (class | synthetase | | synthetases class I | | storage and |
| | | 1b)[Pyrococcus | [Pyrococcus furiosus | | (W and Y) 7.60e−54 | | processing; Translation, |
| | | furiosus] | DSM 3638]. | | :tyrS tyrS tyrosyl- | | ribosomal |
| | | | | | tRNA synthetase | | structure and |
| | | | | | 6.40e−17 | | biogenesis; Tyrosyl- |
| | | | | | | | tRNA |
| | | | | | | | synthetase |
| | | | | | | | 1e−100 |
| 766, | NEQ392 | hypothetical protein | potassium channel | 0.14 | 0 | | |
| 767 | | | subunit [Gallus | | | | |
| | | | gallus]. | | | | |
| 768, | NEQ391 | hypothetical protein | AMV156 [Amsacta | 7.00E−10 | 0 | | |
| 769 | | | moorei | | | | |
| | | | entomopoxvirus]. | | | | |
| 770, | NEQ393 | translation initiation | translation initiation | 1.00E−19 | eIF_5A eIF_5A translation | | Information |
| 771 | | factor eIF-5A(eif5A) | factor eIF-5A (eif5A) | | initiation factor eIF-5A 8.80e−21 | | storage and |
| | | [Archaeoglobus | [Archaeoglobus | | | | processing; Translation, |
| | | fulgidus] | fulgidus]. | | | | ribosomal |
| | | | | | | | structure and |
| | | | | | | | biogenesis; Translation |
| | | | | | | | elongation |
| | | | | | | | factor |
| | | | | | | | P/translation |
| | | | | | | | initiation |
| | | | | | | | factor eIF-5A |
| | | | | | | | 2e−20 |
| 772, | NEQ396 | Glu-tRNA | Glu-tRNA | 3.00E−49 | GAD GAD domain | 6.3.5.— | Information |
| 773 | | amidotransferase | amidotransferase | | 1.30e−4 :GatB | | storage and |
| | | (gatE) | (gatB) | | PET112 family, C | | processing; Translation, |
| | | | [Methanococcus | | terminal region | | ribosomal |
| | | | jannaschii]. | | 5.50e−06 :gatB_rel | | structure and |
| | | | | | gatB_rel aspartyl- | | biogenesis; Archaeal |
| | | | | | tRNA(Asn) | | Glu- |
| | | | | | amidotransferase, B | | tRNAGln |
| | | | | | subunit, putative | | amidotransferase |
| | | | | | 1.70e−46 | | subunit E |
| | | | | | | | (contains |
| | | | | | | | GAD domain) |
| | | | | | | | 2e−50 |
| 774, | NEQ395 | hypothetical protein | conserved | 4.00E−05 | primase_sml primase_sml DNA primase, | | |
| 775 | | | hypothetical protein | | eukaryotic-type, small subunit, | | |
| | | | [Thermoplasma | | putative 4.30e−4 | | |
| | | | acidophilum]. | | | | |
| 776, | NEQ397 | LSU ribosomal | LSU ribosomal protein | 9.00E−25 | Ribosomal_L21e Ribosomal | | Information |
| 777 | | protein | L21E; (rpl21E) | | protein L21e 2.20e−25 | | storage and |
| | | L21E; (rpl21E) | [Pyrococcus furiosus | | | | processing; Translation, |
| | | [Pyrococcus | DSM 3638]. | | | | ribosomal |
| | | furiosus] | | | | | structure and |
| | | | | | | | biogenesis; Ribosomal |
| | | | | | | | protein L21E |
| | | | | | | | 2e−25 |
| 778, | NEQ398 | endonuclease III | endonuclease III | 4.00E−40 | HhH-GPD HhH- | 4.2.99. | Information |
| 779 | | [Aquifexaeolicus]Split; | [Aquifex aeolicus]. | | GPD superfamily | 18 | storage and |
| | | see SEQ ID | | | base excision DNA | | processing; DNA |
| | | NOS: 254, 255 | | | repair protein 2.00e−17 | | replication, |
| | | | | | :nth nth | | recombination |
| | | | | | endonuclease III | | and |
| | | | | | 2.80e−40 | | repair; Predicted |
| | | | | | | | EndoIII- |
| | | | | | | | related |
| | | | | | | | endonuclease |
| | | | | | | | 3e−41 |
| 780, | NEQ399 | methionine | methionine | 2.00E−58 | Peptidase_M24 | 3.4.11. | Information |
| 781 | | aminopeptidase | aminopeptidase (map) | | metallopeptidase | 18 | storage and |
| | | (map)(EC 3.4.11.18) | (EC 3.4.11.18) | | family M24 6.00e−12 | | processing; Translation, |
| | | [Pyrococcus abyssi] | [Pyrococcus abyssi]. | | :crvDNA_42K | | ribosomal |
| | | | | | crvDNA_42K DNA- | | structure and |
| | | | | | binding protein, 42 kDa | | biogenesis; Methionine |
| | | | | | 3.60e−4 | | aminopeptidase |
| | | | | | :met_pdase_I | | 2e−59 |
| | | | | | met_pdase_I | | |
| | | | | | methionine | | |
| | | | | | aminopeptidase, | | |
| | | | | | type I 1.80e−11 | | |
| | | | | | :met_pdase_II | | |
| | | | | | met_pdase_II | | |
| | | | | | methionine | | |
| | | | | | aminopeptidase, | | |
| | | | | | type II 8.20e−81 | | |
| 782, | NEQ401 | Mg-chelatase | Mg-chelatase subunit | 2.00E−77 | AAA ATPase family | | Metabolism; Coenzyme |
| 783 | | subunit Chll and | Chll and Chld (MoxR- | | associated with various | | metabolism; Mg- |
| | | Chld(MoxR-like | like ATPase and vWF | | cellular activities (AAA) | | chelatase |
| | | ATPase and vWF | domain) | | 6.90e−06 :Mg_chelatase | | subunit Chll |
| | | | [Methanopyrus | | Magnesium chelatase, | | 5e−36 |
| | | | kandleri AV19]. | | subunit Chll 3.60e−08 | | |
| | | | | | :Sigma54_activat Sigma-54 | | |
| | | | | | interaction domain 3.10e−3 | | |
| 784, | NEQ403 | hypothetical protein | conserved | 4.00E−05 | vwa von Willebrand factor type | | |
| 785 | | | hypothetical protein | | A domain 1.70e−3 | | |
| | | | [Chlorobium tepidum | | | | |
| | | | TLS]. | | | | |
| 786, | NEQ402 | deoxyhypusine | deoxyhypusine | 4.00E−89 | DS Deoxyhypusine | 2.5.1. | Information |
| 787 | | synthase | synthase related | | synthase 5.90e−73 | 46 | storage and |
| | | (catalyzingalso the | protein | | :dhys dhys | | processing; Translation, |
| | | synthesis of | [Thermoplasma | | deoxyhypusine | | ribosomal |
| | | homospermidine) | acidophilum]. | | synthase, putative | | structure and |
| | | | | | 8.50e−67 | | biogenesis; Deoxyhypusine |
| | | | | | | | synthase 3e−90 |
| 788, | NEQ404 | hypothetical protein | Uncharacterized | 0.01 | 0 | | |
| 789 | | | protein conserved in | | | | |
| | | | bacteria | | | | |
| | | | [Wigglesworthia | | | | |
| | | | brevipalpis]. | | | | |
| 790, | NEQ405 | Translation initiation | Translation initiation | 1.00E−30 | S1 S1 RNA binding domain | | Information |
| 791 | | factor eIF2- | factor eIF2-alpha | | 7.60e−07 | | storage and |
| | | alpha[Methanopyrus | [Methanopyrus | | | | processing; Translation, |
| | | kandleri AV19] | kandleri AV19]. | | | | ribosomal |
| | | | | | | | structure and |
| | | | | | | | biogenesis; Translation |
| | | | | | | | initiation |
| | | | | | | | factor |
| | | | | | | | eIF2alpha 2e−29 |
| 792, | NEQ406 | hypothetical | similar to | 3.00E−06 | 0 | | |
| 793 | | proteinsimilar to | phosphoenolpyruvate | | | | |
| | | PEP-kinase | synthase [Nostoc sp. | | | | |
| | | | PCC 7120]. | | | | |
| 794, | NEQ408 | hypothetical protein | hypothetical protein | 0.01 | 0 | | |
| 795 | | | [Aeropyrum pernix]. | | | | |
| 796, | NEQ407 | hypothetical protein | hypothetical protein | 2.00E−04 | 0 | | |
| 797 | | | [Streptococcus | | | | |
| | | | mutans UA159]. | | | | |
| 798, | NEQ409 | large helicase- | large helicase-related | 8.00E−68 | 0 | 3.6.1.— | Poorly |
| 799 | | related protein; (lhr- | protein; (lhr-2) | | | | characterized; |
| | | 2) | [Pyrococcus furiosus | | | | General |
| | | | DSM 3638]. | | | | function |
| | | | | | | | prediction |
| | | | | | | | only; Lhr-like |
| | | | | | | | helicases 4e−68 |
| 800, | NEQ410 | V-type ATP synthase | hypothetical protein | 8.00E−34 | V_ATPase_sub_a | 3.6.3. | Metabolism; Energy |
| 801 | | subunit I (V- | [Clostridium | | V-type ATPase | 14 | production |
| | | typeATPase subunit | thermocellum ATCC | | 116 kDa subunit | | and |
| | | I) [Pc. abyssi] | 27405]. | | family 7.40e−11 | | conversion; Archaeal/ |
| | | | | | | | vacuolar- |
| | | | | | | | type H+- |
| | | | | | | | ATPase |
| | | | | | | | subunit I 8e−33 |
| 802, | NEQ411 | DNA-directed RNA | Chain F, Structure Of | 4.00E−06 | RNA_pol_Rpb4 RNA | | Poorly |
| 803 | | polymerase, subunit | An Archeal Homolog | | polymerase Rpb4 1.60e−4 | | characterized; |
| | | F (rpoF) [S. solfataricus] | Of The Eukaryotic | | | | Function |
| | | | Rna Polymerase li | | | | unknown; Uncharacterized |
| | | | Rpb4RPB7 | | | | ArCR 2e−07 |
| | | | COMPLEX. | | | | |
| 804, | NEQ412 | leucine | leucine | 1.00E−67 | Peptidase_M17 | 3.4.11.1 | Metabolism; Amino |
| 805 | | aminopeptidase | aminopeptidase | | Cytosol | | acid |
| | | [Clostridiumperfringens] | [Clostridium | | aminopeptidase | | transport and |
| | | | perfringens]. | | family, catalytic | | metabolism; Leucyl |
| | | | | | domain 5.10e−123 | | aminopeptidase |
| | | | | | | | 5e−64 |
| 806, | NEQ413 | hypothetical protein | hypothetical protein | 0.91 | 0 | | |
| 807 | | | [Enterococcus | | | | |
| | | | faecium]. | | | | |
| 808, | NEQ415 | hypothetical protein | 185aa long | 2.00E−11 | 0 | | |
| 809 | | [Sulfolobus tokodaii] | hypothetical protein | | | | |
| | | | [Sulfolobus tokodaii]. | | | | |
| 810, | NEQ414 | Archaeal ATP | hypothetical protein | 5.00E−20 | 0 | | Poorly |
| 811 | | dependent | [Pyrococcus furiosus | | | | characterized; |
| | | serineprotease | DSM 3638]. | | | | General |
| | | {secretory signal | | | | | function |
| | | sequence} | | | | | prediction |
| | | | | | | | only; Archaeal |
| | | | | | | | serine |
| | | | | | | | proteases 5e−21 |
| 812, | NEQ416 | hypothetical protein | Predicted ATPase | 0.06 | 0 | | |
| 813 | | | involved in biogenesis | | | | |
| | | | of flagella | | | | |
| | | | [Thermoplasma | | | | |
| | | | volcanium]. | | | | |
| 814, | NEQ417 | glycyl-tRNA | glycyl-tRNA | 2.00E−98 | HGTP_anticodon | 6.1.1. | Information |
| 815 | | synthetase (class | synthetase | | Anticodon binding | 14 | storage and |
| | | 2)[Pyrococcus | [Pyrococcus abyssi]. | | domain 4.00e−30 | | processing; Translation, |
| | | abyssi] | | | :tRNA-synt_2b tRNA | | ribosomal |
| | | | | | synthetase class II | | structure and |
| | | | | | core domain (G, H, | | biogenesis; Glycyl- |
| | | | | | P, S and T) 3.40e−41 | | tRNA |
| | | | | | :glyS_dimeric | | synthetase, |
| | | | | | glyS_dimeric glycyl- | | class II 1e−99 |
| | | | | | tRNA synthetase | | |
| | | | | | 2.00e−151 | | |
| 816, | NEQ418 | hypothetical protein | hypothetical protein | 0.02 | 0 | | |
| 817 | | | [Plasmodium | | | | |
| | | | falciparum 3D7]. | | | | |
| 818, | NEQ420 | DNA polymerase II | DNA polymerase II | 0 | polC polC DNA | 2.7.7.7 | Information |
| 819 | | subunit 2 | subunit 2 [Pyrococcus | | polymerase II, large | | storage and |
| | | (largesubunit) | furiosus DSM 3638]. | | subunit DP2 0.0 | | processing; DNA |
| | | [Pyrococcus | | | :PolC_DP2 DNA | | replication, |
| | | furiosus] | | | polymerase II large | | recombination |
| | | | | | subunit DP2 4.60e−283 | | and repair; NA |
| | | | | | | | polymerase II |
| | | | | | | | large subunit 0 |
| 820, | NEQ419 | hypothetical protein | hypothetical protein | 3.00E−07 | 0 | | Poorly |
| 821 | | [Pyrococcus horikoshii] | [Pyrococcus | | | | characterized; |
| | | | horikoshii]. | | | | Function |
| | | | | | | | unknown; Uncharacterized |
| | | | | | | | ArCR 2e−08 |
| 822, | NEQ421 | ATPase subunit of a | ABC transporter, | 1.00E−56 | ABC_tran ABC | 1.8.—.— | Poorly |
| 823 | | ABC-typetransport | ATP-binding protein | | transporter 2.00e−58 | | characterized; |
| | | system involved in | [Methanosarcina | | :3a0501s02 | | General |
| | | | acetivorans str. C2A]. | | 3a0501s02 Type II | | function |
| | | | | | (General) Secretory | | prediction |
| | | | | | Pathway (IISP) | | only; ABC- |
| | | | | | Family protein | | type transport |
| | | | | | 1.80e−55 | | systems, |
| | | | | | :3a0106s01 | | involved in |
| | | | | | 3a0106s01 sulfate | | lipoprotein |
| | | | | | transport system | | release, |
| | | | | | permease protein | | ATPase |
| | | | | | 3.70e−46 :cbiO cbiO | | components |
| | | | | | cobalt transport | | 1e−55 |
| | | | | | protein ATP-binding | | |
| | | | | | subunit 2.80e−16 | | |
| | | | | | :ntrCD ntrCD nitrate | | |
| | | | | | transport ATP- | | |
| | | | | | binding subunits C | | |
| | | | | | and D 8.00e−20 | | |
| | | | | | :proV proV glycine | | |
| | | | | | betaine/L-proline | | |
| | | | | | transport ATP | | |
| | | | | | binding subunit | | |
| | | | | | 4.10e−09 :potA potA | | |
| | | | | | spermidine/putrescine | | |
| | | | | | ABC transporter | | |
| | | | | | ATP-binding subunit | | |
| | | | | | 1.10e−09 :drrA drrA | | |
| | | | | | daunorubicin | | |
| | | | | | resistance ABC | | |
| | | | | | transporter ATP- | | |
| | | | | | binding subunit | | |
| | | | | | 1.90e−10 :ccmA | | |
| | | | | | ccmA heme | | |
| | | | | | exporter protein | | |
| | | | | | CcmA 3.80e−14 | | |
| | | | | | :thiQ thiQ ABC | | |
| | | | | | transporter, ATP- | | |
| | | | | | binding protein, | | |
| | | | | | ThiQ subfamily | | |
| | | | | | 3.80e−16 :nodl nodl | | |
| | | | | | nodulation ABC | | |
| | | | | | transporter Nodl | | |
| | | | | | 2.70e−05 | | |
| 824, | NEQ422 | DIPHTIN | DIPHTIN SYNTHASE | 5.00E−22 | TP_methylase | 2.1.1. | Information |
| 825 | | SYNTHASE[Encephalitozoon | [Encephalitozoon | | Tetrapyrrole | 98 | storage and |
| | | cuniculi] | cuniculi]. | | (Corrin/Porphyrin) | | processing; Translation, |
| | | | | | Methylases 5.70e−4 | | ribosomal |
| | | | | | :dph5 dph5 | | structure and |
| | | | | | diphthine synthase | | biogenesis; Diphthamide |
| | | | | | 3.40e−13 | | biosynthesis |
| | | | | | | | methyltransferase |
| | | | | | | | DPH5 |
| | | | | | | | 8e−20 |
| 826, | NEQ424 | Holliday-junction | conserved protein | 1.00E−08 | Hjc Archaeal holliday | | Information |
| 827 | | resolvase[Sulfolobus | [Methanothermobacter | | junction resolvase (hjc) | | storage and |
| | | solfataricus] | thermautotrophicus]. | | 1.70e−18 | | processing; DNA |
| | | | | | | | replication, |
| | | | | | | | recombination |
| | | | | | | | and |
| | | | | | | | repair; Holliday |
| | | | | | | | junction |
| | | | | | | | resolvase - |
| | | | | | | | archaeal type |
| | | | | | | | 6e−10 |
| 828, | NEQ423 | Probable thiamine | Thiamine biosynthesis | 2.00E−47 | Thil Thiamine biosynthesis | | Metabolism; Coenzyme |
| 829 | | biosynthesisprotein | ATP pyrophosphatase | | protein (Thil) 5.40e−19 | | metabolism; Thiamine |
| | | thil [Archaeoglobus | [Thermoanaerobacter | | :THUMP THUMP domain | | biosynthesis |
| | | fulgidus] | tengcongensis]. | | 7.10e−18 :TIGR00342 | | ATP |
| | | | | | TIGR00342 thiamine | | pyrophosphatase |
| | | | | | biosynthesis protein Thil | | 8e−43 |
| | | | | | 2.20e−39 | | |
| 830, | NEQ425 | type IV secretion | type IV secretion | 7.00E−76 | GSPII_E Type II/IV secretion | | Cellular |
| 831 | | system | system protein | | system protein 2.40e−3 | | processes; Cell |
| | | protein[Methanosarcina | [Methanosarcina | | | | motility and |
| | | acetivorans str. | acetivorans str. C2A]. | | | | secretion; Type |
| | | C2A] | | | | | IV secretory |
| | | | | | | | pathway, |
| | | | | | | | VirB11 |
| | | | | | | | components, |
| | | | | | | | and related |
| | | | | | | | ATPases |
| | | | | | | | involved in |
| | | | | | | | archaeal |
| | | | | | | | flagella |
| | | | | | | | biosynthesis |
| | | | | | | | 1e−34 |
| 832, | NEQ426 | recombinase, radA | recombinase, radA | 9.00E−97 | HHH Helix-hairpin- | 3.6.1.— | Information |
| 833 | | [Pyrococcusfuriosus | [Pyrococcus furiosus | | helix motif 7.40e−05 | | storage and |
| | | DSM 3638] | DSM 3638]. | | | | processing; DNA |
| | | | | | | | replication, |
| | | | | | | | recombination |
| | | | | | | | and |
| | | | | | | | repair; RecA/RadA |
| | | | | | | | recombinase |
| | | | | | | | 5e−97 |
| 834, | NEQ427 | DNA-directed RNA | DNA-directed RNA | 9.00E−81 | 1.10E−23 | 2.7.7.6 | Information |
| 835 | | polymerase, subunit | polymerase, subunit A | | | | storage and |
| | | A″ (rpoA2) | [Pyrococcus abyssi]. | | | | processing; Transcription; |
| | | [Pyrococcus | | | | | DNA- |
| | | | | | | | directed |
| | | | | | | | RNA |
| | | | | | | | polymerase |
| | | | | | | | beta' |
| | | | | | | | subunit/160 kD |
| | | | | | | | subunit |
| | | | | | | | (split gene in |
| | | | | | | | archaea and |
| | | | | | | | Syn) 6e−82 |
| 836, | NEQ428 | Predicted RNA- | conserved | 8.00E−10 | UPF0044 Uncharacterised | | Information |
| 837 | | binding | hypothetical protein | | protein family UPF0044 | | storage and |
| | | proteincontaining KH | [Methanococcus | | 1.20e−13 :TIGR00253 | | processing; Translation, |
| | | domain, possibly | jannaschii]. | | TIGR00253 conserved | | ribosomal |
| | | | | | hypothetical protein | | structure and |
| | | | | | TIGR00253 4.20e−05 | | biogenesis; Predicted |
| | | | | | | | RNA- |
| | | | | | | | binding |
| | | | | | | | protein |
| | | | | | | | containing KH |
| | | | | | | | domain, |
| | | | | | | | possibly |
| | | | | | | | ribosomal |
| | | | | | | | protein 4e−11 |
| 838, | NEQ429 | hypothetical protein | hypothetical protein | 6.00E−03 | 0 | | |
| 839 | | | [Plasmodium | | | | |
| | | | falciparum 3D7]. | | | | |
| 840, | NEQ430 | activator 1 | activator 1 (replication | 8.00E−73 | AAA ATPase family | 2.7.7.7 | Information |
| 841 | | (replication factor C), | factor C), 53 KD | | associated with | | storage and |
| | | 53 KD subunit [Mc | subunit | | various cellular | | processing; DNA |
| | | jannaschii] | [Methanococcus | | activities (AAA) | | replication, |
| | | | jannaschii]. | | 2.60e−11 :Rad17 | | recombination |
| | | | | | Rad17 cell cycle | | and |
| | | | | | checkpoint protein | | repair; ATPase |
| | | | | | 3.50e−3 | | involved in |
| | | | | | | | DNA |
| | | | | | | | replication 5e−74 |
| 842, | NEQ431 | conserved | hypothetical protein | 5.00E−31 | DUF99 Protein of unknown | | Poorly |
| 843 | | hypothetical | [Methanosarcina | | function DUF99 1.80e−49 | | characterized; |
| | | protein[Pyrococcus | barkeri]. | | | | Function |
| | | furiosus DSM 3638] | | | | | unknown; Uncharacterized |
| | | | | | | | ACR 3e−27 |
| 844, | NEQ433 | LSU ribosomal | ribosomal protein L3 | 5.00E−71 | Ribosomal_L3 Ribosomal | | Information |
| 845 | | protein L3 (E. coli | (E. coli L3) | | protein L3 1.30e−63 | | storage and |
| | | L3)[Mtb | [Methanothermobacter | | | | processing; Translation, |
| | | thermautotrophicus] | thermautotrophicus]. | | | | ribosomal |
| | | | | | | | structure and |
| | | | | | | | biogenesis; Ribosomal |
| | | | | | | | protein L3 3e−72 |
| 846, | NEQ432 | Type I signal | Type I signal | 3.00E−08 | 0 | | Cellular |
| 847 | | peptidase[Methanopyrus | peptidase | | | | processes; Cell |
| | | kandleri AV19] | [Methanopyrus | | | | motility and |
| | | | kandleri AV19]. | | | | secretion; Signal |
| | | | | | | | peptidase |
| | | | | | | | I 6e−07 |
| 848, | NEQ434 | reverse gyrase | reverse gyrase | 1.00E−132 | DEAD DEAD/DEAH | 5.99. | Information |
| 849 | | [Pyrococcus | [Pyrococcus | | box helicase 2.60e−06 | 1.3 | storage and |
| | | | horikoshii]. | | :rgy rgy reverse | | processing; DNA |
| | | | | | gyrase 1.20e−08 | | replication, |
| | | | | | | | recombination |
| | | | | | | | and |
| | | | | | | | repair; Reverse |
| | | | | | | | gyrase 1e−134 |
| 850, | NEQ435 | hypothetical protein | hypothetical protein | 7.00E−25 | DUF460 Protein of unknown | | Poorly |
| 851 | | [Archaeoglobusfulgidus] | [Archaeoglobusfulgidus]. | | function (DUF460) 6.10e−18 | | characterized; |
| | | | | | | | Function |
| | | | | | | | unknown; Uncharacterized |
| | | | | | | | ACR 5e−26 |
| 852, | NEQ436 | protein translocase, | protein translocase, | 2.00E−46 | SecD_SecF Protein export | | Cellular |
| 853 | | subunit SECD(secD) | subunit SECD (secD) | | membrane protein 9.00e−4 | | processes; Cell |
| | | [Methanococcus | [Methanococcus | | :2A0604s01 2A0604s01 | | motility and |
| | | jannaschii] | jannaschii]. | | protein-export membrane | | secretion; Preprotein |
| | | | | | protein (SecDF) Family | | translocase |
| | | | | | 2.20e−08 :3a0501s07 | | subunit SecD |
| | | | | | 3a0501s07 protein-export | | 2e−47 |
| | | | | | membrane protein SecF | | |
| | | | | | 1.50e−05 :secD secD | | |
| | | | | | protein-export membrane | | |
| | | | | | protein SecD 2.60e−10 | | |
| 854, | NEQ437 | protein translocase, | protein translocase, | 4.00E−30 | SecD_SecF Protein export | | Cellular |
| 855 | | subunit SECF(secF) | subunit SECF (secF) | | membrane protein 1.10e−09 | | processes; Cell |
| | | [Methanococcus | [Methanococcus | | :2A0604s01 2A0604s01 | | motility and |
| | | jannaschii] | jannaschii]. | | protein-export membrane | | secretion; Preprotein |
| | | | | | protein (SecDF) Family | | translocase |
| | | | | | 2.00e−06 :3a0501s07 | | subunit SecF |
| | | | | | 3a0501s07 protein-export | | 3e−31 |
| | | | | | membrane protein SecF | | |
| | | | | | 2.20e−3 | | |
| 856, | NEQ438 | predicted RNA- | hypothetical protein | 3.00E−30 | 0 | | Information |
| 857 | | binding protein | [Pyrococcus abyssi]. | | | | storage and |
| | | homologous to | | | | | processing; Transcription; |
| | | eukaryotic snRNP | | | | | Predicted |
| | | [Methanopyrus | | | | | RNA- |
| | | kandleri AV19] | | | | | binding |
| | | SPLIT see SEQ ID | | | | | protein |
| | | NOS: 982, 983 | | | | | homologous |
| | | | | | | | to eukaryotic |
| | | | | | | | snRNP 2e−31 |
| 858, | NEQ440 | Predicted DNA- | methyltransferase | 4.00E−30 | UPF0020 Putative | 2.1.1.— | Information |
| 859 | | modificationmethylase | related protein | | RNA methylase | | storage and |
| | | [Methanopyrus | [Methanothermobacter | | family UPF0020 | | processing; DNA |
| | | kandleri] | thermautotrophicus]. | | 2.00e−30 | | replication, |
| | | | | | :TIGR01177 | | recombination |
| | | | | | TIGR01177 | | and |
| | | | | | conserved | | repair; Predicted |
| | | | | | hypothetical protein | | DNA |
| | | | | | TIGR01177 1.20e−30 | | modification |
| | | | | | | | methylase 3e−31 |
| 860, | NEQ442 | hypothetical protein | hypothetical protein | 0.02 | 0 | | |
| 861 | | | [Plasmodium | | | | |
| | | | falciparum 3D7]. | | | | |
| 862, | NEQ441 | Uncharacterized | hypothetical protein | 8.00E−53 | DUF51 Protein of unknown | | Poorly |
| 863 | | conserved | [Aquifex aeolicus]. | | function DUF51 4.60e−85 | | characterized; |
| | | protein[Methanopyrus | | | :TIGR00296 TIGR00296 | | Function |
| | | kandleri AV19] | | | conserved hypothetical | | unknown; Uncharacterized |
| | | | | | protein TIGR00296 3.50e−52 | | ACR 6e−54 |
| 864, | NEQ443 | hypothetical protein | hypothetical protein | 6.00E−03 | 0 | | |
| 865 | | | [Plasmodium | | | | |
| | | | falciparum 3D7]. | | | | |
| 866, | NEQ444 | hypothetical protein | haemagglutinin | 0.02 | 0 | | |
| 867 | | | [Mycoplasma | | | | |
| | | | gallisepticum]. | | | | |
| 868, | NEQ445 | hypothetical protein | possible HNRNP | 0.21 | 0 | | |
| 869 | | | arginine n- | | | | |
| | | | methyltransferase | | | | |
| | | | [Plasmodium yoelii | | | | |
| | | | yoelii]. | | | | |
| 870, | NEQ446 | SSU Ribosomal | SSU ribosomal | 5.00E−27 | Ribosomal_S9 Ribosomal | | Information |
| 871 | | protein | protein S9P; (rps9P) | | protein S9/S16 1.30e−29 | | storage and |
| | | S9[Methanopyrus | [Pyrococcus furiosus | | | | processing; Translation, |
| | | kandleri AV19] | DSM 3638]. | | | | ribosomal |
| | | | | | | | structure and |
| | | | | | | | biogenesis; Ribosomal |
| | | | | | | | protein S9 7e−28 |
| 872, | NEQ448 | hypothetical protein | OUTER CAPSID | 0.21 | 0 | | |
| 873 | | | PROTEIN VP4 | | | | |
| | | | (HEMAGGLUTININ) | | | | |
| | | | (OUTER LAYER | | | | |
| | | | PROTEIN VP4) | | | | |
| | | | [CONTAINS: OUTER | | | | |
| | | | CAPSID PROTEINS | | | | |
| | | | VP5 AND VP8]. | | | | |
| 874, | NEQ447 | hypothetical protein | M. jannaschii | 9.00E−03 | 0 | | |
| 875 | | | predicted coding | | | | |
| | | | region MJ0027 | | | | |
| | | | [Methanococcus | | | | |
| | | | jannaschii]. | | | | |
| 876, | NEQ449 | hypothetical protein | hypothetical protein | 5.00E−08 | 0 | | Cellular |
| 877 | | [Sulfolobustokodaii] | [Plasmodium | | | | processes; Cell |
| | | | falciparum 3D7]. | | | | division and |
| | | | | | | | chromosome |
| | | | | | | | partitioning; Chromosome |
| | | | | | | | segregation |
| | | | | | | | ATPases 2e−07 |
| 878, | NEQ450 | ubiquinol- | ubiquinol-cytochrome | 7.00E−25 | Ribosomal_L10e Ribosomal | | Information |
| 879 | | cytochrome C | C reductase complex, | | L10 7.20e−31 :L10e L10e | | storage and |
| | | reductasecomplex, | subunit VI requiring | | ribosomal protein L10.e | | processing; Translation, |
| | | subunit VI requiring | protein | | 2.30e−32 | | ribosomal |
| | | protein | [Archaeoglobus | | | | structure and |
| | | | fulgidus]. | | | | biogenesis; Ribosomal |
| | | | | | | | protein |
| | | | | | | | L16/L10E 6e−26 |
| 880, | NEQ451 | Predicted Zn- | hypothetical protein | 1.00E−21 | | | Poorly |
| 881 | | dependent | [Aquifex aeolicus]. | | | | characterized; |
| | | protease[Methanopyrus | | | | | General |
| | | kandleri AV19] | | | | | function |
| | | | | | | | prediction |
| | | | | | | | only; Predicted |
| | | | | | | | Zn- |
| | | | | | | | dependent |
| | | | | | | | proteases 1e−22 |
| 882, | NEQ452 | DNA-directed RNA | DNA-directed RNA | 3.00E−19 | RNA_pol_A_bac | 2.7.7.6 | Information |
| 883 | | polymerase, subunit | polymerase, subunit D | | Bacterial RNA | | storage and |
| | | D (rpoD) | [Pyrococcus abyssi]. | | polymerase, alpha | | processing; Transcription; |
| | | [Pyrococcus abyssi] | | | chain, N terminal | | DNA- |
| | | | | | domain 1.50e−05 | | directed |
| | | | | | | | RNA |
| | | | | | | | polymerase |
| | | | | | | | alpha |
| | | | | | | | subunit/40 kD |
| | | | | | | | subunit 2e−20 |
| 884, | NEQ453 | Predicted | Predicted | 7.00E−10 | V4R V4R domain 1.50e−05 | | Poorly |
| 885 | | transcriptional | transcriptional | | | | characterized; |
| | | regulatorconsisting | regulator consisting of | | | | General |
| | | of a V4R domain and a | a V4R domain and a | | | | function |
| | | | DNA-binding HTH | | | | prediction |
| | | | domain | | | | only; Predicted |
| | | | [Methanopyrus | | | | hydrocarbon |
| | | | kandleri AV19]. | | | | binding |
| | | | | | | | protein |
| | | | | | | | (contains V4R |
| | | | | | | | domain) 3e−08 |
| 886, | NEQ454 | centromere binding | centromere binding | 1.00E−112 | PUA PUA domain | 4.2.1.70 | Information |
| 887 | | proteinhomolog/pseudouridine | protein | | 1.20e−25 :TruB_N | | storage and |
| | | synthase | homolog/pseudouridine | | TruB family | | processing; Translation, |
| | | of | synthase | | pseudouridylate | | ribosomal |
| | | | [Pyrococcus furiosus | | synthase (N terminal | | structure and |
| | | | DSM 3638]. | | domain) 2.40e−57 | | biogenesis; Pseudouridine |
| | | | | | :CBF5 CBF5 rRNA | | synthase 1e−112 |
| | | | | | pseudouridine | | |
| | | | | | synthase, putative | | |
| | | | | | 2.40e−170 :TruB | | |
| | | | | | TruB tRNA | | |
| | | | | | pseudouridine | | |
| | | | | | synthase B 8.60e−26 | | |
| | | | | | :unchar_dom_2 | | |
| | | | | | unchar_dom_2 | | |
| | | | | | uncharacterized | | |
| | | | | | domain 2 4.80e−4 | | |
| 888, | NEQ455 | Predicted membrane | hypothetical protein | 1.00E−18 | DUF112 Integral membrane | | Poorly |
| 889 | | protein[Methanopyrus | [Pyrococcus abyssi]. | | protein DUF112 9.90e−4 | | characterized; |
| | | kandleri AV19] | | | | | Function |
| | | | | | | | unknown; Predicted |
| | | | | | | | membrane |
| | | | | | | | protein 9e−20 |
| 890, | NEQ457 | methionyl-tRNA | methionyl-tRNA | 1.00E−155 | tRNA-synt_1 tRNA | 6.1.1. | Information |
| 891 | | synthetase (class | synthetase | | synthetases class I | 10 | storage and |
| | | 1a)[Pyrococcus | [Pyrococcus | | (I, L, M and V) | | processing; Translation, |
| | | horikoshii] | horikoshii]. | | 2.20e−09 | | ribosomal |
| | | | | | :tRNA_bind Putative | | structure and |
| | | | | | tRNA binding | | biogenesis; Methionyl- |
| | | | | | domain 2.60e−40 | | tRNA |
| | | | | | :metG metG | | synthetase |
| | | | | | methionyl-tRNA | | 1e−115 |
| | | | | | synthetase 2.00e−152 | | |
| | | | | | :metG_C_term | | |
| | | | | | metG_C_term | | |
| | | | | | methionyl-tRNA | | |
| | | | | | synthetase C- | | |
| | | | | | terminal region, beta | | |
| | | | | | subunit 2.20e−28 | | |
| | | | | | :pheT_bact | | |
| | | | | | pheT_bact | | |
| | | | | | phenylalanyl-tRNA | | |
| | | | | | synthetase, beta | | |
| | | | | | subunit 2.30e−4 | | |
| 892, | NEQ456 | Mg-dependent | hypothetical protein | 6.00E−38 | TatD_DNase TatD | 3.1.21.— | Information |
| 893 | | DNase | [Acidianus | | related DNase | | storage and |
| | | [Methanopyruskandleri | ambivalens]. | | 2.80e−69 | | processing; DNA |
| | | AV19] | | | :TIGR00010 | | replication, |
| | | | | | TIGR00010 | | recombination |
| | | | | | deoxyribonuclease, | | and |
| | | | | | TatD family 6.60e−53 | | repair; Mg- |
| | | | | | | | dependent |
| | | | | | | | DNase 3e−28 |
| 894, | NEQ458 | hypothetical protein | hypothetical protein | 1.00E−03 | 0 | | |
| 895 | | | [Plasmodium | | | | |
| | | | falciparum 3D7]. | | | | |
| 896, | NEQ459 | glutaredoxin-like | glutaredoxin-like | 7.00E−23 | | 1.6.4.5 | Cellular |
| 897 | | protein | protein [Pyrococcus | | | | processes; Posttranslational |
| | | [Pyrococcusfuriosus | furiosus DSM 3638]. | | | | modification, |
| | | DSM 3638] | | | | | protein |
| | | | | | | | turnover, |
| | | | | | | | chaperones; Thiol- |
| | | | | | | | disulfide |
| | | | | | | | isomerase |
| | | | | | | | and |
| | | | | | | | thioredoxins |
| | | | | | | | 1e−23 |
| 898, | NEQ460 | Predicted | Predicted | 2.00E−39 | RIO1 RIO1/ZK632.3/MJ0444 | | Cellular |
| 899 | | serine/threonine | serine/threonine | | family 2.10e−57 | | processes; Signal |
| | | proteinkinase | protein kinase | | | | transduction |
| | | [Methanopyrus | [Methanopyrus | | | | mechanisms; |
| | | kandleri AV19] | kandleri AV19]. | | | | Predicted |
| | | | | | | | serine/threonine |
| | | | | | | | protein |
| | | | | | | | kinases 2e−38 |
| 900, | NEQ461 | inorganic pyrophosphatase[Sulfolobus tokodaii] | | | Pyrophosphatase Inorganic | 3.6.1.1 | Metabolism; Energy |
| 901 | | | | | pyrophosphatase 2.20e−63 | | production |
| | | | | | | | and |
| | | | | | | | conversion; Inorganic |
| | | | | | | | pyrophosphatase |
| | | | | | | | 7e−48 |
| 902, | NEQ462 | hypothetical protein | hypothetical protein | 6.00E−04 | 0 | | |
| 903 | | | [Plasmodium | | | | |
| | | | falciparum 3D7]. | | | | |
| 904, | NEQ464 | RIO1-like | RIO1-like | 3.00E−44 | RIO1 RIO1/ZK632.3/MJ0444 | | Cellular |
| 905 | | serine/threonine | serine/threonine | | family 1.10e−22 | | processes; Signal |
| | | proteinkinase fused | protein kinase fused | | | | transduction |
| | | to an N-terminal | to an N-terminal DNA- | | | | mechanisms; |
| | | DNA-binding | binding HTH domain | | | | RIO-like |
| | | | [Methanopyrus | | | | serine/threonine |
| | | | kandleri AV19]. | | | | protein |
| | | | | | | | kinase fused |
| | | | | | | | to N-terminal |
| | | | | | | | HTH domain |
| | | | | | | | 3e−27 |
| 906, | NEQ463 | Predicted GTPase, | GTP binding protein | 1.00E−104 | TGS TGS domain 7.60e−11 | | Poorly |
| 907 | | probabletranslation | [Sulfolobus | | :TIGR00092 TIGR00092 | | characterized; |
| | | factor [Methanopyrus | solfataricus]. | | conserved hypothetical | | General |
| | | | | | protein TIGR00092 1.70e−10 | | function |
| | | | | | | | prediction |
| | | | | | | | only; Predicted |
| | | | | | | | GTPase 4e−95 |
| 908, | NEQ466 | Predicted hydrolase | conserved | 3.00E−98 | lactamase_B | 3.—.—.— | Poorly |
| 909 | | of the metallo-beta- | hypothetical protein | | Metallo-beta- | | characterized; |
| | | lactamase | [Methanococcus | | lactamase | | General |
| | | superfamily | jannaschii]. | | superfamily 1.10e−19 | | function |
| | | | | | :MG423 MG423 | | prediction |
| | | | | | conserved | | only; Predicted |
| | | | | | hypothetical protein | | hydrolase of |
| | | | | | 2.10e−61 | | the metallo- |
| | | | | | | | beta- |
| | | | | | | | lactamase |
| | | | | | | | superfamily |
| | | | | | | | 2e−99 |
| 910, | NEQ467 | SSU Ribosomal | Ribosomal protein | 7.00E−37 | Ribosomal_S13 Ribosomal | | Information |
| 911 | | protein | S13 [Methanopyrus | | protein S13/S18 4.30e−41 | | storage and |
| | | S13[Methanopyrus | kandleri AV19]. | | :FOLD1946 No CATH | | processing; Translation, |
| | | kandleri AV19] | | | Annotation 1.00e−4 | | ribosomal |
| | | | | | | | structure and |
| | | | | | | | biogenesis; Ribosomal |
| | | | | | | | protein S13 |
| | | | | | | | 9e−34 |
| 912, | NEQ468 | hypothetical protein | hypothetical protein | 8.00E−05 | 0 | | |
| 913 | | | [Plasmodium yoelii | | | | |
| | | | yoelii]. | | | | |
| 914, | NEQ469 | SSU ribosomal | SSU ribosomal | 8.00E−35 | Ribosomal_S8e Ribosomal | | Information |
| 915 | | protein S8E | protein S8E (rps8E) | | protein S8e 1.70e−43 :S8e | | storage and |
| | | (rps8E)[Archaeoglobus | [Archaeoglobus | | S8e ribosomal protein S8.e | | processing; Translation, |
| | | fulgidus] | fulgidus]. | | 8.30e−39 | | ribosomal |
| | | | | | | | structure and |
| | | | | | | | biogenesis; Ribosomal |
| | | | | | | | protein S8E |
| | | | | | | | 5e−36 |
| 916, | NEQ470 | hypothetical protein | Uncharacterized | 3.00E−06 | 0 | | Poorly |
| 917 | | | conserved protein | | | | characterized; |
| | | | [Thermoplasma | | | | Function |
| | | | volcanium]. | | | | unknown; Uncharacterized |
| | | | | | | | ArCR 4e−07 |
| 918, | NEQ471 | hypothetical protein | TraD-like protein | 5.00E−03 | 0 | | |
| 919 | | | [Haemophilus | | | | |
| | | | influenzae biotype | | | | |
| | | | aegyptius]. | | | | |
| 920, | NEQ472 | Calcineurin | conserved protein | 3.00E−12 | Metallophos Calcineurin-like | | Poorly |
| 921 | | superfamilyphosphoesterase | [Methanothermobacter | | phosphoesterase 6.30e−18 | | characterized; |
| | | [Methanopyrus | thermautotrophicus]. | | :TIGR00040 TIGR00040 | | General |
| | | | | | conserved hypothetical | | function |
| | | | | | protein TIGR00040 6.60e−05 | | prediction |
| | | | | | | | only; Predicted |
| | | | | | | | phosphoesterases, |
| | | | | | | | related |
| | | | | | | | to the lcc |
| | | | | | | | protein 2e−13 |
| 922, | NEQ473 | cell division protein | cell division protein | 9.00E−77 | tubulin Tubulin/FtsZ | 3.4.24.— | Cellular |
| 923 | | (ftsZ- | (ftsZ-1) | | family, GTPase | | processes; Cell |
| | | 1)[Archaeoglobus | [Archaeoglobus | | domain 5.80e−57 | | division and |
| | | fulgidus] | fulgidus]. | | :ftsZ ftsZ cell | | chromosome |
| | | | | | division protein FtsZ | | partitioning; Cell |
| | | | | | 1.70e−103 | | division |
| | | | | | :tubulin_C | | GTPase 6e−78 |
| | | | | | Tubulin/FtsZ family, | | |
| | | | | | C-terminal domain | | |
| | | | | | 1.20e−15 | | |
| 924, | NEQ475 | cell division control | cell division control | 0 | AAA ATPase family | 2.7.1.— | Cellular |
| 925 | | protein 48, | protein 48, aaa family; | | associated with | | processes; Posttranslational |
| | | aaafamily; (cdc48-2) | (cdc48-2) [Pyrococcus | | various cellular | | modification, |
| | | [P. furiosus] | furiosus DSM 3638]. | | activities (AAA) | | protein |
| | | | | | 1.70e−94 :AAA | | turnover, |
| | | | | | ATPase family | | chaperones; ATPases |
| | | | | | associated with | | of the |
| | | | | | various cellular | | AAA+ class 0 |
| | | | | | activities (AAA) | | |
| | | | | | 8.30e−92 :cdc48_2 | | |
| | | | | | Cell division protein | | |
| | | | | | 48 (CDC48), domain | | |
| | | | | | 2 2.40e−15 | | |
| | | | | | :cdc48_N Cell | | |
| | | | | | division protein 48 | | |
| | | | | | (CDC48), N-terminal | | |
| | | | | | domain 1.20e−32 | | |
| | | | | | :Sigma54_activat | | |
| | | | | | Sigma-54 interaction | | |
| | | | | | domain 3.20e−3 | | |
| | | | | | :FtsH_fam | | |
| | | | | | FtsH_fam ATP- | | |
| | | | | | dependent | | |
| | | | | | metalloprotease | | |
| | | | | | FtsH 1.70e−22 | | |
| | | | | | :26Sp45 26Sp45 | | |
| | | | | | 26S proteasome | | |
| | | | | | subunit P45 family | | |
| | | | | | 1.10e−79 :CDC48 | | |
| | | | | | CDC48 AAA family | | |
| | | | | | ATPase, CDC48 | | |
| | | | | | subfamily 0.0 | | |
| 926, | NEQ474 | hypothetical protein | hypothetical protein | 0.21 | 0 | | |
| 927 | | | [Clostridium | | | | |
| | | | thermocellum ATCC | | | | |
| | | | 27405]. | | | | |
| 928, | NEQ476 | hypothetical protein | M. jannaschii | 0.04 | 0 | | |
| 929 | | | predicted coding | | | | |
| | | | region MJ1254 | | | | |
| | | | [Methanococcus | | | | |
| | | | jannaschii]. | | | | |
| 930, | NEQ477 | hypothetical protein | cytochrome c oxidase | 0.49 | 0 | | |
| 931 | | | subunit III (aa3 type) | | | | |
| | | | [Oceanobacillus | | | | |
| | | | iheyensis]. | | | | |
| 932, | NEQ478 | SSU ribosomal | ribosomal protein S4 | 3.00E−47 | KOW KOW motif 4.50e−06 | | Information |
| 933 | | protein | [Methanothermobacter | | :Ribosomal_S4e Ribosomal | | storage and |
| | | S4E[Methanothermobacter | thermautotrophicus]. | | family S4e 2.70e−18 :S4 S4 | | processing; Translation, |
| | | | | | domain 2.10e−07 | | ribosomal |
| | | | | | | | structure and |
| | | | | | | | biogenesis; Ribosomal |
| | | | | | | | protein S4E |
| | | | | | | | 2e−48 |
| 934, | NEQ479 | phenylalanyl-tRNA | phenylalanyl-tRNA | 4.00E−78 | pheT_arch | 6.1.1. | Information |
| 935 | | synthetase beta- | synthetase beta-chain | | pheT_arch | 20 | storage and |
| | | chain[Pyrococcus | [Pyrococcus furiosus | | phenylalanyl-tRNA | | processing; Translation, |
| | | furiosus] | DSM 3638]. | | synthetase, beta | | ribosomal |
| | | | | | subunit 3.10e−89 | | structure and |
| | | | | | :pheT_bact | | biogenesis; Phenylalanyl- |
| | | | | | pheT_bact | | tRNA |
| | | | | | phenylalanyl-tRNA | | synthetase |
| | | | | | synthetase, beta | | beta subunit |
| | | | | | subunit 2.20e−09 | | 4e−78 |
| | | | | | :B3_4 B3/4 domain | | |
| | | | | | 8.10e−06 :B5 tRNA | | |
| | | | | | synthetase B5 | | |
| | | | | | domain 3.90e−4 | | |
| 936, | NEQ480 | SSU ribosomal | SSU ribosomal | 2.00E−45 | Ribosomal_S19 Ribosomal | | Information |
| 937 | | protein | protein S19P | | protein S19 4.10e−30 | | storage and |
| | | S19P[Pyrococcus | [Pyrococcus abyssi]. | | :rpsS_arch rpsS_arch | | processing; Translation, |
| | | abyssi] | | | ribosomal protein S19 1.70e−67 | | ribosomal |
| | | | | | :rpsS_bact rpsS_bact | | structure and |
| | | | | | ribosomal protein S19 5.50e−19 | | biogenesis; Ribosomal |
| | | | | | | | protein S19 |
| | | | | | | | 3e−46 |
| 938, | NEQ481 | SSU ribosomal | SSU ribosomal | 3.00E−31 | Ribosomal_S3_C Ribosomal | | Information |
| 939 | | protein S3P | protein S3P (rpsC) | | protein S3, C-terminal | | storage and |
| | | (rpsC)[Methanococcus | [Methanococcus | | domain 3.70e−4 :rpsC_E_A | | processing; Translation, |
| | | jannaschii] | jannaschii]. | | rpsC_E_A ribosomal protein | | ribosomal |
| | | | | | S3 1.20e−36 :rpsC_bact | | structure and |
| | | | | | rpsC_bact ribosomal protein | | biogenesis; Ribosomal |
| | | | | | S3 3.00e−05 | | protein S3 2e−32 |
| 940, | NEQ482 | transcription initiation | conserved protein | 2.00E−08 | TFIIE_alpha TFIIE alpha | | Information |
| 941 | | factor IIE, subunit | [Methanothermobacter | | subunit 4.40e−05 | | storage and |
| | | alpha (TFE) [Mc. | thermautotrophicus]. | | :TIGR00373 TIGR00373 | | processing; Transcription; |
| | | jannaschii] | | | conserved hypothetical | | Transcription |
| | | | | | protein TIGR00373 2.40e−05 | | initiation |
| | | | | | | | factor IIE, |
| | | | | | | | large subunit |
| | | | | | | | 2e−09 |
| 942, | NEQ483 | hypothetical protein | hypothetical protein | 0.01 | 0 | | |
| 943 | | | [Cytophaga | | | | |
| | | | hutchinsonii]. | | | | |
| 944, | NEQ485 | hypothetical protein | hypothetical protein | 3.00E−06 | 0 | | |
| 945 | | | [Plasmodium | | | | |
| | | | falciparum 3D7]. | | | | |
| 946, | NEQ484 | hypothetical protein | hypothetical protein | 0.01 | 0 | | |
| 947 | | | [Cytophaga | | | | |
| | | | hutchinsonii]. | | | | |
| 948, | NEQ486 | Na+/Ca+ exchanging | hypothetical | 2.00E−41 | Na_Ca_Ex Sodium/calcium | | Cellular |
| 949 | | protein | conserved protein | | exchanger protein 2.50e−28 | | processes; Inorganic |
| | | related[Pyrococcus | [Oceanobacillus | | :Na_Ca_Ex Sodium/calcium | | ion |
| | | abyssi] | iheyensis]. | | exchanger protein 4.50e−26 | | transport and |
| | | | | | :TIGR00367 TIGR00367 | | metabolism; Ca2+/ |
| | | | | | K+-dependent Na+/Ca+ | | Na+ |
| | | | | | exchanger related-protein | | antiporter 8e−42 |
| | | | | | 2.10e−20 | | |
| 950, | NEQ487 | SSU Ribosomal | Ribosomal protein | 2.00E−43 | Ribosomal_S15 Ribosomal | | Information |
| 951 | | protein | S15P/S13E | | protein S15 4.00e−14 | | storage and |
| | | S15P/S13E[Methanopyrus | [Methanopyrus | | | | processing; Translation, |
| | | kandleri AV19] | kandleri AV19]. | | | | ribosomal |
| | | | | | | | structure and |
| | | | | | | | biogenesis; Ribosomal |
| | | | | | | | protein |
| | | | | | | | S15P/S13E |
| | | | | | | | 3e−44 |
| 952, | NEQ489 | LSU ribosomal | LSU ribosomal protein | 2.00E−20 | L15 Ribosomal protein L15 | | Information |
| 953 | | protein L18E | L18E (rpl18E) | | 1.20e−06 | | storage and |
| | | (rpl18E)[Sulfolobus | [Sulfolobus | | | | processing; Translation, |
| | | solfataricus] | solfataricus]. | | | | ribosomal |
| | | | | | | | structure and |
| | | | | | | | biogenesis; Ribosomal |
| | | | | | | | protein L18E |
| | | | | | | | 1e−19 |
| 954, | NEQ490 | hypothetical protein | Ubie_methyltran, | 2.00E−07 | 0 | | |
| 955 | | | ubiE/COQ5 | | | | |
| | | | methyltransferase | | | | |
| | | | family [Bacillus | | | | |
| | | | anthracis A2012]. | | | | |
| 956, | NEQ491 | NADH | 324aa long | 2.00E−70 | pyr_redox Pyridine | 1.6.4.5 | Cellular |
| 957 | | oxidase/thioredoxin | hypothetical | | nucleotide- | | processes; Posttranslational |
| | | reductase[Sulfolobus | thioredoxin reductase | | disulphide | | modification, |
| | | solfataricus] | [Sulfolobus tokodaii]. | | oxidoreductase | | protein |
| | | | | | 2.80e−72 :gidA gidA | | turnover, |
| | | | | | glucose-inhibited | | chaperones; Thioredoxin |
| | | | | | division protein A | | reductase 3e−69 |
| | | | | | 8.10e−07 | | |
| | | | | | :TRX_reduct | | |
| | | | | | TRX_reduct | | |
| | | | | | thioredoxin | | |
| | | | | | reductase 9.00e−128 | | |
| | | | | | :gltA gltA glutamate | | |
| | | | | | synthase (NADPH), | | |
| | | | | | homotetrameric | | |
| | | | | | 1.10e−3 | | |
| | | | | | :GOGAT_sm_gam | | |
| | | | | | GOGAT_sm_gam | | |
| | | | | | glutamate | | |
| | | | | | synthases, | | |
| | | | | | NADH/NADPH, | | |
| | | | | | small subunit 6.10e−3 | | |
| 958, | NEQ492 | hypothetical protein | ADHESIN AIDA-I | 2.00E−03 | 0 | | |
| 959 | | | PRECURSOR. | | | | |
| 960, | NEQ493 | O-sialoglycoprotein | O-sialoglycoprotein | 1.00E−75 | Peptidase_M22 | 3.4.24. | Cellular |
| 961 | | endopeptidase | endopeptidase | | Glycoprotease | 57 | processes; Posttranslational |
| | | [Pyrococcus | [Pyrococcus | | family 5.90e−61 :gcp | | modification, |
| | | horikoshii]. | horikoshii]. | | gcp | | protein |
| | | | | | metalloendopeptidase, | | turnover, |
| | | | | | putative, | | chaperones; |
| | | | | | glycoprotease family | | Metal- |
| | | | | | 1.20e−81 | | dependent |
| | | | | | | | proteases |
| | | | | | | | with possible |
| | | | | | | | chaperone |
| | | | | | | | activity 1e−76 |
| 962, | NEQ494 | pyruvate formate- | 351aa long conserved | 1.00E−104 | Radical_SAM | 1.97. | Cellular |
| 963 | | lyase | hypothetical protein | | Radical SAM | 1.4 | processes; Posttranslational |
| | | activatingenzyme | [Sulfolobus tokodaii]. | | superfamily 5.00e−25 | | modification, |
| | | homolog [Pb | | | | | protein |
| | | aerophilum] | | | | | turnover, |
| | | | | | | | chaperones; Pyruvate- |
| | | | | | | | formate |
| | | | | | | | lyase- |
| | | | | | | | activating |
| | | | | | | | enzyme 8e−96 |
| 964, | NEQ495 | Predicted P-loop | hypothetical protein | 1.00E−92 | 1.10E−62 | | Poorly |
| 965 | | ATPase fused to | [Pyrococcus | | | | characterized; |
| | | anacetyltransferase | horikoshii]. | | | | General |
| | | [M. kandleri] SPLIT | | | | | function |
| | | see SEQ ID | | | | | prediction |
| | | NOS: 194, 195 | | | | | only; Predicted |
| | | | | | | | P-loop |
| | | | | | | | ATPase fused |
| | | | | | | | to an |
| | | | | | | | acetyltransferase |
| | | | | | | | 9e−94 |
| 966, | NEQ498 | Translation initiation | Translation initiation | 1.00E−166 | GTP_EFTU | 3.6.1. | Information |
| 967 | | factor 2, | factor 2, GTPase | | Elongation factor Tu | 48 | storage and |
| | | GTPase[Methanopyrus | [Methanopyrus | | GTP binding domain | | processing; Translation, |
| | | kandleri AV19] | kandleri AV19]. | | 1.70e−40 :mobB | | ribosomal |
| | | | | | mobB | | structure and |
| | | | | | molybdopterin- | | biogenesis; Translation |
| | | | | | guanine dinucleotide | | initiation |
| | | | | | biosynthesis protein | | factor 2 |
| | | | | | MobB 7.50e−3 | | (GTPase) 1e−150 |
| | | | | | :small_GTP | | |
| | | | | | small_GTP small | | |
| | | | | | GTP-binding protein | | |
| | | | | | domain 8.50e−15 | | |
| | | | | | :selB selB | | |
| | | | | | selenocysteine- | | |
| | | | | | specific translation | | |
| | | | | | elongation factor | | |
| | | | | | 1.20e−4 :EF-Tu EF- | | |
| | | | | | Tu translation | | |
| | | | | | elongation factor Tu | | |
| | | | | | 6.90e−3 :IF-2 IF-2 | | |
| | | | | | translation initiation | | |
| | | | | | factor IF-2 4.40e−08 | | |
| | | | | | :alF-2 alF-2 | | |
| | | | | | translation initiation | | |
| | | | | | factor alF-2 5.50e−213 | | |
| | | | | | :FOLD979 No | | |
| | | | | | CATH Annotation | | |
| | | | | | 2.50e−3 | | |
| 968, | NEQ497 | hypothetical protein | unnamed protein | 3.00E−04 | 0 | | |
| 969 | | | product, putative | | | | |
| | | | [Plasmodium yoelii | | | | |
| | | | yoelii]. | | | | |
| 970, | NEQ499 | hypothetical protein | hypothetical protein | 4.00E−03 | 0 | | |
| 971 | | [Pyrococcusabyssi] | [Pyrococcus abyssi]. | | | | |
| 972, | NEQ500 | hypothetical protein | predicted protein | 1.00E−05 | 0 | | |
| 973 | | | [Methanosarcina | | | | |
| | | | acetivorans str. C2A]. | | | | |
| 974, | NEQ502 | 2′-5′ RNA ligase | conserved | 1.00E−26 | 2_5_ligase 2′,5′ | 6.5.1.— | Information |
| 975 | | [Methanopyruskandleri | hypothetical protein | | RNA ligase family | | storage and |
| | | AV19] | [Methanococcus | | 6.70e−20 | | processing; Translation, |
| | | | jannaschii]. | | :2_5_ligase 2′,5′ | | ribosomal |
| | | | | | RNA ligase family | | structure and |
| | | | | | 3.40e−05 | | biogenesis; 2′- |
| | | | | | | | 5′ RNA ligase |
| | | | | | | | 1e−27 |
| 976, | NEQ501 | magnesium and | magnesium and | 1.00E−06 | 0 | | Cellular |
| 977 | | cobalt | cobalt transport | | | | processes; Inorganic |
| | | transportprotein | protein (corA) | | | | ion |
| | | (corA) [Haemophilus | [Haemophilus | | | | transport and |
| | | | influenzae Rd]. | | | | metabolism; Mg2+ |
| | | | | | | | and |
| | | | | | | | Co2+ |
| | | | | | | | transporters |
| | | | | | | | 9e−08 |
| 978, | NEQ504 | hypothetical protein | Unknown (protein for | 0.06 | 0 | | |
| 979 | | | MGC: 55710) [Danio | | | | |
| | | | rerio]. | | | | |
| 980, | NEQ503 | DNA-directed RNA | DNA-directed RNA | 0 | RNA_pol_Rpb1_2 | 2.7.7.6 | Information |
| 981 | | polymerase, subunit | polymerase, subunit A | | RNA polymerase | | storage and |
| | | A′ (rpoA1) [A. fulgidus] | (rpoA1) | | Rpb1, domain 2 | | processing; Transcription; |
| | | | [Archaeoglobus | | 1.10e−93 : 2.00e−39 | | DNA- |
| | | | fulgidus]. | | : 3.60e−91 : 8.40e−30 | | directed |
| | | | | | | | RNA |
| | | | | | | | polymerase |
| | | | | | | | beta′ |
| | | | | | | | subunit/160 kD |
| | | | | | | | subunit |
| | | | | | | | (split gene in |
| | | | | | | | archaea and |
| | | | | | | | Syn) 0 |
| 982, | NEQ506 | Predicted RNA- | hypothetical protein | 1.00E−41 | 0 | | Information |
| 983 | | binding | [Pyrococcus furiosus | | | | storage and |
| | | proteinhomologous | DSM 3638]. | | | | processing; Transcription; |
| | | to eukaryotic snRNP | | | | | Predicted |
| | | | | | | | RNA- |
| | | | | | | | binding |
| | | | | | | | protein |
| | | | | | | | homologous |
| | | | | | | | to eukaryotic |
| | | | | | | | snRNP 8e−41 |
| 984, | NEQ505 | Phenylalanyl-tRNA | Phenylalanyl-tRNA | 1.00E−74 | tRNA-synt_2d tRNA | 6.1.1. | Information |
| 985 | | synthetase | synthetase alpha | | synthetases class II | 20 | storage and |
| | | alphasubunit (pheS) | subunit (pheS) | | core domain (F) | | processing; Translation, |
| | | [S. solfataricus] | [Sulfolobus | | 4.30e−61 :pheS | | ribosomal |
| | | | solfataricus]. | | pheS phenylalanyl- | | structure and |
| | | | | | tRNA synthetase, | | biogenesis; Phenylalanyl- |
| | | | | | alpha subunit 9.20e−66 | | tRNA |
| | | | | | | | synthetase |
| | | | | | | | alpha subunit |
| | | | | | | | 7e−64 |
| 986, | NEQ507 | DNA-directed RNA | DNA-directed RNA | 2.00E−16 | RNA_pol_Rpb5_C | 2.7.7.6 | Information |
| 987 | | polymerase, subunit | polymerase, subunit H | | RNA polymerase | | storage and |
| | | H (rpoH) [Mc | (rpoH) | | Rpb5, C-terminal | | processing; Transcription; |
| | | jannaschii] | [Methanococcus | | domain 4.00e−26 | | DNA- |
| | | | jannaschii]. | | | | directed |
| | | | | | | | RNA |
| | | | | | | | polymerase, |
| | | | | | | | subunit H, |
| | | | | | | | RpoH/RPB5 |
| | | | | | | | 1e−17 |
| 988, | NEQ508 | SSU ribosomal | 30S ribosomal protein | 1.00E−57 | Ribosomal_S2 Ribosomal | | Information |
| 989 | | protein | S2 [Pyrococcus | | protein S2 3.20e−30 | | storage and |
| | | S2[Pyrococcus | horikoshii]. | | :rpsB_bact rpsB_bact | | processing; Translation, |
| | | horikoshii] | | | ribosomal protein S2 4.40e−06 | | ribosomal |
| | | | | | :Sa_S2_E_A Sa_S2_E_A | | structure and |
| | | | | | ribosomal protein S2 3.70e−98 | | biogenesis; Ribosomal |
| | | | | | | | protein S2 8e−59 |
| 990, | NEQ509 | DNA ligase; ATP | DNA ligase | 1.00E−139 | DNA_ligase ATP | 6.5.1.1 | Information |
| 991 | | dependent[Pyrococcus | [Pyrococcus abyssi]. | | dependent DNA | | storage and |
| | | abyssi] | | | ligase domain | | processing; DNA |
| | | | | | 7.30e−70 :dnl1 dnl1 | | replication, |
| | | | | | DNA ligase I, ATP- | | recombination |
| | | | | | dependent (dnl1) | | and |
| | | | | | 5.60e−163 : 4.10e−08 | | repair; ATP- |
| | | | | | : 1.10e−67 | | dependent |
| | | | | | | | DNA ligase |
| | | | | | | | 1e−140 |
| 992, | NEQ510 | hypothetical protein | putative histidine | 0.02 | 0 | | |
| 993 | | | kinase, possibly | | | | |
| | | | involved in | | | | |
| | | | competence | | | | |
| | | | [Streptococcus | | | | |
| | | | pyogenes]. | | | | |
| 994, | NEQ511 | desuccinylase | desuccinylase | 8.00E−28 | Peptidase_M20 | 3.5.1. | Metabolism; Amino |
| 995 | | [Pyrococcus | [Pyrococcus | | Peptidase family | 18 | acid |
| | | horikoshii] | horikoshii]. | | M20/M25/M40 | | transport and |
| | | | | | 1.10e−63 | | metabolism; Acetylornithine |
| | | | | | :dapE_proteo | | deacetylase/Succinyl- |
| | | | | | dapE_proteo | | diaminopimelate |
| | | | | | succinyl- | | desuccinylase |
| | | | | | diaminopimelate | | and related |
| | | | | | desuccinylase | | deacylases |
| | | | | | 1.30e−11 | | 6e−29 |
| 996, | NEQ512 | Predicted P-loop | conserved | 3.00E−37 | 0 | | Poorly |
| 997 | | ATPase[Methanopyrus | hypothetical protein | | | | characterized; |
| | | kandleri AV19] | [Methanosarcina | | | | General |
| | | | acetivorans str. C2A]. | | | | function |
| | | | | | | | prediction |
| | | | | | | | only; Predicted |
| | | | | | | | ATPase 3e−09 |
| 998, | NEQ513 | hypothetical Glu- | Glu-tRNA | 1.00E−05 | gatC gatC glutamyl- | | Information |
| 999 | | tRNA | amidotransferase, | | tRNA(Gln) | | storage and |
| | | amidotransferase, | subunit C (gatC) | | amidotransferase, C subunit | | processing; Translation, |
| | | subunit C | [Archaeoglobus | | 2.20e−4 | | ribosomal |
| | | | fulgidus]. | | | | structure and |
| | | | | | | | biogenesis; Asp- |
| | | | | | | | tRNAAsn/Glu- |
| | | | | | | | tRNAGln |
| | | | | | | | amidotransferase |
| | | | | | | | C subunit |
| | | | | | | | 7e−07 |
| 1000, | NEQ514 | hypothetical protein | exodeoxyribonuclease | 9.00E−03 | 0 | | |
| 1001 | | | V, beta chain (recB) | | | | |
| | | | [Borrelia burgdorferi]. | | | | |
| 1002, | NEQ515 | Uncharacterized | hypothetical protein | 4.00E−38 | | | |
| 1003 | | protein conserved | [Pyrococcus | | | | |
| | | inarchaea | horikoshii]. | | | | |
| | | [Methanopyrus | | | | | |
| | | kandleri] | | | | | |
| 1004, | NEQ516 | hypothetical protein | hypothetical protein | 1.00E−11 | 0 | | Cellular |
| 1005 | | | [Pyrococcus abyssi]. | | | | processes; Posttranslational |
| | | | | | | | modification, |
| | | | | | | | protein |
| | | | | | | | turnover, |
| | | | | | | | chaperones; Prefoldin, |
| | | | | | | | chaperonin |
| | | | | | | | cofactor 7e−13 |
| 1006, | NEQ518 | hypothetical protein | U30 [Human | 0.25 | 0 | | |
| 1007 | | | herpesvirus 7]. | | | | |
| 1008, | NEQ517 | translation initiation | 223aa long | 4.00E−16 | eIF6 eIF-6 family 2.30e−09 | | Information |
| 1009 | | factor 6[Sulfolobus | hypothetical | | :eIF-6 eIF-6 translation | | storage and |
| | | tokodaii] | translation initiation | | initiation factor eIF-6, | | processing; Translation, |
| | | | factor 6 [Sulfolobus | | putative 2.70e−12 | | ribosomal |
| | | | tokodaii]. | | | | structure and |
| | | | | | | | biogenesis; Eukaryotic |
| | | | | | | | translation |
| | | | | | | | initiation |
| | | | | | | | factor 6 |
| | | | | | | | (EIF6) 4e−12 |
| 1010, | NEQ519 | histidine triad (HIT) | HIT family protein (hit) | 5.00E−29 | HIT HIT family | 3.6.1. | Metabolism; Nucleotide |
| 1011 | | protein MJ0866(cell | [Methanococcus | | 2.10e−44 | 17 | transport and |
| | | cycle regulation)-M. jannaschii | jannaschii]. | | | | metabolism; Diadenosine |
| | | | | | | | tetraphosphate |
| | | | | | | | (Ap4A) |
| | | | | | | | hydrolase and |
| | | | | | | | other HIT |
| | | | | | | | family |
| | | | | | | | hydrolases |
| | | | | | | | 5e−30 |
| 1012, | NEQ520 | hypothetical protein | Sulfur transfer protein | 1.4 | 0 | | |
| 1013 | | | involved in thiamine | | | | |
| | | | biosynthesis [Aquifex | | | | |
| | | | aeolicus]. | | | | |
| 1014, | NEQ521 | Proteasome alpha | Protease subunit of | 8.00E−60 | proteasome | 3.4.25.1 | Cellular |
| 1015 | | subunit(Multicatalytic | the proteasome | | Proteasome A-type | | processes; Posttranslational |
| | | endopeptidase | [Methanopyrus | | and B-type 9.90e−40 | | modification, |
| | | | kandleri AV19]. | | | | protein |
| | | | | | | | turnover, |
| | | | | | | | chaperones; Proteasome |
| | | | | | | | protease |
| | | | | | | | subunit 6e−57 |
| 1016, | NEQ523 | n-type ATP | hypothetical protein | 7.00E−59 | UPF0021 Uncharacterized | | Cellular |
| 1017 | | pyrophosphatasesupperfamily | [Pyrococcus abyssi]. | | protein family UPF0021 | | processes; Cell |
| | | [Pyrococcus | | | 2.00e−17 :TIGR00269 | | division and |
| | | furiosus]; | | | TIGR00269 conserved | | chromosome |
| | | | | | hypothetical protein | | partitioning; Predicted |
| | | | | | TIGR00269 4.90e−13 | | ATPase of |
| | | | | | | | the PP-loop |
| | | | | | | | superfamily |
| | | | | | | | implicated in |
| | | | | | | | cell cycle |
| | | | | | | | control 5e−60 |
| 1018, | NEQ522 | Predicted N6- | conserved | 2.00E−37 | THUMP THUMP | 2.1.1.— | Information |
| 1019 | | adenine-specific | hypothetical protein | | domain 1.50e−11 | | storage and |
| | | RNAmethylase | [Methanococcus | | :UPF00200 Putative | | processing; DNA |
| | | containing THUMP | jannaschii]. | | RNA methylase | | replication, |
| | | | | | family UPF0020 | | recombination |
| | | | | | 1.00e−37 | | and |
| | | | | | :TIGR01177 | | repair; Predicted |
| | | | | | TIGR01177 | | N6- |
| | | | | | conserved | | adenine- |
| | | | | | hypothetical protein | | specific DNA |
| | | | | | TIGR01177 4.30e−19 | | methylases |
| | | | | | | | 1e−38 |
| 1020, | NEQ525 | 3′- | n-type ATP | 2.00E−33 | PAPS_reduct | 1.8.99.4 | Metabolism; Amino |
| 1021 | | phosphoadenosine | pyrophosphatase | | Phosphoadenosine | | acid |
| | | 5′- | superfamily | | phosphosulfate | | transport and |
| | | phosphosulfatesulfotransferase | [Pyrococcus furiosus | | reductase family | | metabolism; 3′- |
| | | | DSM 3638]. | | 9.30e−18 | | phosphoadenosine |
| | | | | | | | 5′- |
| | | | | | | | phosphosulfate |
| | | | | | | | sulfotransferase |
| | | | | | | | (PAPS |
| | | | | | | | reductase)/FAD |
| | | | | | | | synthetase |
| | | | | | | | and related |
| | | | | | | | enzymes 1e−30 |
| 1022, | NEQ524 | Predicted exosome | Predicted exosome | 6.00E−48 | UPF0023 Uncharacterized | | Information |
| 1023 | | subunit[Methanopyrus | subunit | | protein family UPF0023 | | storage and |
| | | kandleri AV19] | [Methanopyrus | | 8.90e−24 :TIGR00291 | | processing; Translation, |
| | | | kandleri AV19]. | | TIGR00291 conserved | | ribosomal |
| | | | | | hypothetical protein | | structure and |
| | | | | | TIGR00291 2.20e−53 | | biogenesis; Predicted |
| | | | | | | | exosome |
| | | | | | | | subunit 2e−47 |
| 1024, | NEQ526 | hypothetical | hypothetical protein | 6.00E−12 | 0 | | Information |
| 1025 | | protein(similar to | [Thermoplasma | | | | storage and |
| | | Queuine tRNA- | volcanium]. | | | | processing; Translation, |
| | | ribosyltransferase) | | | | | ribosomal |
| | | | | | | | structure and |
| | | | | | | | biogenesis; Queuine |
| | | | | | | | tRNA- |
| | | | | | | | ribosyltransferases, |
| | | | | | | | contain |
| | | | | | | | PUA domain |
| | | | | | | | 4e−13 |
| 1026, | NEQ527 | Predicted P-loop | 495aa long conserved | 1.00E−23 | DUF87 Domain of unknown | | Poorly |
| 1027 | | ATPase[Methanopyrus | hypothetical protein | | function DUF87 2.30e−07 | | characterized; |
| | | kandleri AV19] | [Sulfolobus tokodaii]. | | | | General |
| | | | | | | | function |
| | | | | | | | prediction |
| | | | | | | | only; Predicted |
| | | | | | | | ATPase 1e−17 |
| 1028, | NEQ528 | DNA-directed DNA | DNA POLYMERASE. | 4.00E−50 | DNA_pol_B DNA | 2.7.7.7 | Information |
| 1029 | | polymerase I(family | | | polymerase family B | | storage and |
| | | B) SPLIT see SEQ | | | 4.80e−4 | | processing; DNA |
| | | ID NO: 136, 137 | | | | | replication, |
| | | | | | | | recombination |
| | | | | | | | and |
| | | | | | | | repair; DNA |
| | | | | | | | polymerase |
| | | | | | | | elongation |
| | | | | | | | subunit |
| | | | | | | | (family B) 1e−46 |
| 1030, | NEQ529 | hypothetical protein | | | DUF457 Predicted membrane-bound | | |
| | | | | | metal-dependent | | |
| 1031 | | | | | hydrolase (DUF457) 6.00e−06 | | |
| 1032, | NEQ531 | putative integral | hypothetical protein | 2.00E−27 | MS_channel | | Cellular |
| 1033 | | membrane | [Nostoc sp. PCC | | Mechanosensitive ion | | processes; Cell |
| | | protein. [Streptomyces | 7120]. | | channel 1.40e−35 | | envelope |
| | | coelicolor A3(2)] | | | | | biogenesis, |
| | | | | | | | outer |
| | | | | | | | membrane; Small- |
| | | | | | | | conductance |
| | | | | | | | mechanosensitive |
| | | | | | | | channel |
| | | | | | | | 1e−22 |
| 1034, | NEQ530 | LSU ribosomal | LSU ribosomal protein | 9.00E−32 | Ribosomal_L32e Ribosomal | | Information |
| 1035 | | protein | L32E [Pyrococcus | | protein L32 6.60e−40 | | storage and |
| | | L32E[Pyrococcus | abyssi]. | | | | processing; Translation, |
| | | abyssi] | | | | | ribosomal |
| | | | | | | | structure and |
| | | | | | | | biogenesis; Ribosomal |
| | | | | | | | protein L32E |
| | | | | | | | 5e−33 |
| 1036, | NEQ532 | hypothetical protein | similar to cell wall | 4.00E−04 | 0 | | |
| 1037 | | | binding proteins | | | | |
| | | | [Listeria | | | | |
| | | | monocytogenes EGD- | | | | |
| | | | e]. | | | | |
| 1038, | NEQ533 | hypothetical protein | similar to Plasmodium | 1.4 | 0 | | |
| 1039 | | | falciparum. | | | | |
| | | | Hypothetical protein | | | | |
| | | | [Dictyostelium | | | | |
| | | | discoideum]. | | | | |
| 1040, | NEQ534 | Predicted | hypothetical protein | 6.00E−33 | 0 | | Information |
| 1041 | | transcriptional | [Pyrococcus | | | | storage and |
| | | regulator[Methanopyrus | horikoshii]. | | | | processing; Transcription; |
| | | kandleri AV19] | | | | | Predicted |
| | | | | | | | transcriptional |
| | | | | | | | regulators 8e−34 |
| 1042, | NEQ535 | aspartyl-tRNA | aspartyl-tRNA | 2.00E−92 | tRNA-synt_2 tRNA | 6.1.1. | Information |
| 1043 | | synthetase (class | synthetase | | synthetases class II | 12 | storage and |
| | | 2)[Aeropyrum pernix] | [Aeropyrum pernix]. | | (D, K and N) 3.90e−69 | | processing; Translation, |
| | | | | | :tRNA-synt_2d | | ribosomal |
| | | | | | tRNA synthetases | | structure and |
| | | | | | class II core domain | | biogenesis; Aspartyl/ |
| | | | | | (F) 3.70e−3 | | asparaginyl- |
| | | | | | :tRNA_anti OB-fold | | tRNA |
| | | | | | nucleic acid binding | | synthetases |
| | | | | | domain 6.00e−16 | | 2e−93 |
| | | | | | :asnS asnS | | |
| | | | | | asparaginyl-tRNA | | |
| | | | | | synthetase 8.00e−39 | | |
| | | | | | :aspS_arch | | |
| | | | | | aspS_arch aspartyl- | | |
| | | | | | tRNA synthetase | | |
| | | | | | 3.50e−109 | | |
| | | | | | :aspS_bact | | |
| | | | | | aspS_bact aspartyl- | | |
| | | | | | tRNA synthetase | | |
| | | | | | 3.40e−11 :genX | | |
| | | | | | genX lysyl-tRNA | | |
| | | | | | synthetase-related | | |
| | | | | | protein 1.60e−05 | | |
| | | | | | :lysS_bact | | |
| | | | | | lysS_bact lysyl- | | |
| | | | | | tRNA synthetase | | |
| | | | | | 2.00e−05 | | |
| 1044, | NEQ536 | putative nucleolar | putative nucleolar | 2.00E−78 | Nol1_Nop2_Sun | 2.1.1.— | Information |
| 1045 | | protein I (nol1-nop2- | protein I (nol1-nop2- | | NOL1/NOP2/sun | | storage and |
| | | sun family) | sun family) | | family 2.90e−49 | | processing; Translation, |
| | | [Pyrococcus | [Pyrococcus furiosus | | :nop2p nop2p | | ribosomal |
| | | | DSM 3638]. | | NOL1/NOP2/sun | | structure and |
| | | | | | family putative RNA | | biogenesis; tRNA |
| | | | | | methylase 7.50e−101 | | and rRNA |
| | | | | | :rsmB rsmB | | cytosine-C5- |
| | | | | | Sun protein 7.70e−09 | | methylases |
| | | | | | | | 3e−77 |
| 1046, | NEQ538 | acetylpolyamine | acetylpolyamine | 4.00E−40 | Hist_deacetyl | 3.5.1. | Cellular |
| 1047 | | aminohydrolase, putative | aminohydrolase, | | Histone deacetylase | 48 | processes; Signal |
| | | [Pyrobaculum | putative [Pyrobaculum | | family 2.40e−21 | | transduction |
| | | aerophilum] | aerophilum]. | | | | mechanisms; |
| | | | | | | | Deacetylases, |
| | | | | | | | including |
| | | | | | | | yeast histone |
| | | | | | | | deacetylase |
| | | | | | | | and acetoin |
| | | | | | | | utilization |
| | | | | | | | protein 2e−37 |
| 1048, | NEQ537 | proliferating-cell | proliferating-cell | 1.00E−37 | PCNA Proliferating cell | | Information |
| 1049 | | nuclear | nuclear antigen | | nuclear antigen, N-terminal | | storage and |
| | | antigen[Pyrococcus | [Pyrococcus abyssi]. | | domain 5.00e−4 :PCNA_C | | processing; DNA |
| | | abyssi] | | | Proliferating cell nuclear | | replication, |
| | | | | | antigen, C-terminal domain | | recombination |
| | | | | | 2.00e−05 :pcna pcna | | and |
| | | | | | proliferating cell nuclear | | repair; DNA |
| | | | | | antigen (pcna) 3.60e−14 | | polymerase |
| | | | | | | | sliding clamp |
| | | | | | | | subunit |
| | | | | | | | (PCNA |
| | | | | | | | homolog) 8e−39 |
| 1050, | NEQ539 | hypothetical protein | hypothetical protein | 3.00E−08 | 0 | | Cellular |
| 1051 | | [Pyrococcusfuriosus | [Pseudomonas | | | | processes; Cell |
| | | DSM 3638] | syringae pv. syringae | | | | envelope |
| | | | B728a]. | | | | biogenesis, |
| | | | | | | | outer |
| | | | | | | | membrane; Small- |
| | | | | | | | conductance |
| | | | | | | | mechanosensitive |
| | | | | | | | channel |
| | | | | | | | 3e−07 |
| 1052, | NEQ540 | transcription | transcription | 4.00E−27 | KOW KOW motif 2.10e−07 | | Information |
| 1053 | | antitermination | antitermination proteinnusG | | :L26e_arch L26e_arch | | storage and |
| | | proteinnusG | [Pyrococcus | | ribosomal protein L24 5.70e−25 | | processing; Transcription; |
| | | [Pyrococcus | furiosus DSM 3638]. | | | | Transcription |
| | | furiosus] | | | | | antiterminator |
| | | | | | | | 2e−27 |
| 1054, | NEQ541 | hypothetical | 135aa long | 800E−07 | 0 | | |
| 1055 | | transcriptional | hypothetical | | | | |
| | | regulator[Sulfolobus | transcriptional | | | | |
| | | tokodaii] | regulator [Sulfolobus | | | | |
| | | | tokodaii]. | | | | |
| 1056, | NEQ543 | elongation factor 2 | elongation factor 2 | 0 | EFG_C Elongation | 3.6.1. | Information |
| 1057 | | (EF-2); (EF- | (EF-2); (EF-2) | | factor G C-terminus | 48 | storage and |
| | | 2)[Pyrococcus | [Pyrococcus furiosus | | 1.00e−32 | | processing; Translation, |
| | | furiosus] | DSM 3638]. | | :GTP_EFTU | | ribosomal |
| | | | | | Elongation factor Tu | | structure and |
| | | | | | GTP binding domain | | biogenesis; Translation |
| | | | | | 7.10e−76 | | elongation |
| | | | | | :GTP_EFTU_D2 | | and release |
| | | | | | Elongation factor Tu | | factors |
| | | | | | domain 2 6.40e−14 | | (GTPases) 0 |
| | | | | | :small_GTP | | |
| | | | | | small_GTP small | | |
| | | | | | GTP-binding protein | | |
| | | | | | domain 5.20e−12 | | |
| | | | | | :selB selB | | |
| | | | | | selenocysteine- | | |
| | | | | | specific translation | | |
| | | | | | elongation factor | | |
| | | | | | 6.70e−3 :EF-G EF-G | | |
| | | | | | translation | | |
| | | | | | elongation factor G | | |
| | | | | | 3.90e−52 :EF-Tu EF- | | |
| | | | | | Tu translation | | |
| | | | | | elongation factor Tu | | |
| | | | | | 2.30e−4 :aEF-2 aEF- | | |
| | | | | | 2 translation | | |
| | | | | | elongation factor | | |
| | | | | | aEF-2 0.0 :prfC prfC | | |
| | | | | | peptide chain | | |
| | | | | | release factor 3 | | |
| | | | | | 4.30e−05 :EFG_IV | | |
| | | | | | Elongation factor G, | | |
| | | | | | domain IV 8.70e−24 | | |
| 1058, | NEQ542 | DNA topoisomerase | type II DNA | 1.00E−110 | TP6A_N Type IIB | 5.99. | Information |
| 1059 | | VI, subunit A(top6A) | topoisomerase | | DNA topoisomerase | 1.3 | storage and |
| | | [Pyrococcus abyssi] | subunit a [Pyrococcus | | 9.00e−30 | | processing; DNA |
| | | | furiosus DSM 3638]. | | | | replication, |
| | | | | | | | recombination |
| | | | | | | | and |
| | | | | | | | repair; DNA |
| | | | | | | | topoisomerase |
| | | | | | | | VI, subunit |
| | | | | | | | A 1e−111 |
| 1060, | NEQ545 | hypothetical protein | | | 0 | | |
| 1061 | | | | | | | |
| 1062, | NEQ544 | Predicted | Predicted membrane- | 8.00E−29 | Peptidase_M50 Peptidase | | Cellular |
| 1063 | | membrane- | associated Zn- | | family M50 9.90e−25 | | processes; Cell |
| | | associated Zn- | dependent protease | | | | envelope |
| | | dependentprotease | [Methanopyrus | | | | biogenesis, |
| | | [M. kandleri] | kandleri AV19]. | | | | outer |
| | | | | | | | membrane; Predicted |
| | | | | | | | membrane- |
| | | | | | | | associated |
| | | | | | | | Zn-dependent |
| | | | | | | | proteases 1 |
| | | | | | | | 3e−24 |
| 1064, | NEQ546 | LSU ribosomal | LSU ribosomal protein | 1.00E−35 | Ribosomal_L1 Ribosomal | | Information |
| 1065 | | protein L1P | L1P (rpl1P) | | protein L1p/L10e family | | storage and |
| | | (rpl1P)[Pyrococcus | [Pyrococcus abyssi]. | | 8.10e−39 :rplA_bact | | processing; Translation, |
| | | abyssi] | | | rplA_bact ribosomal protein | | ribosomal |
| | | | | | L1 1.40e−07 | | structure and |
| | | | | | | | biogenesis; Ribosomal |
| | | | | | | | protein L1 7e−37 |
| 1066, | NEQ548 | SSU ribosomal | SSU ribosomal | 1.00E−10 | Ribosomal_S24e Ribosomal | | Information |
| 1067 | | protein | protein S24E | | protein S24e 3.60e−11 | | storage and |
| | | S24E[Methanococcus | [Methanococcus | | | | processing; Translation, |
| | | jannaschii] | jannaschii]. | | | | ribosomal |
| | | | | | | | structure and |
| | | | | | | | biogenesis; Ribosomal |
| | | | | | | | protein S24E |
| | | | | | | | 7e−12 |
| 1068, | NEQ547 | alanyl-tRNA | alanyl-tRNA | 3.00E−64 | tRNA-synt_2c tRNA | 6.1.1.7 | Information |
| 1069 | | synthetase [Giardia | synthetase [Giardia | | synthetases class II | | storage and |
| | | | intestinalis]. | | (A) 6.50e−3 | | processing; Translation, |
| | | | | | | | ribosomal |
| | | | | | | | structure and |
| | | | | | | | biogenesis; Alanyl- |
| | | | | | | | tRNA |
| | | | | | | | synthetase |
| | | | | | | | 6e−61 |
| 1070, | NEQ549 | hypothetical protein | DNA helicase II | 4.00E−09 | 0 | | Information |
| 1071 | | | [Fusobacterium | | | | storage and |
| | | | nucleatum subsp. | | | | processing; DNA |
| | | | nucleatum ATCC | | | | replication, |
| | | | 25586]. | | | | recombination |
| | | | | | | | and |
| | | | | | | | repair; RecB |
| | | | | | | | family |
| | | | | | | | exonuclease |
| | | | | | | | 8e−07 |
| 1072, | NEQ550 | hypothetical protein | hypothetical protein | 0.01 | 0 | | |
| 1073 | | | [Plasmodium | | | | |
| | | | falciparum 3D7]. | | | | |

The invention for the first time has isolated and cultivated “Nanoarchaeum equitans,” a new nanosized hyperthermophilic archaeon derived from a submarine hot vent. This archaeon cannot be attached to any known Archaeal group, including the phyla Crenarchaeota, Korarchaeota or Euryarchaeota and therefore must represent an unknown phylum which we have named ‘Nanoarchaeota’ and species, which we name ‘Nanoarchaeum equitans’.

Cells of N. equitans are spherical, and only about 400 nm in diameter. They grow attached to the surface of a specific archaeal host, a new member of the genus Ignicoccus. The natural distribution of the ‘Nanoarchaeota’ is so far unknown. Owing to their unusual single-stranded (ss)rRNA sequence, members remained undetectable by commonly used ecological studies based on the polymerase chain reaction. ‘N. equitans’ harbors the smallest archaeal genome (SEQ ID NO:1); it is only 0.5 megabases in size. The organism of the invention will provide insight into the evolution of thermophily, of tiny genomes and of interspecies communication.

### DEFINITIONS

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The terms “array” or “microarray” or “biochip” or “chip” as used herein is a plurality of target elements, each target element comprising a defined amount of one or more polypeptides (including antibodies) or nucleic acids immobilized onto a defined area of a substrate surface, as discussed in further detail, below.

As used herein, the terms “computer,” “computer program” and “processor” are used in their broadest general contexts and incorporate all such devices, as described in detail, below.

A “coding sequence of” or a “sequence encodes” a particular polypeptide or protein, is a nucleic acid sequence which is transcribed and translated into a polypeptide or protein when placed under the control of appropriate regulatory sequences.

The term “expression cassette” as used herein refers to a nucleotide sequence which is capable of affecting expression of a structural gene (i.e., a protein coding sequence, such as an enzyme of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

“Plasmids” are designated by a lower case “p” preceded and/or followed by capital letters and/or numbers. The starting plasmids herein are either commercially available, publicly available on an unrestricted basis, or can be constructed from available plasmids in accord with published procedures. In addition, equivalent plasmids to those described herein are known in the art and will be apparent to the ordinarily skilled artisan.

The term “gene” means the segment of DNA involved in producing a polypeptide chain, including, inter alia, regions preceding and following the coding region, such as leader and trailer, promoters and enhancers, as well as, where applicable, intervening sequences (introns) between individual coding segments (exons).

The phrases “nucleic acid” or “nucleic acid sequence” as used herein refer to an oligonucleotide, nucleotide, polynucleotide, or to a fragment of any of these, to DNA or RNA (e.g., mRNA, rRNA, tRNA, iRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., double stranded iRNAs, e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

“Amino acid” or “amino acid sequence” as used herein refer to an oligopeptide, peptide, polypeptide, or protein sequence, or to a fragment, portion, or subunit of any of these, and to naturally occurring or synthetic molecules.

The terms “polypeptide” and “protein” as used herein, refer to amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

As used herein, the term “recombinant” means that the nucleic acid is adjacent to a “backbone” nucleic acid to which it is not adjacent in its natural environment. In one aspect, nucleic acids represent 5% or more of the number of nucleic acid inserts in a population of nucleic acid “backbone molecules.” “Backbone molecules” according to the invention include nucleic acids such as expression vectors, self-replicating nucleic acids, viruses, integrating nucleic acids, and other vectors or nucleic acids used to maintain or manipulate a nucleic acid insert of interest. In one aspect, the enriched nucleic acids represent 15%, 20%, 30%, 40%, 50%, 60%, 70%, 80%, 90% or more of the number of nucleic acid inserts in the population of recombinant backbone molecules. “Recombinant” polypeptides or proteins refer to polypeptides or proteins produced by recombinant DNA techniques; e.g., produced from cells transformed by an exogenous DNA construct encoding the desired polypeptide or protein. “Synthetic” polypeptides or protein are those prepared by chemical synthesis, as described in further detail, below.

A promoter sequence is “operably linked to” a coding sequence when RNA polymerase which initiates transcription at the promoter will transcribe the coding sequence into mRNA, as discussed further, below.

“Oligonucleotide” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The phrase “substantially identical” in the context of two nucleic acids or polypeptides, refers to two or more sequences that have at least 50%, 60%, 70%, 75%, 80%, 85%, 90%, 95%, 98% or 99% nucleotide or amino acid residue (sequence) identity, when compared and aligned for maximum correspondence, as measured using one any known sequence comparison algorithm, as discussed in detail below, or by visual inspection. In alternative aspects, the invention provides nucleic acid and polypeptide sequences having substantial identity to an exemplary sequence of the invention over a region of at least about 100 residues, 150 residues, 200 residues, 300 residues, 400 residues, or a region ranging from between about 50 residues to the full length of the nucleic acid or polypeptide. Nucleic acid sequences of the invention can be substantially identical over the entire length of a polypeptide coding region.

Additionally a “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a polypeptide of the invention, resulting in modification of the structure of the polypeptide, without significantly altering its biological activity. For example, amino- or carboxyl-terminal amino acids that are not required for the polypeptide's biological activity can be removed. Modified polypeptide sequences of the invention can be assayed for a biological activity (e.g., enzymatic, binding, structural, and the like) by any number of methods, including contacting the modified polypeptide sequence with an enzyme substrate and determining whether the modified polypeptide decreases the amount of specific substrate in the assay or increases the bioproducts of the enzymatic reaction of a functional enzyme with the substrate, as discussed further, below.

“Hybridization” refers to the process by which a nucleic acid strand joins with a complementary strand through base pairing. Hybridization reactions can be sensitive and selective so that a particular sequence of interest can be identified even in samples in which it is present at low concentrations. Suitably stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

The term “variant” refers to polynucleotides or polypeptides of the invention modified at one or more base pairs, codons, introns, exons, or amino acid residues (respectively) yet still retain the biological activity of a polypeptide (e.g., an enzyme) of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM and any combination thereof. Techniques for producing variant a polypeptide (e.g., an enzyme) having activity at a pH or temperature, for example, that is different from a wild-type a polypeptide (e.g., an enzyme), are included herein.

The term “saturation mutagenesis” or “GSSM” includes a method that uses degenerate oligonucleotide primers to introduce point mutations into a polynucleotide, as described in detail, below.

The term “optimized directed evolution system” or “optimized directed evolution” includes a method for reassembling fragments of related nucleic acid sequences, e.g., related genes, and explained in detail, below.

The term “synthetic ligation reassembly” or “SLR” includes a method of ligating oligonucleotide fragments in a non-stochastic fashion, and explained in detail, below.

### Generating and Manipulating Nucleic Acids

The invention provides nucleic acids (e.g., the exemplary SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073), including expression cassettes such as expression vectors, encoding polypeptides (e.g., enzymes) of the invention. The invention also includes methods for discovering new polypeptide (e.g., enzyme) sequences using the nucleic acids of the invention. Also provided are methods for modifying the nucleic acids of the invention by, e.g., synthetic ligation reassembly, optimized directed evolution system and/or saturation mutagenesis.

The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lac, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Expression Vectors and Cloning Vehicles

The invention provides expression vectors and cloning vehicles comprising nucleic acids of the invention, e.g., sequences encoding polypeptides (e.g., enzymes) of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector may comprise a promoter, a ribosome-binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

A DNA sequence may be inserted into a vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector may be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, pDR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

Host Cells and Transformed Cells

The invention also provides a transformed cell comprising a nucleic acid sequence of the invention, e.g., a sequence encoding polypeptides (e.g., enzymes) of the invention, a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

### Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides (e.g., enzymes) of the invention. In one aspect, the primer pairs are capable of amplifying nucleic acid sequences of the invention, e.g., including the exemplary SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073, or a subsequence thereof, etc. One of skill in the art can design amplification primer sequence pairs for any part of or the full length of these sequences.

The invention provides an amplification primer sequence pair for amplifying a nucleic acid encoding a polypeptide of the invention, wherein the primer pair is capable of amplifying a nucleic acid comprising a sequence of the invention, or fragments or subsequences thereof. One or each member of the amplification primer sequence pair can comprise an oligonucleotide comprising at least about 10 to 50 consecutive bases of the sequence, or about 12, 13, 14, 15, 16, 17, 18, 19, 20, 21, 22, 23, 24, or 25 consecutive bases of the sequence.

The invention provides amplification primer pairs, wherein the primer pair comprises a first member having a sequence as set forth by about the first (the 5′) 12, 13, 14, 15, 16, 17, 18, 19, 20, 21, 22, 23, 24, or 25 residues of a nucleic acid of the invention, and a second member having a sequence as set forth by about the first (the 5′) 12, 13, 14, 15, 16, 17, 18, 19, 20, 21, 22, 23, 24, or 25 residues of the complementary strand of the first member. The invention provides polypeptides (e.g., enzymes) generated by amplification, e.g., polymerase chain reaction (PCR), using an amplification primer pair of the invention. The invention provides methods of making polypeptides (e.g., enzymes) by amplification, e.g., polymerase chain reaction (PCR), using an amplification primer pair of the invention. In one aspect, the amplification primer pair amplifies a nucleic acid from a library, e.g., a gene library, such as an environmental library.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified. The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

### Determining the Degree of Sequence Identity

The invention provides nucleic acids comprising sequences having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention (e.g., SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073, and nucleic acids encoding SEQ ID NO:3, SEQ ID NO:5, SEQ ID NO:7, SEQ ID NO:9, SEQ ID NO:11, etc., and all polypeptides disclosed in the SEQ ID listing, which include all odd numbered SEQ ID NO:s from SEQ ID NO:3 through SEQ ID NO:1073) over a region of at least about 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550 or more, residues. The invention provides polypeptides comprising sequences having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary polypeptide of the invention. The extent of sequence identity (homology) may be determined using any computer program and associated parameters, including those described herein, such as BLAST 2.2.2. or FASTA version 3.0t78, with the default parameters.

In alternative embodiments, the sequence identify can be over a region of at least about 5, 10, 20, 30, 40, 50, 100, 150, 200, 250, 300, 350, 400 consecutive residues, or the full length of the nucleic acid or polypeptide. The extent of sequence identity (homology) may be determined using any computer program and associated parameters, including those described herein, such as BLAST 2.2.2. or FASTA version 3.0t78, with the default parameters.

Homologous sequences also include RNA sequences in which uridines replace the thymines in the nucleic acid sequences. The homologous sequences may be obtained using any of the procedures described herein or may result from the correction of a sequencing error. It will be appreciated that the nucleic acid sequences as set forth herein can be represented in the traditional single character format (see, e.g., Stryer, Lubert. Biochemistry, 3rd Ed., W. H Freeman & Co., New York) or in any other format which records the identity of the nucleotides in a sequence.

Various sequence comparison programs identified herein are used in this aspect of the invention. Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (e.g., an exemplary sequence SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous residues. For example, in alternative aspects of the invention, contiguous residues ranging anywhere from 20 to the full length of an exemplary sequence of the invention are compared to a reference sequence of the same number of contiguous positions after the two sequences are optimally aligned. If the reference sequence has the requisite sequence identity to an exemplary sequence of the invention, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence of the invention, that sequence is within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Several genomes have been sequenced, e.g., M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000). Significant progress has also been made in sequencing the genomes of model organism, such as mouse, C. elegans, and Arabadopsis sp. Databases containing genomic information annotated with some functional information are maintained by different organization, and are accessible via the internet.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used. default options to blastp. There are about 38 setting options in the BLAST 2.2.2 program. In this exemplary aspect of the invention, all default values are used except for the default filtering setting (i.e., all parameters set to default except filtering which is set to OFF); in its place a “-F F” setting is used, which disables filtering. Use of default filtering often results in Karlin-Altschul violations due to short length of sequence.

The default values used in this exemplary aspect of the invention include:
“Filter for low complexity: ON
>Word Size: 3
>Matrix: Blosum62
>Gap Costs: Existence: 11
>Extension: 1”

Other default settings are: filter for low complexity OFF, word size of 3 for protein, BLOSUM62 matrix, gap existence penalty of −11 and a gap extension penalty of −1.

An exemplary NCBI BLAST 2.2.2 program setting is set forth in Example 1, below. Note that the “-W” option defaults to 0. This means that, if not set, the word size defaults to 3 for proteins and 11 for nucleotides.

### Computer Systems and Computer Program Products

To determine and identify sequence identities, structural homologies, motifs and the like in silico the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention, e.g., an exemplary sequence of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

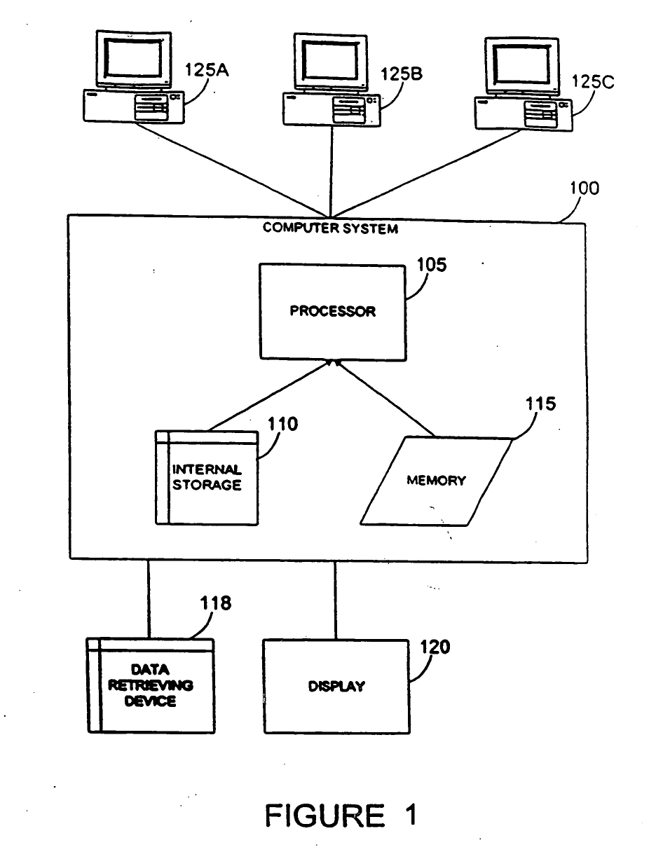

Aspects of the invention include systems (e.g., internet based systems), particularly computer systems, which store and manipulate the sequences and sequence information described herein. One example of a computer system 100 is illustrated in block diagram form in FIG. 1. As used herein, “a computer system” refers to the hardware components, software components, and data storage components used to analyze a nucleotide or polypeptide sequence of the invention. The computer system 100 can include a processor for processing, accessing and manipulating the sequence data. The processor 105 can be any well-known type of central processing unit, such as, for example, the Pentium III from Intel Corporation, or similar processor from Sun, Motorola, Compaq, AMD or International Business Machines. The computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one aspect, the computer system 100 includes a processor 105 connected to a bus which is connected to a main memory 115 (preferably implemented as RAM) and one or more internal data storage devices 110, such as a hard drive and/or other computer readable media having data recorded thereon. The computer system 100 can further include one or more data retrieving device 118 for reading the data stored on the internal data storage devices 110.

The data retrieving device 118 may represent, for example, a floppy disk drive, a compact disk drive, a magnetic tape drive, or a modem capable of connection to a remote data storage system (e.g., via the internet) etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100. Software for accessing and processing the nucleotide or amino acid sequences of the invention can reside in main memory 115 during execution.

In some aspects, the computer system 100 may further comprise a sequence comparison algorithm for comparing a nucleic acid sequence of the invention. The algorithm and sequence(s) can be stored on a computer readable medium. A “sequence comparison algorithm” refers to one or more programs which are implemented (locally or remotely) on the computer system 100 to compare a nucleotide sequence with other nucleotide sequences and/or compounds stored within a data storage means. For example, the sequence comparison algorithm may compare the nucleotide sequences of an exemplary sequence, e.g., SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073, stored on a computer readable medium to reference sequences stored on a computer readable medium to identify homologies or structural motifs.

The parameters used with the above algorithms may be adapted depending on the sequence length and degree of homology studied. In some aspects, the parameters may be the default parameters used by the algorithms in the absence of instructions from the user.

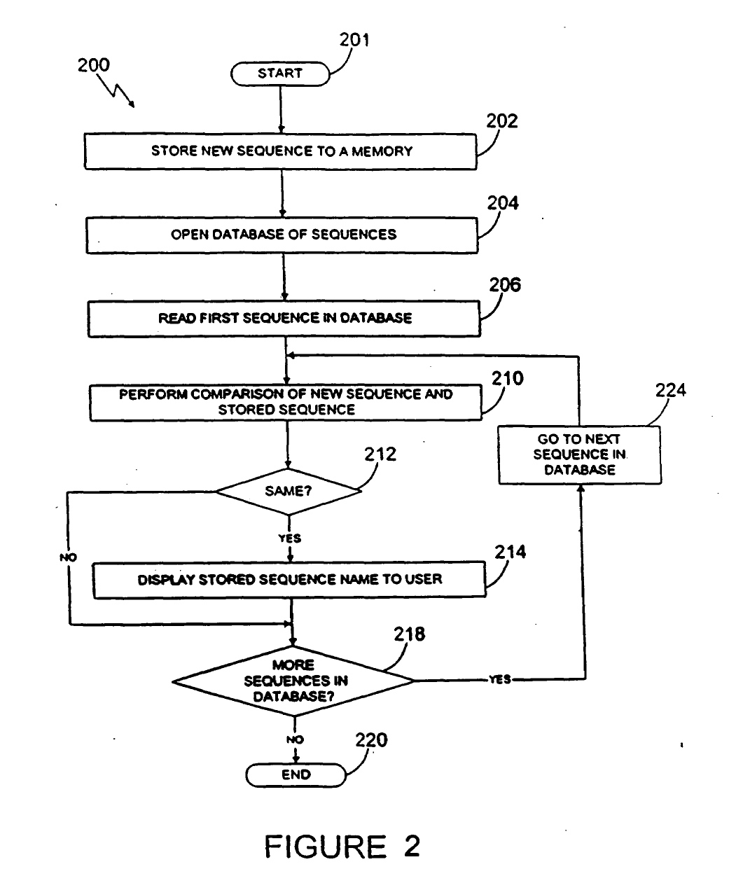

FIG. 2 is a flow diagram illustrating one aspect of a process 200 for comparing a new nucleotide or protein sequence with a database of sequences in order to determine the homology levels between the new sequence and the sequences in the database. The database of sequences can be a private database stored within the computer system 100, or a public database such as GENBANK that is available through the Internet. The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200. If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison. Accordingly, one aspect of the invention is a computer system comprising a processor, a data storage device having stored thereon a nucleic acid sequence of the invention and a sequence comparer for conducting the comparison. The sequence comparer may indicate a homology level between the sequences compared or identify structural motifs, or it may identify structural motifs in sequences which are compared to these nucleic acid codes and polypeptide codes.

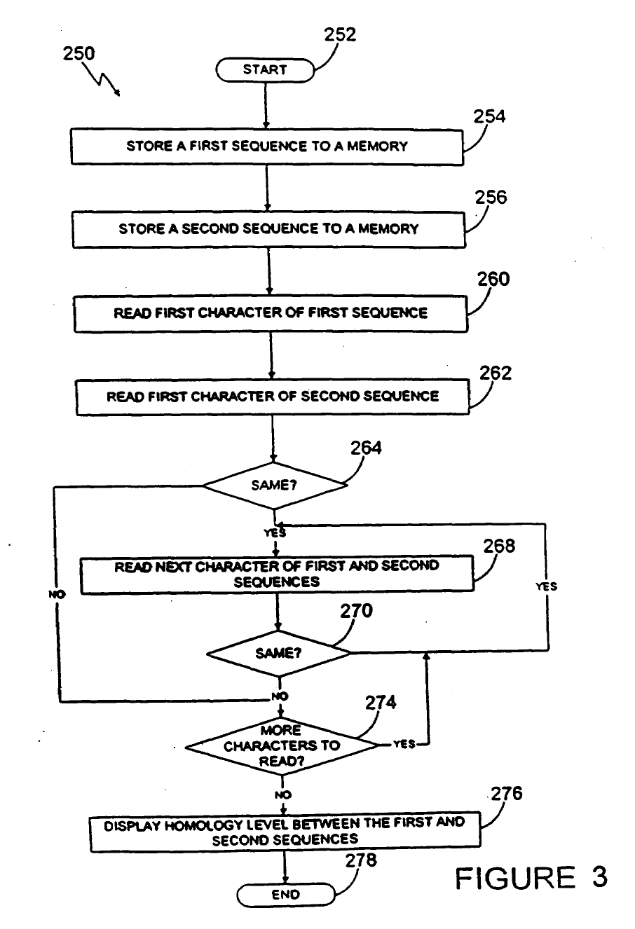

FIG. 3 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it can be a single letter amino acid code so that the first and sequence sequences can be easily compared. A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read. If there are not any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program can compare a reference sequence to a sequence of the invention to determine whether the sequences differ at one or more positions. The program can record the length and identity of inserted, deleted or substituted nucleotides or amino acid residues with respect to the sequence of either the reference or the invention. The computer program may be a program which determines whether a reference sequence contains a single nucleotide polymorphism (SNP) with respect to a sequence of the invention, or, whether a sequence of the invention comprises a SNP of a known sequence. Thus, in some aspects, the computer program is a program which identifies SNPs. The method may be implemented by the computer systems described above and the method illustrated in FIG. 3. The method can be performed by reading a sequence of the invention and the reference sequences through the use of the computer program and identifying differences with the computer program.

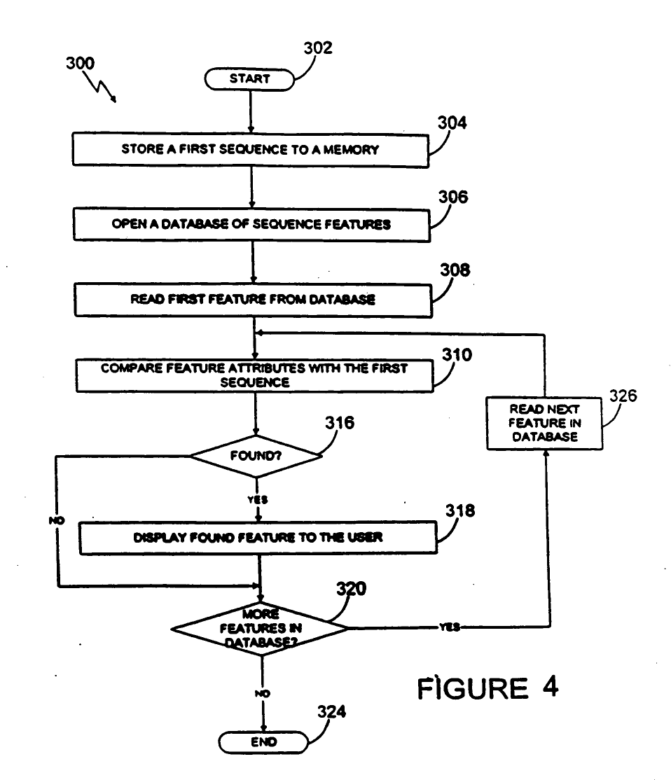

In other aspects the computer based system comprises an identifier for identifying features within a nucleic acid or polypeptide of the invention. An “identifier” refers to one or more programs which identifies certain features within a nucleic acid sequence. For example, an identifier may comprise a program which identifies an open reading frame (ORF) in a nucleic acid sequence. FIG. 4 is a flow diagram illustrating one aspect of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group. Alternatively, the features may be structural polypeptide motifs such as alpha helices, beta sheets, or functional polypeptide motifs such as enzymatic active sites, helix-turn-helix motifs or other motifs known to those skilled in the art. Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user. The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence. If the feature attribute is not found in the first sequence at the decision state 316, the process 300 moves directly to the decision state 320 in order to determine if any more features exist in the database. Thus, in one aspect, the invention provides a computer program that identifies open reading frames (ORFs).

A polypeptide or nucleic acid sequence of the invention may be stored and manipulated in a variety of data processor programs in a variety of formats. For example, a sequence can be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparison algorithms, identifiers, or sources of reference nucleotide sequences or polypeptide sequences to be compared to a nucleic acid sequence of the invention. The programs and databases used to practice the invention include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403, 1990), FASTA (Pearson and Lipman, Proc. Natl. Acad. Sci. USA, 85: 2444, 1988), FASTDB (Brutlag et al. Comp. App. Biosci. 6:237-245, 1990), Catalyst (Molecular Simulations Inc.), Catalyst/SHAPE (Molecular Simulations Inc.), Cerius2.DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwent's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### Hybridization of Nucleic Acids

The invention provides isolated or recombinant nucleic acids that hybridize under stringent conditions to a sequence of the invention, e.g., a sequence as set forth in SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073, or a nucleic acid that encodes a polypeptide of the invention, e.g., SEQ ID NO:3, SEQ ID NO:5, SEQ ID NO:7, SEQ ID NO:9, SEQ ID NO:11, etc., and all polypeptides disclosed in the SEQ ID listing, which include all odd numbered SEQ ID NO:s from SEQ ID NO:3 through SEQ ID NO:1073. The stringent conditions can be highly stringent conditions, medium stringent conditions, low stringent conditions, including the high and reduced stringency conditions described herein. In alternative embodiments, nucleic acids of the invention as defined by their ability to hybridize under stringent conditions can be between about five residues and the full length of the molecule, e.g., an exemplary nucleic acid of the invention. For example, they can be at least 5, 10, 15, 20, 25, 30, 35, 40, 50, 55, 60, 65, 70, 75, 80, 90, 100, 150, 200, 250, 300, 350, 400 residues in length. Nucleic acids shorter than full length are also included. These nucleic acids are useful as, e.g., hybridization probes, labeling probes, PCR oligonucleotide probes, iRNA (single or double stranded), antisense or sequences encoding antibody binding peptides (epitopes), motifs, active sites and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C. Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions are well known in the art. Hybridization conditions are discussed further, below.

### Oligonucleotides Probes and Methods for Using Them

The invention also provides nucleic acid probes for identifying nucleic acids encoding polypeptides (e.g., enzymes) of the invention, and polypeptides having the same activity as a polypeptide of the invention. In one aspect, the probe comprises at least 10 consecutive bases of a sequence of the invention, e.g., the exemplary SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073. Alternatively, a probe of the invention can be at least about 5, 6, 7, 8, 9, 10, 11, 12, 13, 14, 15, 16, 17, 18, 19, 20, 21, 22, 23, 24, 25, 30, 35, 40, 50, 55, 60, 65, 70, 75, 80, 90, 100, 150, about 10 to 50, about 20 to 60 about 30 to 70, consecutive bases of a sequence as set forth in a sequence of the invention. The probes identify a nucleic acid by binding or hybridization. The probes can be used in arrays of the invention, see discussion below, including, e.g., capillary arrays. The probes of the invention can also be used to isolate other nucleic acids or polypeptides.

The probes of the invention can be used to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions which permit the probe to specifically hybridize to any complementary sequences present in the sample. Where necessary, conditions which permit the probe to specifically hybridize to complementary sequences may be determined by placing the probe in contact with complementary sequences from samples known to contain the complementary sequence, as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids (see discussion on specific hybridization conditions).

If the sample contains the organism from which the nucleic acid was isolated, specific hybridization of the probe is then detected. Hybridization may be detected by labeling the probe with a detectable agent such as a radioactive isotope, a fluorescent dye or an enzyme capable of catalyzing the formation of a detectable product. Many methods for using the labeled probes to detect the presence of complementary nucleic acids in a sample are familiar to those skilled in the art. These include Southern Blots, Northern Blots, colony hybridization procedures, and dot blots. Protocols for each of these procedures are provided in Ausubel and Sambrook.

Alternatively, more than one probe (at least one of which is capable of specifically hybridizing to any complementary sequences which are present in the nucleic acid sample), may be used in an amplification reaction to determine whether the sample contains an organism containing a nucleic acid sequence of the invention (e.g., an organism from which the nucleic acid was isolated). In one aspect, the probes comprise oligonucleotides. In one aspect, the amplification reaction may comprise a PCR reaction. PCR protocols are described in Ausubel and Sambrook (see discussion on amplification reactions). In such procedures, the nucleic acids in the sample are contacted with the probes, the amplification reaction is performed, and any resulting amplification product is detected. The amplification product may be detected by performing gel electrophoresis on the reaction products and staining the gel with an intercalator such as ethidium bromide. Alternatively, one or more of the probes may be labeled with a radioactive isotope and the presence of a radioactive amplification product may be detected by autoradiography after gel electrophoresis.

Probes derived from sequences near the 3′ or 5′ ends of a nucleic acid sequence of the invention can also be used in chromosome walking procedures to identify clones containing additional, e.g., genomic sequences. Such methods allow the isolation of genes which encode additional proteins of interest from the host organism.

In one aspect, nucleic acid sequences of the invention are used as probes to identify and isolate related nucleic acids. In some aspects, the so-identified related nucleic acids may be cDNAs or genomic DNAs from organisms other than the one from which the nucleic acid of the invention was first isolated. In such procedures, a nucleic acid sample is contacted with the probe under conditions which permit the probe to specifically hybridize to related sequences. Hybridization of the probe to nucleic acids from the related organism is then detected using any of the methods described above.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter. Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH2PO4, pH 7.0, 5.0 mM Na2EDTA, 0.5% SDS, 10×Denhardt's, and 0.5 mg/ml polyriboadenylic acid. Approximately 2×107 cpm (specific activity 4−9×108 cpm/ug) of ³²P end-labeled oligonucleotide probe are then added to the solution. After 12-16 hours of incubation, the membrane is washed for 30 minutes at room temperature (RT) in 1×SET (150 mM NaCl, 20 mM Tris hydrochloride, pH 7.8, 1 mM Na2EDTA) containing 0.5% SDS, followed by a 30 minute wash in fresh 1×SET at Tm−10° C. for the oligonucleotide probe. The membrane is then exposed to auto-radiographic film for detection of hybridization signals.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tm, is the temperature (under defined ionic strength and pH) at which 50% of the target sequence hybridizes to a perfectly complementary probe. Very stringent conditions are selected to be equal to or about 5° C. lower than the Tm for a particular probe. The melting temperature of the probe may be calculated using the following exemplary formulas. For probes between 14 and 70 nucleotides in length the melting temperature (Tm) is calculated using the formula: Tm=81.5+16.6(log [Na+])+0.41(fraction G+C)−(600/N) where N is the length of the probe. If the hybridization is carried out in a solution containing formamide, the melting temperature may be calculated using the equation: Tm=81.5+16.6(log [Na+])+0.41(fraction G+C)−(0.63% formamide)-(600/N) where N is the length of the probe. Prehybridization may be carried out in 6×SSC, 5×Denhardt's reagent, 0.5% SDS, 100 μg denatured fragmented salmon sperm DNA or 6×SSC, 5×Denhardt's reagent, 0.5% SDS, 100 μg denatured fragmented salmon sperm DNA, 50% formamide. Formulas for SSC and Denhardt's and other solutions are listed, e.g., in Sambrook.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. In one aspect, hybridizations in 6×SSC are conducted at approximately 68° C. In one aspect, hybridizations in 50% formamide containing solutions are conducted at approximately 42° C. All of the foregoing hybridizations would be considered to be under conditions of high stringency.

Following hybridization, the filter is washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content), and the nucleic acid type (e.g., RNA v. DNA). Examples of progressively higher stringency condition washes are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.15M NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes.

Nucleic acids which have hybridized to the probe can be identified by autoradiography or other conventional techniques. The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. An example of “moderate” hybridization conditions is when the above hybridization is conducted at 55° C. An example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

These probes and methods of the invention can be used to isolate nucleic acids having a sequence with at least about 99%, 98%, 97%, at least 95%, at least 90%, at least 85%, at least 80%, at least 75%, at least 70%, at least 65%, at least 60%, at least 55%, or at least 50% homology to a nucleic acid sequence of the invention comprising at least about 10, 15, 20, 25, 30, 35, 40, 50, 75, 100, 150, 200, 250, 300, 350, 400, or 500 consecutive bases thereof, and the sequences complementary thereto. Homology may be measured using an alignment algorithm, as discussed herein. For example, the homologous polynucleotides may have a coding sequence which is a naturally occurring allelic variant of one of the coding sequences described herein. Such allelic variants may have a substitution, deletion or addition of one or more nucleotides when compared to nucleic acids of the invention.

Additionally, the probes and methods of the invention may be used to isolate nucleic acids which encode polypeptides having at least about 99%, at least 95%, at least 90%, at least 85%, at least 80%, at least 75%, at least 70%, at least 65%, at least 60%, at least 55%, or at least 50% sequence identity (homology) to a polypeptide of the invention comprising at least 5, 10, 15, 20, 25, 30, 35, 40, 50, 75, 100, or 150 consecutive amino acids thereof as determined using a sequence alignment algorithm (e.g., such as the FASTA version 3.0t78 algorithm with the default parameters, or a BLAST 2.2.2 program with exemplary settings as set forth herein).

### Inhibiting Expression of Polypeptides

The invention further provides for nucleic acids complementary to (e.g., antisense sequences to) the nucleic acids of the invention, e.g., polypeptide-encoding nucleic acids. Antisense sequences are capable of inhibiting the transport, splicing or transcription of polypeptide-encoding genes. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind polypeptide-coding gene or message, in either case preventing or inhibiting the production or function of the polypeptide. The association can be though sequence-specific hybridization. Another useful class of inhibitors includes oligonucleotides which cause inactivation or cleavage of polypeptide message. The oligonucleotide can have activity which causes such cleavage, such as ribozymes. The oligonucleotide can be chemically modified or conjugated to an enzyme or composition capable of cleaving the complementary nucleic acid. One may screen a pool of many different such oligonucleotides for those with the desired activity. Inhibition of polypeptide (e.g, enzyme) expression can have a variety of industrial applications. These compositions also can be expressed by the plant (e.g., a transgenic plant) or another organism (e.g., a bacterium or other microorganism transformed with an enzyme gene of the invention). The compositions of the invention for the inhibition of polypeptide (e.g., enzyme) expression (e.g., antisense, iRNA, ribozymes, antibodies) can be used as pharmaceutical compositions.

Antisense Oligonucleotides

The invention provides antisense oligonucleotides capable of binding message which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl)glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense enzyme sequences of the invention (see, e.g., Gold (1995) J. of Biol. Chem. 270:13581-13584).

Inhibitory Ribozymes

The invention provides for with ribozymes capable of binding polypeptide-coding message which can inhibit polypeptide activity by targeting mRNA. Strategies for designing ribozymes and selecting the polypeptide-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RNaseP-like RNA (in association with an RNA guide sequence). Examples of such hammerhead motifs are described by Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

RNA Interference (RNAi)

In one aspect, the invention provides an RNA inhibitory molecule, a so-called “RNAi” molecule, comprising an polypeptide-encoding sequence of the invention. The RNAi molecule comprises a double-stranded RNA (dsRNA) molecule. The RNAi can inhibit expression of polypeptide-coding gene, e.g., an enzyme-encoding gene. In one aspect, the RNAi is about 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's of the invention. The process may be practiced in vitro, ex vivo or in vivo. In one aspect, the RNAi molecules of the invention can be used to generate a loss-of-function mutation in a cell, an organ or an animal. Methods for making and using RNAi molecules for selectively degrade RNA are well known in the art, see, e.g., U.S. Pat. Nos. 6,506,559; 6,511,824; 6,515,109; 6,489,127.

### Modification of Nucleic Acids

The invention provides methods of generating variants of the nucleic acids of the invention, e.g., those encoding a polypeptide. These methods can be repeated or used in various combinations to generate enzymes having an altered or different activity or an altered or different stability from that of an enzyme encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

A nucleic acid of the invention can be altered by any means. For example, random or stochastic methods, or, non-stochastic, or “directed evolution,” methods.

Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor headpiece dimer” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751. Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols used in the methods of the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

See also U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION” by del Cardayre et al., filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549).

Non-stochastic, or “directed evolution,” methods include, e.g., saturation mutagenesis (GSSM), synthetic ligation reassembly (SLR), or a combination thereof are used to modify the nucleic acids of the invention to generate polypeptides with new or altered properties (e.g., activity under highly acidic or alkaline conditions, high temperatures, and the like). Polypeptides encoded by the modified nucleic acids can be screened for an activity before testing for an enzyme or other activity. Any testing modality or protocol can be used, e.g., using a capillary array platform. See, e.g., U.S. Pat. Nos. 6,280,926; 5,939,250.

Saturation Mutagenesis, or, GSSM

In one aspect of the invention, non-stochastic gene modification, a “directed evolution process,” is used to generate polypeptides with new or altered properties. Variations of this method have been termed “gene site-saturation mutagenesis,” “site-saturation mutagenesis,” “saturation mutagenesis” or simply “GSSM.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide (e.g., enzyme) molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased enzyme activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

The invention provides a non-stochastic gene modification system termed “synthetic ligation reassembly,” or simply “SLR,” a “directed evolution process,” to generate polypeptides with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled.

In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another embodiment, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecularly homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

The invention provides a non-stochastic gene modification system termed “optimized directed evolution system” to generate polypeptides with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found in U.S. Ser. No. 09/332,835. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding an polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that a oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Determining Crossover Events

Embodiments of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein is preferably performed in MATLAB® (The Mathworks, Natick, Mass.) a programming language and development environment for technical computing.

Iterative Processes

In practicing the invention, these processes can be iteratively repeated. For example a nucleic acid (or, the nucleic acid) responsible for an altered polypeptide (e.g., enzyme) phenotype is identified, re-isolated, again modified, re-tested for activity. This process can be iteratively repeated until a desired phenotype is engineered. For example, an entire biochemical anabolic or catabolic pathway can be engineered into a cell, including enzyme activity.

Similarly, if it is determined that a particular oligonucleotide has no affect at all on the desired trait (e.g., a new enzyme phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

In Vivo Shuffling

In vivo shuffling of molecules is use in methods of the invention that provide variants of polypeptides of the invention, e.g., antibodies, enzymes, and the like. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In one aspect, the invention provides a method for producing a hybrid polynucleotide from at least a first polynucleotide and a second polynucleotide. The invention can be used to produce a hybrid polynucleotide by introducing at least a first polynucleotide and a second polynucleotide which share at least one region of partial sequence homology into a suitable host cell. The regions of partial sequence homology promote processes which result in sequence reorganization producing a hybrid polynucleotide. The term “hybrid polynucleotide”, as used herein, is any nucleotide sequence which results from the method of the present invention and contains sequence from at least two original polynucleotide sequences. Such hybrid polynucleotides can result from intermolecular recombination events which promote sequence integration between DNA molecules. In addition, such hybrid polynucleotides can result from intramolecular reductive reassortment processes which utilize repeated sequences to alter a nucleotide sequence within a DNA molecule.

Producing Sequence Variants

The invention also provides methods of making sequence variants of the nucleic acid and polypeptide sequences of the invention or isolating polypeptides, e.g., enzymes, sequence variants using the nucleic acids and polypeptides of the invention. In one aspect, the invention provides for variants of an polypeptide-encoding gene of the invention, which can be altered by any means, including, e.g., random or stochastic methods, or, non-stochastic, or “directed evolution,” methods, as described above.

The isolated variants may be naturally occurring. Variant can also be created in vitro. Variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl2, MnCl2, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 110 mM Tris HCl (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/ul in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some embodiments, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate variants. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some embodiments, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

The invention also provides variants of polypeptides of the invention comprising sequences in which one or more of the amino acid residues (e.g., of an exemplary polypeptide of the invention) are substituted with a conserved or non-conserved amino acid residue (e.g., a conserved amino acid residue) and such substituted amino acid residue may or may not be one encoded by the genetic code. Conservative substitutions are those that substitute a given amino acid in a polypeptide by another amino acid of like characteristics. Thus, polypeptides of the invention include those with conservative substitutions of sequences of the invention, including but not limited to the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. Other variants are those in which one or more of the amino acid residues of the polypeptides of the invention includes a substituent group.

Other variants within the scope of the invention are those in which the polypeptide is associated with another compound, such as a compound to increase the half-life of the polypeptide, for example, polyethylene glycol.

Additional variants within the scope of the invention are those in which additional amino acids are fused to the polypeptide, such as a leader sequence, a secretory sequence, a proprotein sequence or a sequence which facilitates purification, enrichment, or stabilization of the polypeptide.

In some aspects, the variants, fragments, derivatives and analogs of the polypeptides of the invention retain the same biological function or activity as the exemplary polypeptides, e.g., an enzyme activity, as described herein. In other aspects, the variant, fragment, derivative, or analog includes a proprotein, such that the variant, fragment, derivative, or analog can be activated by cleavage of the proprotein portion to produce an active polypeptide.

### Optimizing Codons to Achieve High Levels of Protein Expression in Host Cells

The invention provides methods for modifying polypeptide-encoding nucleic acids to modify codon usage. In one aspect, the invention provides methods for modifying codons in a nucleic acid encoding a polypeptide to increase or decrease its expression in a host cell. The invention also provides nucleic acids encoding a polypeptide modified to increase its expression in a host cell, polypeptides so modified, and methods of making the modified polypeptides. The method comprises identifying a “non-preferred” or a “less preferred” codon in polypeptide-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli and Pseudomonas fluorescens; gram positive bacteria, such as Streptomyces diversa, Lactobacillus gasseri, Lactococcus lactis, Lactococcus cremoris, Bacillus subtilis. Exemplary host cells also include eukaryotic organisms, e.g., various yeast, such as Saccharomyces sp., including Saccharomyces cerevisiae, Schizosaccharomyces pombe, Pichia pastoris, and Kluyveromyces lactis, Hansenula polymorpha, Aspergillus niger, and mammalian cells and cell lines and insect cells and cell lines. Thus, the invention also includes nucleic acids and polypeptides optimized for expression in these organisms and species.

For example, the codons of a nucleic acid encoding an polypeptide isolated from a bacterial cell are modified such that the nucleic acid is optimally expressed in a bacterial cell different from the bacteria from which the polypeptide was derived, a yeast, a fungi, a plant cell, an insect cell or a mammalian cell. Methods for optimizing codons are well known in the art, see, e.g., U.S. Pat. No. 5,795,737; Baca (2000) Int. J. Parasitol. 30:113-118; Hale (1998) Protein Expr. Purif. 12:185-188; Narum (2001) Infect. Immun. 69:7250-7253. See also Narum (2001) Infect. Immun. 69:7250-7253, describing optimizing codons in mouse systems; Outchkourov (2002) Protein Expr. Purif. 24:18-24, describing optimizing codons in yeast; Feng (2000) Biochemistry 39:15399-15409, describing optimizing codons in E. coli; Humphreys (2000) Protein Expr. Purif. 20:252-264, describing optimizing codon usage that affects secretion in E. coli.

### Transgenic Non-Human Animals

The invention provides transgenic non-human animals comprising a nucleic acid, a polypeptide, an expression cassette or vector or a transfected or transformed cell of the invention. The transgenic non-human animals can be, e.g., goats, rabbits, sheep, pigs, cows, rats and mice, comprising the nucleic acids of the invention. These animals can be used, e.g., as in vivo models to study polypeptide activity, or, as models to screen for modulators of polypeptide activity in vivo. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP).

“Knockout animals” can also be used to practice the methods of the invention. For example, in one aspect, the transgenic or modified animals of the invention comprise a “knockout animal,” e.g., a “knockout mouse,” engineered not to express or to be unable to express a polypeptide.

### Transgenic Plants and Seeds

The invention provides transgenic plants and seeds comprising a nucleic acid, a polypeptide (e.g., an enzyme), an expression cassette or vector or a transfected or transformed cell of the invention. The invention also provides plant products, e.g., oils, seeds, leaves, extracts and the like, comprising a nucleic acid and/or a polypeptide (e.g., an enzyme) of the invention. The transgenic plant can be dicotyledonous (a dicot) or monocotyledonous (a monocot). The invention also provides methods of making and using these transgenic plants and seeds. The transgenic plant or plant cell expressing a polypeptide of the invention may be constructed in accordance with any method known in the art. See, for example, U.S. Pat. No. 6,309,872.

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's enzyme production is regulated by endogenous transcriptional or translational control elements. The invention also provides “knockout plants” where insertion of gene sequence by, e.g., homologous recombination, has disrupted the expression of the endogenous gene. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants, below.

The nucleic acids of the invention can be used to confer desired traits on essentially any plant, e.g., on oil-seed containing plants, such as soybeans, rapeseed, sunflower seeds, sesame and peanuts. Nucleic acids of the invention can be used to manipulate metabolic pathways of a plant in order to optimize or alter host's expression of a polypeptide (e.g., an enzyme). This can change the polypeptide's activity (e.g., enzyme activity) in a plant. Alternatively, polypeptides of the invention can be used in production of a transgenic plant to produce a compound not naturally produced by that plant. This can lower production costs or create a novel product.

In one aspect, the first step in production of a transgenic plant involves making an expression construct for expression in a plant cell. These techniques are well known in the art. They can include selecting and cloning a promoter, a coding sequence for facilitating efficient binding of ribosomes to mRNA and selecting the appropriate gene terminator sequences. One exemplary constitutive promoter is CaMV35S, from the cauliflower mosaic virus, which generally results in a high degree of expression in plants. Other promoters are more specific and respond to cues in the plant's internal or external environment. An exemplary light-inducible promoter is the promoter from the cab gene, encoding the major chlorophyll a/b binding protein.

In one aspect, the nucleic acid is modified to achieve greater expression in a plant cell. For example, a sequence of the invention is likely to have a higher percentage of A-T nucleotide pairs compared to that seen in a plant, some of which prefer G-C nucleotide pairs. Therefore, A-T nucleotides in the coding sequence can be substituted with G-C nucleotides without significantly changing the amino acid sequence to enhance production of the gene product in plant cells.

Selectable marker gene can be added to the gene construct in order to identify plant cells or tissues that have successfully integrated the transgene. This may be necessary because achieving incorporation and expression of genes in plant cells is a rare event, occurring in just a few percent of the targeted tissues or cells. Selectable marker genes encode proteins that provide resistance to agents that are normally toxic to plants, such as antibiotics or herbicides. Only plant cells that have integrated the selectable marker gene will survive when grown on a medium containing the appropriate antibiotic or herbicide. As for other inserted genes, marker genes also require promoter and termination sequences for proper function.

In one aspect, making transgenic plants or seeds comprises incorporating sequences of the invention and, optionally, marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In one aspect, protoplasts can be immobilized and injected with nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

Nucleic acids, e.g., expression constructs, can also be introduced in to plant cells using recombinant viruses. Plant cells can be transformed using viral vectors, such as, e.g., tobacco mosaic virus derived vectors (Rouwendal (1997) Plant Mol. Biol. 33:989-999), see Porta (1996) “Use of viral replicons for the expression of genes in plants,” Mol. Biotechnol. 5:209-221.

Alternatively, nucleic acids, e.g., an expression construct, can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, e.g., Horsch (1984) Science 233:496-498; Fraley (1983) Proc. Natl. Acad. Sci. USA 80:4803 (1983); Gene Transfer to Plants, Potrykus, ed. (Springer-Verlag, Berlin 1995). The DNA in an A. tumefaciens cell is contained in the bacterial chromosome as well as in another structure known as a Ti (tumor-inducing) plasmid. The Ti plasmid contains a stretch of DNA termed T-DNA (˜20 kb long) that is transferred to the plant cell in the infection process and a series of vir (virulence) genes that direct the infection process. A. tumefaciens can only infect a plant through wounds: when a plant root or stem is wounded it gives off certain chemical signals, in response to which, the vir genes of A. tumefaciens become activated and direct a series of events necessary for the transfer of the T-DNA from the Ti plasmid to the plant's chromosome. The T-DNA then enters the plant cell through the wound. One speculation is that the T-DNA waits until the plant DNA is being replicated or transcribed, then inserts itself into the exposed plant DNA. In order to use A. tumefaciens as a transgene vector, the tumor-inducing section of T-DNA have to be removed, while retaining the T-DNA border regions and the vir genes. The transgene is then inserted between the T-DNA border regions, where it is transferred to the plant cell and becomes integrated into the plant's chromosomes.

The invention provides for the transformation of monocotyledonous plants using the nucleic acids of the invention, including important cereals, see Hiei (1997) Plant Mol. Biol. 35:205-218. See also, e.g., Horsch, Science (1984) 233:496; Fraley (1983) Proc. Natl. Acad. Sci USA 80:4803; Thykjaer (1997) supra; Park (1996) Plant Mol. Biol. 32:1135-1148, discussing T-DNA integration into genomic DNA. See also D'Halluin, U.S. Pat. No. 5,712,135, describing a process for the stable integration of a DNA comprising a gene that is functional in a cell of a cereal, or other monocotyledonous plant.

In one aspect, the third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

After the expression cassette is stably incorporated in transgenic plants, it can be introduced into other plants by sexual crossing. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides (e.g., an enzyme) of the invention. The desired effects can be passed to future plant generations by standard propagation means.

The nucleic acids and polypeptides of the invention are expressed in or inserted in any plant or seed. Transgenic plants of the invention can be dicotyledonous or monocotyledonous. Examples of monocot transgenic plants of the invention are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicot transgenic plants of the invention are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, the transgenic plants and seeds of the invention include a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

In alternative embodiments, the nucleic acids of the invention are expressed in plants (e.g., as transgenic plants), such as oil-seed containing plants, e.g., soybeans, rapeseed, sunflower seeds, sesame and peanuts. The nucleic acids of the invention can be expressed in plants which contain fiber cells, including, e.g., cotton, silk cotton tree (Kapok, Ceiba pentandra), desert willow, creosote bush, winterfat, balsa, ramie, kenaf, hemp, roselle, jute, sisal abaca and flax. In alternative embodiments, the transgenic plants of the invention can be members of the genus Gossypium, including members of any Gossypium species, such as G. arboreum; G. herbaceum, G. barbadense, and G. hirsutum.

The invention also provides for transgenic plants to be used for producing large amounts of the polypeptides (e.g., an enzyme or antibody) of the invention. For example, see Palmgren (1997) Trends Genet. 13:348; Chong (1997) Transgenic Res. 6:289-296 (producing human milk protein beta-casein in transgenic potato plants using an auxin-inducible, bidirectional mannopine synthase (mas 1′,2′) promoter with Agrobacterium tumefaciens-mediated leaf disc transformation methods).

Using known procedures, one of skill can screen for plants of the invention by detecting the increase or decrease of transgene mRNA or protein in transgenic plants. Means for detecting and quantitation of mRNAs or proteins are well known in the art.

### Polypeptides and Peptides

The invention provides isolated or recombinant polypeptides having a sequence identity (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity) to an exemplary sequence of the invention, e.g., SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, and all polypeptides disclosed in the SEQ ID listing, which include all odd numbered SEQ ID NO:s from SEQ ID NO:3 through SEQ ID NO:1073. As discussed above, the identity can be over the full length of the polypeptide, or, the identity can be over a subsequence thereof, e.g., a region of at least about 50, 60, 70, 80, 90, 100, 150, 200, 250, 300, 350, 400, 450, 500, 550, 600, 650, 700 or more residues. Polypeptides of the invention can also be shorter than the full length of exemplary polypeptides. In alternative embodiment, the invention provides polypeptides (peptides, fragments) ranging in size between about 5 and the full length of a polypeptide, e.g., an enzyme, of the invention; exemplary sizes being of about 5, 10, 15, 20, 25, 30, 35, 40, 45, 50, 55, 60, 65, 70, 75, 80, 85, 90, 100, 125, 150, 175, 200, 250, 300, 350, 400 or more residues, e.g., contiguous residues of the exemplary polypeptide of the invention. Peptides of the invention can be useful as, e.g., labeling probes, antigens, toleragens, motifs, enzyme active sites.

In one aspect, the polypeptides, e.g., enzymes, of the invention are active at a high and/or at a low temperature, or, over a wide range of temperature, e.g., they can be active in the temperatures ranging between 20° C. to 90° C., between 30° C. to 80° C., or between 40° C. to 70° C. The invention also provides polypeptides, e.g., enzymes, having activity at alkaline pHs or at acidic pHs, e.g., low water acidity. In alternative aspects, the polypeptides, e.g., enzymes, of the invention can have activity in acidic pHs as low as pH 6.5, pH 6.0, pH 5.5, pH 5.0, pH 4.5, pH 4.0 and pH 3.5. In alternative aspects, the polypeptides, e.g., enzymes, of the invention can have activity in alkaline pHs as high as pH 7.5, pH 8.0, pH 8.5, pH 9.0, and pH 9.5. In one aspect, the polypeptide, e.g., enzymes, of the invention are active in the temperature range of between about 40° C. to about 70° C. under conditions of low water activity (low water content).

The invention also provides methods for further modifying the exemplary polypeptides of the invention to generate polypeptide (e.g., enzymes) with desirable properties. For example, enzymes generated by the methods of the invention can have altered substrate specificities, substrate binding specificities, substrate cleavage patterns, thermal stability, pH/activity profile, pH/stability profile (such as increased stability at low, e.g. pH<6 or pH<5, or high, e.g. pH>9, pH values), stability towards oxidation, Ca²⁺ dependency, specific activity and the like. The invention provides for altering any property of interest. For instance, the alteration may result in a variant which, as compared to a parent enzyme, has altered pH and temperature activity profile.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3□13) and automated synthesis may be achieved, e.g., using the ABI 431A Peptide Synthesizer (Perkin Elmer) in accordance with the instructions provided by the manufacturer.

The peptides and polypeptides of the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, in one aspect, a mimetic composition is within the scope of the invention if it has the same activity as a polypeptide of the invention.

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH2-NH), ethylene, olefin (CH═CH), ether (CH2-O), thioether (CH2-S), tetrazole (CN4-), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide of the invention can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R— or S— form.

The invention also provides methods for modifying the polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A™ automated peptide synthesizer. Such equipment provides ready access to the peptides of the invention, either by direct synthesis or by synthesis of a series of fragments that can be coupled using other known techniques.

Enzymes

The invention provides novel enzymes, nucleic acids encoding them, antibodies that bind them, peptides representing the enzyme's antigenic sites (epitopes) and active sites, and methods for making and using them. In one aspect, polypeptides of the invention have an enzyme activity, as described herein, see, e.g., Table 3. In alternative aspects, the enzymes of the invention have activities that have been modified from those of the exemplary enzymes described herein. The invention includes enzymes with and without signal sequences and the signal sequences themselves. The invention includes immobilized enzymes, anti-enzyme antibodies and fragments thereof. The invention includes heterocomplexes, e.g., fusion proteins, heterodimers, etc., comprising the enzymes of the invention.

Any of the many enzyme activity assays known in the art can be used to determine if a polypeptide has an enzyme activity and is within the scope of the invention. Routine protocols for determining enzyme activities are well known in the art.

Determining peptides representing the enzyme's antigenic sites (epitopes), active sites, binding sites, signal sequences, and the like can be done by routine screening protocols.

The enzymes of the invention are highly selective catalysts. As with other enzymes, they catalyze reactions with exquisite stereo-, regio-, and chemo-selectivities that are unparalleled in conventional synthetic chemistry. Moreover, the enzymes of the invention are remarkably versatile. They can be tailored to function in organic solvents, operate at extreme pHs (for example, high pHs and low pHs) extreme temperatures (for example, high temperatures and low temperatures), extreme salinity levels (for example, high salinity and low salinity), and catalyze reactions with compounds that are structurally unrelated to their natural, physiological substrates. Enzymes of the invention can be designed to be reactive toward a wide range of natural and unnatural substrates, thus enabling the modification of virtually any organic lead compound. Enzymes of the invention can also be designed to be highly enantio- and regio-selective. The high degree of functional group specificity exhibited by these enzymes enables one to keep track of each reaction in a synthetic sequence leading to a new active compound. Enzymes of the invention can also be designed to catalyze many diverse reactions unrelated to their native physiological function in nature.

The present invention exploits the unique catalytic properties of enzymes. Whereas the use of biocatalysts (i.e., purified or crude enzymes, non-living or living cells) in chemical transformations normally requires the identification of a particular biocatalyst that reacts with a specific starting compound. The present invention uses selected biocatalysts, i.e., the enzymes of the invention, and reaction conditions that are specific for functional groups that are present in many starting compounds. Each biocatalyst is specific for one functional group, or several related functional groups, and can react with many starting compounds containing this functional group. The biocatalytic reactions produce a population of derivatives from a single starting compound. These derivatives can be subjected to another round of biocatalytic reactions to produce a second population of derivative compounds. Thousands of variations of the original compound can be produced with each iteration of biocatalytic derivatization.

Enzymes react at specific sites of a starting compound without affecting the rest of the molecule, a process that is very difficult to achieve using traditional chemical methods. This high degree of biocatalytic specificity provides the means to identify a single active enzyme within a library. The library is characterized by the series of biocatalytic reactions used to produce it, a so-called “biosynthetic history”. Screening the library for biological activities and tracing the biosynthetic history identifies the specific reaction sequence producing the active compound. The reaction sequence is repeated and the structure of the synthesized compound determined. This mode of identification, unlike other synthesis and screening approaches, does not require immobilization technologies, and compounds can be synthesized and tested free in solution using virtually any type of screening assay. It is important to note, that the high degree of specificity of enzyme reactions on functional groups allows for the “tracking” of specific enzymatic reactions that make up the biocatalytically produced library.

The invention also provides methods of discovering new enzymes using the nucleic acids, polypeptides and antibodies of the invention. In one aspect, lambda phage libraries are screened for expression-based discovery of enzymes. Use of lambda phage libraries in screening allows detection of toxic clones; improved access to substrate; reduced need for engineering a host, by-passing the potential for any bias resulting from mass excision of the library; and, faster growth at low clone densities. Screening of lambda phage libraries can be in liquid phase or in solid phase. Screening in liquid phase gives greater flexibility in assay conditions; additional substrate flexibility; higher sensitivity for weak clones; and ease of automation over solid phase screening.

Many of the procedural steps are performed using robotic automation enabling the execution of many thousands of biocatalytic reactions and screening assays per day as well as ensuring a high level of accuracy and reproducibility (see discussion of arrays, below). As a result, a library of derivative compounds can be produced in a matter of weeks. For further teachings on modification of molecules, including small molecules, see PCT/US94/09174.

Signal Sequences, Prepro Domains and Catalytic Domains

The invention provides signal sequences (e.g., signal peptides (SPs)), prepro domains, and catalytic domains (CDs). The invention provides nucleic acids encoding these catalytic domains (CDs) and signal sequences (SPs, e.g., a peptide having a sequence comprising/consisting of amino terminal residues of a polypeptide of the invention). In one aspect, the invention provides a signal sequence comprising a peptide comprising/consisting of a sequence as set forth in residues 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32 or 1 to 33 of a polypeptide of the invention, e.g., SEQ ID NO:3, SEQ NO:5, etc. as set forth in the SEQ ID listing.

Exemplary signal sequences are set forth as follows (e.g., SEQ ID NO:1038 encodes signal peptide SEQ ID NO:1039, etc.):

| | | |
|---|---|---|
| SEQ ID NO: | Signalp Prediction | Signal Sequence |

| | | | |
|---|---|---|---|
| 1038, 1039 | Cleavage site: | AA1 28 AA2 29 | MIELLLYGLFYILGIFLISFFASHSYSA |
| | Probability: | 0.824 | |
| 108, 109 | Cleavage site: | AA1 36 AA2 37 | MDILKYFKLLLAAILSILIILIIFKLLYYIAFLL |
| | Probability: | 0.674 | YT |
| 146, 147 | Cleavage site: | AA1 34 AA2 35 | MDRSIELSLHLIVYAILSLLVLSLVFMFFSKS |
| | Probability: | 0.876 | QS |
| 172, 173 | Cleavage site: | AA1 24 AA2 25 | MKRTIILLLSALALGVALSQIAFL |
| | Probability: | 1 | |
| 196, 197 | Cleavage site: | AA1 25 AA2 26 | MQRIIPFLFLILVLVFLSLIPKSCK |
| | Probability: | 0.599 | |
| 202, 203 | Cleavage site: | AA1 67 AA2 68 | MHYRRAMSITFKNSKHKRNMRRSIELPISVV |
| | Probability: | 1 | LFAIIGLMVFSILLGFIPKVLGSLLGGFMSSVS |
| | | | ATK |
| 220, 221 | Cleavage site: | AA1 27 AA2 28 | MNSKDILIYASISSLLLMSFILFQTHR |
| | Probability: | 0.524 | |
| 270, 271 | Cleavage site: | AA1 30 AA2 31 | MRGAISTIVYILIGLIGAAFSLILMNSVFE |
| | Probability: | 0.759 | |
| 274, 275 | Cleavage site: | AA1 29 AA2 30 | MFRFSKMISWLGSLIILIMFLGVVSIVFN |
| | Probability: | 0.998 | |
| 276, 277 | Cleavage site: | AA1 39 AA2 40 | MRALSNIIWLISAIVVALMSIAIISFSFFKTVN |
| | Probability: | 0.999 | PLAALS |
| 282, 283 | Cleavage site: | AA1 21 AA2 22 | MFFIFKIFRLLFALPLIMLLA |
| | Probability: | 0.889 | |
| 308, 309 | Cleavage site: | AA1 38 AA2 39 | MIAINYMNLEMRDVVLGLVFVIAMAVAAVI |
| | Probability: | 0.973 | GAPSLALA |
| 310, 311 | Cleavage site: | AA1 38 AA2 39 | MAQTKTKTQIRLKMLEKIEKYKEPVFLILLF |
| | Probability: | 0.796 | LSGFLFK |
| 318, 319 | Cleavage site: | AA1 28 AA2 29 | MEKKVLIAIPLLLSVGFIFYYFSPPSNN |
| | Probability: | 0.962 | |
| 322, 323 | Cleavage site: | AA1 33 AA2 34 | MKLPILILLAVVLIIVFFILLPFIPYIATGAVIA |
| | Probability: | 0.978 | |
| 328, 329 | Cleavage site: | AA1 25 AA2 26 | MDYNIFINIVLSSFVVALASSLVTV |
| | Probability: | 0.782 | |
| 378, 379 | Cleavage site: | AA1 19 AA2 20 | MLPLILILLSALFSSYETA |
| | Probability: | 0.953 | |
| 390, 391 | Cleavage site: | AA1 24 AA2 25 | MFALIIQLSSYALAFILSPLFVLS |
| | Probability: | 0.876 | |
| 394, 395 | Cleavage site: | AA1 18 AA2 19 | MKSLIAFIAFIITGFLAT |
| | Probability: | 0.567 | |
| 430, 431 | Cleavage site: | AA1 31 AA2 32 | MDLALASALAIGLAAFGSAIAQGLAASAAA |
| | Probability: | 1 | A |
| 440, 441 | Cleavage site: | AA1 20 AA2 21 | MRKLLSLPLITFFVLGLSVG |
| | Probability: | 0.864 | |
| 468, 469 | Cleavage site: | AA1 20 AA2 21 | MKKMFVPLMAAMPFLAIGLA |
| | Probability: | 0.997 | |
| 488, 489 | Cleavage site: | AA1 25 AA2 26 | MRRALESVNYLILLALSLFIALFVA |
| | Probability: | 0.954 | |
| 500, 501 | Cleavage site: | AA1 19 AA2 20 | MILLLLLPLALSAVVYTNT |
| | Probability: | 0.999 | |
| 506, 507 | Cleavage site: | AA1 28 AA2 29 | MTLNMKNLRKKVWLFSLILLGIVLVFLS |
| | Probability: | 0.95 | |
| 524, 525 | Cleavage site: | AA1 22 AA2 23 | MKTRFTLLLSLLLLNSIPIAIS |
| | Probability: | 0.997 | |
| 534, 535 | Cleavage site: | AA1 16 AA2 17 | MKKVFLSLLLVSTAFA |
| | Probability: | 0.997 | |
| 546, 547 | Cleavage site: | AA1 34 AA2 35 | MFGFYFKVYGALALLIAIALFAGYLFIDPNT |
| | Probability: | 0.795 | REK |
| 550, 551 | Cleavage site: | AA1 30 AA2 31 | MNKSDIVILAFFFVSLGLGILTLLPANTVK |
| | Probability: | 0.907 | |
| 58, 59 | Cleavage site: | AA1 22 AA2 23 | MKAKKLLIFVPLLLLPLLTLLM |
| | Probability: | 0.608 | |
| 586, 587 | Cleavage site: | AA1 24 AA2 25 | MKTLAKAQVVLASGMLLAGAVAQN |
| | Probability: | 0.998 | |
| 608, 609 | Cleavage site: | AA1 26 AA2 27 | MRAFYSIALFSILTLISLLIAHVLIT |
| | Probability: | 0.972 | |
| 64, 65 | Cleavage site: | AA1 55 AA2 56 | MMNVIDRIMRKIPLPKKFVYKIEEKAKNYIIK |
| | Probability: | 0.984 | EGPKLAPKLASVLTILAGAGLAMA |
| 646, 647 | Cleavage site: | AA1 36 AA2 37 | MSVKKWQEQLKNFLNNLDKHSILIGVAAGII |
| | Probability: | 0.974 | LAVSA |
| 66, 67 | Cleavage site: | AA1 23 AA2 24 | MLRLIILFLTIIGALAINVNVYS |
| | Probability: | 0.986 | |
| 70, 71 | Cleavage site: | AA1 16 AA2 17 | MKRFLPAVVVLGLALA |
| | Probability: | 1 | |
| 718, 719 | Cleavage site: | AA1 20 AA2 21 | MKKLFWVLAIPLALSAVQLK |
| | Probability: | 0.963 | |
| 738, 739 | Cleavage site: | AA1 25 AA2 26 | MRAVSWVLGLVVAIVLSLISFFIVS |
| | Probability: | 0.922 | |
| 746, 747 | Cleavage site: | AA1 46 AA2 47 | MNLILLAEILFSLTSIFFAALILKPFLVFNIFAT |
| | Probability: | 0.713 | LFGKQFACKSFA |
| 768, 769 | Cleavage site: | AA1 18 AA2 19 | MRHYFLASLLIFTPIAVS |
| | Probability: | 0.946 | |
| 796, 797 | Cleavage site: | AA1 18 AA2 19 | MLKFIALLIVSYIMELLA |
| | Probability: | 0.538 | |
| 80, 81 | Cleavage site: | AA1 20 AA2 21 | MHELVFALISILFLSLIAFK |
| | Probability: | 0.657 | |
| 806, 807 | Cleavage site: | AA1 42 AA2 43 | MMKKKFEEALTKFEEFGLVTLALAFVFLVL |
| | Probability: | 0.83 | VLYPLFMGVAFA |
| 810, 811 | Cleavage site: | AA1 22 AA2 23 | MKLNTLKNLLPIIFFFLGYFFA |
| | Probability: | 0.859 | |
| 854, 855 | Cleavage site: | AA1 23 AA2 24 | MKKKLIGLAFSLSILLFSIYVLV |
| | Probability: | 1 | |
| 866, 867 | Cleavage site: | AA1 22 AA2 23 | MFRRSITPIIAVVMMLMMTVMA |
| | Probability: | 1 | |
| 868, 869 | Cleavage site: | AA1 24 AA2 25 | MKSQSHLIEFVLVIGIALAGLSSA |
| | Probability: | 0.838 | |
| 888, 889 | Cleavage site: | AA1 27 AA2 28 | MGILTKILIKHKLLPFILFIIMGYLFA |
| | Probability: | 0.979 | |
| 894, 895 | Cleavage site: | AA1 20 AA2 21 | MRGINALIAIIVLVAIVGLA |
| | Probability: | 0.991 | |
| 902, 903 | Cleavage site: | AA1 29 AA2 30 | MKYSKFLFTWLVLSSALSLVWPANFPLVS |
| | Probability: | 0.969 | |
| 912, 913 | Cleavage site: | AA1 26 AA2 27 | MRKINMYYLLLLGILFMLSGCVNLSN |
| | Probability: | 0.998 | |
| 930, 931 | Cleavage site: | AA1 19 AA2 20 | MRRSSIFTLVFYALVGATA |
| | Probability: | 0.814 | |
| 948, 949 | Cleavage site: | AA1 22 AA2 23 | MLVSTLFNSFLILVSLFFLLFG |
| | Probability: | 0.86 | |
| 958, 959 | Cleavage site: | AA1 19 AA2 20 | MRTKVGLILFALPIVPALA |
| | Probability: | 1 | |
| 96, 97 | Cleavage site: | AA1 29 AA2 30 | MRSVSLTLNTIVMIALAISVFSILFIVLS |
| | Probability: | 0.97 | |
| 972, 973 | Cleavage site: | AA1 20 AA2 21 | MRSITPIIAIVILLLVTISA |
| | Probability: | 0.941 | |

The signal sequences of the invention can be isolated peptides, or, sequences joined to another polypeptide, e.g., as a fusion protein. In one aspect, the invention provides polypeptides comprising signal sequences of the invention. In one aspect, polypeptides comprising signal sequences of the invention comprise sequences heterologous to an enzyme of the invention (e.g., a fusion protein comprising a signal sequence of the invention and sequences from another polypeptide or a non-enzyme protein). In one aspect, the invention provides polypeptides of the invention with heterologous signal sequences, e.g., sequences with a yeast signal sequence. An enzyme of the invention can comprise a heterologous signal sequence, e.g., in a vector, e.g., a pPIC series vector (Invitrogen, Carlsbad, Calif.).

In one aspect, the signal sequences of the invention are identified following identification of novel polypeptides, e.g., enzymes. The pathways by which proteins are sorted and transported to their proper cellular location are often referred to as protein targeting pathways. One of the most important elements in all of these targeting systems is a short amino acid sequence at the amino terminus of a newly synthesized polypeptide called the signal sequence. This signal sequence directs a protein to its appropriate location in the cell and is removed during transport or when the protein reaches its final destination. Most lysosomal, membrane, or secreted proteins have an amino-terminal signal sequence that marks them for translocation into the lumen of the endoplasmic reticulum. More than 100 signal sequences for proteins in this group have been determined. The signal sequences can vary in length from 13 to 36 amino acid residues. Various methods of recognition of signal sequences are known to those of skill in the art. For example, in one aspect, novel polypeptide signal peptides are identified by a method referred to as SignalP. SignalP uses a combined neural network which recognizes both signal peptides and their cleavage sites. (Nielsen, et al., “Identification of prokaryotic and eukaryotic signal peptides and prediction of their cleavage sites.” Protein Engineering, vol. 10, no. 1, p. 1-6 (1997).

It should be understood that in some aspects polypeptides of the invention may not have signal sequences. In one aspect, the invention provides the polypeptides of the invention lacking all or part of a signal sequence. In one aspect, the invention provides a nucleic acid sequence encoding a signal sequence from one polypeptide operably linked to a nucleic acid sequence of a different.

The invention also provides isolated or recombinant polypeptides comprising signal sequences (SPs) and catalytic domains (CDs) of the invention and heterologous sequences. The heterologous sequences are sequences not naturally associated (e.g., to an enzyme) with an SP and/or CD. The sequence to which the SP and/or CD are not naturally associated can be on the SP's, and/or CD's amino terminal end, carboxy terminal end, and/or on both ends of the SP and/or CD. In one aspect, the invention provides an isolated or recombinant polypeptide comprising (or consisting of) a polypeptide comprising a signal sequence (SP) and/or catalytic domain (CD) of the invention with the proviso that it is not associated with any sequence to which it is naturally associated (e.g., an enzyme sequence). Similarly in one aspect, the invention provides isolated or recombinant nucleic acids encoding these polypeptides. Thus, in one aspect, the isolated or recombinant nucleic acid of the invention comprises coding sequence for a signal sequence (SP) and/or catalytic domain (CD) of the invention and a heterologous sequence (i.e., a sequence not naturally associated with the a signal sequence (SP) and/or catalytic domain (CD) of the invention). The heterologous sequence can be on the 3′ terminal end, 5′ terminal end, and/or on both ends of the SP and/or CD coding sequence.

### Hybrid (Chimeric) Polypeptides and Peptide Libraries

In one aspect, the invention provides hybrid polypeptides and fusion proteins, including peptide libraries, comprising sequences of the invention. The peptide libraries of the invention can be used to isolate peptide modulators (e.g., activators or inhibitors) of targets, such as enzyme substrates, receptors, enzymes. The peptide libraries of the invention can be used to identify formal binding partners of targets, such as ligands, e.g., cytokines, hormones and the like. In one aspect, the invention provides chimeric proteins comprising a signal sequence (SP) and/or catalytic domain (CD) of the invention and a heterologous sequence (see above).

The invention also provides methods for generating “improved” and hybrid polypeptides using the nucleic acids and polypeptides of the invention. The invention provides methods for generating hybrid polypeptides (e.g., hybrid enzymes).

In one aspect, the methods of the invention produce new hybrid polypeptides by utilizing cellular processes that integrate the sequence of a first polynucleotide such that resulting hybrid polynucleotides encode polypeptides demonstrating activities derived from the first biologically active polypeptides. For example, the first polynucleotides can be an exemplary nucleic acid sequence (e.g., SEQ ID NO:1, SEQ ID NO:2, SEQ ID NO:4, SEQ ID NO:6, SEQ ID NO:8, SEQ ID NO:10, etc., including all nucleic acids disclosed in the SEQ ID listing, which include all even numbered SEQ ID NO:s from SEQ ID NO:2 through SEQ ID NO:1073) encoding an exemplary polypeptide of the invention. The first nucleic acid can encode a polypeptide from one organism that functions effectively under a particular environmental condition, e.g. high salinity. It can be “integrated” with an polypeptide encoded by a second polynucleotide from a different organism that functions effectively under a different environmental condition, such as extremely high temperatures. For example, when the two nucleic acids can produce a hybrid molecule by e.g., recombination and/or reductive reassortment. A hybrid polynucleotide containing sequences from the first and second original polynucleotides may encode an polypeptide that exhibits characteristics of both polypeptides encoded by the original polynucleotides. Thus, the polypeptide encoded by the hybrid polynucleotide may function effectively under environmental conditions shared by each of the polypeptides encoded by the first and second polynucleotides, e.g., high salinity and extreme temperatures.

Alternatively, a hybrid polypeptide resulting from this method of the invention may exhibit specialized polypeptide activity not displayed in the original polypeptides. For example, following recombination and/or reductive reassortment of polynucleotides encoding polypeptide activities, the resulting hybrid polypeptide encoded by a hybrid polynucleotide can be screened for specialized activities obtained from each of the original polypeptides, for example, the type of bond on which an enzyme acts and the temperature at which the enzyme functions. Thus, for example, an enzyme may be screened to ascertain those chemical functionalities which distinguish the hybrid enzyme from the original enzymes, such as: (a) amide (peptide bonds), i.e., enzymes; (b) ester bonds, i.e., amylases and lipases; (c) acetals, i.e., glycosidases and, for example, the temperature, pH or salt concentration at which the hybrid polypeptide functions.

Sources of the polynucleotides to be “integrated” with nucleic acids of the invention may be isolated from individual organisms (“isolates”), collections of organisms that have been grown in defined media (“enrichment cultures”), or, uncultivated organisms (“environmental samples”). The use of a culture-independent approach to derive polynucleotides encoding novel bioactivities from environmental samples is most preferable since it allows one to access untapped resources of biodiversity. “Environmental libraries” are generated from environmental samples and represent the collective genomes of naturally occurring organisms archived in cloning vectors that can be propagated in suitable prokaryotic hosts. Because the cloned DNA is initially extracted directly from environmental samples, the libraries are not limited to the small fraction of prokaryotes that can be grown in pure culture. Additionally, a normalization of the environmental DNA present in these samples could allow more equal representation of the DNA from all of the species present in the original sample. This can dramatically increase the efficiency of finding interesting genes from minor constituents of the sample that may be under-represented by several orders of magnitude compared to the dominant species.

For example, gene libraries generated from one or more uncultivated microorganisms are screened for an activity of interest. Potential pathways encoding bioactive molecules of interest are first captured in prokaryotic cells in the form of gene expression libraries. Polynucleotides encoding activities of interest are isolated from such libraries and introduced into a host cell. The host cell is grown under conditions that promote recombination and/or reductive reassortment creating potentially active biomolecules with novel or enhanced activities.

The microorganisms from which hybrid polynucleotides may be prepared include prokaryotic microorganisms, such as Eubacteria and Archaebacteria, and lower eukaryotic microorganisms such as fungi, some algae and protozoa. Polynucleotides may be isolated from environmental samples. Nucleic acid may be recovered without culturing of an organism or recovered from one or more cultured organisms. In one aspect, such microorganisms may be extremophiles, such as hyperthermophiles, psychrophiles, psychrotrophs, halophiles, barophiles and acidophiles. In one aspect, polynucleotides encoding polypeptides isolated from extremophilic microorganisms are used to make hybrid polypeptides. Such polypeptides may function at temperatures above 100° C. in, e.g., terrestrial hot springs and deep sea thermal vents, at temperatures below 0° C. in, e.g., arctic waters, in the saturated salt environment of, e.g., the Dead Sea, at pH values around 0 in, e.g., coal deposits and geothermal sulfur-rich springs, or at pH values greater than 11 in, e.g., sewage sludge. For example, polypeptides cloned and expressed from extremophilic organisms can show high activity throughout a wide range of temperatures and pHs.

Polynucleotides selected and isolated as described herein, including at least one nucleic acid of the invention, are introduced into a suitable host cell. A suitable host cell is any cell that is capable of promoting recombination and/or reductive reassortment. The selected polynucleotides can be in a vector that includes appropriate control sequences. The host cell can be a higher eukaryotic cell, such as a mammalian cell, or a lower eukaryotic cell, such as a yeast cell, or preferably, the host cell can be a prokaryotic cell, such as a bacterial cell. Introduction of the construct into the host cell can be effected by calcium phosphate transfection, DEAE-Dextran mediated transfection, or electroporation (Davis et al., 1986).

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; and plant cells. The selection of an appropriate host for recombination and/or reductive reassortment or just for expression of recombinant protein is deemed to be within the scope of those skilled in the art from the teachings herein. Mammalian cell culture systems that can be employed for recombination and/or reductive reassortment or just for expression of recombinant protein include, e.g., the COS-7 lines of monkey kidney fibroblasts, described in “SV40-transformed simian cells support the replication of early SV40 mutants” (Gluzman, 1981), the C127, 3T3, CHO, HeLa and BHK cell lines. Mammalian expression vectors can comprise an origin of replication, a suitable promoter and enhancer, and necessary ribosome binding sites, polyadenylation site, splice donor and acceptor sites, transcriptional termination sequences, and 5′ flanking non-transcribed sequences. DNA sequences derived from the SV40 splice, and polyadenylation sites may be used to provide the required non-transcribed genetic elements.

Host cells containing the polynucleotides of interest (for recombination and/or reductive reassortment or just for expression of recombinant protein) can be cultured in conventional nutrient media modified as appropriate for activating promoters, selecting transformants or amplifying genes. The culture conditions, such as temperature, pH and the like, are those previously used with the host cell selected for expression, and will be apparent to the ordinarily skilled artisan. The clones which are identified as having the specified polypeptide activity may then be sequenced to identify the polynucleotide sequence encoding polypeptides having the enhanced activity.

In another aspect, the nucleic acids and methods of the present invention can be used to generate novel polynucleotides for biochemical pathways, e.g., pathways from one or more operons or gene clusters or portions thereof. For example, bacteria and many eukaryotes have a coordinated mechanism for regulating genes whose products are involved in related processes. The genes are clustered, in structures referred to as “gene clusters,” on a single chromosome and are transcribed together under the control of a single regulatory sequence, including a single promoter which initiates transcription of the entire cluster. Thus, a gene cluster is a group of adjacent genes that are either identical or related, usually as to their function.

Gene cluster DNA can be isolated from different organisms and ligated into vectors, particularly vectors containing expression regulatory sequences which can control and regulate the production of a detectable protein or protein-related array activity from the ligated gene clusters. Use of vectors which have an exceptionally large capacity for exogenous DNA introduction are particularly appropriate for use with such gene clusters and are described by way of example herein to include the f-factor (or fertility factor) of E. coli. This f-factor of E. coli is a plasmid which affects high-frequency transfer of itself during conjugation and is ideal to achieve and stably propagate large DNA fragments, such as gene clusters from mixed microbial samples. “Fosmids,” cosmids or bacterial artificial chromosome (BAC) vectors can be used as cloning vectors. These are derived from E. coli f-factor which is able to stably integrate large segments of genomic DNA. When integrated with DNA from a mixed uncultured environmental sample, this makes it possible to achieve large genomic fragments in the form of a stable “environmental DNA library.” Cosmid vectors were originally designed to clone and propagate large segments of genomic DNA. Cloning into cosmid vectors is described in detail in Sambrook et al., Molecular Cloning: A Laboratory Manual, 2nd Ed., Cold Spring Harbor Laboratory Press (1989). Once ligated into an appropriate vector, two or more vectors containing different polyketide synthase gene clusters can be introduced into a suitable host cell. Regions of partial sequence homology shared by the gene clusters will promote processes which result in sequence reorganization resulting in a hybrid gene cluster. The novel hybrid gene cluster can then be screened for enhanced activities not found in the original gene clusters.

Thus, in one aspect, the invention relates to a method for producing a biologically active hybrid polypeptide using a nucleic acid of the invention and screening the polypeptide for an activity (e.g., enhanced activity) by:

(1) introducing at least a first polynucleotide (e.g., a nucleic acid of the invention) in operable linkage and a second polynucleotide in operable linkage, said at least first polynucleotide and second polynucleotide sharing at least one region of partial sequence homology, into a suitable host cell;

(2) growing the host cell under conditions which promote sequence reorganization resulting in a hybrid polynucleotide in operable linkage;

(3) expressing a hybrid polypeptide encoded by the hybrid polynucleotide;

(4) screening the hybrid polypeptide under conditions which promote identification of the desired biological activity (e.g., enhanced enzyme activity); and

(5) isolating the a polynucleotide encoding the hybrid polypeptide.

Methods for screening for various polypeptide (e.g., enzyme) activities are known to those of skill in the art and are discussed throughout the present specification. Such methods may be employed when isolating the polypeptides and polynucleotides of the invention.

In vivo reassortment can be focused on “inter-molecular” processes collectively referred to as “recombination.” In bacteria it is generally viewed as a “RecA-dependent” phenomenon. The invention can rely on recombination processes of a host cell to recombine and re-assort sequences, or the cells' ability to mediate reductive processes to decrease the complexity of quasi-repeated sequences in the cell by deletion. This process of “reductive reassortment” occurs by an “intra-molecular”, RecA-independent process. Thus, in one aspect of the invention, using the nucleic acids of the invention novel polynucleotides are generated by the process of reductive reassortment. The method involves the generation of constructs containing consecutive sequences (original encoding sequences), their insertion into an appropriate vector, and their subsequent introduction into an appropriate host cell. The reassortment of the individual molecular identities occurs by combinatorial processes between the consecutive sequences in the construct possessing regions of homology, or between quasi-repeated units. The reassortment process recombines and/or reduces the complexity and extent of the repeated sequences, and results in the production of novel molecular species.

Various treatments may be applied to enhance the rate of reassortment. These could include treatment with ultra-violet light, or DNA damaging chemicals, and/or the use of host cell lines displaying enhanced levels of “genetic instability”. Thus the reassortment process may involve homologous recombination or the natural property of quasi-repeated sequences to direct their own evolution.

Repeated or “quasi-repeated” sequences play a role in genetic instability. “Quasi-repeats” are repeats that are not restricted to their original unit structure. Quasi-repeated units can be presented as an array of sequences in a construct; consecutive units of similar sequences. Once ligated, the junctions between the consecutive sequences become essentially invisible and the quasi-repetitive nature of the resulting construct is now continuous at the molecular level. The deletion process the cell performs to reduce the complexity of the resulting construct operates between the quasi-repeated sequences. The quasi-repeated units provide a practically limitless repertoire of templates upon which slippage events can occur. The constructs containing the quasi-repeats thus effectively provide sufficient molecular elasticity that deletion (and potentially insertion) events can occur virtually anywhere within the quasi-repetitive units. When the quasi-repeated sequences are all ligated in the same orientation, for instance head to tail or vice versa, the cell cannot distinguish individual units. Consequently, the reductive process can occur throughout the sequences. In contrast, when for example, the units are presented head to head, rather than head to tail, the inversion delineates the endpoints of the adjacent unit so that deletion formation will favor the loss of discrete units. Thus, in one aspect of the invention, the sequences to be reassorted are in the same orientation. Random orientation of quasi-repeated sequences will result in the loss of reassortment efficiency, while consistent orientation of the sequences will offer the highest efficiency. However, while having fewer of the contiguous sequences in the same orientation decreases the efficiency, it may still provide sufficient elasticity for the effective recovery of novel molecules. Constructs can be made with the quasi-repeated sequences in the same orientation to allow higher efficiency.

Sequences can be assembled in a head to tail orientation using any of a variety of methods, including the following: a) Primers that include a poly-A head and poly-T tail which when made single-stranded would provide orientation can be utilized. This is accomplished by having the first few bases of the primers made from RNA and hence easily removed RNase H. b) Primers that include unique restriction cleavage sites can be utilized. Multiple sites, a battery of unique sequences, and repeated synthesis and ligation steps would be required. c) The inner few bases of the primer could be thiolated and an exonuclease used to produce properly tailed molecules.

The recovery of the re-assorted sequences relies on the identification of cloning vectors with a reduced repetitive index (R1). The re-assorted encoding sequences can then be recovered by amplification. The products are re-cloned and expressed. The recovery of cloning vectors with reduced R1 can be affected by: 1) The use of vectors only stably maintained when the construct is reduced in complexity. 2) The physical recovery of shortened vectors by physical procedures. In this case, the cloning vector would be recovered using standard plasmid isolation procedures and size fractionated on either an agarose gel, or column with a low molecular weight cut off utilizing standard procedures. 3) The recovery of vectors containing interrupted genes which can be selected when insert size decreases. 4) The use of direct selection techniques with an expression vector and the appropriate selection.

Encoding sequences (for example, genes) from related organisms may demonstrate a high degree of homology and encode quite diverse protein products. These types of sequences are particularly useful in the present invention as quasi-repeats. However, this process is not limited to such nearly identical repeats.

The following is an exemplary method of the invention. Encoding nucleic acid sequences (quasi-repeats) are derived from three (3) species, including a nucleic acid of the invention. Each sequence encodes a protein with a distinct set of properties, including a polypeptide, e.g., an enzyme, of the invention. Each of the sequences differs by a single or a few base pairs at a unique position in the sequence. The quasi-repeated sequences are separately or collectively amplified and ligated into random assemblies such that all possible permutations and combinations are available in the population of ligated molecules. The number of quasi-repeat units can be controlled by the assembly conditions. The average number of quasi-repeated units in a construct is defined as the repetitive index (R1). Once formed, the constructs may, or may not be size fractionated on an agarose gel according to published protocols, inserted into a cloning vector, and transfected into an appropriate host cell. The cells are then propagated and “reductive reassortment” is effected. The rate of the reductive reassortment process may be stimulated by the introduction of DNA damage if desired. Whether the reduction in R1 is mediated by deletion formation between repeated sequences by an “intra-molecular” mechanism, or mediated by recombination-like events through “inter-molecular” mechanisms is immaterial. The end result is a reassortment of the molecules into all possible combinations. In one aspect, the method comprises the additional step of screening the library members of the shuffled pool to identify individual shuffled library members having the ability to bind or otherwise interact, or catalyze a particular reaction (e.g., such as catalytic domain of an enzyme) with a predetermined macromolecule, such as for example a proteinaceous receptor, an oligosaccharide, virion, or other predetermined compound or structure. The polypeptides, e.g., enzymes, that are identified from such libraries can be used for various purposes, e.g., the industrial processes described herein and/or can be subjected to one or more additional cycles of shuffling and/or selection.

In another aspect, it is envisioned that prior to or during recombination or reassortment, polynucleotides generated by the method of the invention can be subjected to agents or processes which promote the introduction of mutations into the original polynucleotides. The introduction of such mutations would increase the diversity of resulting hybrid polynucleotides and polypeptides encoded therefrom. The agents or processes which promote mutagenesis can include, but are not limited to: (+)-CC-1065, or a synthetic analog such as (+)-CC-1065-(N3-Adenine (See Sun and Hurley, (1992); an N-acetylated or deacetylated 4′-fluoro-4-aminobiphenyl adduct capable of inhibiting DNA synthesis (See, for example, van de Poll et al. (1992)); or a N-acetylated or deacetylated 4-aminobiphenyl adduct capable of inhibiting DNA synthesis (See also, van de Poll et al. (1992), pp. 751-758); trivalent chromium, a trivalent chromium salt, a polycyclic aromatic hydrocarbon (PAH) DNA adduct capable of inhibiting DNA replication, such as 7-bromomethyl-benz[a]anthracene (“BMA”), tris(2,3-dibromopropyl)phosphate (“Tris-BP”), 1,2-dibromo-3-chloropropane (“DBCP”), 2-bromoacrolein (2BA), benzo[a]pyrene-7,8-dihydrodiol-9-10-epoxide (“BPDE”), a platinum(II) halogen salt, N-hydroxy-2-amino-3-methylimidazo[4,5-f]-quinoline (“N-hydroxy-IQ”), and N-hydroxy-2-amino-1-methyl-6-phenylimidazo[4,5-f]-pyridine (“N-hydroxy-PhIP”). Especially preferred means for slowing or halting PCR amplification consist of UV light (+)-CC-1065 and (+)-CC-1065-(N3-Adenine). Particularly encompassed means are DNA adducts or polynucleotides comprising the DNA adducts from the polynucleotides or polynucleotides pool, which can be released or removed by a process including heating the solution comprising the polynucleotides prior to further processing.

### Screening Methodologies and “On-Line” Monitoring Devices

In practicing the methods of the invention, a variety of apparatus and methodologies can be used to in conjunction with the polypeptides and nucleic acids of the invention, e.g., to screen polypeptides for enzyme activity, to screen compounds as potential modulators of activity (e.g., potentiation or inhibition of enzyme activity), for antibodies that bind to a polypeptide of the invention, for nucleic acids that hybridize to a nucleic acid of the invention, and the like.

Immobilized Polypeptide Solid Supports

The polypeptides, fragments thereof and nucleic acids that encode the enzymes and fragments can be affixed to a solid support. This is often economical and efficient in the use of polypeptides, e.g., antibodies, enzymes, ligands, in industrial processes. For example, a consortium or cocktail of polypeptides (e.g., enzymes or active fragments thereof), which are used in a specific chemical reaction, can be attached to a solid support and dunked into a process vat. The enzymatic reaction can occur. Then, the solid support can be taken out of the vat, along with the enzymes affixed thereto, for repeated use. In one embodiment of the invention, an isolated nucleic acid of the invention is affixed to a solid support. In another embodiment of the invention, the solid support is selected from the group of a gel, a resin, a polymer, a ceramic, a glass, a microelectrode and any combination thereof.

For example, solid supports useful in this invention include gels. Some examples of gels include Sepharose, gelatin, glutaraldehyde, chitosan-treated glutaraldehyde, albumin-glutaraldehyde, chitosan-Xanthan, toyopearl gel (polymer gel), alginate, alginate-polylysine, carrageenan, agarose, glyoxyl agarose, magnetic agarose, dextran-agarose, poly(Carbamoyl Sulfonate) hydrogel, BSA-PEG hydrogel, phosphorylated polyvinyl alcohol (PVA), monoaminoethyl-N-aminoethyl (MANA), amino, or any combination thereof.

Another solid support useful in the present invention are resins or polymers. Some examples of resins or polymers include cellulose, acrylamide, nylon, rayon, polyester, anion-exchange resin, AMBERLITE™ XAD-7, AMBERLITE™ XAD-8, AMBERLITE™ IRA-94, AMBERLITE™ IRC-50, polyvinyl, polyacrylic, polymethacrylate, or any combination thereof.

Another type of solid support useful in the present invention is ceramic. Some examples include non-porous ceramic, porous ceramic, SiO₂, Al₂O₃. Another type of solid support useful in the present invention is glass. Some examples include non-porous glass, porous glass, aminopropyl glass or any combination thereof. Another type of solid support that can be used is a microelectrode. An example is a polyethyleneimine-coated magnetite. Graphitic particles can be used as a solid support.

Another example of a solid support is a cell, such as a red blood cell.

Methods of Immobilization

There are many methods that would be known to one of skill in the art for immobilizing polypeptides (e.g., antibodies, enzymes, binding proteins, or fragments thereof), or nucleic acids, onto a solid support. Some examples of such methods include, e.g., electrostatic droplet generation, electrochemical means, via adsorption, via covalent binding, via cross-linking, via a chemical reaction or process, via encapsulation, via entrapment, via calcium alginate, or via poly(2-hydroxyethyl methacrylate). Like methods are described in Methods in Enzymology, Immobilized Enzymes and Cells, Part C. 1987. Academic Press. Edited by S. P. Colowick and N, O. Kaplan. Volume 136; and Immobilization of Enzymes and Cells. 1997. Humana Press. Edited by G. F. Bickerstaff. Series: Methods in Biotechnology, Edited by J. M. Walker.

Capillary Arrays

Capillary arrays, such as the GIGAMATRIX™, Diversa Corporation, San Diego, Calif., can be used to in the methods of the invention. Nucleic acids or polypeptides of the invention can be immobilized to or applied to an array, including capillary arrays. Arrays can be used to screen for or monitor libraries of compositions (e.g., small molecules, antibodies, nucleic acids, etc.) for their ability to bind to or modulate the activity of a nucleic acid or a polypeptide of the invention. Capillary arrays provide another system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or nucleic acid, e.g., a ligand, can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a microtiter plate having about 100,000 or more individual capillaries bound together.

Arrays, or “BioChips”

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a polypeptide-encoding (e.g., an enzyme) gene. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. “Polypeptide arrays” can also be used to simultaneously quantify a plurality of proteins.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

### Antibodies and Antibody-Based Screening Methods

The invention provides isolated or recombinant antibodies that specifically bind to a polypeptide (e.g., an enzyme) of the invention. These antibodies can be used to isolate, identify or quantify the polypeptides of the invention or related polypeptides. These antibodies can be used to inhibit the activity of a polypeptide (e.g., an enzyme) of the invention. These antibodies can be used to isolated polypeptides related to those of the invention, e.g., related polypeptides.

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention.

Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

The polypeptides can be used to generate antibodies which bind specifically to the polypeptides of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to an animal, for example, a nonhuman. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention may be used in screening for similar polypeptides from other organisms and samples. In such techniques, polypeptides from the organism are contacted with the antibody and those polypeptides which specifically bind the antibody are detected. Any of the procedures described above may be used to detect antibody binding.

### Kits

The invention provides kits comprising the compositions, e.g., nucleic acids, expression cassettes, vectors, cells, polypeptides (e.g., enzymes) and/or antibodies of the invention. The kits also can contain instructional material teaching the methodologies and industrial uses of the invention, as described herein.

The invention will be further described with reference to the following examples; however, it is to be understood that the invention is not limited to such examples.

### EXAMPLES

### Example 1

### Blast Program Used for Sequence Identify Profiling

This example describes an exemplary sequence identity program to determine if a nucleic acid is within the scope of the invention. An NCBI BLAST 2.2.2 program is used, default options to blastp. All default values were used except for the default filtering setting (i.e., all parameters set to default except filtering which is set to OFF); in its place a “-F F” setting is used, which disables filtering. Use of default filtering often results in Karlin-Altschul violations due to short length of sequence. The default values used in this example:
“Filter for low complexity: ON
>Word Size: 3
>Matrix: Blosum62
>Gap Costs: Existence: 11
>Extension: 1”

Other default settings were: filter for low complexity OFF, word size of 3 for protein, BLOSUM62 matrix, gap existence penalty of −11 and a gap extension penalty of −1. The “-W” option was set to default to 0. This means that, if not set, the word size defaults to 3 for proteins and 11 for nucleotides. The settings read:

<<README.bls.txt>>

| | |
|---|---|
| > | -------------------------------------------------------------------------- |
| > | blastall arguments: |
| > | |
| > | -p Program Name [String] |
| > | -d Database [String] |
| > | default = nr |
| > | -i Query File [File In] |
| > | default = stdin |
| > | -e Expectation value (E) [Real] |
| > | default = 10.0 |
| > | -m alignment view options: |
| > | 0 = pairwise, |
| > | 1 = query-anchored showing identities, |
| > | 2 = query-anchored no identities, |
| > | 3 = flat query-anchored, show identities, |
| > | 4 = flat query-anchored, no identities, |
| > | 5 = query-anchored no identities and blunt ends, |
| > | 6 = flat query-anchored, no identities and blunt ends, |
| > | 7 = XML Blast output, |
| > | 8 = tabular, |
| > | 9 tabular with comment lines [Integer] |
| > | default = 0 |
| > | -o BLAST report Output File [File Out] Optional |
| > | default = stdout |
| > | -F Filter query sequence (DUST with blastn, SEG with others) |
| | [String] |
| > | default = T |
| > | -G Cost to open a gap (zero invokes default behavior) [Integer] |
| > | default = 0 |
| > | -E Cost to extend a gap (zero invokes default behavior) [Integer] |
| > | default = 0 |
| > | -X X dropoff value for gapped alignment (in bits) (zero invokes |
| > | default behavior) [Integer] |
| > | default = 0 |
| > | -I Show GI's in deflines [T/F] |
| > | default = F |
| > | -q Penalty for a nucleotide mismatch (blastn only) [Integer] |
| > | default = −3 |
| > | -r Reward for a nucleotide match (blastn only) [Integer] |
| > | default = 1 |
| > | -v Number of database sequences to show one-line descriptions for |
| | (V) |
| > | [Integer] |
| > | default = 500 |
| > | -b Number of database sequence to show alignments for (B) |
| | [Integer] |
| > | default = 250 |
| > | -f Threshold for extending hits, default if zero [Integer] |
| > | default = 0 |
| > | -g Perform gapped alignment (not available with tblastx) [T/F] |
| > | default = T |
| > | -Q Query Genetic code to use [Integer] |
| > | default = 1 |
| > | -D DB Genetic code (for tblast[nx] only) [Integer] |
| > | default = 1 |
| > | -a Number of processors to use [Integer] |
| > | default = 1 |
| > | -O SeqAlign file [File Out] Optional |
| > | -J Believe the query defline [T/F] |
| > | default = F |
| > | -M Matrix [String] |
| > | default = BLOSUM62 |
| > | -W Word size, default if zero [Integer] |
| > | default = 0 |
| > | -z Effective length of the database (use zero for the real size) |
| > | [String] |
| > | default = 0 |
| > | -K Number of best hits from a region to keep (off by default, if |
| > | used a value of 100 is recommended) [Integer] |
| > | default = 0 |
| > | -P 0 for multiple hits 1-pass, 1 for single hit 1-pass, 2 for 2-pass |
| > | [Integer] |
| > | default = 0 |
| > | -Y Effective length of the search space (use zero for the real size) |
| > | [Real] |
| > | default = 0 |
| > | -S Query strands to search against database (for blast[nx], and |
| > | tblastx). 3 is both, 1 is top, 2 is bottom [Integer] |
| > | default = 3 |
| > | -T Produce HTML output [T/F] |
| > | default = F |
| > | -l Restrict search of database to list of GI's [String] Optional |
| > | -U Use lower case filtering of FASTA sequence [T/F] Optional |
| > | default = F |
| > | -y Dropoff (X) for blast extensions in bits (0.0 invokes default |
| > | behavior) [Real] |
| > | default = 0.0 |
| > | -Z X dropoff value for final gapped alignment (in bits) [Integer] |
| > | default = 0 |
| > | -R PSI-TBLASTN checkpoint file [File In] Optional |
| > | -n MegaBlast search [T/F] |
| > | default = F |
| > | -L Location on query sequence [String] Optional |
| > | -A Multiple Hits window size (zero for single hit algorithm) |
| | [Integer] |
| > | default = 40 |

### Example 2

### Isolation and Characterization of the Hyperthermophile Nanoarchaeum equitans and its Genome

This example describes the isolation and characterization of the hyperthermophile Nanoarchaeum equitans of the invention and its genome (SEQ ID NO:1).

To investigate hot submarine vent microbial communities, experiments were carried out to cultivate hyperthermophiles from samples of originally hot rocks and gravel taken at the Kolbeinsey ridge, north of Iceland, see, e.g., Fricke (1989) “Hydrothermal vent communities at the shallow subpolar mid-Atlantic ridge.” Mar. Biol. 102, 425-429. By anaerobic incubation at 90° C. in the presence of S, H2 and CO₂, a new autotrophic sulphur-reducing species of the archaeal genus Ignicoccus could be enriched. In contrast to known species, several large spherical Ignicoccus cells were covered by very tiny cocci which could be stained by DNA-specific fluorescence microscopy, using DAPI, see, e.g., Huber (1985) Syst. Appl. Microbiol. 6:105-106. Few such tiny cocci existed in the free state. These tiny cocci could be physically isolated either by using ‘optical tweezers’ (see, e.g., Ashkin (1987) Nature 330:769-771; Huber (1995) Nature 376:57-58; or, or by ultrafiltration, pore width, 0.45 mm. All attempts to grow these organisms in pure culture employing various inorganic and organic energy sources failed. However, isolation of a combination of a tiny coccus attached to an Ignicoccus sphere after incubation under enrichment conditions, resulted in a defined co-culture in which about half of the Ignicoccus cells appeared to be colonized by the tiny cocci. The final density of both the tiny cocci and the Ignicoccus cells was about 3×10⁷ cells ml⁻¹ resulting in about two tiny cocci per Ignicoccus cell on average. The purified co-culture was used in all further investigations.

Cloning of single Ignicoccus cells gave rise to cultures which never contained tiny cocci. By electron microscopy, a close attachment of the tiny cocci to the surface of the Ignicoccus cells became evident. The tiny cocci consistently exhibited a cell diameter of about 400 nm. In contrast to Ignicoccus, they were covered by a regular surface layer (S-layer) with six-fold symmetry and a lattice constant of 15 nm. Ultra-thin sections showed the presence of cytoplasmic membranes inside the tiny cocci and the Ignicoccus cells. No specific attachment structures could be detected at the sites of contact which suggested a loose connection of the tiny cocci with the Ignicoccus cells. In line with this assumption, the tiny cocci could be removed by mild sonification (30W).

The phylogenetic relationships of the tiny cocci were investigated by ss rRNA sequence comparisons (see, e.g., Woese (1987) Microbiol. Rev. 51:221-271). Total DNA of the co-culture was extracted and ss rRNA genes were PCR-amplified using primers addressed to highly conserved gene sections. Surprisingly, however, only the Ignicoccus ss rRNA gene sequence was amplified even when primers considered general for all Archaea as well as for all organisms (‘universal’) had been employed. More directly, the co-culture-derived DNA for ss rRNA genes was examined by Southern blot hybridization (see, e.g., Sambrook, J. Molecular cloning: a laboratory manual. (Cold Spring Harbor Laboratory Press, New York, 1989), taking advantage of the generally high sequence homology of all ss rRNA genes. Two different hybridization signals became visible, indicating two non-identical ss rRNA genes. As a control, DNA isolated from the separate Ignicoccus sp. by the same procedure yielded only one hybridization signal which was identical with one from the mixed culture. Therefore, the tiny cocci must have given rise to the second hybridization signal, indicating that they hold a single-stranded (ss)rRNA gene different from Ignicoccus.

The corresponding DNA fragment was cloned and sequenced. Its sequence turned out to be unique, harboring many base exchanges even in the so-called ‘highly conserved regions’ that are usually employed as primer targets for ss rDNA PCR. This explains our initial failure to amplify this gene by PCR. Comparison of the new single-stranded (ss)rRNA sequence with those of other microorganisms established the tiny cocci as previously unknown members of the archaeal domain indicated by sequence identities of 0.69 to 0.81 with the Archaea in contrast to sequence identities of only 0.59 to 0.70 with bacterial species.

The secondary structure of the ss rRNA sequence conformed to the standard two-dimensional structural model (see, e.g., Woese (1987) supra). It exhibited features characteristic of Archaea, especially helices numbers 17, 18, 36 and 47. The sequence identities to the Crenarchaeota (0.73-0.81), the Euryarchaeota (0.69-0.81), and the ‘Korarchaeota’ (0.73-0.75) were in the same range as those between these three known phyla of Archaea (0.69-0.83). Therefore, the tiny cocci represent a new archaeal phylum. On the basis of its extremely small cell size, we named it ‘Nanoarchaeota’ (the dwarf archaea) and the corresponding species ‘Nanoarchaeum equitans’ (riding the fire sphere).

In ss rRNA phylogenetic trees that are founded on the maximum parsimony, distance matrix, and maximum likelihood methods (see, e.g., Ludwig (1998) supra), ‘N. equitans’ represents an isolated, very deeply branching lineage. However, because of its unique ss rRNA, a large variation of its branching point, challenged by insignificant bootstrap values below 50%, was observed. Therefore, the determination of the accurate branching position of the ‘Nanoarchaeota’ must await the discovery of further members of this group. Owing to their great divergences in ss rRNA, in contrast to the Ignicoccus host, cells of the ‘Nanoarchaeota’ did not stain by fluorescence in situ hybridization using ss rRNA-targeted oligonucleotide probes directed against Crenarchaeota and Euryarchaeota). However, after redesigning these oligonucleotide probes on the basis of the ‘Nanoarchaeota’ sequence (SEQ ID NO:1), the tiny cocci exhibited a bright fluorescence, indicating that they possessed ribosomal RNA harboring the target sequence.

Few physiological properties of the ‘Nanoarchaeum’ strain are known thus far. Isolated cells did not grow on cell homogenates of Ignicoccus sp., so they require an actively growing Ignicoccus culture. Within pure cultures of Ignicoccus, ‘Nanoarchaeum’ cells locally separated by dialysis bags were unable to propagate. Therefore, a direct cell-cell contact with the host appears to be a prerequisite for growth. No negative influence on Ignicoccus, such as slower growth, lower final cell density, or increased cell lysis, could be observed in the co-culture compared to the pure culture, rendering a parasitic mode of living of ‘Nanoarchaeum’ unlikely. Therefore, the organism is most probably symbiotic. So far, archaeal symbionts are only known at mesophilic temperatures in association with eukaryotes (see, e.g., Preston (1996) Proc. Natl. Acad. Sci. USA 93, 6241-6246) and bacteria (see, e.g., Boetius (2000) Nature 407:623-626). Co-cultures of ‘Nanoarchaeum’ grew within the same temperature range (70° C. to 98° C.) as its host. During mass-culturing in a 300-litre enamel-protected fermentor3, final cell concentrations of ‘Nanoarchaeum’ could be improved about ten-fold by an increased gassing rate (20 liter/min; H₂:CO₂=80:20), while that of Ignicoccus remained unchanged. This procedure improved hydrogen supply and efficiently removed H₂S, the main metabolic end product of Ignicoccus. During the late exponential growth phase, about 80% of the ‘Nanoarchaeum’ cells detached from their host cells and occurred freely in suspension. They could be collected (for example, 1.5 g wet weight) by high-speed centrifugation, after removal of the Ignicoccus cells at low speed. By this method, cell masses of ‘Nanoarchaeum’ became available for biochemical and molecular investigations.

A first analysis of its genome size by adding up the sizes of restriction fragments determined after pulse field gel electrophoresis resulted in only 500 kilobases (kb), one of the smallest genomes of all prokaryotes known so far. With its tiny cell and genome size, ‘Nanoarchaeum’ resembles an intermediate between the smallest living organisms like Mycoplasma genitalium (see, e.g., Fraser (1995) Science 270:397-403) and big viruses like the pox virus and Chlorella virus CVK2 (see, e.g., Rohozinski (1989) Virology 168:363-369; Murphy, F. A. et al. (eds) Virus Taxonomy: Sixth Report of the International Committee on Taxonomy of Viruses, Springer, Vienna/New York, 1995. Nature Vol. 417. 2 MAY 2002), and is close to the theoretical minimum genome size calculated for a living being (see, e.g., Hutchinson (1999) Science 286:2165-2169).

Small cell size and reduced genomes are common features observed in many symbiotic or parasitic bacteria, but have so far not been reported for Archaea or hyperthermophiles. No final conclusions can be drawn about a primitive or derived state of evolution of ‘Nanoarchaeum’. However, its high growth temperature and anaerobic mode of life correlates with probable early environmental conditions which suggest that the ‘Nanoarchaeota’ are possibly still a primitive form of microbial life.

Cultivation conditions: Cultures were grown anaerobically in serum bottles as described by Huber (2000) Int. J. Syst. Evol. Microbiol. 50:2093-2100. Mass culturing was carried out in the same medium in a 300-litre enamel-protected fermentor (gassing rate routinely 2 liter/min H₂:CO₂=80:20). Cell mixtures were collected by centrifugation (9,000 r.p.m.; 30 min using rotor GS3 (Sorvall)) and resuspended in sulphur-free culture medium. The Ignicoccus cells were removed by centrifugation at 2,000 r.p.m. for 30 min using rotor SS34 (Sorvall). Then the ‘Nanoarchaeum’ cells were precipitated at 15,000 r.p.m. for 15 min using rotor SS34. Axenic cultivation of ‘Nanoarchaeum equitans’ was tested autotrophically in the presence of H₂:CO₂ (80:20) and elemental sulphur, nitrate, nitrite, and sulphate (each 0.1% w/v) as possible electron acceptors, and heterotrophically on single and complex organic nutrients such as sugars, amino acids, yeast extract, peptone, bacterial and archaeal (for example, from Ignicoccus) cell extracts (each 0.1% w/v; gas phase: H₂:CO₂=80:20 and N₂:CO₂=80:20).

Light and electron microscopy: Phase-contrast and electron microscopy were carried out as described by Huber (2000) supra. Confocal image series were recorded on a LEICA TCS SP2 laser scanning microscope using the laser wavelengths 488 nm (argon) and 543 nm (HeNe) excitation. Fluorescence in situ hybridization (FISH) was performed as described15. CY3- and rhodamine-green-labeled oligonucleotides were obtained from Metabion (Germany).

Southern blot experiments: DNAs were digested with restriction endonucleases EcoRI and HindIII. The fragments were separated by electrophoresis (1% Seakem ME agarose) and transferred to a positively charged nylon membrane by Southern blot13. The membrane was hybridized with a digoxigenine-labelled single-stranded DNA fragment of an archaeal ss rRNA gene (Metallosphaera sedula; length 600 nucleotides, position 519 to 1119, Escherichia coli).

Estimation of the genome size: For pulse field gel electrophoresis ‘Nanoarchaeum’ cells were embedded in 100-ml agarose plugs (0.8% Incert agarose, FMC; final concentration: 3£108 cells per plug). After cell lysis according to lysis method ‘three’ (see, e.g., Baumann (1998) Extremophiles 2:101-108) genomic DNA was digested using the restriction enzymes AscI, BssHII, NotI, or SacII (each 100 units, 18 h). The restriction fragments were separated as described by Baumann (1998) supra.

Small subunit rDNA sequence analysis: The nearly complete ss rRNA gene of the new Ignicoccus species was PCR-amplified and sequenced as described by Huber (2000) supra. To obtain the ss rRNA gene of ‘N. equitans’, genomic DNA of the co-culture was digested with EcoRI and separated as described (see ‘Southern blot’). DNA fragments of the correct size, identified by Southern blot, were extracted, cloned into a plasmid (pBluescript SK(2)) and amplified in Escherichia coli (strain Dh5a). These inserts were sequenced by primer walking. The new sequences were aligned with approximately 10,000 homologous sequences available in public databases using the automatic alignment tools of the ARB package (Technical University of Munich, Del.). Distance matrix (Jukes-Cantor correction), maximum parsimony, and maximum likelihood (fastDNAml) methods were used for tree reconstruction as implemented in the ARB package. The secondary structure of the ss rRNA of ‘N. equitans’ was displayed using the program RnaViz28.

Library Construction and DNA Sequencing: N. equitans was grown in a 300 liter fermentor in a co-culture with Ignicoccus sp. and the N. equitans cells were purified from Ignicoccus as described above. The cell pellet was lysed by enzymatic and chemical digestion, followed by the isolation and purification of genomic DNA, as described by Zhou (1996) Appl. Environ. Microbiol. 62:316; Robertson (1996) Soc. Indust. Microbiol. News 46:3; Short (1997) Nat. Biotechnol. 15:1322. Genomic DNA was either digested with restriction enzymes or sheared to provide clonable fragments. Two plasmid libraries were made by subcloning randomly sheared fragments of this DNA into a high-copy number vector (˜2.8 kbp library) or low-copy number vector (˜6.3 kbp library). DNA sequence was obtained from both ends of the plasmid inserts to create ‘mate-pairs’—a pair of reads from a single clone that should be adjacent to one another in the genome. Library construction, DNA sequencing and assembly methods were essentially as described by Adams (2000) Science 287:2185; Myers (2000) Science 287:2196; Venter (2001) Science 291:1304. The assembly procedures resulted in a single scaffold of four contigs comprising 489,082 base pairs. The gaps between the four contigs were then sequenced with 10× coverage which resulted in a single circular sequence. A set of computational methods was applied to the N. equitans genome of the invention (SEQ ID NO:1). Two gene prediction programs, Glimmer (see, e.g., Delcher (1999) Nucleic Acids Res. 27:4636) and Critica (see, e.g., Badger (1999) Mol. Biol. Evol. 16:4512), were run on the assembled sequences. The results of the two programs were merged to generate a unique set of genes. When the two programs selected different start codons for genes with the same stop codon, the longer gene was included in the set for further analysis. This unique set of genes was then translated into amino acid sequences and subjected to BlastP searches (with an E-value cutoff of 1e-10) against the non-redundant amino acid (nraa) protein database, as described by the NCBI, NLM, NIH website. The predicted protein set was then searched against the InterPro database release 3.1 (see, e.g., Apweiler (2001) Nucleic Acids Res. 29, 37) using software modified from the original ipr scan programs provided by InterPro (The European Bioinformatics Institute (EBI), European Molecular Biology Laboratory (EMBL), Cambridge, UK). The predicted protein set was also searched against the NCBI Clusters of Othologous Groups (COGs) database mid-2001 update (see, e.g., Tatusov (2001) Nucleic Acids Res. 29:22). Lastly, gene family analysis was performed using the NCBI blast clust program. tRNA genes were identified by the tRNA scan-SE program (see, e.g., Lowe (1997) Nucleic Acids Res. 25:955) and rRNAs were identified by searching the genomic sequences against a set of known rRNAs with BlastN and verified by profile alignment to the multiple alignments from known rRNA sequences. Protein sets from the main scaffold and small scaffolds were compared to the protein sequences from all finished genomes deposited in the GenBank using the blast program.

Preparation of alanyl-tRNA synthetases and aminoacylation assay: The methods were adapted from Ahel et al., as described in Ahel (2002) J. Biol. Chem. 277:34743. N. equitans alaS1 (NEQ547), N. equitans alaS2 (NEQ211), and M. jannaschii alaS genes were PCR amplified from the respective genomic DNA and cloned into the pCR2.1 TOPO vector. Correct sequences were subsequently re-cloned into pET11a (Invitrogen) for expression of the proteins in the E. coli BL21-Codon Plus (DE3)-RIL strain. Cultures were grown at 37° C. in Luria-Bertani medium supplemented with 100 μg/ml ampicillin and 34 μg/ml chloramphenicol. Expression of the recombinant proteins was induced for 3 hours (h) at 30° C. by addition of 1 mM isopropyl-1-thio-D-galactopyranoside before cell harvesting. Cells were resuspended in buffer containing 50 mM Tris-HCl, pH 7.5, and 300 mM NaCl, and broken by sonication. S-100 fractions were extensively flocculated at 70° C. for 45 min, and then centrifuged for 30 min at 20,000×g. Supernatants were collected and stored at 4° C. before use in aminoacylation assays.

Aminoacylation was performed in a 0.1 ml reaction at 70° C. in 50 mM HEPES, pH 7.2, 50 mM KCl, 10 mM ATP, 50 μM [³H] alanine (52 Ci/ml), 15 mM MgCl₂ and 5 mM 2-mercaptoethanol, unfractionated M. jannaschii tRNA (3 mg per ml of reaction) and the different alanyl-tRNA synthetases (100 nM). Aliquots of 20 μl were removed at the time intervals indicated in FIG. 5 and radioactivity measured as described. FIG. 5 summarizes data from the alanylation of unfractionated M. jannaschii tRNA by alanyl-tRNA synthetase. The purification and aminoacylation procedures were adapted from Ahel (2002) J. Biol. Chem. 277:34743. The enzymes used are: M. jannaschii AlaRS (filled squares), N. equitans AlaRS1—N-terminal part (open circles), N. equitans AlaRS2—C-terminal part (filled triangles), N. equitans AlaRS1+AlaRS2 (filled circles).

Phylogenetic Analysis: A concatenated alignment of 35 ribosomal proteins was obtained from Matte-Tailliez (see e.g., Matte-Tailliez (2002) Mol. Biol. Evol. 13:631). To this alignment we added the N. equitans, Methanopyrus kandleri and the eukaryotic outgroup sequences (Arabidopsis thaliana and Saccharomyces cerevisiae). The alignment was then recalculated with ClustalW (see, e.g., Thompson (1994) Nucleic Acids Res. 22:4673) and optimized by hand using BioEdit (Hall (1999) Nucleic Acids Symp. Ser. 41:95; North Carolina State University, Raleigh, N.C.). The program RASA (University of Massachusetts, Lowell, Mass.) was used to evaluate the alignment for the presence of long branches (see, e.g., Lyons-Weiler (1996) Mol. Biol. Evol. 13:749). Maximum likelihood analysis was performed with Protml from PHYLIP v. 3.6a2.3 (University of Washington, Seattle, Wash.). Parameters used were: the Jones-Taylor-Thornton model, rate variation among sites, constant rate of change, global rearrangements, randomized input order of sequences with three jumbles. One hundred bootstrap resamplings were performed to assess the support for individual branches. Bayseian analysis of the dataset was done with the MRBAYES software (University of California, San Diego, Calif.; Huelsenbeck (2001) Bioinformatics 17:754). Four simultaneous MCMC chains were run for two hundred thousand generations after the convergence of the likelihood values, using the default settings of the program. A 50% majority-rule consensus tree was generated based on the resulted 2000 trees and the bipartition values (percentage representation of a particular clade) were recorded at the nodes. The program PAUP 4.*™ (see, e.g., Swafford (1996) PAUP*. Sinauer Associates, Sunderland, Mass.) was used for the parsimony analysis. The alignment was sampled for 500 bootstrap replicates. Each bootstrap replicate was analyzed with ten random addition sequence replicates with TBR branch swapping and equal weighting for all sites.

1. An isolated or recombinant nucleic acid comprising a nucleic acid sequence having at least 50% sequence identity to SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, over a region of at least about 100 residues, wherein the nucleic acid encodes at least one polypeptide having an enzyme, structural or binding activity, and the sequence identities are determined by analysis with a sequence comparison algorithm or by a visual inspection.

2-7. (canceled)

8. The isolated or recombinant nucleic acid of claim 1, wherein the enzyme, structural or binding activity comprises a recombinase activity, a helicase activity, a DNA replication activity, a DNA recombination activity, an isomerase, a transisomerase activity, topoisomerase activity, a methyl transferase activity, an aminotransferase activity, a uracil-5-methyl transferase activity, a cysteinyl tRNA synthetase activity, a hydrolase, an esterase activity, a phosphoesterase activity, an acetylmuramyl pentapeptide phosphotransferase activity, a glycosyltransferase activity, an acetyltransferase activity, an acetylglucosamine phosphate transferase activity, a centromere binding activity, a telomerase activity, a transcriptional regulatory activity, a heat shock protein activity, a protease activity, a proteinase activity, a peptidase activity, a carboxypeptidase activity, an endonuclease activity, an exonuclease activity, a RecB family exonuclease activity, a polymerase activity, a carbamoyl phosphate synthetase activity, a methyl-thioadenine synthetase activity, an oxidoreductase activity, an Fe—S oxidoreductase activity, a flavodoxin reductase activity, a permease activity, a thymidylate activity, a dehydrogenase activity, a pyrophosphorylase activity, a coenzyme metabolism activity, a dinucleotide-utilizing enzyme activity, a molybdopterin or thiamine biosynthesis activity, a beta-lactamase activity, a ligand binding activity, an ion transport activity, an ion metabolism activity, a tellurite resistance protein activity, an inorganic ion transport activity, a nucleotide transport activity, a nucleotide metabolism activity, an actin, a myosin activity, a lipase activity or a lipid acyl hydrolase (LAH) activity, a cell envelop biogenesis activity, an outer membrane synthesis activity, a ribosomal structure synthesis activity, a translational processing activity, a transcriptional initiation activity, a TATA-binding activity, a signal transduction activity, an energy metabolism activity, an ATPase activity, an information storage activity, a processing activity, or a combination thereof.

9-23. (canceled)

24. An isolated or recombinant nucleic acid, wherein the nucleic acid comprises a sequence that hybridizes under stringent conditions to a nucleic acid comprising SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, wherein the nucleic acid encodes a polypeptide having an enzyme, structural or binding activity.

25-26. (canceled)

27. A nucleic acid probe for identifying a nucleic acid encoding a polypeptide with enzyme, structural or binding activity, wherein the probe comprises at least 10 consecutive bases of a sequence comprising SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, wherein the probe identifies the nucleic acid by binding or hybridization.

28. (canceled)

29. A nucleic acid probe for identifying a nucleic acid encoding a polypeptide having an enzyme, structural or binding activity, wherein the probe comprises a nucleic acid comprising at least about 10 consecutive residues of SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, wherein the sequence identities are determined by analysis with a sequence comparison algorithm or by visual inspection.

30. (canceled)

31. An amplification primer sequence pair for amplifying a nucleic acid encoding a polypeptide having an enzyme, structural or binding activity, wherein the primer pair is capable of amplifying a nucleic acid comprising a sequence as set forth in claim 1, or a subsequence thereof.

32. (canceled)

33. An amplification primer pair, wherein the primer pair comprises a first member having a sequence as set forth by about the first (the 5′) 12, 13, 14, 15, 16, 17, 18, 19, 20, 21, 22, 23, 24, 25, 26, 27, 28, 29, 30 or more residues of SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, and a second member having a sequence as set forth by about the first (the 5′) 12, 13, 14, 15, 16, 17, 18, 19, 20, 21, 22, 23, 24, 25, 26, 27, 28, 29 or 30 or more residues of the complementary strand of the first member.

34. A polypeptide-encoding nucleic acid generated by amplification of a polynucleotide using an amplification primer pair as set forth in claim 33.

35-37. (canceled)

38. An isolated or recombinant polypeptide encoded by a nucleic acid as set forth in claim 34.

39. A method of amplifying a nucleic acid encoding a polypeptide having an enzyme, structural or binding activity comprising amplification of a template nucleic acid with an amplification primer sequence pair capable of amplifying a nucleic acid sequence as set forth in claim 1, or a subsequence thereof.

40. A method for making an polypeptide comprising amplification of a nucleic acid with an amplification primer pair as set forth in claim 33 and expression of the amplified nucleic acid.

41. An expression cassette comprising a nucleic acid comprising a sequence as set forth in claim 1.

42. A vector comprising a nucleic acid comprising a sequence as set forth in claim 1.

43. A cloning vehicle comprising a nucleic acid comprising a sequence as set forth in claim 1, wherein the cloning vehicle comprises a viral vector, a plasmid, a phage, a phagemid, a cosmid, a fosmid, a bacteriophage or an artificial chromosome.

44-45. (canceled)

46. A transformed cell comprising a nucleic acid comprising a sequence as set forth in claim 1.

47-48. (canceled)

49. A transgenic non-human animal comprising a sequence as set forth in claim 1.

50. (canceled)

51. A transgenic plant comprising a sequence as set forth in claim 1.

52. (canceled)

53. A transgenic seed comprising a sequence as set forth in claim 1.

54. (canceled)

55. An antisense oligonucleotide comprising a nucleic acid sequence complementary to or capable of hybridizing under stringent conditions to a sequence as set forth in claim 1, or a subsequence thereof.

56. (canceled)

57. A method of inhibiting the translation of an polypeptide message in a cell comprising administering to the cell or expressing in the cell an antisense oligonucleotide comprising a nucleic acid sequence complementary to or capable of hybridizing under stringent conditions to a sequence as set forth in claim 1.

58. A double-stranded inhibitory RNA (RNAi) molecule comprising a subsequence of a sequence as set forth in claim 1.

59. (canceled)

60. A method of inhibiting the expression of an polypeptide in a cell comprising administering to the cell or expressing in the cell a double-stranded inhibitory RNA (iRNA), wherein the RNA comprises a subsequence of a sequence as set forth in claim 1.

61. An isolated or recombinant polypeptide (i) having at least 50% sequence identity to SEQ ID NO:3, SEQ ID NO:5, SEQ ID NO:7, SEQ ID NO:9, SEQ ID NO:11, SEQ ID NO:13, SEQ ID NO:15; SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, and all polypeptides disclosed in the SEQ ID listing, which include all odd numbered SEQ ID NO:s from SEQ ID NO:3 through SEQ ID NO:1073, over a region of at least about 100 residues, wherein the sequence identities are determined by analysis with a sequence comparison algorithm or by a visual inspection, or, (ii) encoded by a nucleic acid having at least 50% sequence identity to a sequence as set forth in SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, over a region of at least about 100 residues, and the sequence identities are determined by analysis with a sequence comparison algorithm or by a visual inspection, or encoded by a nucleic acid capable of hybridizing under stringent conditions to a sequence as set forth in SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073.

62-64. (canceled)

65. The isolated or recombinant polypeptide of claim 61, wherein the polypeptide has an enzyme, structural or binding activity.

66. The isolated or recombinant polypeptide of claim 65, wherein the enzyme, structural or binding activity comprises a recombinase activity, a helicase activity, a DNA replication activity, a DNA recombination activity, an isomerase, a transisomerase activity, topoisomerase activity, a methyl transferase activity, an aminotransferase activity, a uracil-5-methyl transferase activity, a cysteinyl tRNA synthetase activity, a hydrolase, an esterase activity, a phosphoesterase activity, an acetylmuramyl pentapeptide phosphotransferase activity, a glycosyltransferase activity, an acetyltransferase activity, an acetylglucosamine phosphate transferase activity, a centromere binding activity, a telomerase activity, a transcriptional regulatory activity, a heat shock protein activity, a protease activity, a proteinase activity, a peptidase activity, a carboxypeptidase activity, an endonuclease activity, an exonuclease activity, a RecB family exonuclease activity, a polymerase activity, a carbamoyl phosphate synthetase activity, a methyl-thioadenine synthetase activity, an oxidoreductase activity, an Fe—S oxidoreductase activity, a flavodoxin reductase activity, a permease activity, a thymidylate activity, a dehydrogenase activity, a pyrophosphorylase activity, a coenzyme metabolism activity, a dinucleotide-utilizing enzyme activity, a molybdopterin or thiamine biosynthesis activity, a beta-lactamase activity, a ligand binding activity, an ion transport activity, an ion metabolism activity, a tellurite resistance protein activity, an inorganic ion transport activity, a nucleotide transport activity, a nucleotide metabolism activity, an actin, a myosin activity, a lipase activity or a lipid acyl hydrolase (LAH) activity, a cell envelop biogenesis activity, an outer membrane synthesis activity, a ribosomal structure synthesis activity, a translational processing activity, a transcriptional initiation activity, a TATA-binding activity, a signal transduction activity, an energy metabolism activity, an ATPase activity, an information storage activity, a processing activity, or a combination thereof.

67-82. (canceled)

83. An isolated or recombinant polypeptide comprising a polypeptide as set forth in claim 61 and lacking a signal sequence.

84. An isolated or recombinant polypeptide comprising a polypeptide as set forth in claim 61 and having a heterologous signal sequence.

85-92. (canceled)

93. A protein preparation comprising a polypeptide as set forth in claim 61, wherein the protein preparation comprises a liquid, a solid or a gel.

94. A heterodimer comprising a polypeptide as set forth in claim 61 and a second domain.

95. The heterodimer of claim 94, wherein the second domain is a polypeptide and the heterodimer is a fusion protein.

96. (canceled)

97. A homodimer comprising a polypeptide as set forth in claim 61.

98. An immobilized polypeptide, wherein the polypeptide comprises a sequence as set forth in claim 61, or a subsequence thereof.

99. (canceled)

100. An array comprising an immobilized polypeptide as set forth in claim 61 or comprising an immobilized nucleic acid as set forth in claim 1.

101. (canceled)

102. An isolated or recombinant antibody that specifically binds to a polypeptide as set forth in claim 61.

103. (canceled)

104. A hybridoma comprising an antibody that specifically binds to a polypeptide as set forth in claim 61.

105. A method of isolating or identifying a polypeptide comprising the steps of:
(a) providing an antibody as set forth in claim 102;
(b) providing a sample comprising polypeptides; and
(c) contacting the sample of step (b) with the antibody of step (a) under conditions wherein the antibody can specifically bind to the polypeptide, thereby isolating or identifying the polypeptide.

106-107. (canceled)

108. A method of producing a recombinant polypeptide comprising the steps of: (a) providing a nucleic acid operably linked to a promoter, wherein the nucleic acid comprises a sequence as set forth in claim 1 or claim 24; and (b) expressing the nucleic acid of step (a) under conditions that allow expression of the polypeptide, thereby producing a recombinant polypeptide.

109. (canceled)

110. A method for identifying a polypeptide having an enzyme activity comprising the following steps:
(a) providing a polypeptide as set forth in claim 65;
(b) providing an enzyme substrate; and
(c) contacting the polypeptide with the substrate of step (b) and detecting a decrease in the amount of substrate or an increase in the amount of a reaction product, wherein a decrease in the amount of the substrate or an increase in the amount of the reaction product detects a polypeptide having an enzyme activity.

111. A method for identifying an enzyme substrate comprising the following steps:
(a) providing a polypeptide as set forth in claim 65;
(b) providing a test substrate; and
(c) contacting the polypeptide of step (a) with the test substrate of step (b) and detecting a decrease in the amount of substrate or an increase in the amount of reaction product, wherein a decrease in the amount of the substrate or an increase in the amount of a reaction product identifies the test substrate as an enzyme substrate.

112. A method of determining whether a test compound specifically binds to a polypeptide comprising the following steps:
(a) expressing a nucleic acid or a vector comprising the nucleic acid under conditions permissive for translation of the nucleic acid to a polypeptide, wherein the nucleic acid has a sequence as set forth in claim 1;
(b) providing a test compound;
(c) contacting the polypeptide with the test compound; and
(d) determining whether the test compound of step (b) specifically binds to the polypeptide.

113. A method of determining whether a test compound specifically binds to a polypeptide comprising the following steps:
(a) providing a polypeptide as set forth in claim 61;
(b) providing a test compound;
(c) contacting the polypeptide with the test compound; and
(d) determining whether the test compound of step (b) specifically binds to the polypeptide.

114. A method for identifying a modulator of an enzyme activity comprising the following steps:
(a) providing a polypeptide as set forth in claim 65;
(b) providing a test compound;
(c) contacting the polypeptide of step (a) with the test compound of step (b) and measuring an activity of the enzyme, wherein a change in the enzyme activity measured in the presence of the test compound compared to the activity in the absence of the test compound provides a determination that the test compound modulates the enzyme activity.

115-117. (canceled)

118. A computer system comprising a processor and a data storage device wherein said data storage device has stored thereon a polypeptide sequence or a nucleic acid sequence, wherein the polypeptide sequence comprises sequence as set forth in claim 61, or a polypeptide encoded by a nucleic acid as set forth in claim 1.

119-121. (canceled)

122. A computer readable medium having stored thereon a polypeptide sequence or a nucleic acid sequence, wherein the polypeptide sequence comprises a polypeptide as set forth in claim 61; or, a polypeptide encoded by a nucleic acid as set forth in claim 1.

123. A method for identifying a feature in a sequence comprising the steps of: (a) reading the sequence using a computer program which identifies one or more features in a sequence, wherein the sequence comprises a polypeptide sequence or a nucleic acid sequence, wherein the polypeptide sequence comprises a polypeptide as set forth in claim 61; a polypeptide encoded by a nucleic acid as set forth in claim 1; and (b) identifying one or more features in the sequence with the computer program.

124. A method for comparing a first sequence to a second sequence comprising the steps of: (a) reading the first sequence and the second sequence through use of a computer program which compares sequences, wherein the first sequence comprises a polypeptide sequence or a nucleic acid sequence, wherein the polypeptide sequence comprises a polypeptide as set forth in claim 61 or a polypeptide encoded by a nucleic acid as set forth in claim 1; and (b) determining differences between the first sequence and the second sequence with the computer program.

125-127. (canceled)

128. A method for isolating or recovering a nucleic acid encoding a polypeptide with an enzyme activity from an environmental sample comprising the steps of:
(a) providing an amplification primer sequence pair as set forth in claim 33;
(b) isolating a nucleic acid from the environmental sample or treating the environmental sample such that nucleic acid in the sample is accessible for hybridization to the amplification primer pair; and,
(c) combining the nucleic acid of step (b) with the amplification primer pair of step (a) and amplifying nucleic acid from the environmental sample, thereby isolating or recovering a nucleic acid encoding a polypeptide with an enzyme activity from an environmental sample.

129. (canceled)

130. A method for isolating or recovering a nucleic acid encoding a polypeptide with an enzyme activity from an environmental sample comprising the steps of:
(a) providing a polynucleotide probe comprising a sequence as set forth in claim 1, or a subsequence thereof;
(b) isolating a nucleic acid from the environmental sample or treating the environmental sample such that nucleic acid in the sample is accessible for hybridization to a polynucleotide probe of step (a);
(c) combining the isolated nucleic acid or the treated environmental sample of step (b) with the polynucleotide probe of step (a); and
(d) isolating a nucleic acid that specifically hybridizes with the polynucleotide probe of step (a), thereby isolating or recovering a nucleic acid encoding a polypeptide with an enzyme activity from an environmental sample.

131-132. (canceled)

133. A method of generating a variant of a nucleic acid encoding a polypeptide with an enzyme activity comprising the steps of:
(a) providing a template nucleic acid comprising a sequence as set forth in claim 1; and
(b) modifying, deleting or adding one or more nucleotides in the template sequence, or a combination thereof, to generate a variant of the template nucleic acid.

134-142. (canceled)

143. A method for modifying codons in a nucleic acid encoding a polypeptide with an enzyme activity to increase its expression in a host cell, the method comprising the following steps:
(a) providing a nucleic acid encoding a polypeptide with an enzyme activity comprising a sequence as set forth in claim 1; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

144. A method for modifying codons in a nucleic acid encoding an enzyme polypeptide, the method comprising the following steps:
(a) providing a nucleic acid encoding a polypeptide with an enzyme activity comprising a sequence as set forth in claim 1; and,
(b) identifying a codon in the nucleic acid of step (a) and replacing it with a different codon encoding the same amino acid as the replaced codon, thereby modifying codons in a nucleic acid encoding an enzyme.

145. A method for modifying codons in a nucleic acid encoding an enzyme polypeptide to increase its expression in a host cell, the method comprising the following steps:
(a) providing a nucleic acid encoding an enzyme polypeptide comprising a sequence as set forth in claim 1; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

146. A method for modifying a codon in a nucleic acid encoding a polypeptide having an enzyme activity to decrease its expression in a host cell, the method comprising the following steps:
(a) providing a nucleic acid encoding an enzyme polypeptide comprising a sequence as set forth in claim 1; and
(b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell.

147. (canceled)

148. A method for producing a library of nucleic acids encoding a plurality of modified enzyme active sites or substrate binding sites, wherein the modified active sites or substrate binding sites are derived from a first nucleic acid comprising a sequence encoding a first active site or a first substrate binding site the method comprising the following steps:
(a) providing a first nucleic acid encoding a first active site or first substrate binding site, wherein the first nucleic acid sequence comprises a sequence that hybridizes under stringent conditions to a sequence as set forth in SEQ ID NO:1, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ 1D NO:92, SEQ ID NO:94, SEQ ID NO:96, SEQ ID NO:98, SEQ ID NO:100, SEQ ID NO:102, SEQ ID NO:104, SEQ ID NO:106, and all nucleic acids disclosed in the SEQ ID listing, which include all even numbered SEQ ID NO:s from SEQ ID NO:2 through SEQ ID NO:1073, or a subsequence thereof, and the nucleic acid encodes an enzyme active site or an enzyme substrate binding site;
(b) providing a set of mutagenic oligonucleotides that encode naturally-occurring amino acid variants at a plurality of targeted codons in the first nucleic acid; and,
(c) using the set of mutagenic oligonucleotides to generate a set of active site-encoding or substrate binding site-encoding variant nucleic acids encoding a range of amino acid variations at each amino acid codon that was mutagenized, thereby producing a library of nucleic acids encoding a plurality of modified enzyme active sites or substrate binding sites.

149-151. (canceled)

152. A method for making a small molecule comprising the following steps:
(a) providing a plurality of biosynthetic enzymes capable of synthesizing or modifying a small molecule, wherein one of the enzymes comprises an enzyme encoded by a nucleic acid comprising a sequence as set forth in claim 1;
(b) providing a substrate for at least one of the enzymes of step (a); and
(c) reacting the substrate of step (b) with the enzymes under conditions that facilitate a plurality of biocatalytic reactions to generate a small molecule by a series of biocatalytic reactions.

153. A method for modifying a small molecule comprising the following steps:
(a) providing an enzyme, wherein the enzyme comprises a polypeptide as set forth in claim 65, or a polypeptide encoded by a nucleic acid comprising a nucleic acid sequence as set forth in claim 1;
(b) providing a small molecule; and
(c) reacting the enzyme of step (a) with the small molecule of step (b) under conditions that facilitate an enzymatic reaction catalyzed by the enzyme, thereby modifying a small molecule by an enzyme enzymatic reaction.

154-157. (canceled)

158. A method for determining a functional fragment of an enzyme comprising the steps of:
(a) providing an enzyme, wherein the enzyme comprises a polypeptide as set forth in claim 65, or a polypeptide encoded by a nucleic acid as set forth in claim 1; and
(b) deleting a plurality of amino acid residues from the sequence of step (a) and testing the remaining subsequence for an enzyme activity, thereby determining a functional fragment of an enzyme.

159. (canceled)

160. A method for whole cell engineering of new or modified phenotypes by using real-time metabolic flux analysis, the method comprising the following steps:
(a) making a modified cell by modifying the genetic composition of a cell, wherein the genetic composition is modified by addition to the cell of a nucleic acid comprising a sequence as set forth in claim 1;
(b) culturing the modified cell to generate a plurality of modified cells;
(c) measuring at least one metabolic parameter of the cell by monitoring the cell culture of step (b) in real time; and,
(d) analyzing the data of step (c) to determine if the measured parameter differs from a comparable measurement in an unmodified cell under similar conditions, thereby identifying an engineered phenotype in the cell using real-time metabolic flux analysis.

161-163. (canceled)

164. A method for whole cell engineering of new or modified phenotypes by using real-time metabolic flux analysis, the method comprising the following steps:
(a) making a modified cell by modifying the genome of a cell, wherein the genome comprises a sequence as set forth in SEQ ID NO:1;
(b) culturing the modified cell to generate a plurality of modified cells;
(c) measuring at least one metabolic parameter of the cell by monitoring the cell culture of step (b) in real time; and,
(d) analyzing the data of step (c) to determine if the measured parameter differs from a comparable measurement in an unmodified cell under similar conditions, thereby identifying an engineered phenotype in the cell using real-time metabolic flux analysis.

165-166. (canceled)

167. An isolated or recombinant signal sequence consisting of a sequence as set forth in residues 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30 or 1 to 31, 1 to 32, 1 to 33, 1 to 34, 1 to 35, 1 to 36, 1 to 37, 1 to 38, 1 to 39, 1 to 40, 1 to 41, 1 to 42, 1 to 43, 1 to 44, 1 to 45, 1 to 46, 1 to 47, 1 to 48, 1 to 49, 1 to 50, 1 to 51, 1 to 52, 1 to 53, 1 to 54, 1 to 55, of SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, and all polypeptides disclosed in the SEQ ID listing, which include all odd numbered SEQ ID NO:s from SEQ ID NO:3 through SEQ ID NO:1073.

168. A chimeric polypeptide comprising at least a first domain comprising signal peptide (SP) having a sequence as set forth in claim 164, and at least a second domain comprising a heterologous polypeptide or peptide, wherein the heterologous polypeptide or peptide is not naturally associated with the signal peptide (SP).

169-170. (canceled)

171. An isolated or recombinant nucleic acid encoding a chimeric polypeptide, wherein the chimeric polypeptide comprises at least a first domain comprising signal peptide (SP having a sequence as set forth in claim 164 and at least a second domain comprising a heterologous polypeptide or peptide, wherein the heterologous polypeptide or peptide is not naturally associated with the signal peptide (SP).

172. A method of increasing thermotolerance or thermostability of an enzyme polypeptide, the method comprising glycosylating an enzyme, wherein the polypeptide comprises at least thirty contiguous amino acids of a polypeptide as set forth in claim 61, or a polypeptide encoded by a nucleic acid as set forth in claim 1, thereby increasing the thermotolerance or thermostability of the enzyme.

173. A method for overexpressing a recombinant enzyme in a cell comprising expressing a vector comprising a nucleic acid sequence as set forth in claim 1, wherein overexpression is effected by use of a high activity promoter, a dicistronic vector or by gene amplification of the vector.

174. A method of making a transgenic plant comprising the following steps:
(a) introducing a heterologous nucleic acid sequence into the cell, wherein the heterologous nucleic sequence comprises a sequence as set forth in claim 1, thereby producing a transformed plant cell; and
(b) producing a transgenic plant from the transformed cell.

175-176. (canceled)

177. A method of expressing a heterologous nucleic acid sequence in a plant cell comprising the following steps:
(a) transforming the plant cell with a heterologous nucleic acid sequence operably linked to a promoter, wherein the heterologous nucleic sequence comprises a sequence as set forth in claim 1; and
(b) growing the plant under conditions wherein the heterologous nucleic acids sequence is expressed in the plant cell.

178. An isolated hyperthermophile Nanoarchaeum equitans deposited as ATCC accession no.

179. A method for a building a genome having a desired biological requirement, a desired biological property or a desired metabolic pathway comprising the following steps:
(a) providing a minimal autonomous genome, wherein the genome comprises a sequence as set forth in SEQ ID NO:1; and
(b) adding back to the minimal autonomous genome of step (a) one or more desired genes, thereby building a genome having a desired biological requirement, a desired biological property or a desired metabolic pathway.

180. (canceled)

181. A method for a building a minimal autonomous genome comprising the following steps:
(a) providing a genome comprising a sequence as set forth in SEQ ID NO:1; and
(b) performing global knockout mutagenesis on the genome, or adding genes to the genome, and determining whether a cell comprising the genome can survive or replicate autonomously, thereby building an autonomous minimal genome.
